SEQUENCE LISTING (21 sequences)

SEQ ID NO: 1 - protein, 766 aa, synthetic construct [sequence omitted]

SEQ ID NO: 2 - protein, 5 aa, synthetic construct
SYDMS

SEQ ID NO: 3 - protein, 16 aa, synthetic construct
LISGGGSQTYYAESVK

SEQ ID NO: 4 - protein, 11 aa, synthetic construct
PSGHYFYAMDV

SEQ ID NO: 5 - protein, 11 aa, synthetic construct
RASQGISNWLA

SEQ ID NO: 6 - protein, 7 aa, synthetic construct
AASSLQS

SEQ ID NO: 7 - protein, 9 aa, synthetic construct
QQAESFPHT

SEQ ID NO: 8 - protein, 120 aa, synthetic construct
EVQLLESGGGLVQPGGSLRLSCAASGFTFSSYDMSWVRQAPGKGLEWVSLISGGGSQTYYAESVKGRFTISRDNSKNTLYLQMNSLRAEDTAVYFCASPSGHYFYAMDVWGQGTTVTVSS

SEQ ID NO: 9 - protein, 107 aa, synthetic construct
DIQMTQSPSSVSASVGDRVTITCRASQGISNWLAWYQQKPGKAPKLLIFAASSLQSGVPSRFSGSGSGTDFTLTISSLQPEDFATYYCQQAESFPHTFGGGTKVEIK

SEQ ID NO: 10 - protein, 472 aa, synthetic construct
MDMRVPAQLLGLLLLWLRGARCEVQLLESGGGLVQPGGSLRLSCAASGFTFSSYDMSWVRQAPGKGLEWVSLISGGGSQTYYAESVKGRFTISRDNSKNTLYLQMNSLRAEDTAVYFCASPSGHYFYAMDVWGQGTTVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPCEEQYGSTYRCVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 11 - protein, 236 aa, synthetic construct
MDMRVPAQLLGLLLLWLRGARCDIQMTQSPSSVSASVGDRVTITCRASQGISNWLAWYQQKPGKAPKLLIFAASSLQSGVPSRFSGSGSGTDFTLTISSLQPEDFATYYCQQAESFPHTFGGGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 12 - DNA, 15 bp, synthetic construct
  source 1..15: mol_type = other DNA
agctatgacatgagc

SEQ ID NO: 13 - DNA, 51 bp, synthetic construct
  source 1..51: mol_type = other DNA
cttattagtggtggtggtagtcaaacatactacgcagaatccgtgaagggc

SEQ ID NO: 14 - DNA, 33 bp, synthetic construct
  source 1..33: mol_type = other DNA
cccagtggccactacttctacgctatggacgtc

SEQ ID NO: 15 - DNA, 33 bp, synthetic construct
  source 1..33: mol_type = other DNA
cgggcgagtcagggtattagcaactggttagcc

SEQ ID NO: 16 - DNA, 21 bp, synthetic construct
  source 1..21: mol_type = other DNA
gctgcatccagtttgcaaagt

SEQ ID NO: 17 - DNA, 26 bp, synthetic construct
  source 1..26: mol_type = other DNA
caacaggctgaaagtaccctcacact

SEQ ID NO: 18 - DNA, 360 bp, synthetic construct
  source 1..360: mol_type = other DNA
gaggtgcagctgttggagtctgggggaggcttggtacagcctggggggtccctgagactctcctgtgcagcctctggattcacctttagcagctatgacatgagctgggtccgccaggctccagggaaggggctggaatgggtctcacttattagtggtggtggtagtcaaacatactacgcagaatccgtgaagggccggttcaccatctccagagacaattccaagaacacgctgtatctgcaaatgaacagcctgagagccgaggacacggccgtatatttctgtgcgtcccccagtggccactacttctacgctatggacgtctggggccaagggaccacggtcaccgtctcctca

SEQ ID NO: 19 - DNA, 321 bp, synthetic construct
  source 1..321: mol_type = other DNA
gacatccagatgacccagtctccatcttccgtgtctgcatctgttggagacagagtcaccatcacttgtcgggcgagtcagggtattagcaactggttagcctggtatcagcagaaaccagggaaagcccctaagctcctgatctttgctgcatccagtttgcaaagtggggtcccatcaaggttcagcggcagtggatctgggacagatttcaccctcaccatcagcagcctgcagcctgaagattttgcaacttactattgtcaacaggctgaaagtttccctcacactttcggcggagggaccaaggtggagatcaaa

SEQ ID NO: 20 - DNA, 1416 bp, synthetic construct
  source 1..1416: mol_type = other DNA
atggacatgagggtgcccgctcagctcctggggctcctgctgctgtggctgagaggtgcgcgctgtgaggtgcagctgttggagtctgggggaggcttggtacagcctggggggtccctgagactctcctgtgcagcctctggattcacctttagcagctatgacatgagctgggtccgccaggctccagggaaggggctggaatgggtctcacttattagtggtggtggtagtcaaacatactacgcagaatccgtgaagggccggttcaccatctccagagacaattccaagaacacgctgtatctgcaaatgaacagcctgagagccgaggacacggccgtatatttctgtgcgtcccccagtggccactacttctacgctatggacgtctggggccaagggaccacggtcaccgtctcctcagcctccaccaagggcccatcggtcttccccctggcaccctcctccaagagcacctctgggggcacagcggccctgggctgcctggtcaaggactacttccccgaaccggtgacggtgtcgtggaactcaggcgccctgaccagcggcgtgcacaccttcccggctgtcctacagtcctcaggactctactccctcagcagcgtggtgaccgtgccctccagcagcttgggcacccagacctacatctgcaacgtgaatcacaagcccagcaacaccaaggtggacaagaaagttgagcccaaatcttgtgacaaaactcacacatgcccaccgtgcccagcacctgaactcctggggggaccgtcagtcttcctcttccccccaaaacccaaggacaccctcatgatctcccggacccctgaggtcacatgcgtggtggtggacgtgagccacgaagaccctgaggtcaagttcaactggtacgtggacggcgtggaggtgcataatgccaagacaaagccgtgcgaggagcagtacggcagcacgtaccgttgcgtcagcgtcctcaccgtcctgcaccaggactggctgaatggcaaggagtacaagtgcaaggtgtccaacaaagccctcccagcccccatcgagaaaaccatctccaaagccaaagggcagccccgagaaccacaggtgtacaccctgcccccatcccgggaggagatgaccaagaaccaggtcagcctgacctgcctggtcaaaggcttctatcccagcgacatcgccgtggagtgggagagcaatgggcagccggagaacaactacaagaccacgcctcccgtgctggactccgacggctccttcttcctctatagcaagctcaccgtggacaagagcaggtggcagcaggggaacgtcttctcatgctccgtgatgcatgaggctctgcacaaccactacacgcagaagagcctctccctgtctccgggtaaa

SEQ ID NO: 21 - DNA, 708 bp, synthetic construct
  source 1..708: mol_type = other DNA
atggacatgagggtgcccgctcagctcctggggctcctgctgctgtggctgagaggtgcgcgctgtgacatccagatgacccagtctccatcttccgtgtctgcatctgttggagacagagtcaccatcacttgtcgggcgagtcagggtattagcaactggttagcctggtatcagcagaaaccagggaaagcccctaagctcctgatctttgctgcatccagtttgcaaagtggggtcccatcaaggttcagcggcagtggatctgggacagatttcaccctcaccatcagcagcctgcagcctgaagattttgcaacttactattgtcaacaggctgaaagtttccctcacactttcggcggagggaccaaggtggagatcaaacgaacggtggctgcaccatctgtcttcatcttcccgccatctgatgagcagttgaaatctggaactgcctctgttgtgtgcctgctgaataacttctatcccagagaggccaaagtacagtggaaggtggataacgccctccaatcgggtaactcccaggagagtgtcacagagcaggacagcaaggacagcacctacagcctcagcagcaccctgacgctgagcaaagcagactacgagaaacacaaagtctacgcctgcgaagtcacccatcagggcctgagctcgcccgtcacaaagagcttcaacaggggagagtgt

(19) United States
(12) Patent Application Publication
(10) Pub. No.: US 2026/0355169 A1
(43) Pub. Date: Nov. 12, 2026

(54) METHODS OF TREATING CANCERS USING ISOQUINOLINE OR 6-AZA-QUINOLINE DERIVATIVES

(71) Applicant: Black Diamond Therapeutics, Inc., Cambridge, MA (US)

(72) Inventors: Yoon-Chi HAN, New York, NY (US); Tai-An LIN, Jersey City, NJ (US); Luisa SHIN OGAWA, Auburndale, MA (US); Anthony F. TROMBINO, Cambridge, MA (US); Agata JURCZYK, Millbury, MA (US); Pui Yee NG, Lexington, MA (US); Sergey YURASOV, San Francisco, CA (US); Sudharshan EATHIRAJ, Shrewsbury, MA (US); Julio José HAJDENBERG, Boston, MA (US); David M. EPSTEIN, Belmont, MA (US)

(21) Appl. No.: 19/120,169

(22) PCT Filed: Oct. 13, 2023
(86) PCT No.: PCT/US2023/076892

§ 371 Date: Apr. 10, 2025

Related U.S. Application Data
(60) Provisional application No. 63/416,305, filed on Oct. 14, 2022.
(60) Provisional application No. 63/431,997, filed on Dec. 12, 2022.
(60) Provisional application No. 63/449,757, filed on Mar. 3, 2023.
(60) Provisional application No. 63/472,037, filed on Jun. 9, 2023.

(51) Int. Cl. A61K 31/444; A61K 31/4184; A61K 31/4725; A61K 31/501; A61K 31/55; A61P 35/00; C07D 401/12; C07D 471/04; C07D 471/14; C07D 519/00

(52) U.S. Cl. CPC A61K 31/444; A61K 31/4184; A61K 31/4725; A61K 31/501; A61K 31/55; A61P 35/00; C07D 401/12; C07D 471/04; C07D 471/14; C07D 519/00

(57) ABSTRACT

The present disclosure relates to methods of treating or preventing cancer using a compound of Formula (0):

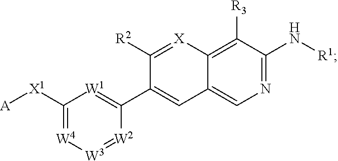

or a pharmaceutically acceptable salt thereof. The present disclosure also relates to pharmaceutical compositions and pharmaceutical kits suitable for the treatment or prevention.

### RELATED APPLICATIONS

This application claims priority to and the benefit of U.S. Provisional Application No. 63/416,305, filed on Oct. 14, 2022, 63/431,997, filed on Dec. 12, 2022, 63/449,757, filed on Mar. 3, 2023, and 63/472,037, filed on Jun. 9, 2023, which are incorporated by reference herein in their entireties.

### REFERENCE TO AN ELECTRONIC SEQUENCE LISTING

The contents of the electronic sequence listing (ASET-041_001WO_SeqList ST26.xml; Size 23,649 bytes; and Date of Creation: Oct. 12, 2023) are herein incorporated by reference in their entireties.

### BACKGROUND

Specific mutations in the human gene BRAF, which encodes for the protein B-Raf, are known to drive oncogenic activity in a variety of different cancers. Targeted inactivation of mutant B-Raf proteins by the administration of protein kinase inhibitors has been used to treat a number of different cancers in patients. However, there are subsets of patients administered these treatments that either fail to respond, eventually relapse or experience secondary lesions/pathway rebound. More specifically, many of the existing B-Raf-targeting kinase inhibitors either exhibit low specificity for B-Raf, leading to undesirable off-target effects, or only target a specific subset of BRAF/B-Raf mutation(s). Thus, there is a long-felt need in the art for new therapies that target specific oncogenic forms of B-Raf produced by mutations or alterations of the BRAF gene. The present disclosure provides compositions and methods for preventing or treating cancer in patients with oncogenic mutations in the BRAF gene and B-Raf protein.

### SUMMARY

In some aspects, the present disclosure provides a method of treating or preventing cancer in a subject, the method comprising administering to the subject a compound of the present disclosure (e.g., in a therapeutically effective amount).

In some aspects, the present disclosure provides a method of treating cancer in a subject, the method comprising administering to the subject a compound of the present disclosure (e.g., in a therapeutically effective amount).

In some aspects, the present disclosure provides a compound of the present disclosure for treating or preventing cancer in a subject.

In some aspects, the present disclosure provides a compound of the present disclosure for treating cancer in a subject.

In some aspects, the present disclosure provides a use of a compound of the present disclosure in the manufacture of a medicament for treating or preventing cancer in a subject.

In some aspects, the present disclosure provides a use of a compound of the present disclosure in the manufacture of a medicament for treating cancer in a subject.

In some aspects, the present disclosure provides a combination comprising a compound of the present disclosure and one or more additional therapeutic agents.

In some aspects, the present disclosure provides a method of treating or preventing cancer in a subject, the method comprising administering to the subject a combination comprising a compound of the present disclosure (e.g., in a therapeutically effective amount) and one or more additional therapeutic agents (e.g., in a therapeutically effective amount).

In some aspects, the present disclosure provides a method of treating cancer in a subject, the method comprising administering to the subject a combination comprising a compound of the present disclosure (e.g., in a therapeutically effective amount) and one or more additional therapeutic agents (e.g., in a therapeutically effective amount).

In some aspects, the present disclosure provides a combination comprising a compound of the present disclosure and one or more additional therapeutic agents, for treating or preventing cancer in a subject.

In some aspects, the present disclosure provides a combination comprising a compound of the present disclosure and one or more additional therapeutic agents, for treating cancer in a subject.

In some aspects, the present disclosure provides a use of a combination comprising a compound of the present disclosure and one or more additional therapeutic agents, in the manufacture of a medicament for treating or preventing cancer in a subject.

In some aspects, the present disclosure provides a compound of the present disclosure for use in combination with one or more additional therapeutic agents in treating or preventing cancer in a subject.

In some aspects, the present disclosure provides a compound of the present disclosure for use in combination with one or more additional therapeutic agents in treating cancer in a subject.

In some aspects, the present disclosure provides a use of a combination comprising a compound of the present disclosure and one or more additional therapeutic agents, in the manufacture of a medicament for treating or preventing cancer in a subject.

In some aspects, the present disclosure provides a use of a combination comprising a compound of the present disclosure and one or more additional therapeutic agents, in the manufacture of a medicament for treating cancer in a subject.

Unless otherwise defined, all technical and scientific terms used herein have the same meaning as commonly understood by one of ordinary skill in the art to which this disclosure belongs. In the specification, the singular forms also include the plural unless the context clearly dictates otherwise. Although methods and materials similar or equivalent to those described herein can be used in the practice or testing of the present disclosure, suitable methods and materials are described below. All publications, patent applications, patents and other references mentioned herein are incorporated by reference. The references cited herein are not admitted to be prior art to the claimed invention. In the case of conflict, the present specification, including definitions, will control. In addition, the materials, methods and examples are illustrative only and are not intended to be limiting. In the case of conflict between the chemical structures and names of the compounds disclosed herein, the chemical structures will control.

Other features and advantages of the disclosure will be apparent from the following detailed description and claims.

### BRIEF DESCRIPTIONS OF FIGURES

FIG. 1 is a graph depicting the schema of an exemplary study of a compound of the present disclosure.

FIG. 2 is a graph depicting the anti-tumor activity of a compound of the present disclosure (the “Compound”) in combination with binimetinib in mice with KRAS G12D NSCLC patient-derived xenograft (PDX) model.

FIG. 3 is a graph depicting the anti-tumor activity of a compound of the present disclosure (the “Compound”) in combination with binimetinib in mice with melanoma cell line with BRAF V600E mutation.

### DETAILED DESCRIPTION

The present disclosure relates to methods of using compounds, and pharmaceutically acceptable salts and stereoisomers thereof, in the treatment or prevention of cancers associated with B-Raf oncogenic activity, and compositions and kits suitable for the methods.

BRAF is a human gene located on the long arm of chromosome 7 (7q34) that encodes for a protein known as B-Raf. B-Raf is a serine/threonine kinase that resides in the cytoplasm of cells. B-Raf is an effector molecule within the mitogen-activated protein kinase (MAPK)/extracellular signal-regulated kinase (ERK) signaling pathway, a pathway that is known to regulate various cellular processes including, but not limited to, growth, proliferation, differentiation and apoptosis.

Briefly, and as would be appreciated by the skilled artisan, in the MAPK/ERK signaling pathway, certain external stimuli, such as growth factors, activate receptors located on the cellular membrane, including receptor tyrosine kinases (RTKs). These receptors in turn activate RAS, causing an exchange of GDP to GTP, thereby producing RAS-GTP. RAS-GTP then activates a Mitogen Activated Protein kinase kinase kinase (MAPKKK or MAP3K). The activated MAPKKK then activates a MAP kinase kinase (MAPKK). The activated MAPKK then activates a MAP kinase (MAPK). Activated MAPK then activates downstream effectors, including transcriptions factors, causing changes in gene expression, thereby regulating the various cellular processes described above, including, but not limited to, cellular growth, proliferation, differentiation and apoptosis.

Examples of MAPKKKs include members of the rapidly accelerated fibrosarcoma (Raf) family, including Raf-1 (also known as C-Raf), B-Raf and A-Raf.

Raf proteins, including B-raf, have three conserved domains denoted conserved region 1 (CR1), conserved region 2 (CR2) and conserved region 3 (CR3). CR1 is an autoinhibitory domain that inhibits the Raf protein's kinase domain (CR3). CR1 includes a binding site for RAS-GTP's effector domain. Upon CR1 binding to RAS-GTP's effector domain, CR1 releases the CR3, relieving autoinhibition of the kinase domain. CR2 is flexible linker that acts as a hinge to connected CR1 and CR3. CR3 is an enzymatic kinase domain.

In its active form, B-Raf forms a dimer and functions as a serine/threonine-specific protein kinase. Under activating conditions, the regulatory protein 14-3-3 is displaced from CR2, of B-Raf, allowing for a de-clamping of CR1 and CR2. Additionally, RAS-GTP binds to CR1 of B-Raf, causing CR1 to release CR3. The overall effect is that the autoinhibition of the kinase domain of B-Raf is relieved. Subsequently, B-Raf is phosphorylated at T599 and S602, which results in the kinase domain switching to the active confirmation. Dimerization can then occur, which further stabilizes the active form of B-Raf.

Mutations in the BRAF gene have been implicated in a variety of different cancers, including, but not limited to, melanoma, non-Hodgkin's lymphoma, colorectal cancer, papillary thyroid carcinoma, non small cell lung cancer (NSCLC) and glioblastoma. As of 2019, approximately 200 BRAF-mutant alleles have been identified in human tumors, with at least 30 distinct mutations having been functionally characterized. BRAF mutations are typically categorized into one of three classes based on the mutations effect on B-Raf activity.

Class I (or Class 1) mutations are mutations that result in the expression of mutant B-Raf that can become active in the monomeric form, independent of RAS activity. That is, Class I mutations in BRAF yield the expression of B-Raf proteins that are RAS-independent, active monomers. These RAS-independent, active monomers typically demonstrate elevated levels of kinase activity.

Class II (or Class 2) mutations are mutations that result in the expression of mutant B-Raf that can form active dimers independent of RAS. That is, Class II mutations in BRAF yield the expression of B-Raf proteins that are RAS-independent, active dimers. These RAS-independent, active dimers also display intermediate to high levels of kinase activity, but their activity levels are typically lower compared to the RAS-independent, active monomers produced by Class I BRAF mutations.

Class III (or Class 3) mutations are mutations that result in the expression of mutant B-Raf that are RAS dependent (i.e. must be activated by RAS-GTP) and that can form heterodimers with other MAPK proteins such as C-Raf. Class III mutations in BRAF typically yield B-Raf with low or impaired kinase activity.

As would be appreciated by the skilled artisan, since Class I BRAF mutations and Class II BRAF mutations are RAS-independent, the mutant B-Raf proteins harboring Class I or Class II mutations are uncoupled to any upstream signals, resulting in constitutive activation that can result in unchecked cellular growth and eventually oncogenic proliferation.

### Methods and Uses of the Present Disclosure

In some aspects, the present disclosure provides a method of treating or preventing cancer in a subject, the method comprising administering to the subject a compound of the present disclosure (e.g., in a therapeutically effective amount).

In some aspects, the present disclosure provides a method of treating cancer in a subject, the method comprising administering to the subject a compound of the present disclosure (e.g., in a therapeutically effective amount).

In some aspects, the present disclosure provides a compound of the present disclosure for treating or preventing cancer in a subject.

In some aspects, the present disclosure provides a compound of the present disclosure for treating cancer in a subject.

In some aspects, the present disclosure provides a use of a compound of the present disclosure in the manufacture of a medicament for treating or preventing cancer in a subject.

In some aspects, the present disclosure provides a use of a compound of the present disclosure in the manufacture of a medicament for treating cancer in a subject.

In some embodiments, one or more inhibitors of one or more component of the MAPK pathways are further administered to the subject.

### Suitable Subjects and Diseases

In some embodiments, the subject is a mammal.

In some embodiments, the subject is a human.

In some embodiments, the subject is a mouse.

In some embodiments, the subject is a rat.

In some embodiments, the subject is a dog.

In some embodiments, the subject is a monkey.

In some embodiments, the cancer is characterized by at least one oncogenic mutation in the BRAF gene.

It is understood that a cancer that is characterized by at least one oncogenic mutation in the BRAF gene is a cancer that is typically associated with at least one oncogenic mutation in the BRAF gene, including, but not limited to, cancers whose primary oncogenic activity is thought to be driven by the at least one oncogenic mutation in the BRAF gene.

In some embodiments, the cancer is characterized by at least one oncogenic variant of B-Raf.

It is understood that a cancer that is characterized by least one oncogenic variant of B-Raf is a cancer that is typically associated with at least one oncogenic variant of B-Raf, including, but not limited to, cancers whose primary oncogenic activity is thought to be driven by the at least one oncogenic variant of B-Raf.

It is understood that an oncogenic variant of B-Raf is a B-Raf protein that comprises at least one oncogenic mutation and that is produced as the result of the expression of a BRAF gene that comprises at least one oncogenic mutation.

In some embodiments, the subject has at least one oncogenic mutation in the BRAF gene.

In some embodiments, the subject has at least one tumor and/or cancerous cell that expresses an oncogenic variant of B-Raf.

As would be appreciated by the skilled artisan, in the context of a gene (e.g. BRAF), an oncogenic mutation can include, but is not limited to a mutation that results in the substitution of one amino acid for another at a specific position within B-Raf, a mutation that results in the substitution of one or more amino acids for one or more amino acids between two specific positions within B-Raf, a mutation that results in an insertion of one or more amino acids between two positions within B-Raf, a mutation that results in the deletion of one more amino acids between two positions within B-Raf, and mutation that results in a fusion of B-Raf, or portion thereof, with another protein, or portion thereof, or any combination thereof. As would be appreciated by the skilled artisan, in the context of a gene, an oncogenic mutation can include, but is not limited to, a missense mutation, a nonsynonymous mutation, an insertion of one or more nucleotides, a deletion of one or more nucleotides, an inversion and a deletion-insertion. As would be appreciated by the skilled artisan, in the context of a gene (e.g. BRAF), the gene can have one or more of the aforementioned types of oncogenic mutations, including combinations of different types of oncogenic mutations.

As would be appreciated by the skilled artisan, in the context of a protein (e.g. B-Raf), an oncogenic mutation can include, but is not limited to, the substitution of one amino acid for another at a specific position within B-Raf, the substitution of one or more amino acids for one or more amino acids between two specific positions within B-Raf, an insertion of one or more amino acids between two positions within B-Raf, a deletion of one more amino acids between two positions within B-Raf, and a fusion of B-Raf, or portion thereof, with another protein, or portion thereof, or any combination thereof. As would be appreciated by the skilled artisan, in the context of a protein (e.g. B-Raf), the protein can have one or more of the aforementioned types of oncogenic mutations, including combinations of different types of oncogenic mutations.

In some embodiments, an oncogenic mutation of B-Raf can be any of the B-Raf mutations put forth in Table 1a. An oncogenic variant of B-Raf can comprise one or more than one of the oncogenic mutations put forth in Table 1a in any combination. In a non-limiting example, an oncogenic variant of B-Raf can comprise the oncogenic mutations K601E and S363F.

TABLE 1a
B-Raf mutations (numbering corresponding to SEQ ID NO: 1)

| S363F | G464V | N581S | V600K |
|---|---|---|---|
| F247L | G466E | D594G | V600R |
| | G466V | D594N | K601E |
| G464R | S467L | L597Q | K601N |
| L597R | G469A | L597V | L485-P490 > F |
| G469R | G469V | V600E | L485-P490 Deletion |
| G466A | L584F | K601T | L485-P490 > Y |
| | | | (L485-P490Y) |
| KIAA Fusion | SND-1 | SKAP2 | 487-492 Deletion |
| (KIAA1549 fusion) | Fusion | Fusion | |
| CUL-1 Fusion | E275K | Ex10-18dup | V600E + ex10- |
| 487-493 Deletion | Deletion | | 18dup |
| | around 487- | | |
| | 493 | | |

As would be appreciated by the skilled Artisan, L485-P490>Y and L485-P490Y refers to the substitution residues L485 through P490 of B-Raf (SEQ ID NO: 1) with a Tyrosine (Y) residue.

In some embodiments, an oncogenic mutation of B-Raf can comprise a deletion of any combination of one or more amino acids between L485 and P490 of B-Raf (SEQ ID NO: 1). In some embodiments, an oncogenic mutation of B-Raf can comprise a deletion of any combination of one or more amino acids between L485 and Q494 of B-Raf (SEQ ID NO: 1). In some embodiments, an oncogenic mutation of B-Raf can comprise a deletion of any combination of one or more amino acids between A481 and Q494 of B-Raf (SEQ ID NO: 1). In some embodiments, an oncogenic mutation of B-Raf can comprise a deletion of any combination of one or more amino acids between K475 and N500 of B-Raf (SEQ ID NO: 1). In some embodiments, any of the preceding deletions can further comprise any combination of one or more substitutions and/or insertions within the range of residues indicated.

In some embodiments, the oncogenic mutation “Deletion around 487-493” refers to a deletion of one or more amino acids between residue 487±3 and residue 498±3 (e.g. a deletion between residues 489 and 497).

A wild type B-Raf sequence of the present disclosure may comprise, consist essentially of, or consist of the amino acid sequence of:

| (SEQ ID NO: 1) | |
|---|---|
| 1 | MAALSGGGGG GAEPGQALFN GDMEPEAGAG AGAAASSAAD PAIPEEVWNI |
| 51 | KQMIKLTQEH IEALLDKEGG EHNPPSIYLE AYEEYTSKLD ALQQREQQLL |
| 101 | ESLGNGTDES VSSSASMDTV TSSSSSSLSV LPSSLSVFQN PTDVARSNPK |
| 151 | SPQKPIVRVF LPNKQRTVVP ARCGVTVRDS LKKALMMRGL IPECCAVYRI |
| 201 | QDGEKKPIGW DTDISWLIGE ELHVEVLENV PLTTHNFVRK TFFTLAFCDE |
| 251 | CRKLLFQGFR CQTCGYKFHQ RCSTEVPLMC VNYDQLDLLF VSKFFEHHPI |
| 301 | PQEEASLAET ALTSGSSPSA PASDSIGPQI LTSPSPSKSI PIPQPFRPAD |
| 351 | EDHRNQFGQR DRSSSAPNVH INTIEPVNID DLIRDQGERG DGGSTTGLSA |
| 401 | TPPASLPGSL TNVKALQKSP GPQRERKSSS SSEDRNRMKT LGRRDSSDDW |
| 451 | EIPDGQITVG QRIGSGSFGT VYKGKWHGDV AVKMLNVTAP TPQQLQAFKN |
| 501 | EVGVLRKTRH VNILLEMGYS TKPQLAIVTQ WCEGSSLYHH LHIIETKFEM |
| 551 | IKLIDIARQT AQGMDYLHAK SIIHRDLKSN NIFLHEDLTV KIGDFGLATV |
| 601 | KSRWSGSHQF EQLSGSILWM APEVIRMQDK NPYSFQSDVY AFGIVLYELM |
| 651 | TGQLPYSNIN NRDQIIFMVG RGYLSPDLSK VRSNCPKAMK RLMAECLKKK |
| 701 | RDERPLFPQI LASIELLARS LPKIHRSASE PSLNRAGFQT EDESLYACAS |
| 751 | PKTPIQAGGY GAFPVH |

In some embodiments, the oncogenic mutation is a class I mutation. Accordingly, in some embodiments, the oncogenic variant of B-Raf comprises a class I mutation.

In some embodiments, the oncogenic mutation is a class II mutation. Accordingly, in some embodiments, the oncogenic variant of B-Raf comprises a class II mutation.

In some embodiments, the oncogenic mutation is a class III mutation. Accordingly, in some embodiments, the oncogenic variant of B-Raf comprises a class III mutation.

In some embodiments, the oncogenic variant of B-Raf can be any of the B-Raf variants put forth in Table 1b. Any of the variants put forth in Table 1b can be combined with any of the other variants put forth in Table 1b. Thus, in a non-limiting example an oncogenic variant of B-Raf can be B-Raf-K601E+S363F.

TABLE 1b
B-Raf oncogenic variants (numbering
corresponding to SEQ ID NO: 1)

| B-Raf-K601E- | B-Raf-G464V | B-Raf-N581S | B-Raf-V600K |
|---|---|---|---|
| S363F | | | |
| B-Raf-F247L | B-Raf-G466E | B-Raf-D594G | B-Raf-V600R |
| | B-Raf-G466V | B-Raf-D594N | B-Raf-K601E |
| B-Raf-G464R | B-Raf-S467L | B-Raf-L597Q | B-Raf-K601N |
| B-Raf-L597R | B-Raf-G469A | B-Raf-L597V | B-Raf-L485- |
| | | | P490 > F |
| | | | (B-Raf-L485- |
| | | | P490F) |
| B-Raf-G469R | B-Raf-G469V | B-Raf-V600E | B-RAF-L485- |
| | | | P490 Deletion |
| B-Raf-G466A | B-Raf-L584F | B-Raf-K601T | B-Raf-L485- |
| | | | P490 > Y |
| | | | (B-Raf-L485- |
| | | | P490Y) |
| B-Raf-KIAA | B-Raf-SND-1 | B-Raf-SKAP2 | B-Raf-487- |
| Fusion | Fusion | Fusion | 492 Deletion |
| (B-Raf- | | | |
| KIAA1549 | | | |
| fusion) | | | |
| B-Raf-CUL-1 | B-Raf-E275K | B-Raf-ex10- | B-Raf- |
| Fusion | | 18dup | V600E + ex10- |
| | | | 18dup |
| B-Raf-487-492 | B-Raf-deletion | | |
| Deletion | around 487- | | |
| | 493 | | |

In some embodiments, an oncogenic variant of B-Raf can comprise a deletion of any combination of one or more amino acids between L485 and P490 of B-Raf (SEQ ID NO: 1). In some embodiments, an oncogenic variant of B-Raf can comprise a deletion of any combination of one or more amino acids between L485 and Q494 of B-Raf (SEQ ID NO: 1). In some embodiments, an oncogenic variant of B-Raf can comprise a deletion of any combination of one or more amino acids between A481 and Q494 of B-Raf (SEQ ID NO: 1). In some embodiments, an oncogenic variant of B-Raf can comprise a deletion of any combination of one or more amino acids between K475 and N500 of B-Raf (SEQ ID NO: 1). In some embodiments, any of the preceding deletions can further comprise any combination of one or more substitutions and/or insertions within the range of residues indicated.

As would be appreciated by the skilled artisan, B-Raf-ex10-18dup refers to an oncogenic variant of B-Raf that comprises a duplication of exons 10 through 18 (see e.g. Kemper et al. Cell Rep. 2016 Jun. 28; 16(1): 263-277, incorporated by reference herein for all purposes). As would be appreciated by the skilled artisan, the oncogenic B-Raf-ex10-18dup can emerge after treatment with a BRAF inhibitor and/or MEK inhibitor. Without wishing to be bound by theory, the B-Raf-ex10-18dup can mediate resistance to the BRAF inhibitor and/or MEK inhibitor.

In some aspects, an oncogenic variant of B-Raf can comprise both a duplication of exons 10 through 18 and the V600E mutation (B-Raf-V600E+ex10-18dup).

In some embodiments, a subject has at least one tumor and/or cancerous cell that expresses an oncogenic variant of B-Raf and at least one additional protein in the RAF and/or MAPK/ERK signaling pathways that comprises at least one mutation. In some embodiments, the at least one additional protein can be selected from N-Ras, K-Ras, Neurofibromin 1 (NF1). In some embodiments, the at least one mutation in the at least one additional protein can be an oncogenic mutation.

In some embodiments, a subject has at least one tumor and/or cancerous cell that expresses an oncogenic variant of B-Raf and an N-Ras protein comprising at least one mutation. In some as embodiments, an N-Ras protein comprising at least one mutation can be N-Ras-G12D, N-Ras-Q61K, and/or N-Ras-Q61R. In some embodiment, an N-Ras protein comprising at least one mutation can be N-Ras-Q61L and/or N-Ras-G13D. In a non-limiting example, a subject can have at least one tumor and/or cancerous cell that expresses B-Raf-D594G and N-Ras-G12D.

In some embodiments, a subject has at least one tumor and/or cancerous cell that expresses an oncogenic variant of B-Raf and a K-Ras protein comprising at least one mutation. In some embodiments, a K-Ras protein comprising at least one mutation can be K-Ras-G12V, K-Ras-G12D, K-Ras-G12A, K-Ras-G12S, K-Ras-G12C, K-Ras-Q61H, K-Ras-Q61L, K-Ras-G13C and/or K-Ras-G13D.

In some embodiments, a subject has at least one tumor and/or cancerous cell that expresses an oncogenic variant of B-Raf and an NF1 protein comprising at least one mutation. Exemplary mutations within NF1 proteins include, but are not limited to missense mutations, nonsense mutations, frameshift mutations, and splice site mutations, insertions, deletions, and translocations.

In some embodiments, the cancer is characterized by at least one oncogenic mutation in at least one protein in the RAF and/or MAPK/ERK signaling pathways. In some embodiments, the at least one protein in the RAF and/or MAPK/ERK signaling pathways can be selected from N-Ras, K-Ras, and NF1.

In some embodiments, the cancer is characterized by at least one oncogenic mutation in the KRAS gene.

It is understood that a cancer that is characterized by at least one oncogenic mutation in the KRAS gene is a cancer that is typically associated with at least one oncogenic mutation in the KRAS gene, including, but not limited to, cancers whose primary oncogenic activity is thought to be driven by the at least one oncogenic mutation in the KRAS gene.

In some embodiments, an oncogenic mutation in the KRAS gene induces the formation of one or more homodimers and/or heterodimers of Raf proteins. In a non-limiting example, an oncogenic mutation in the KRAS gene can induce the formation of A-Raf homodimers. In a non-limiting example, an oncogenic mutation in the KRAS gene can induce the formation of B-Raf homodimers. In a non-limiting example, an oncogenic mutation in the KRAS gene can induce the formation of C-Raf homodimers. In a non-limiting example, an oncogenic mutation in the KRAS gene can induce the formation of B-Raf/C-Raf heterodimers, A-Raf/B-Raf heterodimers, and/or A-Raf/C-Raf heterodimers.

In some embodiments, the cancer is characterized by at least one oncogenic variant of K-Ras.

In some embodiment, an oncogenic variant of K-Ras induces the formation of one or more homodimers and/or heterodimers of Raf proteins. In a non-limiting example, the at least one oncogenic variant of K-Ras can induce the formation of A-Raf homodimers. In a non-limiting example, the at least one oncogenic variant of K-Ras can induce the formation of B-Raf homodimers. In a non-limiting example, the at least one oncogenic variant of K-Ras can induce the formation of C-Raf homodimers. In a non-limiting example, the at least one oncogenic variant of K-Ras can induce the formation of B-Raf/C-Raf heterodimers, A-Raf/B-Raf heterodimers, and/or A-Raf/C-Raf heterodimers.

It is understood that a cancer that is characterized by least one oncogenic variant of K-Ras is a cancer that is typically associated with at least one oncogenic variant of K-Ras, including, but not limited to, cancers whose primary oncogenic activity is thought to be driven by the at least one oncogenic variant of K-Ras.

It is understood that an oncogenic variant of K-Ras is a K-Ras protein that comprises at least one oncogenic mutation and that is produced as the result of the expression of a KRAS gene that comprises at least one oncogenic mutation.

In some embodiments, the subject has at least one oncogenic mutation in the KRAS gene.

In some embodiments, the subject has at least one tumor and/or cancerous cell that expresses an oncogenic variant of K-Ras.

In some embodiments, an oncogenic mutation of K-Ras can be selected from K-Ras-G12V, K-Ras-G12D, K-Ras-G12A, K-Ras-G12S, K-Ras-G12C, K-Ras-Q61H, K-Ras-Q61L, K-Ras-G13C and K-Ras-G13D. In some embodiments, an oncogenic mutation of K-Ras can be a mutation that induces constitutive RAF dimer activation. In some embodiments, an oncogenic mutation of K-Ras is a mutation that is not K-Ras-G12C. In some embodiments, an oncogenic mutation of K-Ras can be K-Ras-G12D. In some embodiments, an oncogenic mutation of K-ras can be K-Ras-G12V.

In some embodiments, the subject has at least one oncogenic mutation in the KRAS gene, wherein the at least one oncogenic mutation in the KRAS gene induces the formation of one or more homodimers and/or heterodimers of Raf proteins. In a non-limiting example, the at least one oncogenic mutation in the KRAS gene can induce the formation of A-Raf homodimers. In a non-limiting example, the at least one oncogenic mutation in the KRAS gene can induce the formation of B-Raf homodimers. In a non-limiting example, the at least one oncogenic mutation in the KRAS gene can induce the formation of C-Raf homodimers. In a non-limiting example, the at least one oncogenic mutation in the KRAS gene can induce the formation of B-Raf/C-Raf heterodimers, A-Raf/B-Raf heterodimers, and/or A-Raf/C-Raf heterodimers.

In some embodiments, the subject has at least one tumor and/or cancerous cell that expresses an oncogenic variant of K-Ras, wherein oncogenic variant of K-Ras induces the formation of one or more homodimers and/or heterodimers of Raf proteins. In a non-limiting example, the oncogenic variant of K-Ras can induce the formation of A-Raf homodimers. In a non-limiting example, the oncogenic variant of K-Ras can induce the formation of B-Raf homodimers. In a non-limiting example, the oncogenic variant of K-Ras can induce the formation of C-Raf homodimers. In a non-limiting example, the a oncogenic variant of K-Ras can induce the formation of B-Raf/C-Raf heterodimers, A-Raf/B-Raf heterodimers, and/or A-Raf/C-Raf heterodimers.

In some embodiments, the cancer is characterized by at least one oncogenic mutation in the NRAS gene.

It is understood that a cancer that is characterized by at least one oncogenic mutation in the NRAS gene is a cancer that is typically associated with at least one oncogenic mutation in the NRAS gene, including, but not limited to, cancers whose primary oncogenic activity is thought to be driven by the at least one oncogenic mutation in the NRAS gene.

In some embodiments, the cancer is characterized by at least one oncogenic variant of N-Ras.

It is understood that a cancer that is characterized by least one oncogenic variant of N-Ras is a cancer that is typically associated with at least one oncogenic variant of N-Ras, including, but not limited to, cancers whose primary oncogenic activity is thought to be driven by the at least one oncogenic variant of N-Ras.

It is understood that an oncogenic variant of N-Ras is a N-Ras protein that comprises at least one oncogenic mutation and that is produced as the result of the expression of a NRAS gene that comprises at least one oncogenic mutation.

In some embodiments, the subject has at least one oncogenic mutation in the NRAS gene.

In some embodiments, the subject has at least one tumor and/or cancerous cell that expresses an oncogenic variant of N-Ras.

In some embodiments, an oncogenic mutation of N-Ras can be selected from N-Ras-G12D, N-Ras-Q61K, N-Ras-Q61R, N-Ras-Q61L and N-Ras-G13D. In some embodiments, an oncogenic mutation of N-Ras can be a mutation that induces constitutive RAF dimer activation.

In some embodiments, the cancer is characterized by at least one oncogenic mutation in the NF1 gene. In some embodiments, an oncogenic mutation in the NF1 gene can be a loss-of-function mutation.

It is understood that a cancer that is characterized by at least one oncogenic mutation in the NF1 gene is a cancer that is typically associated with at least one oncogenic mutation in the NF1 gene, including, but not limited to, cancers whose primary oncogenic activity is thought to be driven by the at least one oncogenic mutation in the NF1 gene.

In some embodiments, the cancer is characterized by at least one oncogenic variant of NF1.

It is understood that a cancer that is characterized by least one oncogenic variant of NF1 is a cancer that is typically associated with at least one oncogenic variant of NF1, including, but not limited to, cancers whose primary oncogenic activity is thought to be driven by the at least one oncogenic variant of N-Ras.

It is understood that an oncogenic variant of NF1 is a NF1 protein that comprises at least one oncogenic mutation and that is produced as the result of the expression of a NF1 gene that comprises at least one oncogenic mutation. In some aspects, an oncogenic mutation in an NF1 protein can be a loss-of-function mutation in the NF1 protein.

In some embodiments, the subject has at least one oncogenic mutation in the NF1 gene.

In some embodiments, the subject has at least one tumor and/or cancerous cell that expresses an oncogenic variant of NF1.

In some embodiments, the cancer is characterized by any combination of: at least one oncogenic mutation in the NRAS gene, at least one oncogenic mutation in the KRAS gene, and at least one oncogenic mutation in the NF1 gene. The oncogenic mutations in can be any of the oncogenic mutations described herein.

Accordingly, in some embodiments, the cancer is characterized by any combination of at least one oncogenic variant of K-Ras, at least one oncogenic variant of N-Ras, and at least one oncogenic variant of NF1.

In some embodiments, the cancer is characterized by at least one oncogenic mutation in a gene encoding a RAS GTPase.

It is understood that a cancer that is characterized by at least one oncogenic mutation in a gene encoding a RAS GTPase is a cancer that is typically associated with at least one oncogenic mutation in a gene encoding a RAS GTPase, including, but not limited to, cancers whose primary oncogenic activity is thought to be driven by the at least one oncogenic mutation the gene encoding a RAS GTPase.

In some embodiments, the cancer is characterized by at least one oncogenic variant of a RAS GTPase.

It is understood that a cancer that is characterized by least one oncogenic variant of a RAS GTPase is a cancer that is typically associated with at least one oncogenic variant of a RAS GTPase, including, but not limited to, cancers whose primary oncogenic activity is thought to be driven by the at least one oncogenic variant of a RAS GTPase.

It is understood that an oncogenic variant of a RAS GTPase is a RAS GTPase protein that comprises at least one oncogenic mutation and that is produced as the result of the expression of a gene encoding a RAS GTPase that comprises at least one oncogenic mutation.

In some embodiments, the subject has at least one oncogenic mutation in a gene encoding a RAS GTPase.

In some embodiments, the subject has at least one tumor and/or cancerous cell that expresses an oncogenic variant of a RAS GTPase.

In some embodiments, a RAS GTPase can be NRAS, KRAS or HRAS.

In some embodiments, the cancer is characterized by at least one oncogenic mutation in the HRAS gene.

It is understood that a cancer that is characterized by at least one oncogenic mutation in the HRAS gene is a cancer that is typically associated with at least one oncogenic mutation in the HRAS gene, including, but not limited to, cancers whose primary oncogenic activity is thought to be driven by the at least one oncogenic mutation in the HRAS gene.

In some embodiments, the cancer is characterized by at least one oncogenic variant of H-Ras.

It is understood that a cancer that is characterized by least one oncogenic variant of H-Ras is a cancer that is typically associated with at least one oncogenic variant of H-Ras, including, but not limited to, cancers whose primary oncogenic activity is thought to be driven by the at least one oncogenic variant of H-Ras.

It is understood that an oncogenic variant of H-Ras is a H-Ras protein that comprises at least one oncogenic mutation and that is produced as the result of the expression of a HRAS gene that comprises at least one oncogenic mutation.

In some embodiments, the subject has at least one oncogenic mutation in the HRAS gene.

In some embodiments, the subject has at least one tumor and/or cancerous cell that expresses an oncogenic variant of H-Ras.

In some embodiments, an oncogenic mutation of H-Ras can be H-Ras-G13D.

In some embodiments, the cancer is a carcinoma, a lymphoma, a blastoma, a sarcoma, a leukemia, a brain cancer, a breast cancer, a blood cancer, a bone cancer, a lung cancer, a skin cancer, a liver cancer, an ovarian cancer, a bladder cancer, a renal cancer, a kidney cancer, a gastric cancer, a thyroid cancer, a pancreatic cancer, an esophageal cancer, a prostate cancer, a cervical cancer, a uterine cancer, a stomach cancer, a soft tissue cancer, a laryngeal cancer, a small intestine cancer, a testicular cancer, an anal cancer, a vulvar cancer, a joint cancer, an oral cancer, a pharynx cancer or a colorectal cancer.

In some embodiments, the cancer is adrenocortical carcinoma, bladder urothelial carcinoma, breast invasive carcinoma, cervical squamous cell carcinoma, endocervical adenocarcinoma, cholangiocarcinoma, colon adenocarcinoma, lymphoid neoplasm diffuse large B-cell lymphoma, esophageal carcinoma, glioblastoma multiforme, head and neck squamous cell carcinoma, kidney chrormophobe, kidney renal clear cell carcinoma, kidney renal papillary cell carcinoma, acute myeloid leukemia, brain lower grade glioma, liver hepatocellular carcinoma, lung adenocarcinoma, lung squamous cell carcinoma, mesothelioma, ovarian serous cystadenocarcinoma, pancreatic adenocarcinoma, pheochromocytorna, paraganglioma, prostate adenocarcinoma, rectum adenocarcinoma, sarcoma, skin cutaneous melanoma, stomach adenocarcinoma, testicular germ cell tumors, thyroid carcinoma, thymoma, uterine carcinosarcoma, uveal melanoma. Other examples include breast cancer, lung cancer, lymphoma, melanoma, liver cancer, colorectal cancer, ovarian cancer, bladder cancer, renal cancer or gastric cancer. Further examples of cancer include neuroendocrine cancer, non-small cell lung cancer (NSCLC), small cell lung cancer, thyroid cancer, endometrial cancer, biliary cancer, esophageal cancer, anal cancer, salivary, cancer, vulvar cancer, cervical cancer, Acute lymphoblastic leukemia (ALL), Acute myeloid leukemia (AML), Adrenal gland tumors, Anal cancer, Bile duct cancer, Bladder cancer, Bone cancer, Bowel cancer, Brain tumors, Breast cancer, cancer of unknown primary (CUP), cancer spread to bone, cancer spread to brain, cancer spread to liver, cancer spread to lung, carcinoid, cervical cancer, children's cancers, chronic lymphocytic leukemia (CLL), chrome myeloid leukemia (CML), colorectal cancer, ear cancer, endometrial cancer, eye cancer, Follicular dendritic cell sarcoma, gallbladder cancer, gastric cancer, gastro esophageal junction cancers, germ cell tumors, gestational trophoblastic disease (GIT)), hairy cell leukemia, head and neck cancer, Hodgkin lymphoma, Kaposi's sarcoma, kidney cancer, laryngeal cancer, leukemia, gastric linitis plastica, liver cancer, lung cancer, lymphoma, malignant schrwannoma, mediastinal germ cell tumors, melanoma skin cancer, men's cancer, merkel cell skin cancer, rnesothelioma, molar pregnancy, mouth and oropharyngeal cancer, myeloma, nasal and paranasal sinus cancer, nasopharyngeal cancer, neuroblastoma, neuroendocrine tumors, non-Hodgkin lymphoma (NIL), esophageal cancer, ovarian cancer, pancreatic cancer, penile cancer, persistent trophoblastic disease and choriocarcinoma, pheochromocytoma, prostate cancer, pseudomyxoma peritonei, rectal cancer, retinoblastoma, salivary gland cancer, secondary' cancer, signet cell cancer, skin cancer, small bowel cancer, soft tissue sarcoma, stomach cancer, T cell childhood non Hodgkin lymphoma (NHL), testicular cancer, thymus gland cancer, thyroid cancer, tongue cancer, tonsil cancer, tumors of the adrenal gland, uterine cancer. vaginal cancer, vulval cancer, Wilms' tumor, womb cancer and gynaecological cancer.

Examples of cancer also include, but are not limited to, hematologic malignancies, lymphoma, cutaneous T-cell lymphoma, peripheral T-cell lymphoma, Hodgkin's lymphoma, non-Hodgkin's lymphoma, multiple myeloma, chrome lymphocytic leukemia, chronic myeloid leukemia, acute myeloid leukemia, myelodysplastic syndromes, myelofibrosis, biliary tract cancer, hepatocellular cancer, colorectal cancer, breast cancer, lung cancer, non-small cell lung cancer, ovarian cancer, thyroid carcinoma, renal cell carcinoma, pancreatic cancer, bladder cancer, skin cancer, malignant melanoma, merkel cell carcinoma, uveal melanoma or glioblastoma multiforme.

In some embodiments, the cancer is a hematological cancer.

In some embodiments, the cancer is a solid cancer (also referred to as a solid malignancy or a solid tumor).

In some embodiments, the cancer is melanoma, breast cancer, head and neck cancer, esophagogastric cancer, stomach and small intestine cancer, lung cancer, mesothelioma, hepatobiliary cancer, pancreatic cancer, kidney cancer, colorectal cancer, endometrial cancer, cervical cancer, ovarian cancer, bladder cancer, prostate cancer, soft tissue sarcoma, CNS and brain cancer, or thyroid cancer.

In some embodiments, the cancer is non-small cell lung cancer (NSCLC), colorectal cancer, melanoma, thyroid cancer, histiocytosis, small bowel cancer, gastrointestinal neuroendocrine cancer, carcinoma of unknown primary, non-melanoma skin cancer, prostate cancer, gastric cancer, non-Hodgkin's lymphoma, papillary thyroid carcinoma or glioblastoma.

In some embodiments, the cancer is NSCLC. In some embodiments, the NSCLC has not undergone small cell lung cancer transformation. In some embodiments, the cancer is lung non-small cell carcinoma.

In some embodiments, the cancer is a histiocytic neoplasm. In some embodiments, the histiocytic neoplasm is Langerhans cell histiocytosis (LCH). In some embodiments, the histiocytic neoplasm is Erdheim Chester disease (ECD). In some embodiments, the cancer is a histiocytic and dendritic cell neoplasm.

In some embodiments, the cancer is melanoma.

In some embodiments, the cancer is thyroid cancer.

In some embodiments, the cancer is thyroid carcinoma.

In some embodiments, the cancer is colorectal cancer.

In some embodiments, the cancer is colorectal carcinoma.

In some embodiments, the cancer is glioma.

In some embodiments, the cancer is astrocytoma, brain stem glioma, ependymoma, oligo-astrocytoma, oligodendroglioma, or optic pathway glioma.

In some embodiments, the cancer is low-grade glioma (e.g., glioma arising from astrocytes and/or oligodendrocytes).

In some embodiments, the cancer is glioma (e.g., low-grade glioma) in a subject having an age of 18 years or older.

In some embodiments, the cancer is glioma (e.g., low-grade glioma) in a subject having an age of younger than 18 years.

In some embodiments, the cancer is glioblastoma.

In some embodiments, the cancer is a recurrent cancer.

In some embodiments, the cancer is an advanced cancer.

In some embodiments, the cancer is a metastatic cancer.

In some embodiments, the tumor is a recurrent tumor.

In some embodiments, the tumor is an advanced tumor.

In some embodiments, the tumor is a metastatic tumor.

In some embodiments, the cancer is brain cancer.

In some embodiments, the cancer is recurrent brain cancer.

In some embodiments, the cancer is advanced brain cancer.

In some embodiments, the cancer is metastatic brain cancer.

In some embodiments, the cancer is lung cancer.

In some embodiments, the cancer is recurrent lung cancer.

In some embodiments, the cancer is advanced lung cancer.

In some embodiments, the cancer is metastatic lung cancer.

In some embodiments, the cancer is non-small cell lung cancer (NSCLC).

In some embodiments, the cancer is recurrent non-small cell lung cancer (NSCLC).

In some embodiments, the cancer is advanced non-small cell lung cancer (NSCLC).

In some embodiments, the cancer is metastatic non-small cell lung cancer (NSCLC).

In some embodiments, the cancer is NSCLC and the subject has at least one tumor and/or cancerous cell that expresses an oncogenic variant of B-Raf. In some aspects, the oncogenic variant of B-Raf is a B-Raf protein comprising at least one class I mutation. In some aspects, the oncogenic variant of B-Raf is a B-Raf protein comprising at least one class II mutation. In some aspects, the oncogenic variant of B-Raf is a B-Raf protein comprising at least one class III mutation. In some embodiments, the oncogenic variant is B-Raf-V600E. In some embodiments, the NSCLC can be recurrent, advanced, metastatic, or any combination thereof.

In some embodiments, the cancer is NSCLC and the subject has at least one tumor and/or cancerous cell that expresses an oncogenic variant of K-Ras. In some embodiments, the oncogenic variant of K-Ras is a K-Ras protein comprising an oncogenic mutation that is not K-Ras-G12C. In some embodiments, the NSCLC can be recurrent, advanced, metastatic, or any combination thereof.

In some embodiments, the cancer is a histiocytic neoplasm, and the subject has at least one tumor and/or cancerous cell that expresses an oncogenic variant of B-Raf. In some aspects, the oncogenic variant of B-Raf is a B-Raf protein comprising at least one class I mutation, at least one class II mutation, at least one class III mutation, or any combination thereof. In some embodiments, the cancer is a histiocytic neoplasm, and the subject has at least one tumor and/or cancerous cell that expresses an oncogenic variant of N-Ras. In some embodiments, the histiocytic neoplasm can be recurrent.

In some embodiments, the cancer is melanoma and the subject has at least one tumor and/or cancerous cell that expresses an oncogenic variant of B-Raf. In some aspects, the oncogenic variant of B-Raf is a B-Raf protein comprising at least one class I mutation, at least one class II mutation, at least one class III mutation, or any combination thereof. In some embodiments, the oncogenic variant is B-Raf-V600E. In some embodiments, the melanoma can be recurrent, advanced, metastatic, or any combination thereof.

In some embodiments, the cancer is melanoma and the subject has at least one tumor and/or cancerous cell that expresses an oncogenic variant of N-Ras. In some embodiments, the melanoma can be recurrent, advanced, metastatic, or any combination thereof.

In some embodiments, the cancer is thyroid carcinoma and the subject has at least one tumor and/or cancerous cell that expresses and oncogenic variant of B-Raf. In some aspects, the oncogenic variant of B-Raf is a B-Raf protein comprising at least one class I mutation, at least one class II mutation, at least one class III mutation, or any combination thereof.

In some embodiments, the cancer is colorectal carcinoma and the subject has at least one tumor and/or cancerous cell that expresses and oncogenic variant of B-Raf. In some aspects, the oncogenic variant of B-Raf is a B-Raf protein comprising at least one class II mutation, at least one class III mutation, or any combination thereof.

In some embodiments, the administration of a compound of the present disclosure does not induce paradoxical activation of wild-type B-Raf.

In some embodiments, the administration of a compound of the present disclosure does not substantially increase the amount of p-ERK in the subject.

In some embodiments, the administration of a compound of the present disclosure results in an amount of p-ERK in the subject that is at least about 10% lower, at least about 20% lower, at least about 30% lower, at least about 40% lower, at least about 50% lower, at least about 60% lower, at least about 70% lower, at least about 80% lower, at least about 90% lower, or at least about 95% lower as compared to a comparable subject being administered with vemurafenib or encorafenib.

In some embodiments, the administration of a compound of the present disclosure results in an amount of p-ERK in the subject that is at least about 10% lower, at least about 20% lower, at least about 30% lower, at least about 40% lower, at least about 50% lower, at least about 60% lower, at least about 70% lower, at least about 80% lower, at least about 90% lower, or at least about 95% lower as compared to a comparable subject without administration.

In some embodiments, the administration of a compound of the present disclosure reduces the tumor volume in the subject by at least about 10% lower, at least about 20%, at least about 30%, at least about 40%, at least about 50%, at least about 60%, at least about 70%, at least about 80%, at least about 90%, at least about 95%, at least about 98%, or at least about 99%.

In some embodiments, the cancer is insensitive or resistant to treatment with one or more inhibitors of the MAPK pathways.

In some embodiments, the cancer is insensitive or resistant to treatment with a BRAF inhibitor, a MEK inhibitor, or a combination thereof.

In some embodiments, the cancer is insensitive or resistant to treatment with a combination comprising a BRAF inhibitor and a MEK inhibitor.

In some embodiments, the cancer is insensitive to treatment with a combination comprising a BRAF inhibitor and a MEK inhibitor.

In some embodiments, the cancer is resistant to treatment with a combination comprising a BRAF inhibitor and a MEK inhibitor.

In some embodiments, the subject has an adverse reaction to treatment with one or more inhibitors of the MAPK pathways.

In some embodiments, the subject has an adverse reaction to treatment with a BRAF inhibitor, a MEK inhibitor, or a combination thereof.

In some embodiments, the subject has an adverse reaction to treatment with a combination comprising a BRAF inhibitor and a MEK inhibitor.

In some embodiments, the adverse reaction is a new cancer (e.g., skin cancer), bleeding, a gastrointestinal effect, an effect on the kidney and/or liver, an ocular issue, a lung issue, a fever, or any combination thereof.

In some embodiments, the subject has been previously administered one or more inhibitors of the MAPK pathways, and the subject has experienced disease progression despite the previous administration.

In some embodiments, the subject has been previously administered a BRAF inhibitor, a MEK inhibitor, or a combination thereof, and the subject has experienced disease progression despite the previous administration.

In some embodiments, the subject has been previously administered a combination comprising a BRAF inhibitor and a MEK inhibitor, and the subject has experienced disease progression despite the previous administration.

In some embodiments, the subject has at least one central nervous system metastasis. In some embodiments, the at least one central nervous system metastases is stable.

In some embodiments, the subject has no central nervous system metastases.

In some embodiments, the subject has at least one brain metastasis. In some embodiments, the at least one brain metastasis is stable.

In some embodiments, the subject has no brain metastases.

In some embodiments, the subject has histiocytosis.

### Inhibitors of MAPK Pathways

In some embodiments, the one or more inhibitors of the MAPK pathways comprise a BRAF inhibitor, a MEK inhibitor, or any combination thereof.

In some embodiments, the one or more inhibitors of the MAPK pathways comprise a combination comprising a BRAF inhibitor and a MEK inhibitor.

In some embodiments, the BRAF inhibitor is vemurafenib, dabrafenib, or encorafenib.

In some embodiments, the BRAF inhibitor is vemurafenib.

In some embodiments, the BRAF inhibitor is dabrafenib.

In some embodiments, the BRAF inhibitor is encorafenib.

In some embodiments, the MEK inhibitor is trametinib, cobimetinib, or binimetinib.

In some embodiments, the MEK inhibitor is trametinib.

In some embodiments, the MEK inhibitor is cobimetinib.

In some embodiments, the MEK inhibitor is binimetinib.

### Administration of the Compounds

In some embodiments, the compound is administered to the subject by oral administration or parenteral administration.

In some embodiments, the compound is administered to the subject by oral administration.

In some embodiments, a pharmaceutical composition comprising the compound is administered to the subject.

In some embodiments, the pharmaceutical composition is an oral formulation.

In some embodiments, a pharmaceutical composition is a tablet or a capsule.

In some embodiments, the pharmaceutical composition comprises a unit dose of the compound.

In some embodiments, the pharmaceutical composition comprises about 3±1 mg, about 3±0.9 mg, about 3±0.8 mg, about 3±0.7 mg, about 3±0.6 mg, about 3±0.5 mg, about 3±0.4 mg, about 3±0.3 mg, about 3±0.2 mg, or about 3±0.1 mg (e.g., about 3 mg) of the compound.

In some embodiments, the pharmaceutical composition comprises about 25±10 mg, about 25±9 mg, about 25±8 mg, about 25±7 mg, about 25±6 mg, about 25±5 mg, about 25±4 mg, about 25±3 mg, about 25±2 mg, or about 25±1 mg (e.g., about 25 mg) of the compound.

In some embodiments, the compound is administered at a dosage (e.g., a daily dosage) of:
about 6±3 mg, about 6±2 mg, about 6±1 mg, about 6±0.9 mg, about 6±0.8 mg, about 6±0.7 mg, about 6±0.6 mg, about 6±0.5 mg, about 6±0.4 mg, about 6±0.3 mg, about 6±0.2 mg, or about 6±0.1 mg (e.g., about 6 mg);
about 12±6 mg, about 12±5 mg, about 12±4 mg, about 12±3 mg, about 12±2 mg, about 12±1 mg, about 12±0.9 mg, about 12±0.8 mg, about 12±0.7 mg, about 12±0.6 mg, about 12±0.5 mg, about 12±0.4 mg, about 12±0.3 mg, about 12±0.2 mg, or about 12±0.1 mg (e.g., about 12 mg);
about 25±10 mg, about 25±9 mg, about 25±8 mg, about 25±7 mg, about 25±6 mg, about 25±5 mg, about 25±4 mg, about 25±3 mg, about 25±2 mg, or about 25±1 mg (e.g., about 25 mg);
about 50±20 mg, about 50±10 mg, about 50±9 mg, about 50±8 mg, about 50±7 mg, about 50±6 mg, about 50±5 mg, about 50±4 mg, about 50±3 mg, about 50±2 mg, or about 50±1 mg (e.g., about 50 mg);
about 100±50 mg, about 100±40 mg, about 100±30 mg, about 100±20 mg, about 100±10 mg, about 100±9 mg, about 100±8 mg, about 100±7 mg, about 100±6 mg, about 100±5 mg, about 100±4 mg, about 100±3 mg, about 100±2 mg, or about 100±1 mg (e.g., about 100 mg);
about 150±70 mg, about 150±60 mg, about 150±50 mg, about 150±40 mg, about 150±30 mg, about 150±20 mg, about 150±10 mg, about 150±5 mg, about 150±4 mg, or about 150±3 mg (e.g., about 150 mg); about 200±100 mg, about 200±90 mg, about 200±80 mg, about 200±70 mg, about 200±60 mg, about 200±50 mg, about 200±40 mg, about 200±30 mg, about 200±20 mg, or about 200±10 mg (e.g., about 200 mg);
about 300±150 mg, about 300±120 mg, about 300±100 mg, about 300±80 mg, about 300±60 mg, about 300±50 mg, about 300±40 mg, about 300±30 mg, about 300±20 mg, or about 300±10 mg (e.g., about 300 mg); about 400±200 mg, about 400±100 mg, about 400±90 mg, about 400±80 mg, about 400±70 mg, about 400±60 mg, about 400±50 mg, about 400±40 mg, about 400±30 mg, about 400±20 mg, or about 400±10 mg (e.g., about 400 mg);
about 600±300 mg, about 600±200 mg, about 600±100 mg, about 600±90 mg, about 600±80 mg, about 600±70 mg, about 600±60 mg, about 600±50 mg, about 600±40 mg, about 600±30 mg, about 600±20 mg, or about 600±10 mg (e.g., about 600 mg);
about 800±400 mg, about 800±300 mg, about 800±200 mg, about 800±100 mg, about 800±90 mg, about 800±80 mg, about 800±70 mg, about 800±60 mg, about 800±50 mg, about 800±40 mg, about 800±30 mg, about 800±20 mg, or about 800±10 mg (e.g., about 800 mg);
about 1000±500 mg, about 1000±400 mg, about 1000±300 mg, about 1000±200 mg, about 1000±100 mg, about 1000±90 mg, about 1000±80 mg, about 1000±70 mg, about 1000±60 mg, about 1000±50 mg, about 1000±40 mg, about 1000±30 mg, about 1000±20 mg, or about 1000±10 mg (e.g., about 1000 mg); or
about 1200±600 mg, about 1200±500 mg, about 1200±400 mg, about 1200±300 mg, about 1200±200 mg, about 1200±100 mg, about 1200±90 mg, about 1200±80 mg, about 1200±70 mg, about 1200±60 mg, about 1200±50 mg, about 1200±40 mg, about 1200±30 mg, about 1200±20 mg, or about 1200±10 mg (e.g., about 1200 mg).

In some embodiments, the compound is administered at a dosage (e.g., a daily dosage) of about 6±3 mg, about 6±2 mg, about 6±1 mg, about 6±0.9 mg, about 6±0.8 mg, about 6±0.7 mg, about 6±0.6 mg, about 6±0.5 mg, about 6±0.4 mg, about 6±0.3 mg, about 6±0.2 mg, or about 6±0.1 mg (e.g., about 6 mg).

In some embodiments, the compound is administered at a dosage (e.g., a daily dosage) of about 12±6 mg, about 12±5 mg, about 12±4 mg, about 12±3 mg, about 12±2 mg, about 12±1 mg, about 12±0.9 mg, about 12±0.8 mg, about 12±0.7 mg, about 12±0.6 mg, about 12±0.5 mg, about 12±0.4 mg, about 12±0.3 mg, about 12±0.2 mg, or about 12±0.1 mg (e.g., about 12 mg).

In some embodiments, the compound is administered at a dosage (e.g., a daily dosage) of about 25±10 mg, about 25±9 mg, about 25±8 mg, about 25±7 mg, about 25±6 mg, about 25±5 mg, about 25±4 mg, about 25±3 mg, about 25±2 mg, or about 25±1 mg (e.g., about 25 mg).

In some embodiments, the compound is administered at a dosage (e.g., a daily dosage) of about 50±20 mg, about 50±10 mg, about 50±9 mg, about 50±8 mg, about 50±7 mg, about 50±6 mg, about 50±5 mg, about 50±4 mg, about 50±3 mg, about 50±2 mg, or about 50±1 mg (e.g., about 50 mg).

In some embodiments, the compound is administered at a dosage (e.g., a daily dosage) of about 100±50 mg, about 100±40 mg, about 100±30 mg, about 100±20 mg, about 100±10 mg, about 100±9 mg, about 100±8 mg, about 100±7 mg, about 100±6 mg, about 100±5 mg, about 100±4 mg, about 100±3 mg, about 100±2 mg, or about 100±1 mg (e.g., about 100 mg).

In some embodiments, the compound is administered at a dosage (e.g., a daily dosage) of about 150±70 mg, about 150±60 mg, about 150±50 mg, about 150±40 mg, about 150±30 mg, about 150±20 mg, about 150±10 mg, about 150±5 mg, about 150±4 mg, or about 150±3 mg (e.g., about 150 mg).

In some embodiments, the compound is administered at a dosage (e.g., a daily dosage) of about 200±100 mg, about 200±90 mg, about 200±80 mg, about 200±70 mg, about 200±60 mg, about 200±50 mg, about 200±40 mg, about 200±30 mg, about 200±20 mg, or about 200±10 mg (e.g., about 200 mg).

In some embodiments, the compound is administered at a dosage (e.g., a daily dosage) of about 300±150 mg, about 300±120 mg, about 300±100 mg, about 300±80 mg, about 300±60 mg, about 300±50 mg, about 300±40 mg, about 300±30 mg, about 300±20 mg, or about 300±10 mg (e.g., about 300 mg).

In some embodiments, the compound is administered at a dosage (e.g., a daily dosage) of about 400±200 mg, about 400±100 mg, about 400±90 mg, about 400±80 mg, about 400±70 mg, about 400±60 mg, about 400±50 mg, about 400±40 mg, about 400±30 mg, about 400±20 mg, or about 400±10 mg (e.g., about 400 mg).

In some embodiments, the compound is administered at a dosage (e.g., a daily dosage) of about 600±300 mg, about 600±200 mg, about 600±100 mg, about 600±90 mg, about 600±80 mg, about 600±70 mg, about 600±60 mg, about 600±50 mg, about 600±40 mg, about 600±30 mg, about 600±20 mg, or about 600±10 mg (e.g., about 600 mg).

In some embodiments, the compound is administered at a dosage (e.g., a daily dosage) of about 800±400 mg, about 800±300 mg, about 800±200 mg, about 800±100 mg, about 800±90 mg, about 800±80 mg, about 800±70 mg, about 800±60 mg, about 800±50 mg, about 800±40 mg, about 800±30 mg, about 800±20 mg, or about 800±10 mg (e.g., about 800 mg).

In some embodiments, the compound is administered at a dosage (e.g., a daily dosage) of about 1000±500 mg, about 1000±400 mg, about 1000±300 mg, about 1000±200 mg, about 1000±100 mg, about 1000±90 mg, about 1000±80 mg, about 1000±70 mg, about 1000±60 mg, about 1000±50 mg, about 1000±40 mg, about 1000±30 mg, about 1000±20 mg, or about 1000±10 mg (e.g., about 1000 mg).

In some embodiments, the compound is administered at a dosage (e.g., a daily dosage) of about 1200±600 mg, about 1200±500 mg, about 1200±400 mg, about 1200±300 mg, about 1200±200 mg, about 1200±100 mg, about 1200±90 mg, about 1200±80 mg, about 1200±70 mg, about 1200±60 mg, about 1200±50 mg, about 1200±40 mg, about 1200±30 mg, about 1200±20 mg, or about 1200±10 mg (e.g., about 1200 mg).

In some embodiments, the compound is administered at a dosage (e.g., a daily dosage) of:
about 3±1 mg/kg, about 3±0.9 mg/kg, about 3±0.8 mg/kg, about 3±0.7 mg/kg, about 3±0.6 mg/kg, about 3±0.5 mg/kg, about 3±0.4 mg/kg, about 3±0.3 mg/kg, about 3±0.2 mg/kg, or about 3±0.1 mg/kg (e.g., about 3 mg/kg);
about 5±2 mg/kg, about 5±1 mg/kg, about 5±0.9 mg/kg, about 5±0.8 mg/kg, about 5±0.7 mg/kg, about 5±0.6 mg/kg, about 5±0.5 mg/kg, about 5±0.4 mg/kg, about 5±0.3 mg/kg, about 5±0.2 mg/kg, or about 5±0.1 mg/kg (e.g., about 5 mg/kg); or
about 10±5 mg/kg, about 10±4 mg/kg, about 10±3 mg/kg, about 10±2 mg/kg, about 10±1 mg/kg, about 10±0.9 mg/kg, about 10±0.8 mg/kg, about 10±0.7 mg/kg, about 10±0.6 mg/kg, about 10±0.5 mg/kg, about 10±0.4 mg/kg, about 10±0.3 mg/kg, about 10±0.2 mg/kg, or about 10±0.1 mg/kg (e.g., about 10 mg/kg).

In some embodiments, the compound is administered at a dosage (e.g., a daily dosage) of about 3±1 mg/kg, about 3±0.9 mg/kg, about 3±0.8 mg/kg, about 3±0.7 mg/kg, about 3±0.6 mg/kg, about 3±0.5 mg/kg, about 3±0.4 mg/kg, about 3±0.3 mg/kg, about 3±0.2 mg/kg, or about 3±0.1 mg/kg (e.g., about 3 mg/kg).

In some embodiments, the compound is administered at a dosage (e.g., a daily dosage) of about 5±2 mg/kg, about 5±1 mg/kg, about 5±0.9 mg/kg, about 5±0.8 mg/kg, about 5±0.7 mg/kg, about 5±0.6 mg/kg, about 5±0.5 mg/kg, about 5±0.4 mg/kg, about 5±0.3 mg/kg, about 5±0.2 mg/kg, or about 5±0.1 mg/kg (e.g., about 5 mg/kg).

In some embodiments, the compound is administered at a dosage (e.g., a daily dosage) of about 10±5 mg/kg, about 10±4 mg/kg, about 10±3 mg/kg, about 10±2 mg/kg, about 10±1 mg/kg, about 10±0.9 mg/kg, about 10±0.8 mg/kg, about 10±0.7 mg/kg, about 10±0.6 mg/kg, about 10±0.5 mg/kg, about 10±0.4 mg/kg, about 10±0.3 mg/kg, about 10±0.2 mg/kg, or about 10±0.1 mg/kg (e.g., about 10 mg/kg).

In some embodiments, the compound is administered at a dosage (e.g., a daily dosage) of:
about 0.2±0.2 mg/kg, about 0.2±0.1 mg/kg, about 0.2±0.09 mg/kg, about 0.2±0.08 mg/kg, about 0.2±0.07 mg/kg, about 0.2±0.06 mg/kg, about 0.2±0.05 mg/kg, about 0.2±0.04 mg/kg, about 0.2±0.03 mg/kg, about 0.2±0.02 mg/kg, or about 0.2±0.01 mg/kg (e.g., about 0.2 mg/kg);
about 0.4±0.2 mg/kg, about 0.4±0.1 mg/kg, about 0.4±0.09 mg/kg, about 0.4±0.08 mg/kg, about 0.4±0.07 mg/kg, about 0.4±0.06 mg/kg, about 0.4±0.05 mg/kg, about 0.4±0.04 mg/kg, about 0.4±0.03 mg/kg, about 0.4±0.02 mg/kg, or about 0.4±0.01 mg/kg (e.g., about 0.4 mg/kg); or
about 0.8±0.5 mg/kg, about 0.8±0.4 mg/kg, about 0.8±0.3 mg/kg, about 0.8±0.2 mg/kg, about 0.8±0.1 mg/kg, about 0.8±0.09 mg/kg, about 0.8±0.08 mg/kg, about 0.8±0.07 mg/kg, about 0.8±0.06 mg/kg, about 0.8±0.05 mg/kg, about 0.8±0.04 mg/kg, about 0.8±0.03 mg/kg, about 0.8±0.02 mg/kg, or about 0.8±0.01 mg/kg (e.g., about 0.8 mg/kg).

In some embodiments, the compound is administered at a dosage (e.g., a daily dosage) of about 0.2±0.2 mg/kg, about 0.2±0.1 mg/kg, about 0.2±0.09 mg/kg, about 0.2±0.08 mg/kg, about 0.2±0.07 mg/kg, about 0.2±0.06 mg/kg, about 0.2±0.05 mg/kg, about 0.2±0.04 mg/kg, about 0.2±0.03 mg/kg, about 0.2±0.02 mg/kg, or about 0.2±0.01 mg/kg (e.g., about 0.2 mg/kg).

In some embodiments, the compound is administered at a dosage (e.g., a daily dosage) of about 0.4±0.2 mg/kg, about 0.4±0.1 mg/kg, about 0.4±0.09 mg/kg, about 0.4±0.08 mg/kg, about 0.4±0.07 mg/kg, about 0.4±0.06 mg/kg, about 0.4±0.05 mg/kg, about 0.4±0.04 mg/kg, about 0.4±0.03 mg/kg, about 0.4±0.02 mg/kg, or about 0.4±0.01 mg/kg (e.g., about 0.4 mg/kg).

In some embodiments, the compound is administered at a dosage (e.g., a daily dosage) of about 0.8±0.5 mg/kg, about 0.8±0.4 mg/kg, about 0.8±0.3 mg/kg, about 0.8±0.2 mg/kg, about 0.8±0.1 mg/kg, about 0.8±0.09 mg/kg, about 0.8±0.08 mg/kg, about 0.8±0.07 mg/kg, about 0.8±0.06 mg/kg, about 0.8±0.05 mg/kg, about 0.8±0.04 mg/kg, about 0.8±0.03 mg/kg, about 0.8±0.02 mg/kg, or about 0.8±0.01 mg/kg (e.g., about 0.8 mg/kg).

In some embodiments, the pharmaceutical composition has a unit dose strength of about 1 mg, about 2 mg, about 3 mg, about 5 mg, about 10 mg, about 15 mg, about 20 mg, about 25 mg, about 50 mg, about 75 mg, or about 100 mg.

In some embodiments, the compound is administered with one or more drug holidays.

In some embodiments, the compound is administered without any drug holiday.

In some embodiments, prior to the administration, the subject is fasted for at least about 30 minutes, at least about 1 hour, at least about 2 hours, at least about 3 hours, at least about 4 hours, at least about 5 hours, at least about 6 hours, at least about 7 hours, at least about 8 hours, at least about 9 hours, at least about 10 hours, at least about 11 hours, or at least about 12 hours.

In some embodiments, prior to the administration, the subject is fed with about 30 minutes, about 1 hour, about 2 hours, about 3 hours, about 4 hours, about 5 hours, about 6 hours, about 7 hours, about 8 hours, about 9 hours, about 10 hours, about 11 hours, or about 12 hours.

In some embodiments, the compound is administered once daily (QD).

In some embodiments, the compound is administered twice daily (BID).

In some embodiments, the compound is administered for about 21 days, about 42 days, about 63 days, about 84 days, about 105 days, about 126 days, about 147 days, about 168 days, about 189 days, or about 210 days.

In some embodiments, the compound is administered for longer than 210 days.

In some embodiments, the compound is administered until a progression of cancer or an adverse effect (e.g., an intolerable toxicity) is observed.

### Combination Therapies

In some aspects, the present disclosure provides a combination comprising a compound of the present disclosure and one or more additional therapeutic agents.

In some aspects, the present disclosure provides a method of treating or preventing cancer in a subject, the method comprising administering to the subject a combination comprising a compound of the present disclosure (e.g., in a therapeutically effective amount) and one or more additional therapeutic agents (e.g., in a therapeutically effective amount).

In some aspects, the present disclosure provides a method of treating cancer in a subject, the method comprising administering to the subject a combination comprising a compound of the present disclosure (e.g., in a therapeutically effective amount) and additional therapeutic agents (e.g., in a therapeutically effective amount).

In some aspects, the present disclosure provides a combination comprising a compound of the present disclosure and one or more additional therapeutic agents, for treating or preventing cancer in a subject.

In some aspects, the present disclosure provides a combination comprising a compound of the present disclosure and one or more additional therapeutic agents, for treating cancer in a subject.

In some aspects, the present disclosure provides a compound of the present disclosure for use in combination with one or more additional therapeutic agents in treating cancer in a subject.

In some aspects, the present disclosure provides a use of a combination comprising a compound of the present disclosure and one or more additional therapeutic agents, in the manufacture of a medicament for treating or preventing cancer in a subject.

In some aspects, the present disclosure provides a use of a combination comprising a compound of the present disclosure and one or more additional therapeutic agents, in the manufacture of a medicament for treating or preventing cancer in a subject.

In some aspects, the present disclosure provides a use of a combination comprising a compound of the present disclosure and additional therapeutic agents, in the manufacture of a medicament for treating cancer in a subject.

In some embodiments, the one or more additional therapeutic agents comprise one or more SHP2 (src homology-2 domain-containing protein tyrosine phosphatase-2) inhibitors, one or more SOS1 inhibitors, one or more KRAS (kirsten rat sarcoma virus) inhibitors, one or more ERK (extracellular signal-regulated kinase) inhibitors, one or more immune checkpoint inhibitors, one or more chemotherapies, one or more EGFR (epidermal growth factor receptor) inhibitors, one or more MET (mesenchymal-epithelial transition) inhibitors, one or more TEAD (transcriptional enhanced associate domain) inhibitors, one or more YAP (yes-associated protein) inhibitors, one or more PI3K (phosphoinositide 3-kinase) inhibitors, one or more mTOR (mammalian target of rapamycin) inhibitors, one or more metabolic inhibitors, or one or more MEK (mitogen-activated protein kinase kinase) inhibitors.

In some embodiments, the one or more additional therapeutic agents comprise one or more SHP2 inhibitors.

In some embodiments, the one or more SHP2 inhibitors comprises JAB-3068, JAB-3312, TNO-155, RLY-1971, or RMC-4630.

In some embodiments, the one or more additional therapeutic agents comprise one or more SOS1 inhibitors.

In some embodiments, the one or more SOS1 inhibitors comprises BI-1701963, BI-1703880, or MRTX0902.

In some embodiments, the one or more additional therapeutic agents comprise one or more KRAS inhibitors.

In some embodiments, the one or more KRAS inhibitors comprises sotorasib, adagrasib, LY3537982, divarasib, JDQ443, BI-1823911, MRTX1133, RMC-9805, or RMC-6236.

In some embodiments, the one or more additional therapeutic agents comprise one or more ERK inhibitors.

In some embodiments, the one or more ERK inhibitors comprises ulixertinib, MK-8353, LY3214996, ASTX029, ASN007, LTT462, or KO-947.

In some embodiments, the one or more additional therapeutic agents comprise one or more immune checkpoint inhibitors.

In some embodiments, the one or more immune checkpoint inhibitors comprises pembrolizumab, ipilimumab, nivolumab, or atezolizumab.

In some embodiments, the one or more additional therapeutic agents comprise one or more chemotherapies.

In some embodiments, the one or more chemotherapies comprises oxaliplatin or irinotecan.

In some embodiments, the one or more additional therapeutic agents comprise one or more EGFR inhibitors.

In some embodiments, the one or more EGFR inhibitors comprises erlotinib, osimertinib, neratinib, gefitinib, cetuximab, panitumumab, dacomitinib, lapatinib, necitumumab, mobocertinib, or vandetanib.

In some embodiments, the one or more additional therapeutic agents comprise one or more MET inhibitors.

In some embodiments, the one or more MET inhibitors comprises crizotinib, capmatinib, tepotinib, savolitinib. Cabozantinib, glesatinib, foretinib, merestinib, tivantinib, SAR125844, onartuzumab, telisotuzumab, or JNJ-61186372.

In some embodiments, the one or more additional therapeutic agents comprise one or more TEAD inhibitors.

In some embodiments, the one or more TEAD inhibitors comprises VT3989, IK-930, or IAG933.

In some embodiments, the one or more additional therapeutic agents comprise one or more YAP inhibitors.

In some embodiments, the one or more YAP inhibitors comprises verteporfin.

In some embodiments, the one or more additional therapeutic agents comprise one or more PI3K inhibitors.

In some embodiments, the one or more PI3K inhibitors comprises idelalisib, alpelisib, alpelisib, leniolisib, duvelisib, or copanlisib.

In some embodiments, the one or more additional therapeutic agents comprise one or more mTOR inhibitors.

In some embodiments, the one or more mTOR inhibitors comprises everolimus, sirolimus, temsirolimus, everolimus, sirolimus, sirolimus protein-bound, or everolimus.

In some embodiments, the one or more additional therapeutic agents comprise one or more metabolic inhibitors.

In some embodiments, the one or more metabolic inhibitors comprises trifluridine, gemcitabine, fluorouracil, pentostatin, clofarabine, azacitidine, cytarabine, mercaptopurine, fludarabine, or capecitabine.

In some embodiments, the one or more metabolic inhibitors comprises one or more MEK inhibitors.

In some embodiments, the one or more MEK inhibitors comprises trametinib, cobimetinib, or binimetinib.

In some aspects, the present disclosure provides a combination comprising a compound of the present disclosure and one or more inhibitors of the MAPK pathways.

In some aspects, the present disclosure provides a combination comprising a compound of the present disclosure and one or more MEK inhibitors.

In some aspects, the present disclosure provides a combination comprising:
a compound of the present disclosure; and
one or more of trametinib, cobimetinib, or binimetinib.

In some aspects, the present disclosure provides a combination comprising a compound of the present disclosure and trametinib.

In some aspects, the present disclosure provides a combination comprising a compound of the present disclosure and cobimetinib.

In some aspects, the present disclosure provides a combination comprising a compound of the present disclosure and binimetinib.

In some aspects, the present disclosure provides a method of treating or preventing cancer in a subject, the method comprising administering to the subject a combination comprising a compound of the present disclosure (e.g., in a therapeutically effective amount) and one or more inhibitors of the MAPK pathways (e.g., in a therapeutically effective amount).

In some aspects, the present disclosure provides a method of treating or preventing cancer in a subject, the method comprising administering to the subject a combination comprising:
a compound of the present disclosure (e.g., in a therapeutically effective amount); and
one or more of trametinib, cobimetinib, or binimetinib (e.g., in a therapeutically effective amount).

In some aspects, the present disclosure provides a method of treating or preventing cancer in a subject, the method comprising administering to the subject a combination comprising a compound of the present disclosure (e.g., in a therapeutically effective amount) and trametinib (e.g., in a therapeutically effective amount).

In some aspects, the present disclosure provides a method of treating or preventing cancer in a subject, the method comprising administering to the subject a combination comprising a compound of the present disclosure (e.g., in a therapeutically effective amount) and cobimetinib (e.g., in a therapeutically effective amount).

In some aspects, the present disclosure provides a method of treating or preventing cancer in a subject, the method comprising administering to the subject a combination comprising a compound of the present disclosure (e.g., in a therapeutically effective amount) and binimetinib (e.g., in a therapeutically effective amount).

In some aspects, the present disclosure provides a method of treating cancer in a subject, the method comprising administering to the subject a combination comprising a compound of the present disclosure (e.g., in a therapeutically effective amount) and one or more inhibitors of the MAPK pathways (e.g., in a therapeutically effective amount).

In some aspects, the present disclosure provides a method of treating cancer in a subject, the method comprising administering to the subject a combination comprising:
a compound of the present disclosure (e.g., in a therapeutically effective amount); and
one or more of trametinib, cobimetinib, or binimetinib (e.g., in a therapeutically effective amount).

In some aspects, the present disclosure provides a method of treating cancer in a subject, the method comprising administering to the subject a combination comprising a compound of the present disclosure (e.g., in a therapeutically effective amount) and trametinib (e.g., in a therapeutically effective amount).

In some aspects, the present disclosure provides a method of treating cancer in a subject, the method comprising administering to the subject a combination comprising a compound of the present disclosure (e.g., in a therapeutically effective amount) and cobimetinib (e.g., in a therapeutically effective amount).

In some aspects, the present disclosure provides a method of treating cancer in a subject, the method comprising administering to the subject a combination comprising a compound of the present disclosure (e.g., in a therapeutically effective amount) and binimetinib (e.g., in a therapeutically effective amount).

In some aspects, the present disclosure provides a combination comprising a compound of the present disclosure and one or more inhibitors of the MAPK pathways, for treating or preventing cancer in a subject.

In some aspects, the present disclosure provides a combination comprising:
a compound of the present disclosure, and
one or more of trametinib, cobimetinib, or binimetinib,
   for treating or preventing cancer in a subject.

In some aspects, the present disclosure provides a combination comprising a compound of the present disclosure and trametinib, for treating or preventing cancer in a subject.

In some aspects, the present disclosure provides a combination comprising a compound of the present disclosure and cobimetinib, for treating or preventing cancer in a subject.

In some aspects, the present disclosure provides a combination comprising a compound of the present disclosure and binimetinib, for treating or preventing cancer in a subject.

In some aspects, the present disclosure provides a combination comprising a compound of the present disclosure and one or more inhibitors of the MAPK pathways, for treating cancer in a subject.

In some aspects, the present disclosure provides a combination comprising:
a compound of the present disclosure, and
one or more of trametinib, cobimetinib, or binimetinib,
   for treating cancer in a subject.

In some aspects, the present disclosure provides a combination comprising a compound of the present disclosure and trametinib, for treating cancer in a subject.

In some aspects, the present disclosure provides a combination comprising a compound of the present disclosure and cobimetinib, for treating cancer in a subject.

In some aspects, the present disclosure provides a combination comprising a compound of the present disclosure and binimetinib, for treating cancer in a subject.

In some aspects, the present disclosure provides a use of a combination comprising a compound of the present disclosure and one or more inhibitors of the MAPK pathways, in the manufacture of a medicament for treating or preventing cancer in a subject.

In some aspects, the present disclosure provides a use of a combination comprising:
a compound of the present disclosure, and
one or more of trametinib, cobimetinib, or binimetinib,
   in the manufacture of a medicament for treating or preventing cancer in a subject.

In some aspects, the present disclosure provides a use of a combination comprising a compound of the present disclosure and trametinib, in the manufacture of a medicament for treating or preventing cancer in a subject.

In some aspects, the present disclosure provides a use of a combination comprising a compound of the present disclosure and cobimetinib, in the manufacture of a medicament for treating or preventing cancer in a subject.

In some aspects, the present disclosure provides a use of a combination comprising a compound of the present disclosure and binimetinib, in the manufacture of a medicament for treating or preventing cancer in a subject.

In some aspects, the present disclosure provides a use of a combination comprising a compound of the present disclosure and one or more inhibitors of the MAPK pathways, in the manufacture of a medicament for treating cancer in a subject.

In some aspects, the present disclosure provides a use of a combination comprising:
a compound of the present disclosure, and
one or more of trametinib, cobimetinib, or binimetinib,
   in the manufacture of a medicament for treating cancer in a subject.

In some aspects, the present disclosure provides a use of a combination comprising a compound of the present disclosure and trametinib, in the manufacture of a medicament for treating cancer in a subject.

In some aspects, the present disclosure provides a use of a combination comprising a compound of the present disclosure and cobimetinib, in the manufacture of a medicament for treating cancer in a subject.

In some aspects, the present disclosure provides a use of a combination comprising a compound of the present disclosure and binimetinib, in the manufacture of a medicament for treating cancer in a subject.

In some embodiments, the compound and the one or more inhibitors of the MAPK pathways are administered simultaneously.

In some embodiments, the compound and the one or more inhibitors of the MAPK pathways are administered in a co-formulation.

In some embodiments, the compound and the one or more inhibitors of the MAPK pathways are administered in separate formulations.

In some embodiments, the compound and the one or more inhibitors of the MAPK pathways are administered sequentially or in alternation.

In some embodiments, the compound and the one or more inhibitors of the MAPK pathways are administered sequentially.

In some embodiments, the compound and the one or more inhibitors of the MAPK pathways are administered in temporal proximity.

In some embodiments, the compound is administered prior to the administration of the one or more inhibitors of the MAPK pathways.

In some embodiments, compound is administered in temporal proximity prior to the administration of the one or more inhibitors of the MAPK pathways

In some embodiments, the compound is administered at about 10 minutes, about 20 minutes, about 30 minutes, about 40 minutes, about 50 minutes, about 1 hour, about 2 hours, about 3 hours, about 4 hours, about 5 hours, about 6 hours, about 7 hours, about 8 hours, about 9 hours, about 10 hours, about 11 hours, or about 12 hours prior to the administration of the one or more inhibitors of the MAPK pathways.

In some embodiments, the one or more inhibitors of the MAPK pathways are administered prior to the administration of the compound.

In some embodiments, the one or more inhibitors of the MAPK pathways are administered in temporal proximity prior to the administration of the compound.

In some embodiments, the one or more inhibitors of the MAPK pathways are administered at about 10 minutes, about 20 minutes, about 30 minutes, about 40 minutes, about 50 minutes, about 1 hour, about 2 hours, about 3 hours, about 4 hours, about 5 hours, about 6 hours, about 7 hours, about 8 hours, about 9 hours, about 10 hours, about 11 hours, or about 12 hours prior to the administration of the compound.

In some embodiments, the compound and the one or more inhibitors of the MAPK pathways are administered in alternation.

In some embodiments, the compound and the one or more inhibitors of the MAPK pathways are administered by a same route of administration.

In some embodiments, the compound and the one or more inhibitors of the MAPK pathways are administered by different routes of administration.

In some embodiments, at least two inhibitors of the MAPK pathways are administered.

In some embodiments, binimetinib is administered once daily.

In some embodiments, binimetinib is administered twice daily.

In some embodiments, the compound is administered at a dosage (e.g., a daily dosage) of about 6±3 mg, about 6±2 mg, about 6±1 mg, about 6±0.9 mg, about 6±0.8 mg, about 6±0.7 mg, about 6±0.6 mg, about 6±0.5 mg, about 6±0.4 mg, about 6±0.3 mg, about 6±0.2 mg, or about 6±0.1 mg (e.g., about 6 mg); about 12±6 mg, about 12±5 mg, about 12±4 mg, about 12±3 mg, about 12±2 mg, about 12±1 mg, about 12±0.9 mg, about 12±0.8 mg, about 12±0.7 mg, about 12±0.6 mg, about 12±0.5 mg, about 12±0.4 mg, about 12±0.3 mg, about 12±0.2 mg, or about 12±0.1 mg (e.g., about 12 mg); about 25±10 mg, about 25±9 mg, about 25±8 mg, about 25±7 mg, about 25±6 mg, about 25±5 mg, about 25±4 mg, about 25±3 mg, about 25±2 mg, or about 25±1 mg (e.g., about 25 mg); about 50±20 mg, about 50±10 mg, about 50±9 mg, about 50±8 mg, about 50±7 mg, about 50±6 mg, about 50±5 mg, about 50±4 mg, about 50±3 mg, about 50±2 mg, or about 50±1 mg (e.g., about 50 mg); about 100±50 mg, about 100±40 mg, about 100±30 mg, about 100±20 mg, about 100±10 mg, about 100±9 mg, about 100±8 mg, about 100±7 mg, about 100±6 mg, about 100±5 mg, about 100±4 mg, about 100±3 mg, about 100±2 mg, or about 100±1 mg (e.g., about 100 mg); about 150±70 mg, about 150±60 mg, about 150±50 mg, about 150±40 mg, about 150±30 mg, about 150±20 mg, about 150±10 mg, about 150±5 mg, about 150±4 mg, or about 150±3 mg (e.g., about 150 mg); about 200±100 mg, about 200±90 mg, about 200±80 mg, about 200±70 mg, about 200±60 mg, about 200±50 mg, about 200±40 mg, about 200±30 mg, about 200±20 mg, or about 200±10 mg (e.g., about 200 mg); about 300±150 mg, about 300±120 mg, about 300±100 mg, about 300±80 mg, about 300±60 mg, about 300±50 mg, about 300±40 mg, about 300±30 mg, about 300±20 mg, or about 300±10 mg (e.g., about 300 mg); about 400±200 mg, about 400±100 mg, about 400±90 mg, about 400±80 mg, about 400±70 mg, about 400±60 mg, about 400±50 mg, about 400±40 mg, about 400±30 mg, about 400±20 mg, or about 400±10 mg (e.g., about 400 mg); about 600±300 mg, about 600±200 mg, about 600±100 mg, about 600±90 mg, about 600±80 mg, about 600±70 mg, about 600±60 mg, about 600±50 mg, about 600±40 mg, about 600±30 mg, about 600±20 mg, or about 600±10 mg (e.g., about 600 mg); about 800±400 mg, about 800±300 mg, about 800±200 mg, about 800±100 mg, about 800±90 mg, about 800±80 mg, about 800±70 mg, about 800±60 mg, about 800±50 mg, about 800±40 mg, about 800±30 mg, about 800±20 mg, or about 800±10 mg (e.g., about 800 mg); about 1000±500 mg, about 1000±400 mg, about 1000±300 mg, about 1000±200 mg, about 1000±100 mg, about 1000±90 mg, about 1000±80 mg, about 1000±70 mg, about 1000±60 mg, about 1000±50 mg, about 1000±40 mg, about 1000±30 mg, about 1000±20 mg, or about 1000±10 mg (e.g., about 1000 mg); or about 1200±600 mg, about 1200±500 mg, about 1200±400 mg, about 1200±300 mg, about 1200±200 mg, about 1200±100 mg, about 1200±90 mg, about 1200±80 mg, about 1200±70 mg, about 1200±60 mg, about 1200±50 mg, about 1200±40 mg, about 1200±30 mg, about 1200±20 mg, or about 1200±10 mg (e.g., about 1200 mg); and
binimetinib is administered at a dosage (e.g., a daily dosage) of about 15±6 mg, about 15±5 mg, about 15±4 mg, about 15±3 mg, about 15±2 mg, about 15±1 mg, about 15±0.9 mg, about 15±0.8 mg, about 15±0.7 mg, about 15±0.6 mg, about 15±0.5 mg, about 15±0.4 mg, about 15±0.3 mg, about 15±0.2 mg, or about 15±0.1 mg (e.g., about 15 mg); about 30±10 mg, about 30±9 mg, about 30±8 mg, about 30±7 mg, about 30±6 mg, about 30±5 mg, about 30±4 mg, about 30±3 mg, about 30±2 mg, or about 30±1 mg (e.g., about 30 mg); about 45±20 mg, about 45±10 mg, about 45±9 mg, about 45±8 mg, about 45±7 mg, about 45±6 mg, about 45±5 mg, about 45±4 mg, about 45±3 mg, about 45±2 mg, or about 45±1 mg (e.g., about 45 mg); about 60±30 mg, about 60±20 mg, about 60±10 mg, about 60±9 mg, about 60±8 mg, about 60±7 mg, about 60±6 mg, about 60±5 mg, about 60±4 mg, about 60±3 mg, about 60±2 mg, or about 60±1 mg (e.g., about 60 mg); about 75±40 mg, about 75±30 mg, about 75±20 mg, about 75±10 mg, about 75±9 mg, about 75±8 mg, about 75±7 mg, about 75±6 mg, about 75±5 mg, about 75±4 mg, about 75±3 mg, about 75±2 mg, or about 75±1 mg (e.g., about 75 mg); or about 90±50 mg, about 90±40 mg, about 90±30 mg, about 90±20 mg, about 90±10 mg, about 90±9 mg, about 90±8 mg, about 90±7 mg, about 90±6 mg, about 90±5 mg, about 90±4 mg, about 90±3 mg, about 90±2 mg, or about 90±1 mg (e.g., about 90 mg).

In some embodiments, the compound is administered at a dosage (e.g., a daily dosage) of about 6±3 mg, about 6±2 mg, about 6±1 mg, about 6±0.9 mg, about 6±0.8 mg, about 6±0.7 mg, about 6±0.6 mg, about 6±0.5 mg, about 6±0.4 mg, about 6±0.3 mg, about 6±0.2 mg, or about 6±0.1 mg (e.g., about 6 mg); about 12±6 mg, about 12±5 mg, about 12±4 mg, about 12±3 mg, about 12±2 mg, about 12±1 mg, about 12±0.9 mg, about 12±0.8 mg, about 12±0.7 mg, about 12±0.6 mg, about 12±0.5 mg, about 12±0.4 mg, about 12±0.3 mg, about 12±0.2 mg, or about 12±0.1 mg (e.g., about 12 mg); about 25±10 mg, about 25±9 mg, about 25±8 mg, about 25±7 mg, about 25±6 mg, about 25±5 mg, about 25±4 mg, about 25±3 mg, about 25±2 mg, or about 25±1 mg (e.g., about 25 mg); about 50±20 mg, about 50±10 mg, about 50±9 mg, about 50±8 mg, about 50±7 mg, about 50±6 mg, about 50±5 mg, about 50±4 mg, about 50±3 mg, about 50±2 mg, or about 50±1 mg (e.g., about 50 mg); about 100±50 mg, about 100±40 mg, about 100±30 mg, about 100±20 mg, about 100±10 mg, about 100±9 mg, about 100±8 mg, about 100±7 mg, about 100±6 mg, about 100±5 mg, about 100±4 mg, about 100±3 mg, about 100±2 mg, or about 100±1 mg (e.g., about 100 mg); about 150±70 mg, about 150±60 mg, about 150±50 mg, about 150±40 mg, about 150±30 mg, about 150±20 mg, about 150±10 mg, about 150±5 mg, about 150±4 mg, or about 150±3 mg (e.g., about 150 mg); about 200±100 mg, about 200±90 mg, about 200±80 mg, about 200±70 mg, about 200±60 mg, about 200±50 mg, about 200±40 mg, about 200±30 mg, about 200±20 mg, or about 200±10 mg (e.g., about 200 mg); about 300±150 mg, about 300±120 mg, about 300±100 mg, about 300±80 mg, about 300±60 mg, about 300±50 mg, about 300±40 mg, about 300±30 mg, about 300±20 mg, or about 300±10 mg (e.g., about 300 mg); about 400±200 mg, about 400±100 mg, about 400±90 mg, about 400±80 mg, about 400±70 mg, about 400±60 mg, about 400±50 mg, about 400±40 mg, about 400±30 mg, about 400±20 mg, or about 400±10 mg (e.g., about 400 mg); about 600±300 mg, about 600±200 mg, about 600±100 mg, about 600±90 mg, about 600±80 mg, about 600±70 mg, about 600±60 mg, about 600±50 mg, about 600±40 mg, about 600±30 mg, about 600±20 mg, or about 600±10 mg (e.g., about 600 mg); about 800±400 mg, about 800±300 mg, about 800±200 mg, about 800±100 mg, about 800±90 mg, about 800±80 mg, about 800±70 mg, about 800±60 mg, about 800±50 mg, about 800±40 mg, about 800±30 mg, about 800±20 mg, or about 800±10 mg (e.g., about 800 mg); about 1000±500 mg, about 1000±400 mg, about 1000±300 mg, about 1000±200 mg, about 1000±100 mg, about 1000±90 mg, about 1000±80 mg, about 1000±70 mg, about 1000±60 mg, about 1000±50 mg, about 1000±40 mg, about 1000±30 mg, about 1000±20 mg, or about 1000±10 mg (e.g., about 1000 mg); or about 1200±600 mg, about 1200±500 mg, about 1200±400 mg, about 1200±300 mg, about 1200±200 mg, about 1200±100 mg, about 1200±90 mg, about 1200±80 mg, about 1200±70 mg, about 1200±60 mg, about 1200±50 mg, about 1200±40 mg, about 1200±30 mg, about 1200±20 mg, or about 1200±10 mg (e.g., about 1200 mg); and
binimetinib is administered at a dosage (e.g., a daily dosage) of about 60±30 mg, about 60±20 mg, about 60±10 mg, about 60±9 mg, about 60±8 mg, about 60±7 mg, about 60±6 mg, about 60±5 mg, about 60±4 mg, about 60±3 mg, about 60±2 mg, or about 60±1 mg (e.g., about 60 mg); or about 90±50 mg, about 90±40 mg, about 90±30 mg, about 90±20 mg, about 90±10 mg, about 90±9 mg, about 90±8 mg, about 90±7 mg, about 90±6 mg, about 90±5 mg, about 90±4 mg, about 90±3 mg, about 90±2 mg, or about 90±1 mg (e.g., about 90 mg).

In some embodiments, the compound is administered at a dosage (e.g., a daily dosage) of about 6±3 mg, about 6±2 mg, about 6±1 mg, about 6±0.9 mg, about 6±0.8 mg, about 6±0.7 mg, about 6±0.6 mg, about 6±0.5 mg, about 6±0.4 mg, about 6±0.3 mg, about 6±0.2 mg, or about 6±0.1 mg (e.g., about 6 mg); and
binimetinib is administered at a dosage (e.g., a daily dosage) of about 60±30 mg, about 60±20 mg, about 60±10 mg, about 60±9 mg, about 60±8 mg, about 60±7 mg, about 60±6 mg, about 60±5 mg, about 60±4 mg, about 60±3 mg, about 60±2 mg, or about 60±1 mg (e.g., about 60 mg); or about 90±50 mg, about 90±40 mg, about 90±30 mg, about 90±20 mg, about 90±10 mg, about 90±9 mg, about 90±8 mg, about 90±7 mg, about 90±6 mg, about 90±5 mg, about 90±4 mg, about 90±3 mg, about 90±2 mg, or about 90±1 mg (e.g., about 90 mg).

In some embodiments, the compound is administered at a dosage (e.g., a daily dosage) of about 12±6 mg, about 12±5 mg, about 12±4 mg, about 12±3 mg, about 12±2 mg, about 12±1 mg, about 12±0.9 mg, about 12±0.8 mg, about 12±0.7 mg, about 12±0.6 mg, about 12±0.5 mg, about 12±0.4 mg, about 12±0.3 mg, about 12±0.2 mg, or about 12±0.1 mg (e.g., about 12 mg); and
binimetinib is administered at a dosage (e.g., a daily dosage) of about 60±30 mg, about 60±20 mg, about 60±10 mg, about 60±9 mg, about 60±8 mg, about 60±7 mg, about 60±6 mg, about 60±5 mg, about 60±4 mg, about 60±3 mg, about 60±2 mg, or about 60±1 mg (e.g., about 60 mg); or about 90±50 mg, about 90±40 mg, about 90±30 mg, about 90±20 mg, about 90±10 mg, about 90±9 mg, about 90±8 mg, about 90±7 mg, about 90±6 mg, about 90±5 mg, about 90±4 mg, about 90±3 mg, about 90±2 mg, or about 90±1 mg (e.g., about 90 mg).

In some embodiments, the compound is administered at a dosage (e.g., a daily dosage) of about 25±10 mg, about 25±9 mg, about 25±8 mg, about 25±7 mg, about 25±6 mg, about 25±5 mg, about 25±4 mg, about 25±3 mg, about 25±2 mg, or about 25±1 mg (e.g., about 25 mg); and
binimetinib is administered at a dosage (e.g., a daily dosage) of about 60±30 mg, about 60±20 mg, about 60±10 mg, about 60±9 mg, about 60±8 mg, about 60±7 mg, about 60±6 mg, about 60±5 mg, about 60±4 mg, about 60±3 mg, about 60±2 mg, or about 60±1 mg (e.g., about 60 mg); or about 90±50 mg, about 90±40 mg, about 90±30 mg, about 90±20 mg, about 90±10 mg, about 90±9 mg, about 90±8 mg, about 90±7 mg, about 90±6 mg, about 90±5 mg, about 90±4 mg, about 90±3 mg, about 90±2 mg, or about 90±1 mg (e.g., about 90 mg).

In some embodiments, the compound is administered at a dosage (e.g., a daily dosage) of about 50±20 mg, about 50±10 mg, about 50±9 mg, about 50±8 mg, about 50±7 mg, about 50±6 mg, about 50±5 mg, about 50±4 mg, about 50±3 mg, about 50±2 mg, or about 50±1 mg (e.g., about 50 mg); and
binimetinib is administered at a dosage (e.g., a daily dosage) of about 60±30 mg, about 60±20 mg, about 60±10 mg, about 60±9 mg, about 60±8 mg, about 60±7 mg, about 60±6 mg, about 60±5 mg, about 60±4 mg, about 60±3 mg, about 60±2 mg, or about 60±1 mg (e.g., about 60 mg); or about 90±50 mg, about 90±40 mg, about 90±30 mg, about 90±20 mg, about 90±10 mg, about 90±9 mg, about 90±8 mg, about 90±7 mg, about 90±6 mg, about 90±5 mg, about 90±4 mg, about 90±3 mg, about 90±2 mg, or about 90±1 mg (e.g., about 90 mg).

In some embodiments, the compound is administered at a dosage (e.g., a daily dosage) of about 100±50 mg, about 100±40 mg, about 100±30 mg, about 100±20 mg, about 100±10 mg, about 100±9 mg, about 100±8 mg, about 100±7 mg, about 100±6 mg, about 100±5 mg, about 100±4 mg, about 100±3 mg, about 100±2 mg, or about 100±1 mg (e.g., about 100 mg); and
binimetinib is administered at a dosage (e.g., a daily dosage) of about 60±30 mg, about 60±20 mg, about 60±10 mg, about 60±9 mg, about 60±8 mg, about 60±7 mg, about 60±6 mg, about 60±5 mg, about 60±4 mg, about 60±3 mg, about 60±2 mg, or about 60±1 mg (e.g., about 60 mg); or about 90±50 mg, about 90±40 mg, about 90±30 mg, about 90±20 mg, about 90±10 mg, about 90±9 mg, about 90±8 mg, about 90±7 mg, about 90±6 mg, about 90±5 mg, about 90±4 mg, about 90±3 mg, about 90±2 mg, or about 90±1 mg (e.g., about 90 mg).

In some embodiments, the compound is administered at a dosage (e.g., a daily dosage) of about 150±70 mg, about 150±60 mg, about 150±50 mg, about 150±40 mg, about 150±30 mg, about 150±20 mg, about 150±10 mg, about 150±5 mg, about 150±4 mg, or about 150±3 mg (e.g., about 150 mg); and
binimetinib is administered at a dosage (e.g., a daily dosage) of about 60±30 mg, about 60±20 mg, about 60±10 mg, about 60±9 mg, about 60±8 mg, about 60±7 mg, about 60±6 mg, about 60±5 mg, about 60±4 mg, about 60±3 mg, about 60±2 mg, or about 60±1 mg (e.g., about 60 mg); or about 90±50 mg, about 90±40 mg, about 90±30 mg, about 90±20 mg, about 90±10 mg, about 90±9 mg, about 90±8 mg, about 90±7 mg, about 90±6 mg, about 90±5 mg, about 90±4 mg, about 90±3 mg, about 90±2 mg, or about 90±1 mg (e.g., about 90 mg).

In some embodiments, the compound is administered at a dosage (e.g., a daily dosage) of about 200±100 mg, about 200±90 mg, about 200±80 mg, about 200±70 mg, about 200±60 mg, about 200±50 mg, about 200±40 mg, about 200±30 mg, about 200±20 mg, or about 200±10 mg (e.g., about 200 mg); and
binimetinib is administered at a dosage (e.g., a daily dosage) of about 60±30 mg, about 60±20 mg, about 60±10 mg, about 60±9 mg, about 60±8 mg, about 60±7 mg, about 60±6 mg, about 60±5 mg, about 60±4 mg, about 60±3 mg, about 60±2 mg, or about 60±1 mg (e.g., about 60 mg); or about 90±50 mg, about 90±40 mg, about 90±30 mg, about 90±20 mg, about 90±10 mg, about 90±9 mg, about 90±8 mg, about 90±7 mg, about 90±6 mg, about 90±5 mg, about 90±4 mg, about 90±3 mg, about 90±2 mg, or about 90±1 mg (e.g., about 90 mg).

In some embodiments, the compound is administered at a dosage (e.g., a daily dosage) of about 300±150 mg, about 300±120 mg, about 300±100 mg, about 300±80 mg, about 300±60 mg, about 300±50 mg, about 300±40 mg, about 300±30 mg, about 300±20 mg, or about 300±10 mg (e.g., about 300 mg); and
binimetinib is administered at a dosage (e.g., a daily dosage) of about 60±30 mg, about 60±20 mg, about 60±10 mg, about 60±9 mg, about 60±8 mg, about 60±7 mg, about 60±6 mg, about 60±5 mg, about 60±4 mg, about 60±3 mg, about 60±2 mg, or about 60±1 mg (e.g., about 60 mg); or about 90±50 mg, about 90±40 mg, about 90±30 mg, about 90±20 mg, about 90±10 mg, about 90±9 mg, about 90±8 mg, about 90±7 mg, about 90±6 mg, about 90±5 mg, about 90±4 mg, about 90±3 mg, about 90±2 mg, or about 90±1 mg (e.g., about 90 mg).

In some embodiments, the compound is administered at a dosage (e.g., a daily dosage) of about 400±200 mg, about 400±100 mg, about 400±90 mg, about 400±80 mg, about 400±70 mg, about 400±60 mg, about 400±50 mg, about 400±40 mg, about 400±30 mg, about 400±20 mg, or about 400±10 mg (e.g., about 400 mg); and
binimetinib is administered at a dosage (e.g., a daily dosage) of about 60±30 mg, about 60±20 mg, about 60±10 mg, about 60±9 mg, about 60±8 mg, about 60±7 mg, about 60±6 mg, about 60±5 mg, about 60±4 mg, about 60±3 mg, about 60±2 mg, or about 60±1 mg (e.g., about 60 mg); or about 90±50 mg, about 90±40 mg, about 90±30 mg, about 90±20 mg, about 90±10 mg, about 90±9 mg, about 90±8 mg, about 90±7 mg, about 90±6 mg, about 90±5 mg, about 90±4 mg, about 90±3 mg, about 90±2 mg, or about 90±1 mg (e.g., about 90 mg).

In some embodiments, the compound is administered at a dosage (e.g., a daily dosage) of about 600±300 mg, about 600±200 mg, about 600±100 mg, about 600±90 mg, about 600±80 mg, about 600±70 mg, about 600±60 mg, about 600±50 mg, about 600±40 mg, about 600±30 mg, about 600±20 mg, or about 600±10 mg (e.g., about 600 mg); and
binimetinib is administered at a dosage (e.g., a daily dosage) of about 60±30 mg, about 60±20 mg, about 60±10 mg, about 60±9 mg, about 60±8 mg, about 60±7 mg, about 60±6 mg, about 60±5 mg, about 60±4 mg, about 60±3 mg, about 60±2 mg, or about 60±1 mg (e.g., about 60 mg); or about 90±50 mg, about 90±40 mg, about 90±30 mg, about 90±20 mg, about 90±10 mg, about 90±9 mg, about 90±8 mg, about 90±7 mg, about 90±6 mg, about 90±5 mg, about 90±4 mg, about 90±3 mg, about 90±2 mg, or about 90±1 mg (e.g., about 90 mg).

In some embodiments, the compound is administered at a dosage (e.g., a daily dosage) of about 800±400 mg, about 800±300 mg, about 800±200 mg, about 800±100 mg, about 800±90 mg, about 800±80 mg, about 800±70 mg, about 800±60 mg, about 800±50 mg, about 800±40 mg, about 800±30 mg, about 800±20 mg, or about 800±10 mg (e.g., about 800 mg); and
binimetinib is administered at a dosage (e.g., a daily dosage) of about 60±30 mg, about 60±20 mg, about 60±10 mg, about 60±9 mg, about 60±8 mg, about 60±7 mg, about 60±6 mg, about 60±5 mg, about 60±4 mg, about 60±3 mg, about 60±2 mg, or about 60±1 mg (e.g., about 60 mg); or about 90±50 mg, about 90±40 mg, about 90±30 mg, about 90±20 mg, about 90±10 mg, about 90±9 mg, about 90±8 mg, about 90±7 mg, about 90±6 mg, about 90±5 mg, about 90±4 mg, about 90±3 mg, about 90±2 mg, or about 90±1 mg (e.g., about 90 mg).

In some embodiments, the compound is administered at a dosage (e.g., a daily dosage) of about 1000±500 mg, about 1000±400 mg, about 1000±300 mg, about 1000±200 mg, about 1000±100 mg, about 1000±90 mg, about 1000±80 mg, about 1000±70 mg, about 1000±60 mg, about 1000±50 mg, about 1000±40 mg, about 1000±30 mg, about 1000±20 mg, or about 1000±10 mg (e.g., about 1000 mg); and
binimetinib is administered at a dosage (e.g., a daily dosage) of about 60±30 mg, about 60±20 mg, about 60±10 mg, about 60±9 mg, about 60±8 mg, about 60±7 mg, about 60±6 mg, about 60±5 mg, about 60±4 mg, about 60±3 mg, about 60±2 mg, or about 60±1 mg (e.g., about 60 mg); or about 90±50 mg, about 90±40 mg, about 90±30 mg, about 90±20 mg, about 90±10 mg, about 90±9 mg, about 90±8 mg, about 90±7 mg, about 90±6 mg, about 90±5 mg, about 90±4 mg, about 90±3 mg, about 90±2 mg, or about 90±1 mg (e.g., about 90 mg).

In some embodiments, the compound is administered at a dosage (e.g., a daily dosage) of about 1200±600 mg, about 1200±500 mg, about 1200±400 mg, about 1200±300 mg, about 1200±200 mg, about 1200±100 mg, about 1200±90 mg, about 1200±80 mg, about 1200±70 mg, about 1200±60 mg, about 1200±50 mg, about 1200±40 mg, about 1200±30 mg, about 1200±20 mg, or about 1200±10 mg (e.g., about 1200 mg); and
binimetinib is administered at a dosage (e.g., a daily dosage) of about 60±30 mg, about 60±20 mg, about 60±10 mg, about 60±9 mg, about 60±8 mg, about 60±7 mg, about 60±6 mg, about 60±5 mg, about 60±4 mg, about 60±3 mg, about 60±2 mg, or about 60±1 mg (e.g., about 60 mg); or about 90±50 mg, about 90±40 mg, about 90±30 mg, about 90±20 mg, about 90±10 mg, about 90±9 mg, about 90±8 mg, about 90±7 mg, about 90±6 mg, about 90±5 mg, about 90±4 mg, about 90±3 mg, about 90±2 mg, or about 90±1 mg (e.g., about 90 mg).

### Other Additional Therapeutic Agents

In some embodiments, the one or more additional therapeutic agents comprises an agent known to modulate other pathways, or other components of the same pathway, or even overlapping sets of target enzymes.

In some embodiments, the one or more additional therapeutic agents comprise chemotherapeutic agents, therapeutic antibodies, and radiation treatment.

Many chemotherapeutics are presently known in the art. In some embodiments, the chemotherapeutic is selected from the group consisting of mitotic inhibitors, alkylating agents, anti-metabolites, intercalating antibiotics, growth factor inhibitors, cell cycle inhibitors, enzymes, topoisomerase inhibitors, biological response modifiers, anti-hormones, angiogenesis inhibitors, and anti-androgens. Non-limiting examples are chemotherapeutic agents, cytotoxic agents, and non-peptide small molecules such as Gleevec® (Imatinib Mesylate), Kyprolis® (carfilzomib), Velcade® (bortezomib), Casodex (bicalutamide), Iressa® (gefitinib), and Adriamycin as well as a host of chemotherapeutic agents. Non-limiting examples of chemotherapeutic agents include alkylating agents such as thiotepa and cyclosphosphamide (CYTOXAN™™); alkyl sulfonates such as busulfan, improsulfan and piposulfan; aziridines such as benzodopa, carboquone, meturedopa, and uredopa; ethylenimines and methylamelamines including altretamine, triethylenemelamine, trietylenephosphoramide, triethylenethiophosphaoramide and trimethylolomelamine; nitrogen mustards such as chlorambucil, chlomaphazine, cholophosphamide, estramustine, ifosfamide, mechlorethamine, mechlorethamine oxide hydrochloride, melphalan, novembichin, phenesterine, prednimustine, trofosfamide, uracil mustard; nitrosureas such as carmustine, chlorozotocin, fotemustine, lomustine, nimustine, ranimustine; antibiotics such as aclacinomysins, actinomycin, authramycin, azaserine, bleomycins, cactinomycin, calicheamicin, carabicin, carminomycin, carzinophilin, Casodex™, chromomycins, dactinomycin, daunorubicin, detorubicin, 6-diazo-5-oxo-L-norleucine, doxorubicin, epirubicin, esorubicin, idarubicin, marcellomycin, mitomycins, mycophenolic acid, nogalamycin, olivomycins, peplomycin, potfiromycin, puromycin, quelamycin, rodorubicin, streptonigrin, streptozocin, tubercidin, ubenimex, zinostatin, zorubicin; anti-metabolites such as methotrexate and 5-fluorouracil (5-FU); folic acid analogues such as denopterin, methotrexate, pteropterin, trimetrexate; purine analogs such as fludarabine, 6-mercaptopurine, thiamiprine, thioguanine; pyrimidine analogs such as ancitabine, azacitidine, 6-azauridine, carmofur, cytarabine, dideoxyuridine, doxifluridine, enocitabine, floxuridine, androgens such as calusterone, dromostanolone propionate, epitiostanol, mepitiostane, testolactone; anti-adrenals such as aminoglutethimide, mitotane, trilostane; folic acid replenisher such as frolinic acid; aceglatone; aldophosphamide glycoside; aminolevulinic acid; amsacrine; bestrabucil; bisantrene; edatraxate; defofamine; demecolcine; diaziquone; elfomithine; elliptinium acetate; etoglucid; gallium nitrate; hydroxyurea; lentinan; lonidamine; mitoguazone; mitoxantrone; mopidamol; nitracrine; pentostatin; phenamet; pirarubicin; podophyllinic acid; 2-ethylhydrazide; procarbazine; PSK; razoxane; sizofiran; spirogermanium; tenuazonic acid; triaziquone; 2,2′,2″-trichlorotriethylamine; urethan; vindesine; dacarbazine; mannomustine; mitobronitol; mitolactol; pipobroman; gacytosine; arabinoside (“Ara-C”); cyclophosphamide; thiotepa; taxanes, e.g. paclitaxel and docetaxel; retinoic acid; esperamicins; capecitabine; and pharmaceutically acceptable salts, acids or derivatives of any of the above.

Also included as suitable chemotherapeutic cell conditioners are anti-hormonal agents that act to regulate or inhibit hormone action on tumors such as anti-estrogens including for example tamoxifen, (Nolvadex™), raloxifene, aromatase inhibiting 4(5)-imidazoles, 4-hydroxytamoxifen, trioxifene, keoxifene, LY 117018, onapristone, and toremifene (Fareston); and anti-androgens such as flutamide, nilutamide, bicalutamide, leuprolide, and goserelin; chlorambucil; gemcitabine; 6-thioguanine; mercaptopurine; methotrexate; platinum analogs such as cisplatin and carboplatin; vinblastine; platinum; etoposide (VP-16); ifosfamide; mitomycin C; mitoxantrone; vincristine; vinorelbine; navelbine; novantrone; teniposide; daunomycin; aminopterin; xeloda; ibandronate; camptothecin-11 (CPT-11); topoisomerase inhibitor RFS 2000; difluoromethylornithine (DMFO).

In some embodiments, the one or more additional therapeutic agents comprise commonly prescribed anti-cancer drugs such as Herceptin®, Avastin®, Erbitux®, Rituxan®, Taxol®, Arimidex®, Taxotere®, ABVD, AVICINE, Abagovomab, Acridine carboxamide, Adecatumumab, 17-N-Allylamino-17-demethoxygeldanamycin, Alpharadin, Alvocidib, 3-Aminopyridine-2-carboxaldehyde thiosemicarbazone, Amonafide, Anthracenedione, Anti-CD22 immunotoxins, Antineoplastic, Antitumorigenic herbs, Apaziquone, Atiprimod, Azathioprine, Belotecan, Bendamustine, BIBW 2992, Biricodar, Brostallicin, Bryostatin, Buthionine sulfoximine, CBV (chemotherapy), Calyculin, cell-cycle nonspecific antineoplastic agents, Dichloroacetic acid, Discodermolide, Elsamitrucin, Enocitabine, Epothilone, Eribulin, Everolimus, Exatecan, Exisulind, Ferruginol, Forodesine, Fosfestrol, ICE chemotherapy regimen, IT-101, Imexon, Imiquimod, Indolocarbazole, Irofulven, Laniquidar, Larotaxel, Lenalidomide, Lucanthone, Lurtotecan, Mafosfamide, Mitozolomide, Nafoxidine, Nedaplatin, Olaparib, Ortataxel, PAC-1, Pawpaw, Pixantrone, Proteasome inhibitor, Rebeccamycin, Resiquimod, Rubitecan, SN-38, Salinosporamide A, Sapacitabine, Stanford V, Swainsonine, Talaporfin, Tariquidar, Tegafur-uracil, Temodar, Tesetaxel, Triplatin tetranitrate, Tris(2-chloroethyl)amine, Troxacitabine, Uramustine, Vadimezan, Vinflunine, ZD6126 or Zosuquidar.

In some embodiments, the one or more additional therapeutic agents comprise radiation therapy for inhibiting abnormal cell growth or treating the hyperproliferative disorder in the mammal. Techniques for administering radiation therapy are known in the art.

Radiation therapy can be administered through one of several methods, or a combination of methods, including without limitation external-beam therapy, internal radiation therapy, implant radiation, stereotactic radiosurgery, systemic radiation therapy, radiotherapy and permanent or temporary interstitial brachytherapy. The term “brachytherapy,” as used herein, refers to radiation therapy delivered by a spatially confined radioactive material inserted into the body at or near a tumor or other proliferative tissue disease site. The term is intended without limitation to include exposure to radioactive isotopes (e.g. At-211, I-131, I-125, Y-90, Re-186, Re-188, Sm-153, Bi-212, P-32, and radioactive isotopes of Lu). Suitable radiation sources for use as a cell conditioner of the present disclosure include both solids and liquids. By way of non-limiting example, the radiation source can be a radionuclide, such as I-125, I-131, Yb-169, Ir-192 as a solid source, I-125 as a solid source, or other radionuclides that emit photons, beta particles, gamma radiation, or other therapeutic rays. The radioactive material can also be a fluid made from any solution of radionuclide(s), e.g., a solution of I-125 or I-131, or a radioactive fluid can be produced using a slurry of a suitable fluid containing small particles of solid radionuclides, such as Au-198, Y-90. Moreover, the radionuclide(s) can be embodied in a gel or radioactive micro spheres.

In some embodiments, the one or more additional therapeutic agents comprise one or more substances selected from anti-angiogenesis agents, signal transduction inhibitors, antiproliferative agents, glycolysis inhibitors, or autophagy inhibitors.

Anti-angiogenesis agents, such as MMP-2 (matrix-metalloproteinase 2) inhibitors, MMP-9 (matrix-metalloprotienase 9) inhibitors, and COX-11 (cyclooxygenase 11) inhibitors, can be used in conjunction with a compound of the disclosure and pharmaceutical compositions described herein. Anti-angiogenesis agents include, for example, rapamycin, temsirolimus (CCI-779), everolimus (RAD001), sorafenib, sunitinib, and bevacizumab. Examples of useful COX-II inhibitors include alecoxib, valdecoxib, and rofecoxib. Examples of useful matrix metalloproteinase inhibitors are described in WO 96/33172 WO 96/27583 European Patent Publication EP0818442, European Patent Publication EP1004578, WO 98/07697, WO 98/03516, WO 98/34918, WO 98/34915, WO 98/33768, WO 98/30566, European Patent Publication 606046, European Patent Publication 931 788, WO 90/05719, WO 99/52910, WO 99/52889, WO 99/29667, WO1999007675, European Patent Publication EP1786785, European Patent Publication No. EP1181017, United States Publication No. US20090012085, United States Publication U.S. Pat. No. 5,863,949, United States Publication U.S. Pat. No. 5,861,510, and European Patent Publication EP0780386, all of which are incorporated herein in their entireties by reference. Preferred MMP-2 and MMP-9 inhibitors are those that have little or no activity inhibiting MMP-1. More preferred, are those that selectively inhibit MMP-2 and/or AMP-9 relative to the other matrix-metalloproteinases (i. e., MAP-1, MMP-3, MMP-4, MMP-5, MMP-6, MMP-7, MMP-8, MMP-10, MMP-11, MMP-12, andMMP-13). Some specific examples of MMP inhibitors useful in the disclosure are AG-3340, RO 32-3555, and RS 13-0830.

In some embodiments, the one or more additional therapeutic agents comprise anti-neoplastic agents, such as acemannan, aclarubicin, aldesleukin, alemtuzumab, alitretinoin, altretamine, amifostine, aminolevulinic acid, amrubicin, amsacrine, anagrelide, anastrozole, ANCER, ancestim, ARGLABIN, arsenic trioxide, BAM 002 (Novelos), bexarotene, bicalutamide, broxuridine, capecitabine, celmoleukin, cetrorelix, cladribine, clotrimazole, cytarabine ocfosfate, DA 3030 (Dong-A), daclizumab, denileukin diftitox, deslorelin, dexrazoxane, dilazep, docetaxel, docosanol, doxercalciferol, doxifluridine, doxorubicin, bromocriptine, carmustine, cytarabine, fluorouracil, HIT diclofenac, interferon alfa, daunorubicin, doxorubicin, tretinoin, edelfosine, edrecolomab, eflomithine, emitefur, epirubicin, epoetin beta, etoposide phosphate, exemestane, exisulind, fadrozole, filgrastim, finasteride, fludarabine phosphate, formestane, fotemustine, gallium nitrate, gemcitabine, gemtuzumab zogamicin, gimeracil/oteracil/tegafur combination, glycopine, goserelin, heptaplatin, human chorionic gonadotropin, human fetal alpha fetoprotein, ibandronic acid, idarubicin, (imiquimod, interferon alfa, interferon alfa, natural, interferon alfa-2, interferon alfa-2a, interferon alfa-2b, interferon alfa-N1, interferon alfa-n3, interferon alfacon-1, interferon alpha, natural, interferon beta, interferon beta-1a, interferon beta-1b, interferon gamma, natural interferon gamma-1a, interferon gamma-1b, interleukin-1 beta, iobenguane, irinotecan, irsogladine, lanreotide, LC 9018 (Yakult), leflunomide, lenograstim, lentinan sulfate, letrozole, leukocyte alpha interferon, leuprorelin, levamisole+fluorouracil, liarozole, lobaplatin, lonidamine, lovastatin, masoprocol, melarsoprol, metoclopramide, mifepristone, miltefosine, mirimostim, mismatched double stranded RNA, mitoguazone, mitolactol, mitoxantrone, molgramostim, nafarelin, naloxone+pentazocine, nartograstim, nedaplatin, nilutamide, noscapine, novel erythropoiesis stimulating protein, NSC 631570 octreotide, oprelvekin, osaterone, oxaliplatin, paclitaxel, pamidronic acid, pegaspargase, peginterferon alfa-2b, pentosan polysulfate sodium, pentostatin, picibanil, pirarubicin, rabbit antithymocyte polyclonal antibody, polyethylene glycol interferon alfa-2a, porfimer sodium, raloxifene, raltitrexed, rasburiembodiment, rhenium Re 186 etidronate, RII retinamide, rituximab, romurtide, samarium (153 Sm) lexidronam, sargramostim, sizofiran, sobuzoxane, sonermin, strontium-89 chloride, suramin, tasonermin, tazarotene, tegafur, temoporfin, temozolomide, teniposide, tetrachlorodecaoxide, thalidomide, thymalfasin, thyrotropin alfa, topotecan, toremifene, tositumomab-iodine 131, trastuzumab, treosulfan, tretinoin, trilostane, trimetrexate, triptorelin, tumor necrosis factor alpha, natural, ubenimex, bladder cancer vaccine, Maruyama vaccine, melanoma lysate vaccine, valrubicin, verteporfin, vinorelbine, VIRULIZIN, zinostatin stimalamer, or zoledronic acid; abarelix; AE 941 (Aeterna), ambamustine, antisense oligonucleotide, bcl-2 (Genta), APC 8015 (Dendreon), cetuximab, decitabine, dexaminoglutethimide, diaziquone, EL 532 (Elan), EM 800 (Endorecherche), eniluracil, etanidazole, fenretinide, filgrastim SD01 (Amgen), fulvestrant, galocitabine, gastrin 17 immunogen, HLA-B7 gene therapy (Vical), granulocyte macrophage colony stimulating factor, histamine dihydrochloride, ibritumomab tiuxetan, ilomastat, IM 862 (Cytran), interleukin-2, iproxifene, LDI200 (Milkhaus), leridistim, lintuzumab, CA 125 MAb (Biomira), cancer MAb (Japan Pharmaceutical Development), HER-2 and Fc MAb (Medarex), idiotypic 105AD7 MAb (CRC Technology), idiotypic CEA MAb (Trilex), LYM-1-iodine 131 MAb (Techniclone), polymorphic epithelial mucin-yttrium 90 MAb (Antisoma), marimastat, menogaril, mitumomab, motexafin gadolinium, MX 6 (Galderma), nelarabine, nolatrexed, P 30 protein, pegvisomant, pemetrexed, porfiromycin, prinomastat, RL 0903 (Shire), rubitecan, satraplatin, sodium phenylacetate, sparfosic acid, SRL 172 (SR Pharma), SU 5416 (SUGEN), TA 077 (Tanabe), tetrathiomolybdate, thaliblastine, thrombopoietin, tin ethyl etiopurpurin, tirapazamine, cancer vaccine (Biomira), melanoma vaccine (New York University), melanoma vaccine (Sloan Kettering Institute), melanoma oncolysate vaccine (New York Medical College), viral melanoma cell lysates vaccine (Royal Newcastle Hospital), or valspodar.

In some embodiments, the one or more additional therapeutic agents comprise VEGFR inhibitors, including compounds described in the following patents and patent applications: U.S. Pat. No. 6,258,812, US 2003/0105091, WO/2001/037820, U.S. Pat. No. 6,235,764, WO/2001/032651, U.S. Pat. Nos. 6,630,500, 6,515,004, 6,713,485, 5,521,184, 5,770,599, and 5,747,498, WO/2002/068406, WO/2002/066470, WO/2002/055501, WO/2004/005279, WO/2004/007481, WO/2004/007458, WO/2004/09784, WO/2002/059110, WO/1999/045009, WO/2000/059509, WO/1999/061422, U.S. Pat. No. 5,990,141, WO/2000/012089, and WO/2000/002871.

In some embodiments, the one or more additional therapeutic agents comprise at least one anti-angiogenic agent. Agents are inclusive of, but not limited to, in vitro synthetically prepared chemical compositions, antibodies, antigen binding regions, radionuclides, and combinations and conjugates thereof. An agent can be an agonist, antagonist, allosteric modulator, toxin or, more generally, may act to inhibit or stimulate its target (e.g., receptor or enzyme activation or inhibition), and thereby promote cell death or arrest cell growth.

Exemplary anti-angiogenic agents include ERBITUX™ (IMC-C225), KDR (kinase domain receptor) inhibitory agents (e.g., antibodies and antigen binding regions that specifically bind to the kinase domain receptor), anti-VEGF agents (e.g., antibodies or antigen binding regions that specifically bind VEGF, or soluble VEGF receptors or a ligand binding region thereof) such as AVASTIN™ or VEGF-TRAP™, and anti-VEGF receptor agents (e.g., antibodies or antigen binding regions that specifically bind thereto), EGFR inhibitory agents (e.g., antibodies or antigen binding regions that specifically bind thereto) such as Vectibix (panitumumab), IRESSA™ (gefitinib), TARCEVA™ (erlotinib), anti-Ang1 and anti-Ang2 agents (e.g., antibodies or antigen binding regions specifically binding thereto or to their receptors, e.g., Tie2/Tek), and anti-Tie2 kinase inhibitory agents (e.g., antibodies or antigen binding regions that specifically bind thereto). The pharmaceutical compositions of the present invention can also include one or more agents (e.g., antibodies, antigen binding regions, or soluble receptors) that specifically bind and inhibit the activity of growth factors, such as antagonists of hepatocyte growth factor (HGF, also known as Scatter Factor), and antibodies or antigen binding regions that specifically bind its receptor “c-met”.

Other anti-angiogenic agents include Campath, IL-8, B-FGF, Tek antagonists (Ceretti et al., U.S. Publication No. 2003/0162712; U.S. Pat. No. 6,413,932), anti-TWEAK agents (e.g., specifically binding antibodies or antigen binding regions, or soluble TWEAK receptor antagonists; see, Wiley, U.S. Pat. No. 6,727,225), ADAM distintegrin domain to antagonize the binding of integrin to its ligands (Fanslow et al., U.S. Publication No. 2002/0042368), specifically binding anti-eph receptor and/or anti-ephrin antibodies or antigen binding regions (U.S. Pat. Nos. 5,981,245; 5,728,813; 5,969,110; 6,596,852; 6,232,447; 6,057,124 and patent family members thereof), and anti-PDGF-BB antagonists (e.g., specifically binding antibodies or antigen binding regions) as well as antibodies or antigen binding regions specifically binding to PDGF-BB ligands, and PDGFR kinase inhibitory agents (e.g., antibodies or antigen binding regions that specifically bind thereto).

Additional anti-angiogenic/anti-tumor agents include: SD-7784 (Pfizer, USA); cilengitide. (Merck KGaA, Germany, EPO 770622); pegaptanib octasodium, (Gilead Sciences, USA); Alphastatin, (BioActa, UK); M-PGA, (Celgene, USA, U.S. Pat. No. 5,712,291); ilomastat, (Arriva, USA, U.S. Pat. No. 5,892,112); emaxanib, (Pfizer, USA, U.S. Pat. No. 5,792,783); vatalanib, (Novartis, Switzerland); 2-methoxyestradiol, (EntreMed, USA); TLC ELL-12, (Elan, Ireland); anecortave acetate, (Alcon, USA); alpha-D148 Mab, (Amgen, USA); CEP-7055, (Cephalon, USA); anti-Vn Mab, (Crucell, Netherlands) DAC:antiangiogenic, (ConjuChem, Canada); Angiocidin, (InKine Pharmaceutical, USA); KM-2550, (Kyowa Hakko, Japan); SU-0879, (Pfizer, USA); CGP-79787, (Novartis, Switzerland, EP 970070); ARGENT technology, (Ariad, USA); YIGSR-Stealth, (Johnson & Johnson, USA); fibrinogen-E fragment, (BioActa, UK); angiogenesis inhibitor, (Trigen, UK); TBC-1635, (Encysive Pharmaceuticals, USA); SC-236, (Pfizer, USA); ABT-567, (Abbott, USA); Metastatin, (EntreMed, USA); angiogenesis inhibitor, (Tripep, Sweden); maspin, (Sosei, Japan); 2-methoxyestradiol, (Oncology Sciences Corporation, USA); ER-68203-00, (IVAX, USA); Benefin, (Lane Labs, USA); Tz-93, (Tsumura, Japan); TAN-1120, (Takeda, Japan); FR-111142, (Fujisawa, Japan, JP 02233610); platelet factor 4, (RepliGen, USA, EP 407122); vascular endothelial growth factor antagonist, (Borean, Denmark); bevacizumab (pINN), (Genentech, USA); angiogenesis inhibitors, (SUGEN, USA); XL 784, (Exelixis, USA); XL 647, (Exelixis, USA); MAb, alpha5beta3 integrin, second generation, (Applied Molecular Evolution, USA and MedImmune, USA); gene therapy, retinopathy, (Oxford BioMedica, UK); enzastaurin hydrochloride (USAN), (Lilly, USA); CEP 7055, (Cephalon, USA and Sanofi-Synthelabo, France); BC 1, (Genoa Institute of Cancer Research, Italy); angiogenesis inhibitor, (Alchemia, Australia); VEGF antagonist, (Regeneron, USA); rBPI 21 and BPI-derived antiangiogenic, (XOMA, USA); PI 88, (Progen, Australia); cilengitide (pINN), (Merck KGaA, German; Munich Technical University, Germany, Scripps Clinic and Research Foundation, USA); cetuximab (INN), (Aventis, France); AVE 8062, (Ajinomoto, Japan); AS 1404, (Cancer Research Laboratory, New Zealand); SG 292, (Telios, USA); Endostatin, (Boston Childrens Hospital, USA); ATN 161, (Attenuon, USA); ANGIOSTATIN, (Boston Childrens Hospital, USA); 2-methoxyestradiol, (Boston Childrens Hospital, USA); ZD 6474, (AstraZeneca, UK); ZD 6126, (Angiogene Pharmaceuticals, UK); PPI 2458, (Praecis, USA); AZD 9935, (AstraZeneca, UK); AZD 2171, (AstraZeneca, UK); vatalanib (pINN), (Novartis, Switzerland and Schering AG, Germany); tissue factor pathway inhibitors, (EntreMed, USA); pegaptanib (Pinn), (Gilead Sciences, USA); xanthorrhizol, (Yonsei University, South Korea); vaccine, gene-based, VEGF-2, (Scripps Clinic and Research Foundation, USA); SPV5.2, (Supratek, Canada); SDX 103, (University of California at San Diego, USA); PX 478, (ProlX, USA); METASTATIN, (EntreMed, USA); troponin I, (Harvard University, USA); SU 6668, (SUGEN, USA); OXI 4503, (OXiGENE, USA); o-guanidines, (Dimensional Pharmaceuticals, USA); motuporamine C, (British Columbia University, Canada); CDP 791, (Celltech Group, UK); atiprimod (pINN), (GlaxoSmithKline, UK); E 7820, (Eisai, Japan); CYC 381, (Harvard University, USA); AE 941, (Aeterna, Canada); vaccine, angiogenesis, (EntreMed, USA); urokinase plasminogen activator inhibitor, (Dendreon, USA); oglufanide (pINN), (Melmotte, USA); HIF-lalfa inhibitors, (Xenova, UK); CEP 5214, (Cephalon, USA); BAY RES 2622, (Bayer, Germany); Angiocidin, (InKine, USA); A6, (Angstrom, USA); KR 31372, (Korea Research Institute of Chemical Technology, South Korea); GW 2286, (GlaxoSmithKline, UK); EHT 0101, (ExonHit, France); CP 868596, (Pfizer, USA); CP 564959, (OSI, USA); CP 547632, (Pfizer, USA); 786034, (GlaxoSmithKline, UK); KRN 633, (Kirin Brewery, Japan); drug delivery system, intraocular, 2-methoxyestradiol, (EntreMed, USA); anginex, (Maastricht University, Netherlands, and Minnesota University, USA); ABT 510, (Abbott, USA); AAL 993, (Novartis, Switzerland); VEGI, (ProteomTech, USA); tumor necrosis factor-alpha inhibitors, (National Institute on Aging, USA); SU 11248, (Pfizer, USA and SUGEN USA); ABT 518, (Abbott, USA); YH16, (Yantai Rongchang, China); S-3APG, (Boston Childrens Hospital, USA and EntreMed, USA); MAb, KDR, (ImClone Systems, USA); MAb, alpha5 betal, (Protein Design, USA); KDR kinase inhibitor, (Celltech Group, UK, and Johnson & Johnson, USA); GFB 116, (South Florida University, USA and Yale University, USA); CS 706, (Sankyo, Japan); combretastatin A4 prodrug, (Arizona State University, USA); chondroitinase AC, (IBEX, Canada); BAY RES 2690, (Bayer, Germany); AGM 1470, (Harvard University, USA, Takeda, Japan, and TAP, USA); AG 13925, (Agouron, USA); Tetrathiomolybdate, (University of Michigan, USA); GCS 100, (Wayne State University, USA) CV 247, (Ivy Medical, UK); CKD 732, (Chong Kun Dang, South Korea); MAb, vascular endothelium growth factor, (Xenova, UK); irsogladine (INN), (Nippon Shinyaku, Japan); RG 13577, (Aventis, France); WX 360, (Wilex, Germany); squalamine (pINN), (Genaera, USA); RPI 4610, (Sirna, USA); cancer therapy, (Marinova, Australia); heparanase inhibitors, (InSight, Israel); KL 3106, (Kolon, South Korea); Honokiol, (Emory University, USA); ZK CDK, (Schering AG, Germany); ZK Angio, (Schering AG, Germany); ZK 229561, (Novartis, Switzerland, and Schering AG, Germany); XMP 300, (XOMA, USA); VGA 1102, (Taisho, Japan); VEGF receptor modulators, (Pharmacopeia, USA); VE-cadherin-2 antagonists, (ImClone Systems, USA); Vasostatin, (National Institutes of Health, USA); vaccine, Flk-1, (ImClone Systems, USA); TZ 93, (Tsumura, Japan); TumStatin, (Beth Israel Hospital, USA); truncated soluble FLT 1 (vascular endothelial growth factor receptor 1), (Merck & Co, USA); Tie-2 ligands, (Regeneron, USA); and, thrombospondin 1 inhibitor, (Allegheny Health, Education and Research Foundation, USA).

Autophagy inhibitors include, but are not limited to chloroquine, 3-methyladenine, hydroxychloroquine (Plaquenil™), bafilomycin A1, 5-amino-4-imidazole carboxamide riboside (AICAR), okadaic acid, autophagy-suppressive algal toxins which inhibit protein phosphatases of type 2A or type 1, analogues of cAMP, and drugs which elevate cAMP levels such as adenosine, LY204002, N6-mercaptopurine riboside, and vinblastine. In addition, antisense or siRNA that inhibits expression of proteins including but not limited to ATG5 (which are implicated in autophagy), may also be used.

In some embodiments, the one or more additional therapeutic agents comprise epoetin alfa; darbepoetin alfa; panitumumab; pegfilgrastim; palifermin; filgrastim; denosumab; ancestim; AMG 102; AMG 386; AMG 479; AMG 655; AMG 745; AMG 951; AMG 706, or a pharmaceutically acceptable salt thereof.

In some embodiments, the one or more additional therapeutic agents comprise a chemotherapeutic agent. Suitable chemotherapeutic agents may include, natural products such as vinca alkaloids (e.g., vinblastine, vincristine, and vinorelbine), paclitaxel, epidipodophyllotoxins (e.g., etoposide and teniposide), antibiotics (e.g., dactinomycin (actinomycin D), daunorubicin, doxorubicin, and idarubicin), anthracyclines, mitoxantrone, bleomycins, plicamycin (mithramycin), mitomycin, enzymes (e.g., L-asparaginase which systemically metabolizes L-asparagine and deprives cells which do not have the capacity to synthesize their own asparagine), antiplatelet agents, antiproliferative/antimitotic alkylating agents such as nitrogen mustards (e.g., mechlorethamine, cyclophosphamide and analogs, melphalan, and chlorambucil), ethylenimines and methylmelamines (e.g., hexaamethylmelaamine and thiotepa), CDK inhibitors (e.g., seliciclib, UCN-01, P1446A-05, PD-0332991, dinaciclib, P27-00, AT-7519, RGB286638, and SCH727965), alkyl sulfonates (e.g., busulfan), nitrosoureas (e.g., carmustine (BCNU) and analogs, and streptozocin), trazenes-dacarbazinine (DTIC), antiproliferative/antimitotic antimetabolites such as folic acid analogs (e.g., methotrexate), pyrimidine analogs (e.g., fluorouracil, floxuridine, and cytarabine), purine analogs and related inhibitors (e.g., mercaptopurine, thioguanine, pentostatin and 2-chlorodeoxyadenosine), aromatase inhibitors (e.g., anastrozole, exemestane, and letrozole), and platinum coordination complexes (e.g., cisplatin and carboplatin), procarbazine, hydroxyurea, mitotane, aminoglutethimide, histone deacetylase (HDAC) inhibitors (e.g., trichostatin, sodium butyrate, apicidan, suberoyl anilide hydroamic acid, vorinostat, LBH 589, romidepsin, ACY-1215, and panobinostat), mTor inhibitors (e.g., temsirolimus, everolimus, ridaforolimus, and sirolimus), KSP(Eg5) inhibitors (e.g., Array 520), DNA binding agents (e.g., Zalypsis), PI3K delta inhibitor (e.g., GS-1101 and TGR-1202), PI3K delta and gamma inhibitor (e.g., CAL-130), multi-kinase inhibitor (e.g., TG02 and sorafenib), hormones (e.g., estrogen) and hormone agonists such as leutinizing hormone releasing hormone (LHRH) agonists (e.g., goserelin, leuprolide and triptorelin), BAFF-neutralizing antibody (e.g., LY2127399), IKK inhibitors, p38MAPK inhibitors, anti-IL-6 (e.g., CNT0328), telomerase inhibitors (e.g., GRN 163L), aurora kinase inhibitors (e.g., MLN8237), cell surface monoclonal antibodies (e.g., anti-CD38 (HUMAX-CD38), anti-CS1 (e.g., elotuzumab), HSP90 inhibitors (e.g., 17 AAG and KOS 953), PI3K/Akt inhibitors (e.g., perifosine), Akt inhibitor (e.g., GSK-2141795), PKC inhibitors (e.g., enzastaurin), FTIs (e.g., Zamestra™), anti-CD138 (e.g., BT062), Torcl/2 specific kinase inhibitor (e.g., INK128), kinase inhibitor (e.g., GS-1101), ER/UPR targeting agent (e.g., MKC-3946), cFMS inhibitor (e.g., ARRY-382), JAK1/2 inhibitor (e.g., CYT387), PARP inhibitor (e.g., olaparib and veliparib (ABT-888)), and BCL-2 antagonist. Other chemotherapeutic agents may include mechlorethamine, camptothecin, ifosfamide, tamoxifen, raloxifene, gemcitabine, navelbine, sorafenib, or any analog or derivative variant of the foregoing.

In some embodiments, the one or more additional therapeutic agents comprise radiation therapy, hormone therapy, surgery and immunotherapy, which therapies are well known to those skilled in the art.

In some embodiments, the one or more additional therapeutic agents comprise a steroid. Suitable steroids may include, but are not limited to, 21-acetoxypregnenolone, alclometasone, algestone, amcinonide, beclomethasone, betamethasone, budesonide, chloroprednisone, clobetasol, clocortolone, cloprednol, corticosterone, cortisone, cortivazol, deflazacort, desonide, desoximetasone, dexamethasone, diflorasone, diflucortolone, difuprednate, enoxolone, fluazacort, flucloronide, flumethasone, flunisolide, fluocinolone acetonide, fluocinonide, fluocortin butyl, fluocortolone, fluorometholone, fluperolone acetate, fluprednidene acetate, fluprednisolone, flurandrenolide, fluticasone propionate, formocortal, halcinonide, halobetasol propionate, halometasone, hydrocortisone, loteprednol etabonate, mazipredone, medrysone, meprednisone, methylprednisolone, mometasone furoate, paramethasone, prednicarbate, prednisolone, prednisolone 25-diethylaminoacetate, prednisolone sodium phosphate, prednisone, prednival, prednylidene, rimexolone, tixocortol, triamcinolone, triamcinolone acetonide, triamcinolone benetonide, triamcinolone hexacetonide, and salts and/or derivatives thereof. In a particular embodiment, the compounds of the present invention can also be used in combination with additional pharmaceutically active agents that treat nausea. Examples of agents that can be used to treat nausea include: dronabinol; granisetron; metoclopramide; ondansetron; and prochlorperazine; or a pharmaceutically acceptable salt thereof.

In some embodiments, the one or more additional therapeutic agents comprise a compound that disrupts or inhibits RAS-RAF-ERK or PI3K-AKT-TOR signaling pathways. In other such combinations, the additional pharmaceutically active compound is a PD-1 and PD-L1 antagonist. The compounds or pharmaceutical compositions of the disclosure can also be used in combination with an amount of one or more substances selected from EGFR inhibitors, MEK inhibitors, PI3K inhibitors, AKT inhibitors, TOR inhibitors, Mcl-1 inhibitors, BCL-2 inhibitors, SHP2 inhibitors, proteasome inhibitors, and immune therapies, including monoclonal antibodies, immunomodulatory imides (IMiDs), anti-PD-1, anti-PDL-1, anti-CTLA4, anti-LAG1, and anti-OX40 agents, GITR agonists, CAR-T cells, and BiTEs.

EGFR inhibitors include, but are not limited to, small molecule antagonists, antibody inhibitors, or specific antisense nucleotide or siRNA. Useful antibody inhibitors of EGFR include cetuximab (Erbitux), panitumumab (Vectibix), zalutumumab, nimotuzumab, and matuzumab. Small molecule antagonists of EGFR include gefitinib, erlotinib (Tarceva), and most recently, lapatinib (TykerB). See e.g., Yan L, et. al., Pharmacogenetics and Pharmacogenomics In Oncology Therapeutic Antibody Development, BioTechniques 2005; 39(4): 565-8, and Paez J G, et. al., EGFR Mutations In Lung Cancer: Correlation With Clinical Response To Gefitinib Therapy, Science 2004; 304(5676): 1497-500.

Non-limiting examples of small molecule EGFR inhibitors include any of the EGFR inhibitors described in the following patent publications, and all pharmaceutically acceptable salts of said EGFR inhibitors: European Patent Application EP 520722, published Dec. 30, 1992; European Patent Application EP 566226, published Oct. 20, 1993; PCT International Publication WO 96/33980, published Oct. 31, 1996; U.S. Pat. No. 5,747,498, issued May 5, 1998; PCT International Publication WO 96/30347, published Oct. 3, 1996; European Patent Application EP 787772, published Aug. 6, 1997; PCT International Publication WO 97/30034, published Aug. 21, 1997; PCT International Publication WO 97/30044, published Aug. 21, 1997; PCT International Publication WO 97/38994, published Oct. 23, 1997; PCT International Publication WO 97/49688, published Dec. 31, 1997; European Patent Application EP 837063, published Apr. 22, 1998; PCT International Publication WO 98/02434, published Jan. 22, 1998; PCT International Publication WO 97/38983, published Oct. 23, 1997; PCT International Publication WO 95/19774, published Jul. 27, 1995; PCT International Publication WO 95/19970, published Jul. 27, 1995; PCT International Publication WO 97/13771, published Apr. 17, 1997; PCT International Publication WO 98/02437, published Jan. 22, 1998; PCT International Publication WO 98/02438, published Jan. 22, 1998; PCT International Publication WO 97/32881, published Sep. 12, 1997; German Application DE 19629652, published Jan. 29, 1998; PCT International Publication WO 98/33798, published Aug. 6, 1998; PCT International Publication WO 97/32880, published Sep. 12, 1997; PCT International Publication WO 97/32880 published Sep. 12, 1997; European Patent Application EP 682027, published Nov. 15, 1995; PCT International Publication WO 97/02266, published Jan. 23, 197; PCT International Publication WO 97/27199, published Jul. 31, 1997; PCT International Publication WO 98/07726, published Feb. 26, 1998; PCT International Publication WO 97/34895, published Sep. 25, 1997; PCT International Publication WO 96/31510′, published Oct. 10, 1996; PCT International Publication WO 98/14449, published Apr. 9, 1998; PCT International Publication WO 98/14450, published Apr. 9, 1998; PCT International Publication WO 98/14451, published Apr. 9, 1998; PCT International Publication WO 95/09847, published Apr. 13, 1995; PCT International Publication WO 97/19065, published May 29, 1997; PCT International Publication WO 98/17662, published Apr. 30, 1998; U.S. Pat. No. 5,789,427, issued Aug. 4, 1998; U.S. Pat. No. 5,650,415, issued Jul. 22, 1997; U.S. Pat. No. 5,656,643, issued Aug. 12, 1997; PCT International Publication WO 99/35146, published Jul. 15, 1999; PCT International Publication WO 99/35132, published Jul. 15, 1999; PCT International Publication WO 99/07701, published Feb. 18, 1999; and PCT International Publication WO 92/20642 published Nov. 26, 1992. Additional non-limiting examples of small molecule EGFR inhibitors include any of the EGFR inhibitors described in Traxler, P., 1998, Exp. Opin. Ther. Patents 8(12): 1599-1625.

Antibody-based EGFR inhibitors include any anti-EGFR antibody or antibody fragment that can partially or completely block EGFR activation by its natural ligand. Non-limiting examples of antibody-based EGFR inhibitors include those described in Modjtahedi, H., et al., 1993, Br. J. Cancer 67:247-253; Teramoto, T., et al., 1996, Cancer 77:639-645; Goldstein et al., 1995, Clin. Cancer Res. 1:1311-1318; Huang, S. M., et al., 1999, Cancer Res. 15:59(8):1935-40; and Yang, X., et al., 1999, Cancer Res. 59:1236-1243. Thus, the EGFR inhibitor can be monoclonal antibody Mab E7.6.3 (Yang, 1999 supra), or Mab C225 (ATCC Accession No. HB-8508), or an antibody or antibody fragment having the binding specificity thereof.

MEK inhibitors include, but are not limited to, CI-1040, AZD6244, PD318088, PD98059, PD334581, RDEA119, ARRY-142886, ARRY-438162, and PD-325901.

PI3K inhibitors include, but are not limited to, wortmannin, 17-hydroxywortmannin analogs described in WO 06/044453, 4-[2-(1H-Indazol-4-yl)-6-[[4-(methylsulfonyl)piperazin-1-yl]methyl]thieno[3,2-d]pyrimidin-4-yl]morpholine (also known as GDC 0941 and described in PCT Publication Nos. WO 09/036,082 and WO 09/055,730), 2-Methyl-2-[4-[3-methyl-2-oxo-8-(quinolin-3-yl)-2,3-dihydroimidazo[4,5-c]quinolin-1-yl]phenyl]propionitrile (also known as BEZ 235 or NVP-BEZ 235, and described in PCT Publication No. WO 06/122806), (S)-1-(4-((2-(2-aminopyrimidin-5-yl)-7-methyl-4-morpholinothieno[3,2-d]pyrimidin-6-yl)methyl)piperazin-1-yl)-2-hydroxypropan-1-one (described in PCT Publication No. WO 2008/070740), LY294002 (2-(4-Morpholinyl)-8-phenyl-4H-1-benzopyran-4-one available from Axon Medchem), PI 103 hydrochloride (3-[4-(4-morpholinylpyrido-[3′,2′:4,5]furo[3,2-d]pyrimidin-2-yl]phenol hydrochloride available from Axon Medchem), PIK 75 (N′-[(1E)-(6-bromoimidazo[1,2-a]pyridin-3-yl)methylene]-N,2-dimethyl-5-nitrobenzenesulfono-hydrazide hydrochloride available from Axon Medchem), PIK 90 (N-(7,8-dimethoxy-2,3-dihydroimidazo[1,2-c]quinazolin-5-yl)-nicotinamide available from Axon Medchem), GDC-0941 bismesylate (2-(1H-Indazol-4-yl)-6-(4-methanesulfonyl-piperazin-1-ylmethyl)-4-morpholin-4-yl-thieno[3,2-d]pyrimidine bismesylate available from Axon Medchem), AS-252424 (5-[1-[5-(4-Fluoro-2-hydroxy-phenyl)-furan-2-yl]-meth-(Z)-ylidene]-thiazolidine-2,4-dione available from Axon Medchem), and TGX-221 (7-Methyl-2-(4-morpholinyl)-9-[1-(phenylamino)ethyl]-4H-pyrido-[1,2-a]pyrimidin-4-one available from Axon Medchem), XL-765, and XL-147. Other PI3K inhibitors include demethoxyviridin, perifosine, CAL101, PX-866, BEZ235, SF1126, INK1117, IPI-145, BKM120, XL147, XL765, Palomid 529, GSK1059615, ZSTK474, PWT33597, IC87114, TG100-115, CAL263, PI-103, GNE-477, CUDC-907, and AEZS-136.

AKT inhibitors include, but are not limited to, Akt-1-1 (inhibits Akt1) (Barnett et al. (2005) Biochem. J., 385 (Pt. 2), 399-408); Akt-1-1,2 (inhibits Akl and 2) (Barnett et al. (2005) Biochem. J 385 (Pt. 2), 399-408); API-59CJ-Ome (e.g., Jin et al. (2004) Br. J. Cancer 91, 1808-12); 1-H-imidazo[4,5-c]pyridinyl compounds (e.g., WO05011700); indole-3-carbinol and derivatives thereof (e.g., U.S. Pat. No. 6,656,963; Sarkar and Li (2004) J Nutr. 134(12 Suppl), 3493S-3498S); perifosine (e.g., interferes with Akt membrane localization; Dasmahapatra et al. (2004) Clin. Cancer Res. 10(15), 5242-52, 2004); phosphatidylinositol ether lipid analogues (e.g., Gills and Dennis (2004) Expert. Opin. Investig. Drugs 13, 787-97); and triciribine (TCN or API-2 or NCI identifier: NSC 154020; Yang et al. (2004) Cancer Res. 64, 4394-9).

TOR inhibitors include, but are not limited to, inhibitors include AP-23573, CCI-779, everolimus, RAD-001, rapamycin, temsirolimus, ATP-competitive TORC1/TORC2 inhibitors, including PI-103, PP242, PP30 and Torin 1. Other TOR inhibitors in FKBP12 enhancer; rapamycins and derivatives thereof, including: CCI-779 (temsirolimus), RAD001 (Everolimus; WO 9409010) and AP23573; rapalogs, e.g. as disclosed in WO 98/02441 and WO 01/14387, e.g. AP23573, AP23464, or AP23841; 40-(2-hydroxyethyl)rapamycin, 40-[3-hydroxy(hydroxymethyl)methylpropanoate]-rapamycin (also called CC1779), 40-epi-(tetrazolyt)-rapamycin (also called ABT578), 32-deoxorapamycin, 16-pentynyloxy-32(S)-dihydrorapanycin, and other derivatives disclosed in WO 05005434; derivatives disclosed in U.S. Pat. No. 5,258,389, WO 94/090101, WO 92/05179, U.S. Pat. Nos. 5,118,677, 5,118,678, 5,100,883, 5,151,413, 5,120,842, WO 93/111130, WO 94/02136, WO 94/02485, WO 95/14023, WO 94/02136, WO 95/16691, WO 96/41807, WO 96/41807 and U.S. Pat. No. 5,256,790; phosphorus-containing rapamycin derivatives (e.g., WO 05016252); 4H-1-benzopyran-4-one derivatives (e.g., U.S. Provisional Application No. 60/528,340).

MCL-1 inhibitors include, but are not limited to, AMG-176, MIK665, and S63845. The myeloid cell leukemia-1 (MCL-1) protein is one of the key anti-apoptotic members of the B-cell lymphoma-2 (BCL-2) protein family. Over-expression of MCL-1 has been closely related to tumor progression as well as to resistance, not only to traditional chemotherapies but also to targeted therapeutics including BCL-2 inhibitors such as ABT-263.

SHP inhibitors include, but are not limited to, SHP099.

Proteasome inhibitors include, but are not limited to, Kyprolis® (carfilzomib), Velcade® (bortezomib), and oprozomib.

Immune therapies include, but are not limited to, anti-PD-1 agents, anti-PDL-1 agents, anti-CTLA-4 agents, anti-LAG1 agents, and anti-OX40 agents.

Monoclonal antibodies include, but are not limited to, Darzalex® (daratumumab), Herceptin® (trastuzumab), Avastin® (bevacizumab), Rituxan® (rituximab), and Lucentis® (ranibizumab).

Fusion proteins include, but are not limited to, Eylea® (aflibercept).

Immunomodulatory agents (IMiDs) are a class of immunomodulatory drugs (drugs that adjust immune responses) containing an imide group. The IMiD class includes thalidomide and its analogues (lenalidomide, pomalidomide, and apremilast).

Exemplary anti-PD-1 antibodies and methods for their use are described by Goldberg et al., Blood 110(1):186-192 (2007), Thompson et al., Clin. Cancer Res. 13(6):1757-1761 (2007), and Korman et al., International Application No. PCT/JP2006/309606 (publication no. WO 2006/121168 A1), each of which are expressly incorporated by reference herein. include: Yervoy™ (ipilimumab) or Tremelimumab (to CTLA-4), galiximab (to B7.1), BMS-936558 (to PD-1), MK-3475 (to PD-1), AMP224 (to B7DC), BMS-936559 (to B7-H1), MPDL3280A (to B7-H1), MEDI-570 (to ICOS), AMG557 (to B7H2), MGA271 (to B7H3), IMP321 (to LAG-3), BMS-663513 (to CD137), PF-05082566 (to CD137), CDX-1127 (to CD27), anti-OX40 (Providence Health Services), huMAbOX40L (to OX40L), Atacicept (to TACI), CP-870893 (to CD40), Lucatumumab (to CD40), Dacetuzumab (to CD40), Muromonab-CD3 (to CD3), Ipilumumab (to CTLA-4). Immune therapies also include genetically engineered T-cells (e.g., CAR-T cells) and bispecific antibodies (e.g., BiTEs).

In some embodiments, the one or more additional therapeutic agents comprise an anti-PD-1 antibody.

In some embodiments, the anti-PD-1 antibody (or antigen binding antibody fragment thereof) comprises 1, 2, 3, 4, 5, or all 6 the complementarity determining region (CDR) amino acid sequences of SEQ ID NOs: 2-7 (representing HC CDR1, HC CDR2, HC CDR3, LC CDR1, LC CDR2, and LC CDR3, in that order). In specific embodiments, the anti-PD-1 antibody (or antigen binding antibody fragment thereof) comprises all 6 of the CDR amino acid sequences of SEQ ID NOs: 2-7.

In some embodiments, the anti-PD-1 antibody (or antigen binding antibody fragment thereof) comprises (a) the heavy chain variable region (VH) amino acid sequence in SEQ ID NO: 8, or a variant sequence thereof which differs by only one or two amino acids or which has at least or about 70% sequence identity, or (b) the light chain variable region (VL) amino acid sequence in SEQ ID NO: 9 or a variant sequence thereof which differs by only one or two amino acids or which has at least or about 70% sequence identity. In some embodiments, the anti-PD-1 antibody (or antigen binding antibody fragment thereof) comprises the heavy chain variable region amino acid sequence in SEQ ID NO: 8 and the light chain variable region amino acid sequence in SEQ ID NO: 9.

In some embodiments, the anti-PD-1 antibody (or antigen binding antibody fragment thereof) comprises (a) the heavy chain (HC) amino acid sequence of SEQ ID NO: 10 or a variant sequence thereof which differs by only one or two amino acids or which has at least or about 70% sequence identity; or (b) the light chain (LC) amino acid sequence of SEQ ID NO: 11 or a variant sequence thereof which differs by only one or two amino acids or which has at least or about 70% sequence identity. In some embodiments, the anti-PD-1 antibody comprises the heavy chain amino acid sequence of SEQ ID NO: 10 and the light chain amino acid sequence of SEQ ID NO: 11.

In some embodiments, the antibody comprises 1, 2, 3, 4, 5, or all 6 CDRs encoded by the nucleic acid(s) of SEQ ID NOs: 12-17 (representing HC CDR1, HC CDR2, HC CDR3, LC CDR1, LC CDR2, and LC CDR3, in that order). In some embodiments, the antibody comprises all 6 CDRs encoded by the nucleic acids of SEQ ID NOs: 12-17.

In some embodiments, the anti-PD-1 antibody (or an antigen binding portion thereof) comprises (a) a heavy chain variable region encoded by SEQ ID NO: 18 or a variant sequence thereof which differs by only 1, 2, 3, 4, 5, or 6 nucleic acids or which has at least or about 70%, 85%, 90%, or 95% sequence identity, or (b) a light chain variable region encoded by SEQ ID NO: 19 or a variant sequence thereof which differs by only 1, 2, 3, 4, 5, or 6 nucleic acids or which has at least or about 70%, 85%, 90%, or 95% sequence identity. In some embodiments, the anti-PD-1 antibody (or an antigen binding portion thereof) comprises a heavy chain variable region encoded by SEQ ID NO: 18 and a light chain variable region encoded by SEQ ID NO: 19.

In some embodiments, the anti-PD-1 antibody (or an antigen binding portion thereof) comprises (a) a heavy chain encoded by SEQ ID NO: 20 or a variant sequence thereof which differs by only 1, 2, 3, 4, 5, or 6 nucleic acids or which has at least or about 70%, 85%, 90%, or 95% sequence identity, or (b) a light chain encoded by SEQ ID NO: 21 or a variant sequence thereof which differs by only 1, 2, 3, 4, 5, or 6 nucleic acids or which has at least or about 70%, 85%, 90%, or 95% sequence identity. In some embodiments, the anti-PD-1 antibody (or an antigen binding portion thereof) comprises a heavy chain encoded by SEQ ID NO: 20 and a light chain encoded by SEQ ID NO: 21.

Sequences of an exemplary anti-PD-1 antibody are provided in Table 2.

TABLE 2
Sequences of an exemplary anti-PD-1 antibody.

| Description | Sequence |
|---|---|
| HC CDR1 | SYDMS |
| amino acid | (SEQ ID NO: 2) |
| sequence | |
| HC CDR2 | LISGGGSQTY YAESVK |
| amino acid | (SEQ ID NO: 3) |
| sequence | |
| HC CDR3 | PSGHYFYAMD V |
| amino acid | (SEQ ID NO: 4) |
| sequence | |
| LC CDR1 | RASQGISNWL A |
| amino acid | (SEQ ID NO: 5) |
| sequence | |
| LC CDR2 | AASSLQS |
| amino acid | (SED ID NO: 6) |
| sequence | |
| LC CDR3 | QQAESFPHT |
| amino acid | (SEQ ID NO: 7) |
| sequence | |
| Heavy | EVQLLESGGG LVQPGGSLRL SCAASGETFS SYDMSWVRQA PGKGLEWVSL |
| Chain | ISGGGSQTYY AESVKGRFTI SRDNSKNTLY LQMNSLRAED TAVYFCASPS |
| Variable | GHY FYAMDVW GQGTTVTVSS |
| Region | (SEQ ID NO: 8) |
| amino acid | |
| sequence | |
| Light Chain | DIQMTQSPSS VSASVGDRVT ITCRASQGIS NWLAWYQQKP GKAPKLLIFA |
| Variable | ASSLQSGVPS RFSGSGSGTD FTLTISSLQP EDFATYYCQQ AESFPHTEGG |
| Romain | GTKVEIK |
| amino acid | (SEQ ID NO: 9) |
| sequence | |
| Heavy | MDMRVPAQLL GLLLLWLRGA RCEVQLLESG GGLVQPGGSL RLSCAASGET |
| Chain | FSSYDMSWVR QAPGKGLEWV SLISGGGSQT YYAESVKGRE TISRDNSKNT |
| amino acid | LYLQMNSLRA EDTAVYFCAS PSGHYFYAMD VWGQGTTVTV SSASTKGPSV |
| sequence | FPLAPSSKST SGGTAALGCL VKDYFPEPVT VSWNSGALTS GVHTFPAVLQ |
| | SSGLYSLSSV VTVPSSSLGT QTYICNVNHK PSNTKVDKKV EPKSCDKTHT |
| | CPPCPAPELL GGPSVFLFPP KPKDTLMISR TPEVTCVVVD VSHEDPEVKE |
| | NWYVDGVEVH NAKTKPCEEQ YGSTYRCVSV LTVLHQDWLN GKEYKCKVSN |
| | KALPAPIEKT ISKAKGQPRE PQVYTIPPSR EEMTKNQVSL TCLVKGFYPS |
| | DIAVEWESNG QPENNYKTTP PVLDSDGSFF LYSKLTVDKS RWQQGNVFSC |
| | SVMHEALHNH YTQKSLSLSP GK |
| | (SEQ ID NO: 10) |
| Light Chain | MDMRVPAQLL GLLLLWLRGA RCDIQMTQSP SSVSASVGDR VTITCRASQG |
| amino acid | ISNWLAWYQQ KPGKAPKLLI FAASSLQSGV PSRFSGSGSG TDFTLTISSL |
| sequence | QPEDFATYYC QQAESEPHTE GGGIKVEIKR TVAAPSVFIE PPSDEQLKSG |
| | TASVVCLLNN FYPREAKVQW KVDNALQSGN SQESVTEQDS KDSTYSLSST |
| | LTLSKADYEK HKVYACEVTH QGLSSPVTKS ENRGEC |
| | (SEQ ID NO: 11) |
| HC CDR1 | agctatgaca tgagc |
| DNA | (SEQ ID NO: 12) |
| sequence | |
| HC CDR2 | cttattagtg gtggtggtag tcaaacatac tacgcagaat ccgtgaaggg |
| DNA | c |
| sequence | (SEQ ID NO: 13) |
| HC CDR3 | cccagtqgcc actacttcta cgctatggac gtc |
| DNA | (SEQ ID NO: 14) |
| sequence | |
| LC CDR1 | cgggcgagtc agggtattag caactggtta gcc |
| DNA | (SEQ ID NO: 15) |
| sequence | |
| LC CDR2 | gctgcatcca gtttgcaaag t |
| DNA | (SEQ ID NO: 16) |
| sequence | |
| LC CDR3 | caacaggctg aaagtaccc tcacact |
| DNA | (SEQ ID NO: 17) |
| sequence | |
| Heavy | gaggtgcagc tgttggagtc tgggggaggc ttggtacagc ctggggggtc |
| Chain | cctgagactc tcctgtgcag cctctggatt cacctttagc agctatgaca |
| Variable | tgagctgggt ccgccaggct ccagggaagg ggctggaatg ggtctcactt |
| Region | attagtggtg gtggtagtca aacatactac gcagaatccg tgaagggccg |
| DNA | gttcaccatc tccagagaca attccaagaa cacgctgtat ctgcaaatga |
| sequence | acagcctgag agccgaggac acggccgtat atttctgtgc gtcccccagt |
| | ggccactact tctacgctat ggacgtctgg ggccaaggga ccacggtcac |
| | cgtctcctca |
| | (SEQ ID NO: 18) |
| Light Chain | gacatccaga tgacccagtc tccatcttcc gtgtctgcat ctgttggaga |
| Variable | cagagtcacc atcacttgtc gggcgagtca gggtattagc aactggttag |
| Romain | cctggtatca gcagaaacca gggaaagccc ctaagctcct gatctttgct |
| DNA | gcatccagtt tgcaaagtgg ggtcccatca aggttcagcg gaagattttg |
| sequence | tgggacagat ttcaccctca ccatcagcag cctgcagcct gcagtggatc |
| | caacttacta ttgtcaacag gctgaaagtt tccctcacac tttcggcgga |
| | gggaccaagg tggagatcaa a |
| | (SEQ ID NO: 19) |
| Heavy | atggacatga gggtgcccgc tcagctcctg gggctcctgc tgctgtggct |
| Chain | gagaggtgcg cgctgtgagg tgcagctgtt ggagtctggg ggaggcttgg |
| DNA | tacagcctgg ggggtccctg agactctcct gtgcagcctc tggattcacc |
| sequence | tttagcagct atgacatgag ctgggtccgc caggctccag ggaaggggct |
| | ggaatgggtc tcacttatta gtggtggtgg tagtcaaaca tactacgcag |
| | aatccgtgaa gggccggttc accatctcca gagacaattc caagaacacg |
| | ctgtatctgc aaatgaacag cctgagagcc gaggacacgg ccgtatattt |
| | ctqtgcgtcc cccagtggcc actacttcta cgctatggac gtctggggcc |
| | aagggaccac ggtcaccgtc tcctcagcct ccaccaaggg cccatcggtc |
| | ttccccctgg caccctcctc caagagcacc tctgggggca cagcggccct |
| | gggctgcctg gtcaaggact acttccccga accggtgacg gtgtcgtgga |
| | actcaggcgc cctgaccagc ggcgtgcaca ccttcccggc tgtcctacag |
| | tcctcaggac tctactccct cagcagcgtg gtgaccgtgc cctccagcag |
| | cttgggcacc cagacctaca tctgcaacgt gaatcacaag cccagcaaca |
| | ccaaggtgga caagaaagtt gagcccaaat cttqtgacaa aactcacaca |
| | tgcccaccgt gcccagcacc tgaactcctg gggggaccgt cagtcttcct |
| | cttcccccca aaacccaagg acaccctcat gatctcccgg acccctgagg |
| | tcacatgcgt ggtggtggac gtgagccacg aagaccctga ggtcaagttc |
| | aactggtacg tggacggcgt ggaggtgcat aatgccaaga caaagccgtg |
| | cgaggagcag tacggcagca cgtacccttg cgtcagcgtc ctcaccgtcc |
| | tgcaccagga ctggctgaat ggcaaggagt acaagtgcaa ggtgtccaac |
| | aaagccctcc cagcccccat cgagaaaacc atctccaaag ccaaagggca |
| | gccccgagaa ccacaggtgt acaccctgcc cccatcccgg gaggagatga |
| | ccaagaacca ggtcagcctg acctgcctgg tcaaaggctt ctatcccagc |
| | gacatcgccg tggagtggga gagcaatggg cagccggaga acaactacaa |
| | gaccacgcct cccgtgctgg actccgacgg ctccttcttc ctctatagca |
| | agctcaccct ggacaagagc aggtggcagc aggggaacgt cttctcatgc |
| | tccgtgatgc atgaggctct gcacaaccac tacacgcaga agagcctctc |
| | cctgtctccg ggtaaa |
| | (SEQ ID NO: 20) |
| Light Chain | atggacatga gggtgcccgc tcagctcctg gggctcctgc tgctgtggct |
| DNA | gagaggtgcg cgctgtgaca tccagatgac ccagtctcca tcttccgtgt |
| sequence | ctgcatctgt tggagacaga gtcaccatca cttgtcgggc gagtcagggt |
| | attagcaact ggttagcctg gtatcagcag aaaccaggga aagcccctaa |
| | gctcctgatc tttgctgcat ccagtttgca aagtggggtc ccatcaaggt |
| | tcagcggcag tggatctggg acagatttca Ccctcaccat cagcagcctg |
| | cagcctgaag attttgcaac ttactattgt caacaggctg aaagtttccc |
| | tcacactttc ggcggaggga ccaaggtgga gatcaaacga acggtggctg |
| | caccatctgt cttcatcttc ccgccatctg atgagcagtt gaaatctgga |
| | actgcctctg ttgtgtgcct gctgaataac ttctatccca gagaggccaa |
| | agtacagtgg aaggtggata acgccctcca atcgggtaac tcccaggaga |
| | gtgtcacaga gcaggacagc aaggacagca cctacagcct cagcagcacc |
| | ctgacgctga gcaaagcaga ctacgagaaa cacaaagtct acgcctgcga |
| | agtcacccat cagggcctga gctcgcccgt cacaaagagc ttcaacaggg |
| | gagagtgt |
| | (SEQ ID NO: 21) |

GITR agonists include, but are not limited to, GITR fusion proteins and anti-GITR antibodies (e.g., bivalent anti-GITR antibodies), such as, a GITR fusion protein described in U.S. Pat. No. 6,111,090 box.c, European Patent No.: 090505B1, U.S. Pat. No. 8,586,023, PCT Publication Nos.: WO 2010/003118 and 2011/090754, or an anti-GITR antibody described, e.g., in U.S. Pat. No. 7,025,962, European Patent No.: 1947183B1, U.S. Pat. Nos. 7,812,135, 8,388,967, 8,591,886, European Patent No.: EP 1866339, PCT Publication No.: WO 2011/028683, PCT Publication No.: WO 2013/039954, PCT Publication No.: WO2005/007190, PCT Publication No.: WO 2007/133822, PCT Publication No.: WO2005/055808, PCT Publication No.: WO 99/40196, PCT Publication No.: WO 2001/03720, PCT Publication No.: WO99/20758, PCT Publication No.: WO2006/083289, PCT Publication No.: WO 2005/115451, U.S. Pat. No. 7,618,632, and PCT Publication No.: WO 2011/051726.

### Compounds of the Present Disclosure

In some aspects, the present disclosure provides a compound of Formula (0):

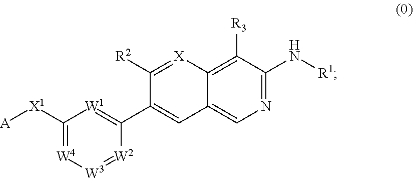

an isomer thereof, or a pharmaceutically acceptable salt thereof, wherein:
X is CR^{X} or N;
R^{X} is H, halogen, cyano, oxo, OH, C₁-C₆ alkyl, C₂-C₆ alkenyl, C₂-C₆ alkynyl, or C₁-C₆ alkoxy, wherein the C₁-C₆ alkyl, C₂-C₆ alkenyl, C₂-C₆ alkynyl, or C₁-C₆ alkoxy is optionally substituted with one or more halogen, cyano, oxo, or OH;
W¹ is N or CR^{W1};
R^{W1} is H, halogen, C₁-C₆ alkyl, C₂-C₆ alkenyl, or C₂-C₆ alkynyl;
W² is N or CR^{W2};
R^{W2} is H, halogen, C₁-C₆ alkyl, C₂-C₆ alkenyl, or C₂-C₆ alkynyl, wherein the C₁-C₆ alkyl, C₂-C₆ alkenyl, or C₂-C₆ alkynyl is optionally substituted with one or more halogen;
W³ is N or CR^{W3};
R^{W3} is H, halogen, C₁-C₆ alkyl, C₂-C₆ alkenyl, or C₂-C₆ alkynyl;
W⁴ is N or CR^{W4};
R^{W4} is H, halogen, C₁-C₆ alkyl, C₂-C₆ alkenyl, C₂-C₆ alkynyl, or S(C₁-C₆ alkyl);
R¹ is H, C₁-C₆ alkyl, C₂-C₆ alkenyl, C₂-C₆ alkynyl, C₁-C₆ alkoxy, C₃-C₁₂ cycloalkyl, 3- to 12-membered heterocycloalkyl, C₆-C₁₀ aryl, or 5- to 10-membered heteroaryl, wherein the C₁-C₆ alkyl, C₂-C₆ alkenyl, C₂-C₆ alkynyl, C₁-C₆ alkoxy, C₃-C₁₂ cycloalkyl, 3- to 12-membered heterocycloalkyl, C₆-C₁₀ aryl, or 5- to 10-membered heteroaryl is optionally substituted with one or more R^{1a};
each R^{1a} independently is halogen, cyano, oxo, OH, NH₂, NHC(═O)O(C₁-C₆ alkyl), N(C₁-C₆ alkyl)₂, C₁-C₆ alkyl, C₂-C₆ alkenyl, C₂-C₆ alkynyl, C₁-C₆ alkoxy, C₃-C₁₂ cycloalkyl, 3- to 12-membered heterocycloalkyl, C₆-C₁₀ aryl, or 5- to 10-membered heteroaryl;
R² is H, halogen, cyano, oxo, OH, C₁-C₆ alkyl, C₂-C₆ alkenyl, C₂-C₆ alkynyl, or C₁-C₆ alkoxy;
R³ is H, halogen, cyano, oxo, OH, C₁-C₆ alkyl, C₂-C₆ alkenyl, C₂-C₆ alkynyl, or C₁-C₆ alkoxy; or
R¹ and R³, together with the intervening atoms, form a 4- to 12-membered heterocycloalkyl optionally substituted with one or more oxo;
X¹ is —NR^{X1}—*, —C(═O)NR^{X1}—*, —NR^{X1}C(═O)—*, —NR^{X1}C(═O)O—*, —NR^{X1}N═C—*, —R^{X1}C(═NR^{X1})—*, —R^{X1}C(═NH)NR^{X1}—*, —NR^{X1}C(═O)NR^{X1}—*, —S(═O)₂NR^{X1}—*, or —NR^{X1}S(═O)₂—*, wherein * denotes attachment to A;
R^{X1} independently is H, S(═O)₂R^{X1a}, C₁-C₆ alkyl, C₂-C₆ alkenyl, C₂-C₆ alkynyl, C₁-C₆ alkoxy, C₃-C₁₂ cycloalkyl, 3- to 12-membered heterocycloalkyl, C₆-C₁₀ aryl, or 5- to 10-membered heteroaryl, wherein the C₁-C₆ alkyl, C₂-C₆ alkenyl, C₂-C₆ alkynyl, C₁-C₆ alkoxy, C₃-C₁₂ cycloalkyl, 3- to 12-membered heterocycloalkyl, C₆-C₁₀ aryl, or 5- to 10-membered heteroaryl is optionally substituted with one or more R^{X1a};
each R^{X1a} independently is halogen, C₁-C₆ alkyl, or 3- to 12-membered heterocycloalkyl, wherein the C₁-C₆ alkyl, or 3- to 12-membered heterocycloalkyl is optionally substituted with one or more halogen;
A is C₁-C₆ alkyl, C₃-C₁₂ cycloalkyl, 3- to 12-membered heterocycloalkyl, C₆-C₁₀ aryl, 5- to 10-membered heteroaryl, —(C₁-C₆ alkyl)-(C₃-C₁₂ cycloalkyl), —(C₁-C₆ alkyl)-(3- to 12-membered heterocycloalkyl), —(C₁-C₆ alkyl)-(C₆-C₁₀ aryl), or —(C₁-C₆ alkyl)-(5- to 10-membered heteroaryl), wherein the C₁-C₆ alkyl, C₃-C₁₂ cycloalkyl, 3- to 12-membered heterocycloalkyl, C₆-C₁₀ aryl, 5- to 10-membered heteroaryl, —(C₁-C₆ alkyl)-(C₃-C₁₂ cycloalkyl), —(C₁-C₆ alkyl)-(3- to 12-membered heterocycloalkyl), —(C₁-C₆ alkyl)-(C₆-C₁₀ aryl), or —(C₁-C₆ alkyl)-(5- to 10-membered heteroaryl) is optionally substituted with one or more R^{A};
each R^{A} independently is halogen, cyano, oxo, OH, OR^{A1}, NH₂, NHR^{A1}, N(R^{A1})₂, (═N)R^{A1}, C₁-C₆ alkyl, C₂-C₆ alkenyl, C₂-C₆ alkynyl, C₁-C₆ alkoxy, C₃-C₁₂ cycloalkyl, 3- to 12-membered heterocycloalkyl, C₆-C₁₀ aryl, or 5- to 10-membered heteroaryl, wherein the C₁-C₆ alkyl, C₂-C₆ alkenyl, C₂-C₆ alkynyl, C₁-C₆ alkoxy, C₃-C₁₂ cycloalkyl, 3- to 12-membered heterocycloalkyl, C₆-C₁₀ aryl, or 5- to 10-membered heteroaryl is optionally substituted with one or more R^{A1};
each R^{A1} independently is halogen, cyano, oxo, OH, OR^{A2}, NH₂, NHR^{A2}, N(R^{A2})₂, C(═O)R^{A2}, C₁-C₆ alkyl, C₂-C₆ alkenyl, C₂-C₆ alkynyl, C₁-C₆ alkoxy, C₃-C₁₂ cycloalkyl, 3- to 12-membered heterocycloalkyl, C₆-C₁₀ aryl, or 5- to 10-membered heteroaryl, wherein the C₁-C₆ alkyl, C₂-C₆ alkenyl, C₂-C₆ alkynyl, C₁-C₆ alkoxy, C₃-C₁₂ cycloalkyl, 3- to 12-membered heterocycloalkyl, C₆-C₁₀ aryl, or 5- to 10-membered heteroaryl is optionally substituted with one or more R^{A2}; and
each R^{A2} independently is halogen, cyano, OH, NH₂, N(R^{A3})₂, C(═O)R^{A3}, C₁-C₆ alkyl, C₂-C₆ alkenyl, or C₂-C₆ alkynyl,
wherein R^{A3} is C₁-C₆ alkyl, C₂-C₆ alkenyl, or C₂-C₆ alkynyl.

In some aspects, the present disclosure provides a compound of Formula (I′):

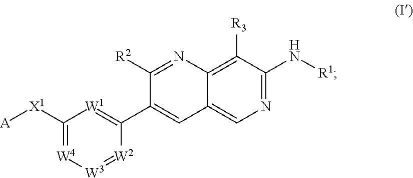

an isomer thereof, or a pharmaceutically acceptable salt thereof.

In some aspects, the present disclosure provides a compound of Formula (I):

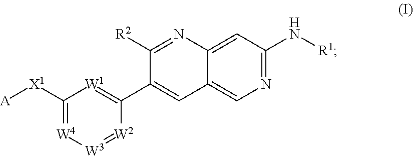

an isomer thereof, or a pharmaceutically acceptable salt thereof.

In some aspects, the present disclosure provides a compound of Formula (II′):

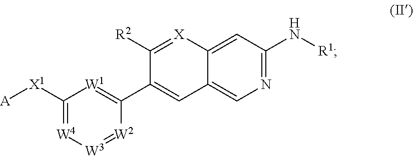

an isomer thereof, or a pharmaceutically acceptable salt thereof.

It is understood that, for a compound of the present disclosure, variables X, R^{X}, W¹, R^{W1}, W², R^{W2}, W³, R^{W3}, W⁴, R^{W4}, R¹, R¹, R², R³, X¹, R^{X1}, R^{X1a}, A, R^{A}, R^{A1}, R^{A2}, R^{A3} can each be, where applicable, selected from the groups described herein, and any group described herein for any of variables X, R^{X}, W¹, R^{W1}, W², R^{W2}, W³, R^{W3}, W⁴, R^{W4}, R¹, R^{1a}, R², R³, X¹, R^{X1}, R^{X1a}, A, R^{A}, R^{A1}, R^{A2}, R^{A3} can be combined, where applicable, with any group described herein for one or more of the remainder of variables X, R^{X}, W¹, R^{W1}, W², R^{W2}, W³, R^{W3}, W⁴, R^{W4}, R¹, R^{1a}, R², R³, X¹, R^{X1}, R^{X1a}, A, R^{A}, R^{A1}, R^{A2}, R^{A3}.

### Variables X and R^{X}

In some embodiments, X is CR^{X} or N. In some embodiments, X is N. In some embodiments, X is CR^{X}. In some embodiments, X is CH. In some embodiments, X is C(CN). In some embodiments, X is CF.

In some embodiments, R^{X} is H, halogen, cyano, oxo, OH, C₁-C₆ alkyl, C₂-C₆ alkenyl, C₂-C₆ alkynyl, or C₁-C₆ alkoxy, wherein the C₁-C₆ alkyl, C₂-C₆ alkenyl, C₂-C₆ alkynyl, or C₁-C₆ alkoxy is optionally substituted with one or more halogen, cyano, oxo, or OH. In some embodiments, R^{X} is H, halogen, cyano, oxo, or OH. In some embodiments, R^{X} is H. In some embodiments, R^{X} is halogen. In some embodiments, R^{X} is fluorine. In some embodiments, R^{X} is chlorine. In some embodiments, R^{X} is bromine. In some embodiments, R^{X} is iodine. In some embodiments, R^{X} is cyano. In some embodiments, R^{X} is C₁-C₆ alkyl, C₂-C₆ alkenyl, C₂-C₆ alkynyl, or C₁-C₆ alkoxy, wherein the C₁-C₆ alkyl, C₂-C₆ alkenyl, C₂-C₆ alkynyl, or C₁-C₆ alkoxy is optionally substituted with one or more halogen, cyano, oxo, or OH. In some embodiments, R^{X} is C₁-C₆ alkoxy, optionally substituted with one or more OH. In some embodiments, R^{X} is C₁ alkoxy, optionally substituted with one or more OH. In some embodiments, R^{X} is C₂ alkoxy, optionally substituted with one or more OH. In some embodiments, R^{X} is C₃ alkoxy, optionally substituted with one or more OH. In some embodiments, R^{X} is C₄ alkoxy, optionally substituted with one or more OH. In some embodiments, R^{X} is C₅ alkoxy, optionally substituted with one or more OH. In some embodiments, R^{X} is C₆ alkoxy, optionally substituted with one or more OH.

Variables W¹, R^{W1}, W², R^{W2}, W³, R^{W3}, W⁴, R^{W4}

In some embodiments, W¹ is N or CR^{W1}. In some embodiments, W¹ is N. In some embodiments, W¹ is CR^{W1}. In some embodiments, W¹ is CH.

In some embodiments, R^{W1} is H, halogen, C₁-C₆ alkyl, C₂-C₆ alkenyl, or C₂-C₆ alkynyl. In some embodiments, R^{W1} is H or halogen, In some embodiments, R^{W1} is H. In some embodiments, R^{W1} is halogen. In some embodiments, R^{W1} is fluorine. In some embodiments, R^{W1} is chlorine. In some embodiments, R^{W1} is bromine. In some embodiments, R^{W1} is iodine. In some embodiments, R^{W1} is C₁-C₆ alkyl, C₂-C₆ alkenyl, or C₂-C₆ alkynyl. In some embodiments, R^{W1} is C₁-C₆ alkyl. In some embodiments, R^{W1} is C₁ alkyl. In some embodiments, R^{W1} is C₂ alkyl. In some embodiments, R^{W1} is C₃ alkyl. In some embodiments, R^{W1} is C₄ alkyl. In some embodiments, R^{W1} is C₅ alkyl. In some embodiments, R^{W1} is C₆ alkyl. In some embodiments, R^{W1} is CH₃.

In some embodiments, W² is N or CR^{W2}. In some embodiments, W² is N. In some embodiments, W² is CR^{W2}. In some embodiments, W² is CH. In some embodiments, W² is C(CH₃).

In some embodiments, R^{W2} is H, halogen, C₁-C₆ alkyl, C₂-C₆ alkenyl, or C₂-C₆ alkynyl, wherein the C₁-C₆ alkyl, C₂-C₆ alkenyl, or C₂-C₆ alkynyl is optionally substituted with one or more halogen.

In some embodiments, R^{W2} is H or halogen. In some embodiments, R^{W2} is H. In some embodiments, R^{W2} is halogen. In some embodiments, R^{W2} is fluorine. In some embodiments, R^{W2} is chlorine. In some embodiments, R^{W2} is bromine. In some embodiments, R^{W2} is iodine. In some embodiments, R^{W2} is C₁-C₆ alkyl, C₂-C₆ alkenyl, or C₂-C₆ alkynyl, wherein the C₁-C₆ alkyl, C₂-C₆ alkenyl, or C₂-C₆ alkynyl is optionally substituted with one or more halogen. In some embodiments, R^{W2} is C₁-C₆ alkyl, C₂-C₆ alkenyl, or C₂-C₆ alkynyl. In some embodiments, R^{W2} is C₁-C₆ alkyl. In some embodiments, R^{W2} is C₁ alkyl. In some embodiments, R^{W2} is C₂ alkyl. In some embodiments, R^{W2} is C₃ alkyl. In some embodiments, R^{W2} is C₄ alkyl. In some embodiments, R^{W2} is C₅ alkyl. In some embodiments, R^{W2} is C₆ alkyl. In some embodiments, R^{W2} is CH₃.

In some embodiments, W³ is N or CR^{W3}. In some embodiments, W³ is N. In some embodiments, W³ is CR^{W3}. In some embodiments, W³ is CH.

In some embodiments, R^{W3} is H, halogen, C₁-C₆ alkyl, C₂-C₆ alkenyl, or C₂-C₆ alkynyl. In some embodiments, R^{W3} is H or halogen. In some embodiments, R^{W3} is H. In some embodiments, R^{W3} is halogen. In some embodiments, R^{W3} is fluorine. In some embodiments, R^{W3} is chlorine. In some embodiments, R^{W3} is bromine. In some embodiments, R^{W3} is iodine. In some embodiments, R^{W3} is C₁-C₆ alkyl, C₂-C₆ alkenyl, or C₂-C₆ alkynyl. In some embodiments, R^{W3} is C₁-C₆ alkyl. In some embodiments, R^{W3} is C₁ alkyl. In some embodiments, R^{W3} is C₂ alkyl. In some embodiments, R^{W3} is C₃ alkyl. In some embodiments, R^{W3} is C₄ alkyl. In some embodiments, R^{W3} is C₅ alkyl. In some embodiments, R^{W3} is C₆ alkyl.

In some embodiments, W⁴ is N or CR^{W4}. In some embodiments, W⁴ is N. In some embodiments, W⁴ is CR^{W4}. In some embodiments, W⁴ is CH.

In some embodiments, R^{W4} is H, halogen, C₁-C₆ alkyl, C₂-C₆ alkenyl, C₂-C₆ alkynyl, or S(C₁-C₆ alkyl). In some embodiments, R^{W4} is H or halogen. In some embodiments, R^{W4} is H. In some embodiments, R^{W4} is halogen. In some embodiments, R^{W4} is fluorine. In some embodiments, R^{W4} is chlorine. In some embodiments, R^{W4} is bromine. In some embodiments, R^{W4} is iodine.

In some embodiments, R^{W4} is C₁-C₆ alkyl, C₂-C₆ alkenyl, C₂-C₆ alkynyl, or S(C₁-C₆ alkyl).

In some embodiments, R^{W4} is C₁-C₆ alkyl. In some embodiments, R^{W4} is C₁ alkyl. In some embodiments, R^{W4} is C₂ alkyl. In some embodiments, R^{W4} is C₃ alkyl. In some embodiments, R^{W4} is C₄ alkyl. In some embodiments, R^{W4} is C₅ alkyl. In some embodiments, R^{W4} is C₆ alkyl. In some embodiments, W¹ is CR^{W1}, W² is CR^{W2}, W³ is CR^{W3} and W⁴ is CR^{W4}.

In some embodiments, W¹ is CH, W² is CH, W³ is CH and W⁴ is CH. In some embodiments, W¹ is CH, W² is C(CH₃), W³ is CH and W⁴ is CH. In some embodiments, W¹ is CR^{W1}, W² is CR^{W2}, W³ is N and W⁴ is CR^{W4}. In some embodiments, W¹ is CH, W² is C(CH₃), W³ is N and W⁴ is CH. In some embodiments, W¹ is CR^{W1}, W² is N, W³ is N and W⁴ is CR^{W4}. In some embodiments, W¹ is CR^{W1}, W² is CR^{W2}, W³ is N and W⁴ is N.

Variables R¹, R^{1a}, R², R³

In some embodiments, R¹ is H, C₁-C₆ alkyl, C₂-C₆ alkenyl, C₂-C₆ alkynyl, C₁-C₆ alkoxy, C₃-C₈ cycloalkyl, 3- to 8-membered heterocycloalkyl, C₆-C₁₀ aryl, or 5- to 10-membered heteroaryl, wherein the C₁-C₆ alkyl, C₂-C₆ alkenyl, C₂-C₆ alkynyl, C₁-C₆ alkoxy, C₃-C₈ cycloalkyl, 3- to 8-membered heterocycloalkyl, C₆-C₁₀ aryl, or 5- to 10-membered heteroaryl is optionally substituted with one or more R^{1a}. In some embodiments, R¹ is H. In some embodiments, R¹ is C₁-C₆ alkyl, wherein the C₁-C₆ alkyl is substituted with one or more R^{1a}. In some embodiments, R¹ is C₁-C₆ alkyl. In some embodiments, R¹ is C₁ alkyl. In some embodiments, R¹ is C₂ alkyl. In some embodiments, R¹ is C₃ alkyl. In some embodiments, R¹ is C₄ alkyl. In some embodiments, R¹ is C₅ alkyl. In some embodiments, R¹ is C₆ alkyl. In some embodiments, R¹ is CH₃. In some embodiments, R¹ is CH₂CH₃.

In some embodiments, R^{1a} is halogen, cyano, oxo, OH, NH₂, NHC(═O)O(C₁-C₆ alkyl), N(C₁-C₆ alkyl)₂, C₁-C₆ alkyl, C₂-C₆ alkenyl, C₂-C₆ alkynyl, C₁-C₆ alkoxy, C₃-C₈ cycloalkyl, 3- to 8-membered heterocycloalkyl, C₆-C₁₀ aryl, or 5- to 10-membered heteroaryl. In some embodiments, R^{1a} is halogen, cyano, oxo, OH or NH₂. In some embodiments, R^{1a} is cyano. In some embodiments, R^{1a} is NHC(═O)O(C₁-C₆ alkyl), N(C₁-C₆ alkyl)₂, C₁-C₆ alkyl, C₂-C₆ alkenyl, C₂-C₆ alkynyl, C₁-C₆ alkoxy, C₃-C₈ cycloalkyl, 3- to 8-membered heterocycloalkyl, C₆-C₁₀ aryl, or 5- to 10-membered heteroaryl.

In some embodiments, R² is H, cyano, oxo, OH, C₁-C₆ alkyl, C₂-C₆ alkenyl, C₂-C₆ alkynyl, or C₁-C₆ alkoxy. In some embodiments, R² is H, cyano, oxo, or OH. In some embodiments, R² is H. In some embodiments, R² is cyano. In some embodiments, R² is OH. In some embodiments, R² is C₁-C₆ alkyl, C₂-C₆ alkenyl, C₂-C₆ alkynyl or C₁-C₆ alkoxy. In some embodiments, R² is H, halogen, cyano, oxo, OH, C₁-C₆ alkyl, C₂-C₆ alkenyl, C₂-C₆ alkynyl, or C₁-C₆ alkoxy.

In some embodiments, R³ is H, halogen, cyano, oxo, OH, C₁-C₆ alkyl, C₂-C₆ alkenyl, C₂-C₆ alkynyl, or C₁-C₆ alkoxy.

In some embodiments, R¹ and R³, together with the intervening atoms, form a 4- to 12-membered heterocycloalkyl optionally substituted with one or more oxo

Variables X¹, R^{X}, R^{X1a}, A, R^{A}, R^{A1}, R^{A2}, R^{A3}

In some embodiments, X¹ is —NR^{X1}—*, —C(═O)NR^{X1}—*, —NR^{X1}C(═O)—*, —NR^{X1}C(═O)O—*, —NR^{X1}N═C—*, —NR^{X1}C(═NH)—*, —R^{X1}C(═NH)NR^{X1}—*, —NR^{X1}C(═O)NR^{X1}—*, —S(═O)₂NR^{X1}—*, or —NR^{X1}S(═O)₂—*, wherein * denotes attachment to A. In some embodiments, X¹ is —NR^{X1}-*, —C(═O)NR^{X1}—*, —NR^{X1}C(═O)—*, —NR^{X1}C(═O)O—*, —NR^{X1}N═C—*, —NR^{X1}C(═NR^{X1})—*, —NR^{X1}C(═NH)NR^{X1}—*, —NR^{X1}C(═O)NR^{X1}—*, —S(═O)₂NR^{X1}—*, or —NR^{X1}S(═O)₂—*, wherein * denotes attachment to A. In some embodiments, is —NR^{X1}—*, wherein * denotes attachment to A. In some embodiments, is —NH—*, wherein * denotes attachment to A. In some embodiments, X¹ is —C(═O)NR^{X1}—* or —NR^{X1}C(═O)—*, wherein * denotes attachment to A. In some embodiments, X¹ is —C(═O)NR^{X1}—*, wherein * denotes attachment to A. In some embodiments, X¹ is —C(═O)NH—*, wherein * denotes attachment to A. In some embodiments, X¹ is —NR^{X1}C(═O)—*, wherein * denotes attachment to A. In some embodiments, X¹ is —NHC(═O)—*, wherein * denotes attachment to A. In some embodiments, X¹ is —NR^{X1}C(═NH)—*, wherein * denotes attachment to A. In some embodiments, X¹ is —NHC(═NH)—*, wherein * denotes attachment to A.

In some embodiments, R^{X1} is H, S(═O)₂R^{X1a}, C₁-C₆ alkyl, C₂-C₆ alkenyl, C₂-C₆ alkynyl, C₁-C₆ alkoxy, C₃-C₈ cycloalkyl, 3- to 8-membered heterocycloalkyl, C₆-C₁₀ aryl, or 5- to 10-membered heteroaryl, wherein the C₁-C₆ alkyl, C₂-C₆ alkenyl, C₂-C₆ alkynyl, C₁-C₆ alkoxy, C₃-C₈ cycloalkyl, 3- to 8-membered heterocycloalkyl, C₆-C₁₀ aryl, or 5- to 10-membered heteroaryl is optionally substituted with one or more R^{X1a}. In some embodiments, R^{X1} is H. In some embodiments, R^{X1} is S(═O)₂R^{X1a}. In some embodiments, R^{X1} is C₁-C₆ alkyl, C₂-C₆ alkenyl, C₂-C₆ alkynyl, C₁-C₆ alkoxy, C₃-C₈ cycloalkyl, 3- to 8-membered heterocycloalkyl, C₆-C₁₀ aryl, or 5- to 10-membered heteroaryl, wherein the C₁-C₆ alkyl, C₂-C₆ alkenyl, C₂-C₆ alkynyl, C₁-C₆ alkoxy, C₃-C₈ cycloalkyl, 3- to 8-membered heterocycloalkyl, C₆-C₁₀ aryl, or 5- to 10-membered heteroaryl is optionally substituted with one or more R^{X1a}. In some embodiments, R^{X1} is C₁-C₆ alkyl, C₂-C₆ alkenyl, C₂-C₆ alkynyl, C₁-C₆ alkoxy, C₃-C₈ cycloalkyl, 3- to 8-membered heterocycloalkyl, C₆-C₁₀ aryl, or 5- to 10-membered heteroaryl. In some embodiments, R^{X1} is C₁-C₆ alkyl. In some embodiments, R^{X1} is CH₃.

In some embodiments, R^{X1a} is halogen, C₁-C₆ alkyl, or 3- to 8-membered heterocycloalkyl, wherein the C₁-C₆ alkyl, or 3- to 8-membered heterocycloalkyl is optionally substituted with one or more halogen. In some embodiments, R^{X1a} is halogen. In some embodiments, R^{X1a} is C₁-C₆ alkyl, optionally substituted with one or more halogen. In some embodiments, R^{X1a} is 3- to 8-membered heterocycloalkyl, optionally substituted with one or more halogen.

In some embodiments, A is C₁-C₆ alkyl, C₃-C₈ cycloalkyl, 3- to 8-membered heterocycloalkyl, C₆-C₁₀ aryl, 5- to 10-membered heteroaryl, —(C₁-C₆ alkyl)-(C₃-C₈ cycloalkyl), —(C₁-C₆ alkyl)-(3- to 8-membered heterocycloalkyl), —(C₁-C₆ alkyl)-(C₆-C₁₀ aryl), or —(C₁-C₆ alkyl)-(5- to 10-membered heteroaryl), wherein the C₁-C₆ alkyl, C₃-C₈ cycloalkyl, 3- to 8-membered heterocycloalkyl, C₆-C₁₀ aryl, 5- to 10-membered heteroaryl, —(C₁-C₆ alkyl)-(C₃-C₈ cycloalkyl), —(C₁-C₆ alkyl)-(3- to 8-membered heterocycloalkyl), —(C₁-C₆ alkyl)-(C₆-C₁₀ aryl), or —(C₁-C₆ alkyl)-(5- to 10-membered heteroaryl) is optionally substituted with one or more R^{A}. In some embodiments, A is C₁-C₆ alkyl, C₃-C₈ cycloalkyl, 3- to 8-membered heterocycloalkyl, C₆-C₁₀ aryl, or 5- to 10-membered heteroaryl, wherein the C₁-C₆ alkyl, C₃-C₈ cycloalkyl, 3- to 8-membered heterocycloalkyl, C₆-C₁₀ aryl, or 5- to 10-membered heteroaryl is optionally substituted with one or more R^{A}. In some embodiments, A is C₃-C₈ cycloalkyl, 3- to 8-membered heterocycloalkyl, C₆-C₁₀ aryl, or 5- to 10-membered heteroaryl, wherein the C₃-C₈ cycloalkyl, 3- to 8-membered heterocycloalkyl, C₆-C₁₀ aryl, or 5- to 10-membered heteroaryl is optionally substituted with one or more R^{A}. In some embodiments, A is C₃-C₈ cycloalkyl, or 3- to 8-membered heterocycloalkyl, wherein the C₃-C₈ cycloalkyl, or 3- to 8-membered heterocycloalkyl is optionally substituted with one or more R^{A}. In some embodiments, A is 3- to 8-membered heterocycloalkyl. In some embodiments, A is tetrahydropyranyl. In some embodiments, A is piperidinyl. In some embodiments, A is 3- to 8-membered heterocycloalkyl optionally substituted with one or more R^{A}. In some embodiments, A is tetrahydropyranyl optionally substituted with one or more R^{A}. In some embodiments, A is piperidinyl optionally substituted with one or more R^{A}. In some embodiments, A is C₆-C₁₀ aryl, or 5- to 10-membered heteroaryl, wherein the C₆-C₁₀ aryl, or 5- to 10-membered heteroaryl is optionally substituted with one or more R^{A}. In some embodiments, A is C₆-C₁₀ aryl, optionally substituted with one or more R^{A}. In some embodiments, A is C₆-C₁₀ aryl. In some embodiments, A is phenyl. In some embodiments, A is phenyl optionally substituted with one or more R^{A}. In some embodiments, A is 5- to 10-membered heteroaryl, optionally substituted with one or more R^{A}. In some embodiments, A is 5- to 10-membered heteroaryl. In some embodiments, A is pyridyl. In some embodiments, A is triazolyl. In some embodiments, A is pyrazolyl. In some embodiments, A is imidazolyl. In some embodiments, A is oxazolyl. In some embodiments, A is imidazo[1,5-a]pyridyl. In some embodiments, A is 2,3-dihydrofuro[2,3-c]pyridyl. In some embodiments, A is 2,3-dihydrofuro[3,2-b]pyridyl. In some embodiments, A is 3,4-dihydro-1H-pyrano[3,4-c]pyridyl. In some embodiments, A is 4,5,6,7-tetrahydrobenzo[d]isoxazolyl. In some embodiments, A is pyridyl optionally substituted with one or more R^{A}. In some embodiments, A is triazolyl optionally substituted with one or more R^{A}. In some embodiments, A is pyrazolyl optionally substituted with one or more R^{A}. In some embodiments, A is imidazolyl optionally substituted with one or more R^{A}. In some embodiments, A is oxazolyl optionally substituted with one or more R^{A}. In some embodiments, A is imidazo[1,5-a]pyridyl optionally substituted with one or more R^{A}. In some embodiments, A is 2,3-dihydrofuro[2,3-c]pyridyl optionally substituted with one or more R^{A}. In some embodiments, A is 2,3-dihydrofuro[3,2-b]pyridyl optionally substituted with one or more R^{A}. In some embodiments, A is 3,4-dihydro-1H-pyrano[3,4-c]pyridyl optionally substituted with one or more R^{A}. In some embodiments, A is 4,5,6,7-tetrahydrobenzo[d]isoxazolyl optionally substituted with one or more R^{A}. In some embodiments, A is —(C₁-C₆ alkyl)-(C₃-C₈ cycloalkyl), —(C₁-C₆ alkyl)-(3- to 8-membered heterocycloalkyl), —(C₁-C₆ alkyl)-(C₆-C₁₀ aryl), or —(C₁-C₆ alkyl)-(5- to 10-membered heteroaryl), wherein the —(C₁-C₆ alkyl)-(C₃-C₈ cycloalkyl), —(C₁-C₆ alkyl)-(3- to 8-membered heterocycloalkyl), —(C₁-C₆ alkyl)-(C₆-C₁₀ aryl), or —(C₁-C₆ alkyl)-(5- to 10-membered heteroaryl) is optionally substituted with one or more R^{A}. In some embodiments, A is —(C₁-C₆ alkyl)-(3- to 8-membered heterocycloalkyl), or —(C₁-C₆ alkyl)-(5- to 10-membered heteroaryl), wherein the —(C₁-C₆ alkyl)-(3- to 8-membered heterocycloalkyl), or —(C₁-C₆ alkyl)-(5- to 10-membered heteroaryl) is optionally substituted with one or more R^{A}. In some embodiments, A is —(C₁-C₆ alkyl)-(3- to 8-membered heterocycloalkyl), optionally substituted with one or more R^{A}. In some embodiments, A is —(C₁-C₆ alkyl)-(3- to 8-membered heterocycloalkyl). In some embodiments, A is —(C₁ alkyl)-(tetrahydropyranyl). In some embodiments, A is —(C₁ alkyl)-(piperidinyl). In some embodiments, A is —(C₁ alkyl)-(tetrahydropyranyl) optionally substituted with one or more R^{A}. In some embodiments, A is —(C₁ alkyl)-(piperidinyl) optionally substituted with one or more R^{A}. In some embodiments, A is —(C₁-C₆ alkyl)-(5- to 10-membered heteroaryl), optionally substituted with one or more R^{A}. In some embodiments, A is —(C₁-C₆ alkyl)-(5- to 10-membered heteroaryl). In some embodiments, A is —(C₁ alkyl)-(triazolyl). In some embodiments, A is —(C₂ alkyl)-(triazolyl). In some embodiments, A is —(C₁ alkyl)-(triazolyl) optionally substituted with one or more R^{A}. In some embodiments, A is —(C₂ alkyl)-(triazolyl) optionally substituted with one or more R^{A}.

In some embodiments, X¹ is —C(═O)NH—*, wherein * denotes attachment to A, and A is phenyl, optionally substituted with one or more R^{A}. In some embodiments, X¹ is —NHC(═O)—*, wherein * denotes attachment to A, and A is phenyl, optionally substituted with one or more R^{A}. In some embodiments, X¹ is —NHC(═NH)—*, wherein * denotes attachment to A, and A is phenyl, optionally substituted with one or more R^{A}. In some embodiments, X¹ is —C(═O)NH—*, wherein * denotes attachment to A, and A is pyridyl, optionally substituted with one or more R^{A}. In some embodiments, X¹ is —NHC(═O)—*, wherein * denotes attachment to A, and A is pyridyl, optionally substituted with one or more R^{A}. In some embodiments, X¹ is —NHC(═NH)—*, wherein * denotes attachment to A, and A is pyridyl, optionally substituted with one or more R^{A}. In some embodiments, X¹ is —C(═O)NH—*, wherein * denotes attachment to A, and A is triazolyl, optionally substituted with one or more R^{A}. In some embodiments, X¹ is —NHC(═O)—*, wherein * denotes attachment to A, and A is triazolyl, optionally substituted with one or more R^{A}. In some embodiments, X¹ is —NHC(═NH)—*, wherein * denotes attachment to A, and A is triazolyl, optionally substituted with one or more R^{A}. In some embodiments, X¹ is —C(═O)NH—*, wherein * denotes attachment to A, and A is tetrahydropyranyl, optionally substituted with one or more R^{A}. In some embodiments, X¹ is —NHC(═O)—*, wherein * denotes attachment to A, and A is tetrahydropyranyl, optionally substituted with one or more R^{A}. In some embodiments, X¹ is —NHC(═NH)—*, wherein * denotes attachment to A, and A is tetrahydropyranyl, optionally substituted with one or more R^{A}. In some embodiments, X¹ is —C(═O)NH—*, wherein * denotes attachment to A, and A is piperidinyl, optionally substituted with one or more R^{A}. In some embodiments, X¹ is —NHC(═O)—*, wherein * denotes attachment to A, and A is piperidinyl, optionally substituted with one or more R^{A}. In some embodiments, X¹ is —NHC(═NH)—*, wherein * denotes attachment to A, and A is piperidinyl, optionally substituted with one or more R^{A}. In some embodiments, X¹ is —C(═O)NH—*, wherein * denotes attachment to A, and A is —(C₁ alkyl)-(tetrahydropyranyl), optionally substituted with one or more R^{A}. In some embodiments, X¹ is —NHC(═O)—*, wherein * denotes attachment to A, and A is —(C₁ alkyl)-(tetrahydropyranyl), optionally substituted with one or more R^{A}. In some embodiments, X¹ is —NHC(═NH)—*, wherein * denotes attachment to A, and A is —(C₁ alkyl)-(tetrahydropyranyl), optionally substituted with one or more R^{A}. In some embodiments, X¹ is —C(═O)NH—*, wherein * denotes attachment to A, and A is —(C₁ alkyl)-(piperidinyl), optionally substituted with one or more R^{A}. In some embodiments, X¹ is —NHC(═O)—*, wherein * denotes attachment to A, and A is —(C₁ alkyl)-(piperidinyl), optionally substituted with one or more R^{A}. In some embodiments, X¹ is —NHC(═NH)—*, wherein * denotes attachment to A, and A is —(C₁ alkyl)-(piperidinyl), optionally substituted with one or more R^{A}. In some embodiments, X¹ is —C(═O)NH—*, wherein * denotes attachment to A, and A is —(C₁ alkyl)-(triazolyl), optionally substituted with one or more R^{A}. In some embodiments, X¹ is —C(═O)NH—*, wherein * denotes attachment to A, and A is —(C₂ alkyl)-(triazolyl), optionally substituted with one or more R^{A}. In some embodiments, X¹ is —NHC(═O)—*, wherein * denotes attachment to A, and A is —(C₁ alkyl)-(triazolyl), optionally substituted with one or more R^{A}. In some embodiments, X¹ is —NHC(═O)—*, wherein * denotes attachment to A, and A is —(C₂ alkyl)-(triazolyl), optionally substituted with one or more R^{A}. In some embodiments, X¹ is —NHC(═NH)—*, wherein * denotes attachment to A, and A is —(C₁ alkyl)-(triazolyl), optionally substituted with one or more R^{A}. In some embodiments, X¹ is —NHC(═NH)—*, wherein * denotes attachment to A, and A is —(C₂ alkyl)-(triazolyl), optionally substituted with one or more R^{A}.

In some embodiments, R^{A} is halogen, cyano, oxo, OH, OR^{A1}, NH₂, NHR^{A1}, N(R^{A1})₂, (═N)R^{A1}, C₁-C₆ alkyl, C₂-C₆ alkenyl, C₂-C₆ alkynyl, C₁-C₆ alkoxy, C₃-C₈ cycloalkyl, 3- to 8-membered heterocycloalkyl, C₆-C₁₀ aryl, or 5- to 10-membered heteroaryl, wherein the C₁-C₆ alkyl, C₂-C₆ alkenyl, C₂-C₆ alkynyl, C₁-C₆ alkoxy, C₃-C₈ cycloalkyl, 3- to 8-membered heterocycloalkyl, C₆-C₁₀ aryl, or 5- to 10-membered heteroaryl is optionally substituted with one or more R^{A1}. In some embodiments, R^{A} is halogen, cyano, oxo, OH, OR^{A1}, NH₂, NHR^{1A}, N(R^{A1})₂, or (═N)R^{A1}. In some embodiments, R^{A} is halogen. In some embodiments, R^{A} is fluorine. In some embodiments, R^{A} is chlorine. In some embodiments, R^{A} is bromine. In some embodiments, R^{A} is iodine. In some embodiments, R^{A} is cyano. In some embodiments, R^{A} is OH. In some embodiments, R^{A} is OR^{A1}. In some embodiments, R^{A} is O(C₁-C₆ alkyl), optionally substituted with one or more R^{A2}. In some embodiments, R^{A} is NHR^{A1}. In some embodiments, R^{A} is N(R^{A1})₂. In some embodiments, R^{A} is N(CH₃)₂. In some embodiments, R^{A} is C₁-C₆ alkyl, C₂-C₆ alkenyl, C₂-C₆ alkynyl, C₁-C₆ alkoxy, C₃-C₈ cycloalkyl, 3- to 8-membered heterocycloalkyl, C₆-C₁₀ aryl, or 5- to 10-membered heteroaryl, wherein the C₁-C₆ alkyl, C₂-C₆ alkenyl, C₂-C₆ alkynyl, C₁-C₆ alkoxy, C₃-C₈ cycloalkyl, 3- to 8-membered heterocycloalkyl, C₆-C₁₀ aryl, or 5- to 10-membered heteroaryl is optionally substituted with one or more R^{A1}. In some embodiments, R^{A} is C₁-C₆ alkyl. In some embodiments, R^{A} is C₁ alkyl. In some embodiments, R^{A} is C₂ alkyl. In some embodiments, R^{A} is C₃ alkyl. In some embodiments, R^{A} is C₄ alkyl. In some embodiments, R^{A} is C₅ alkyl. In some embodiments, R^{A} is C₆ alkyl. In some embodiments, R^{A} is C₁-C₆ alkyl, optionally substituted with one or more R^{A1}. In some embodiments, R^{A} is C₁ alkyl, optionally substituted with one or more R^{A1}. In some embodiments, R^{A} is C₂ alkyl, optionally substituted with one or more R^{A1}. In some embodiments, R^{A} is C₃ alkyl, optionally substituted with one or more R^{A1}. In some embodiments, R^{A} is C₄ alkyl, optionally substituted with one or more R^{A1}. In some embodiments, R^{A} is C₅ alkyl, optionally substituted with one or more R^{A1}. In some embodiments, R^{A} is C₆ alkyl, optionally substituted with one or more R^{A1}. In some embodiments, R^{A} is C₁-C₆ alkyl, optionally substituted with one or more halogen. In some embodiments, R^{A} is C₁ alkyl, optionally substituted with one or more halogen. In some embodiments, R^{A} is C₃ alkyl, optionally substituted with one or more halogen. In some embodiments, R^{A} is CH₃. In some embodiments, R^{A} is CF₃.

In some embodiments, R^{A} is C(CH₃)₂CN. In some embodiments, R^{A} is C₁-C₆ alkoxy. In some embodiments, R^{A} is C₁ alkoxy. In some embodiments, R^{A} is C₂ alkoxy. In some embodiments, R^{A} is C₃ alkoxy. In some embodiments, R^{A} is C₄ alkoxy. In some embodiments, R^{A} is C₅ alkoxy. In some embodiments, R^{A} is C₆ alkoxy. In some embodiments, R^{A} is C₁-C₆ alkoxy, optionally substituted with one or more R^{A1}. In some embodiments, R^{A} is C₁ alkoxy, optionally substituted with one or more R^{A1}. In some embodiments, R^{A} is C₂ alkoxy, optionally substituted with one or more R^{A1}. In some embodiments, R^{A} is C₃ alkoxy, optionally substituted with one or more R^{A1}. In some embodiments, R^{A} is C₄ alkoxy, optionally substituted with one or more R^{A1}. In some embodiments, R^{A} is C₅ alkoxy, optionally substituted with one or more R^{A1}. In some embodiments, R^{A} is C₆ alkoxy, optionally substituted with one or more R^{A1} In some embodiments, R^{A} is C₃-C₈ cycloalkyl. In some embodiments, R^{A} is C₃ cycloalkyl. In some embodiments, R^{A} is C₄ cycloalkyl. In some embodiments, R^{A} is C₅ cycloalkyl. In some embodiments, R^{A} is C₆ cycloalkyl. In some embodiments, R^{A} is C₇ cycloalkyl. In some embodiments, R^{A} is C₈ cycloalkyl.

In some embodiments, R^{A} is C₃-C₈ cycloalkyl, optionally substituted with one or more R^{A1}. In some embodiments, R^{A} is C₃ cycloalkyl, optionally substituted with one or more R^{A1}. In some embodiments, R^{A} is C₄ cycloalkyl, optionally substituted with one or more R^{A1} In some embodiments, R^{A} is C₅ cycloalkyl, optionally substituted with one or more R^{A1}. In some embodiments, R^{A} is C₆ cycloalkyl, optionally substituted with one or more R^{A1}. In some embodiments, R^{A} is C₇ cycloalkyl, optionally substituted with one or more R^{A1}. In some embodiments, R^{A} is C₈ cycloalkyl, optionally substituted with one or more R^{A1}

In some embodiments, R^{A1} is halogen, cyano, oxo, OH, OR^{A2}, NH₂, NHR^{A2}, N(R^{A2})₂, C(═O)R^{A2}, C₁-C₆ alkyl, C₂-C₆ alkenyl, C₂-C₆ alkynyl, C₁-C₆ alkoxy, C₃-C₈ cycloalkyl, 3- to 8-membered heterocycloalkyl, C₆-C₁₀ aryl, or 5- to 10-membered heteroaryl, wherein the C₁-C₆ alkyl, C₂-C₆ alkenyl, C₂-C₆ alkynyl, C₁-C₆ alkoxy, C₃-C₈ cycloalkyl, 3- to 8-membered heterocycloalkyl, C₆-C₁₀ aryl, or 5- to 10-membered heteroaryl is optionally substituted with one or more R^{A2}. In some embodiments, R^{A1} is halogen, cyano, oxo, OH, OR^{A2}, NH₂, NHR^{A2}, N(R^{A2})₂, or C(═O)R^{A2}. In some embodiments, R^{A1} is halogen. In some embodiments, R^{A1} is fluorine. In some embodiments, R^{A1} is chlorine. In some embodiments, R^{A1} is bromine. In some embodiments, R^{A1} is iodine. In some embodiments, R^{A1} is cyano. In some embodiments, R^{A1} is oxo. In some embodiments, R^{A1} is OH. In some embodiments, R^{A1} is OR^{A2}. In some embodiments, R^{A1} is NH₂. In some embodiments, R^{A1} is C₁-C₆ alkyl, C₂-C₆ alkenyl, C₂-C₆ alkynyl, C₁-C₆ alkoxy, C₃-C₈ cycloalkyl, 3- to 8-membered heterocycloalkyl, C₆-C₁₀ aryl, or 5- to 10-membered heteroaryl, wherein the C₁-C₆ alkyl, C₂-C₆ alkenyl, C₂-C₆ alkynyl, C₁-C₆ alkoxy, C₃-C₈ cycloalkyl, 3- to 8-membered heterocycloalkyl, C₆-C₁₀ aryl, or 5- to 10-membered heteroaryl is optionally substituted with one or more R^{A2}. In some embodiments, R^{A1} is C₁-C₆ alkyl. In some embodiments, R^{A1} is C₁ alkyl. In some embodiments, R^{A1} is C₂ alkyl. In some embodiments, R^{A1} is C₃ alkyl. In some embodiments, R^{A1} is C₄ alkyl. In some embodiments, R^{A1} is C₅ alkyl. In some embodiments, R^{A1} is C₆ alkyl. In some embodiments, R^{A1} is C₁-C₆ alkyl, optionally substituted with one or more R^{A2}. In some embodiments, R^{A1} is C₁ alkyl, optionally substituted with one or more R^{A2}. In some embodiments, R^{A1} is C₂ alkyl, optionally substituted with one or more R^{A2}. In some embodiments, R^{A1} is C₃ alkyl, optionally substituted with one or more R^{A2}. In some embodiments, R^{A1} is C₄ alkyl, optionally substituted with one or more R^{A2}. In some embodiments, R^{A1} is C₅ alkyl, optionally substituted with one or more R^{A2}. In some embodiments, R^{A1} is C₆ alkyl, optionally substituted with one or more R^{A2}.

In some embodiments, R^{A} is C₁-C₆ alkyl and R^{A1} is halogen. In some embodiments, R^{A} is C₁ alkyl and R^{A1} is fluorine. In some embodiments, R^{A} is C₁-C₆ alkyl and R^{A1} is cyano. In some embodiments, R^{A} is C₁ alkyl and R^{A1} is cyano.

In some embodiments, R^{A2} is halogen, cyano, OH, NH₂, N(R^{A3})₂, C(═O)R^{A3}, C₁-C₆ alkyl, C₂-C₆ alkenyl, or C₂-C₆ alkynyl. In some embodiments, R^{A2} is halogen, cyano, OH, NH₂, N(R^{A3})₂, or C(═O)R^{A3}. In some embodiments, R^{A2} is halogen. In some embodiments, R^{A2} is fluorine. In some embodiments, R^{A2} is chlorine. In some embodiments, R^{A2} is bromine. In some embodiments, R^{A2} is iodine. In some embodiments, R^{A2} is OH. In some embodiments, R^{A2} is C₁-C₆ alkyl, C₂-C₆ alkenyl, or C₂-C₆ alkynyl. In some embodiments, R^{A2} is C₁-C₆ alkyl. In some embodiments, R^{A2} is C₁ alkyl. In some embodiments, R^{A2} is C₂ alkyl. In some embodiments, R^{A2} is C₃ alkyl. In some embodiments, R^{A2} is C₄ alkyl. In some embodiments, R^{A2} is C₅ alkyl. In some embodiments, R^{A2} is C₆ alkyl.

In some embodiments, R^{A3} is C₁-C₆ alkyl, C₂-C₆ alkenyl, or C₂-C₆ alkynyl.

In some embodiments, the compound is of Formula (I-a), (I-b), (I-c), (I-d), (I-e), or (I-f):

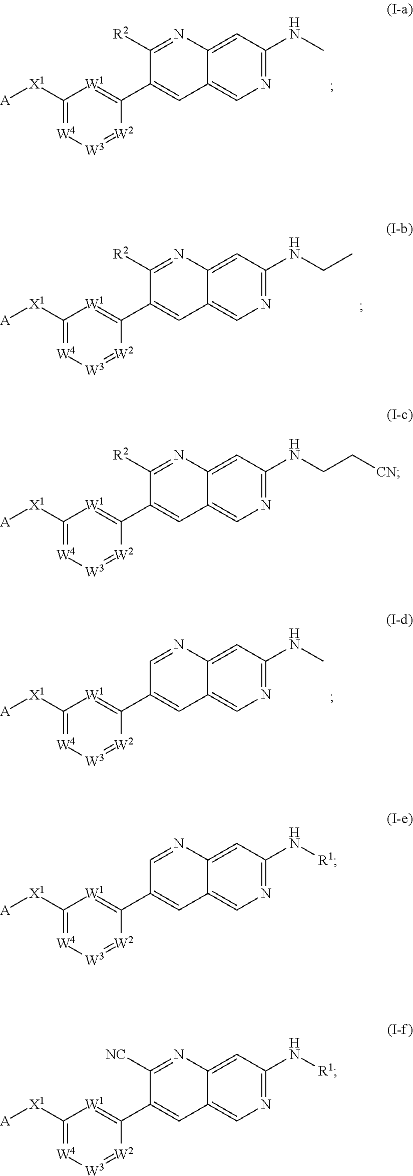

or a pharmaceutically acceptable salt or stereoisomer thereof.

In some embodiments, the compound is of Formula (I-g), (I-h), (I-i), (I-j), (I-k), (I-l), (I-m), (I-n), (I-o), (I-p), (I-q), (I-r), (I-s), or (I-t).

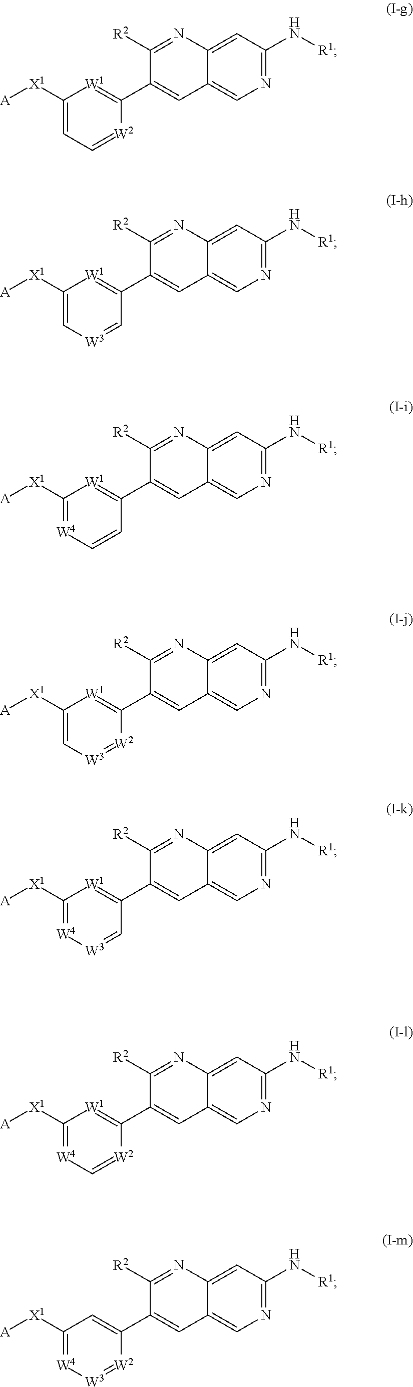

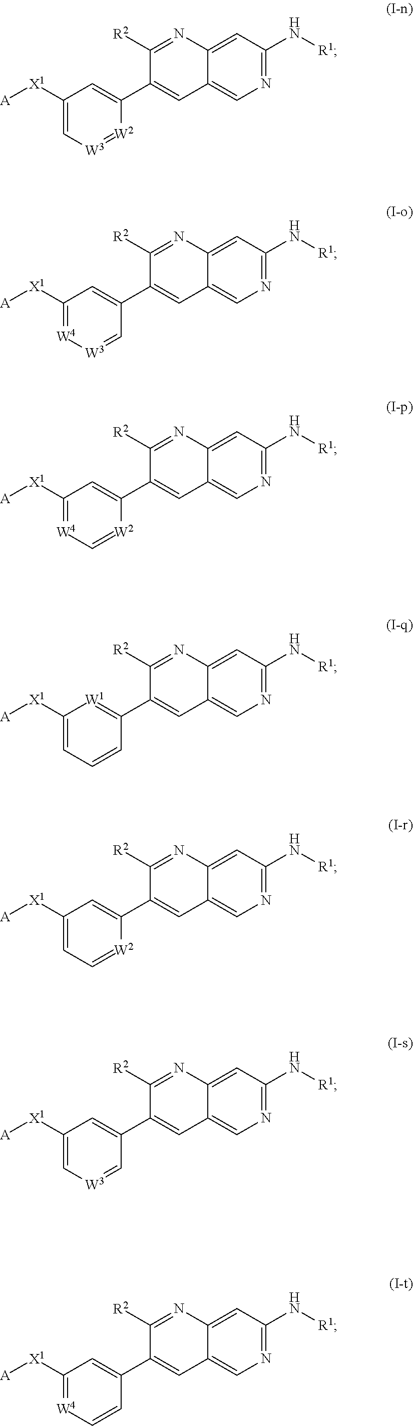

or a pharmaceutically acceptable salt or stereoisomer thereof.

In some embodiments, the compound is of Formula (I-u), (I-v), (I-w), or (I-x):

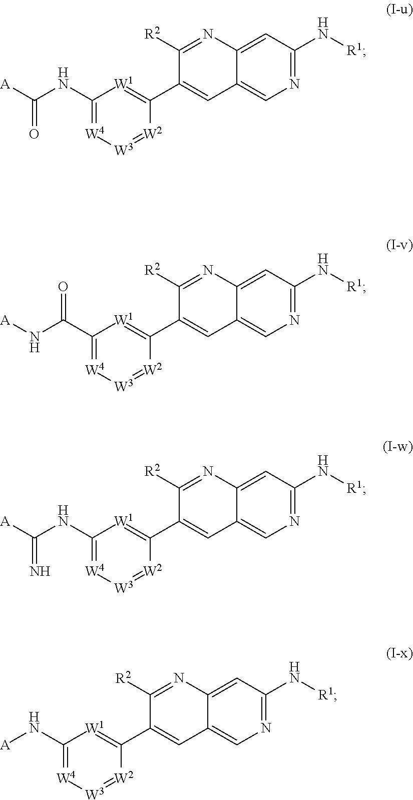

or a pharmaceutically acceptable salt or stereoisomer thereof.

In some embodiments, the compound is of Formula (I-a-i), (I-b-i), (I-c-i), (I-d-i), (I-e-i), or (I-f-i):

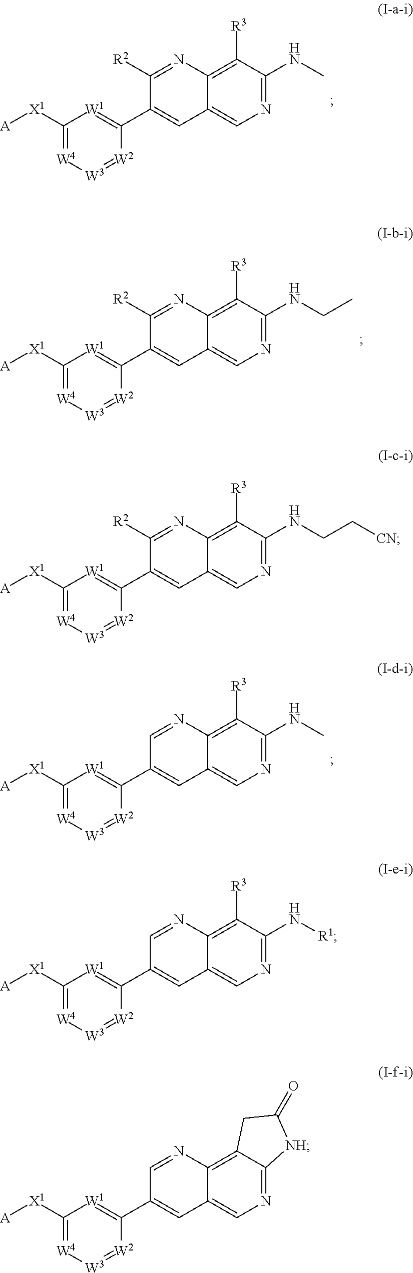

or a pharmaceutically acceptable salt or stereoisomer thereof.

In some embodiments, the compound is of Formula (I-g-i), (I-h-i), (I-i-i), (I-j-i), (I-k-i), (I-l-i), (I-m-i), (I-n-i), (I-o-i), (I-p-i), (I-q-i), (I-r-i), (I-s-i), or (I-t-i).

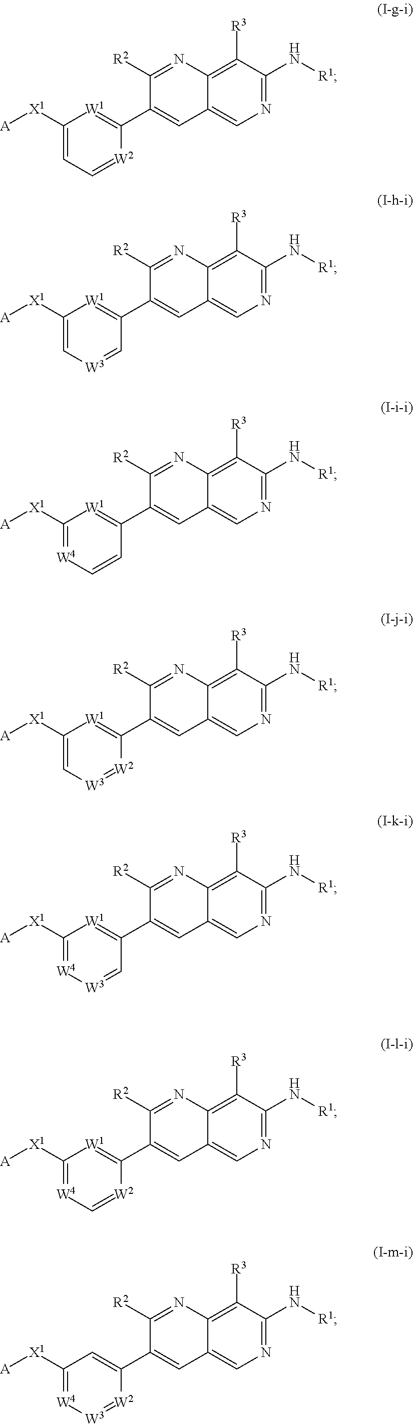

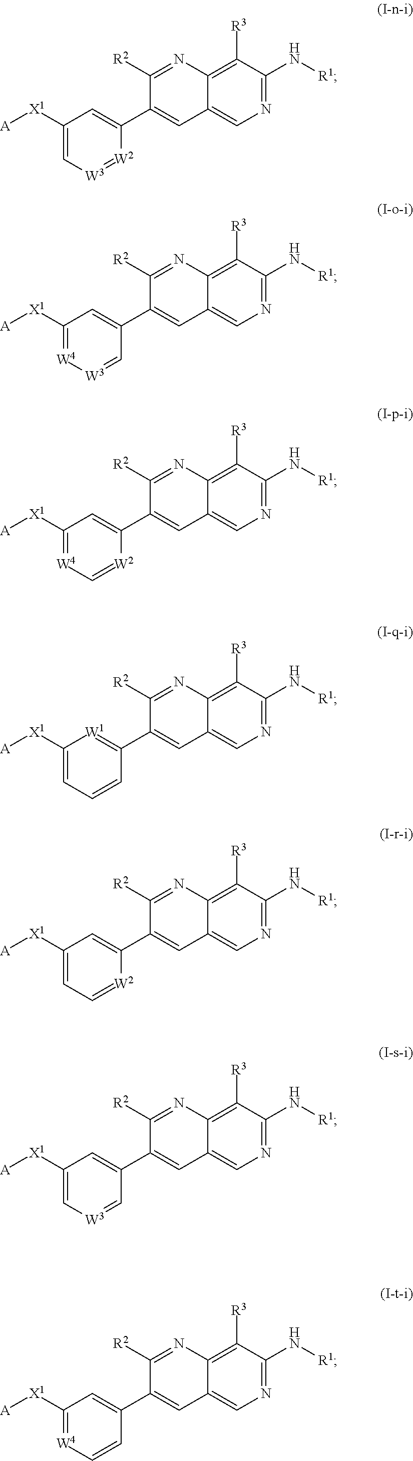

or a pharmaceutically acceptable salt or stereoisomer thereof.

In some embodiments, the compound is of Formula (I-u-i), (I-v-i), (I-w-i), or (I-x-i).

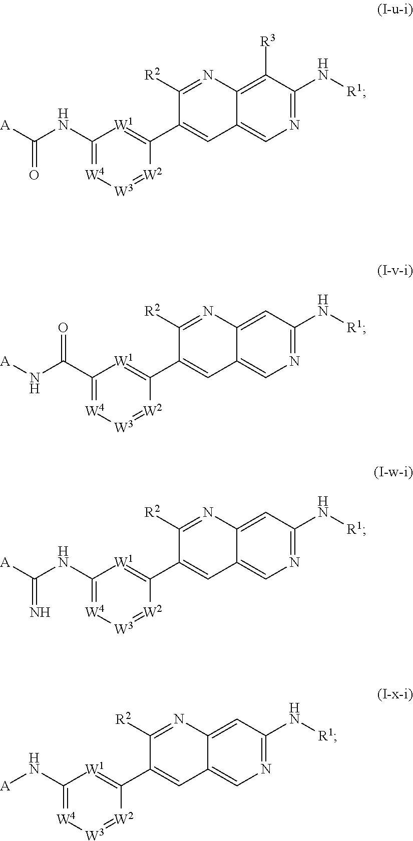

or a pharmaceutically acceptable salt or stereoisomer thereof.

In some embodiments, the compound is of Formula (I-a-ii), (I-b-ii), (I-c-ii), (I-d-ii), (I-e-ii), (I-f-ii), (I-g-ii), (I-h-ii), (I-i-ii), (I-j-ii), (I-k-ii), (I-l-ii), (I-m-ii), (I-n-ii), (I-o-ii), or (I-p-ii).

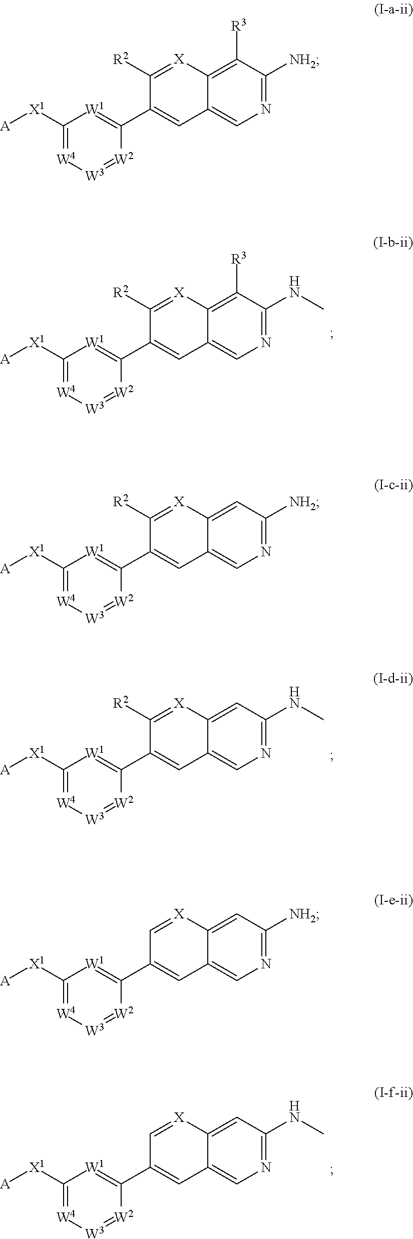

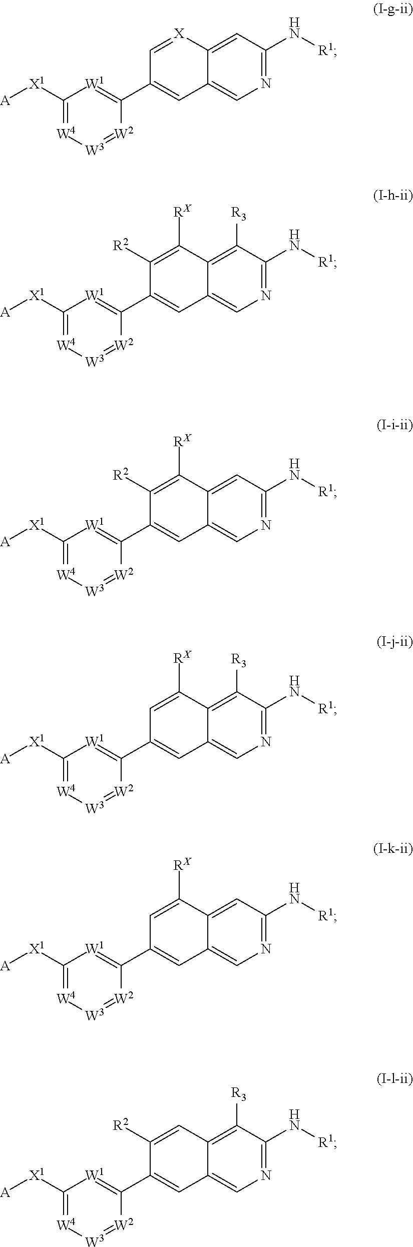

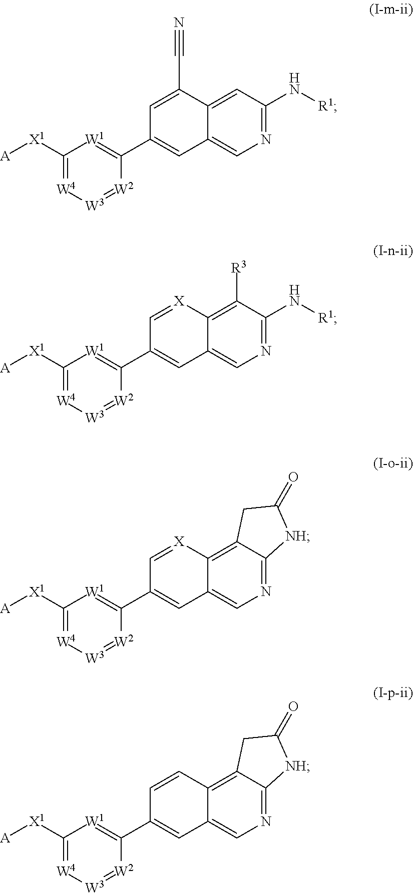

or a pharmaceutically acceptable salt or stereoisomer thereof.

In some embodiments, the compound is of Formula (I-q-ii), (I-r-ii), (I-s-ii), (I-t-ii), (I-u-ii), (I-v-ii), (I-w-ii), (I-x-ii), (I-y-ii), (I-z-ii), (I-aa-ii), (I-bb-ii), (I-cc-ii), or (I-dd-ii).

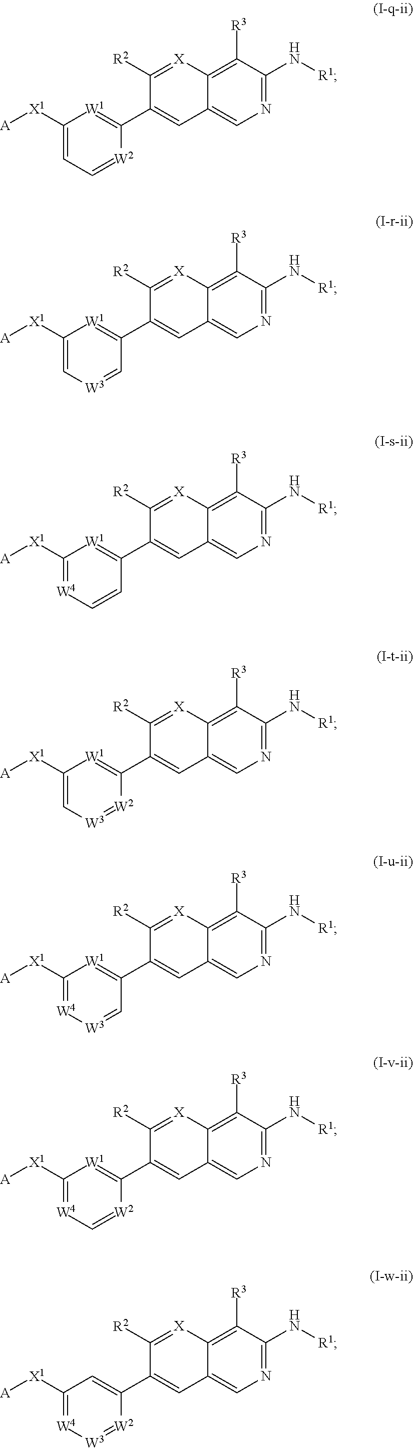

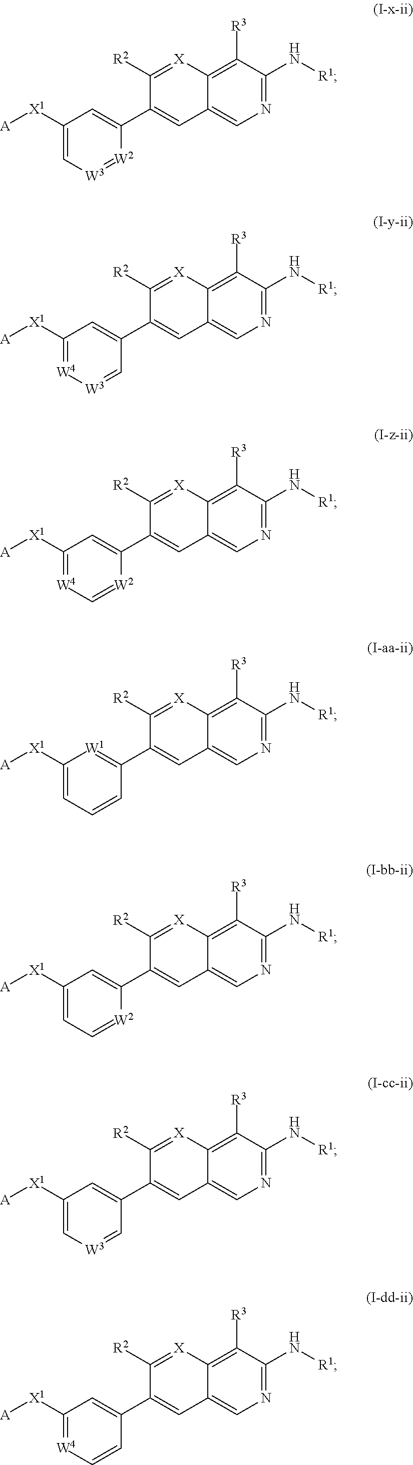

or a pharmaceutically acceptable salt or stereoisomer thereof.

In some embodiments, the compound is of Formula (I-ee-ii), (I-ff-ii), (I-gg-ii), or (I-hh-ii).

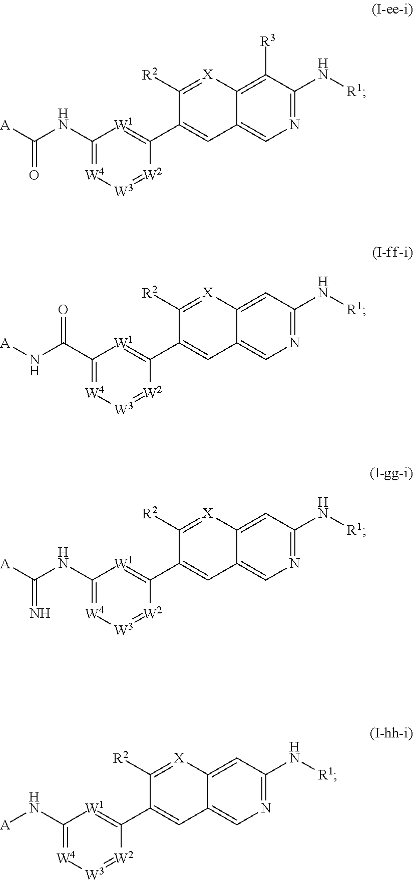

or a pharmaceutically acceptable salt or stereoisomer thereof.

In some embodiments, the compound is selected from the compounds described in Table I, and pharmaceutically acceptable salts and stereoisomers thereof.

In some embodiments, the compound is selected from the compounds described in Table I, and pharmaceutically acceptable salts thereof.

In some embodiments, the compound is selected from the compounds described in Table I.

TABLE I

| Compound No. | Structure |
|---|---|
| 1 | 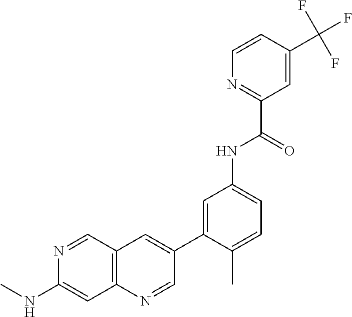 [SMILES: CNc1cc2ncc(-c3cc(NC(=O)c4cc(C(F)(F)F)ccn4)ccc3C)cc2cn1] |
| 2 | 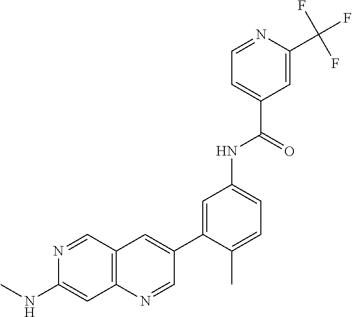 [SMILES: CNc1cc2ncc(-c3cc(NC(=O)c4ccnc(C(F)(F)F)c4)ccc3C)cc2cn1] |
| 3 | 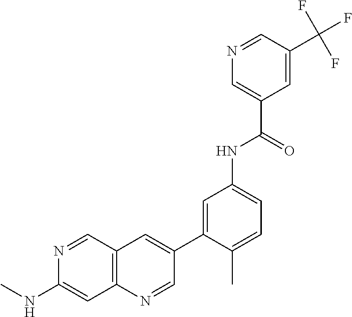 [SMILES: CNc1cc2ncc(-c3cc(NC(=O)c4cncc(C(F)(F)F)c4)ccc3C)cc2cn1] |
| 4 | 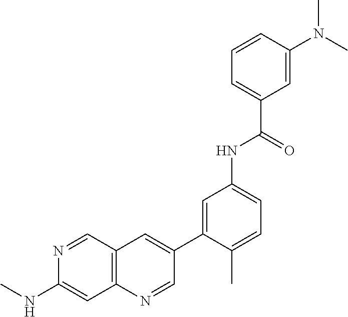 |
| 5 | 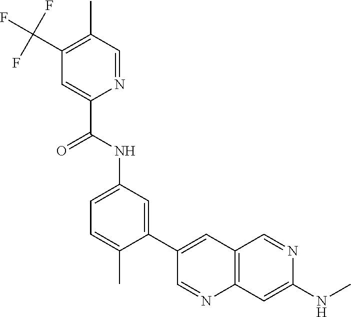 [SMILES: CNc1cc2ncc(-c3cc(NC(=O)c4cc(C(F)(F)F)c(C)cn4)ccc3C)cc2cn1] |
| 6 | 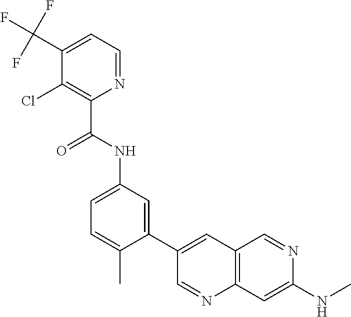 [SMILES: CNc1cc2ncc(-c3cc(NC(=O)c4nccc(C(F)(F)F)c4Cl)ccc3C)cc2cn1] |
| 7 | 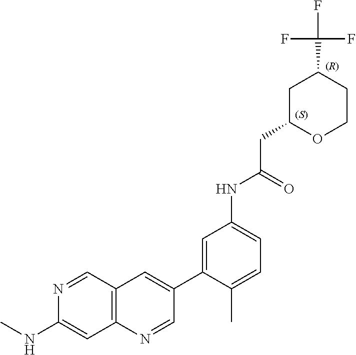 |
| 8 | 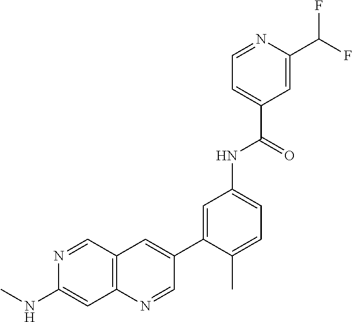 [SMILES: CNc1cc2ncc(-c3cc(NC(=O)c4ccnc(C(F)F)c4)ccc3C)cc2cn1] |
| 9 | 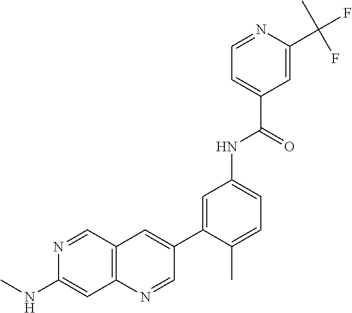 [SMILES: CNc1cc2ncc(-c3cc(NC(=O)c4ccnc(C(C)(F)F)c4)ccc3C)cc2cn1] |
| 10 | 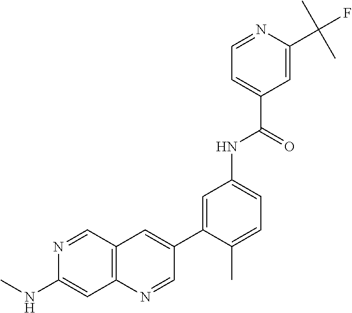 [SMILES: CNc1cc2ncc(-c3cc(NC(=O)c4ccnc(C(C)(C)F)c4)ccc3C)cc2cn1] |
| 11 | 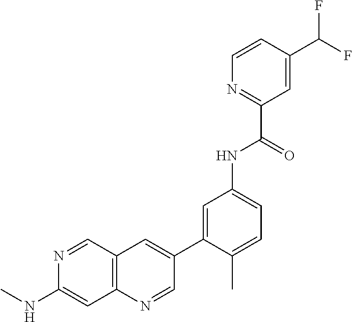 [SMILES: CNc1cc2ncc(-c3cc(NC(=O)c4cc(C(F)F)ccn4)ccc3C)cc2cn1] |
| 12 | 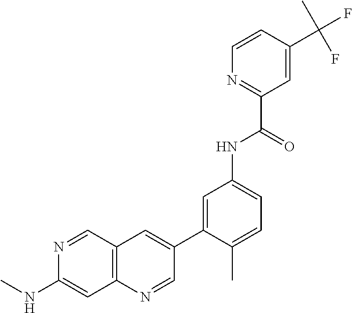 [SMILES: CNc1cc2ncc(-c3cc(NC(=O)c4cc(C(C)(F)F)ccn4)ccc3C)cc2cn1] |
| 13 | 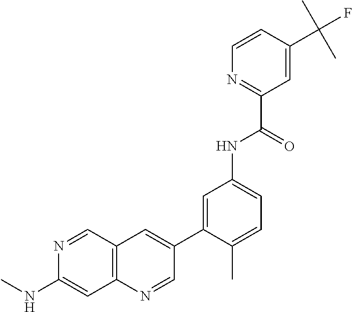 [SMILES: CNc1cc2ncc(-c3cc(NC(=O)c4cc(C(C)(C)F)ccn4)ccc3C)cc2cn1] |
| 14 | 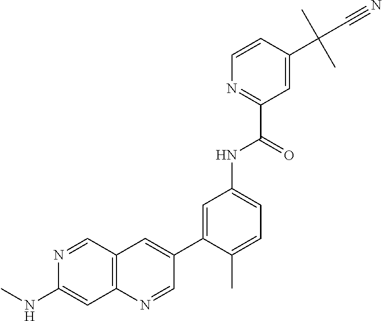 [SMILES: CNc1cc2ncc(-c3cc(NC(=O)c4cc(C(C)(C)C#N)ccn4)ccc3C)cc2cn1] |
| 15 | 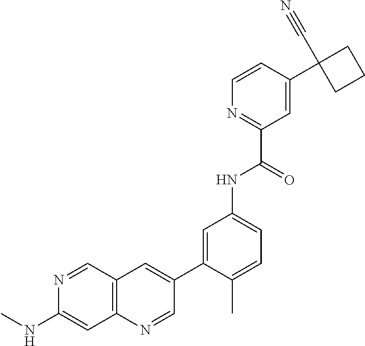 [SMILES: CNc1cc2ncc(-c3cc(NC(=O)c4cc(C5(C#N)CCC5)ccn4)ccc3C)cc2cn1] |
| 16 | 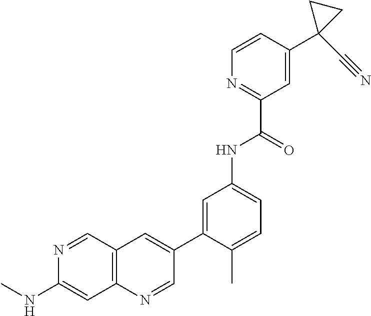 [SMILES: CNc1cc2ncc(-c3cc(NC(=O)c4cc(C5(C#N)CC5)ccn4)ccc3C)cc2cn1] |
| 17 | 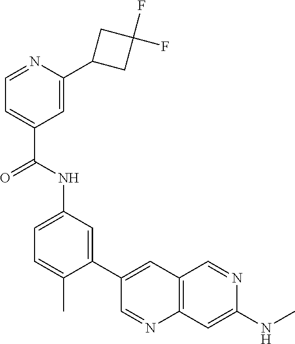 [SMILES: CNc1cc2ncc(-c3cc(NC(=O)c4ccnc(C5CC(F)(F)C5)c4)ccc3C)cc2cn1] |
| 18 | 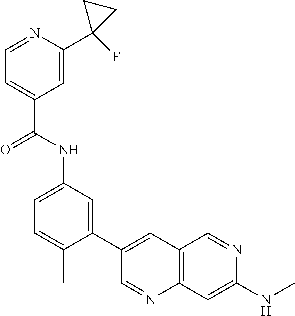 [SMILES: CNc1cc2ncc(-c3cc(NC(=O)c4ccnc(C5(F)CC5)c4)ccc3C)cc2cn1] |
| 19 | 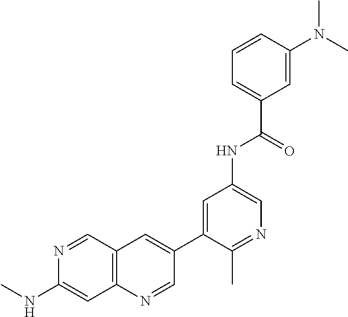 |
| 20 | 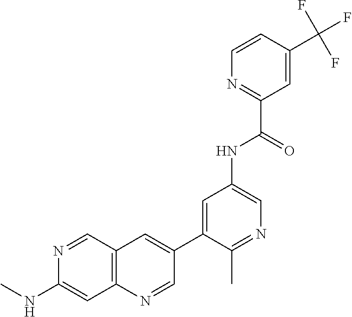 [SMILES: CNc1cc2ncc(-c3cc(NC(=O)c4cc(C(F)(F)F)ccn4)cnc3C)cc2cn1] |
| 21 | 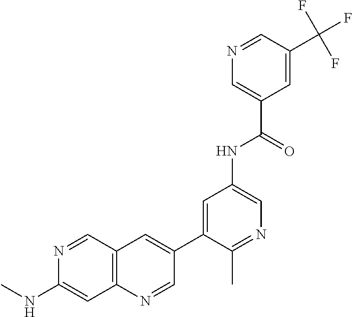 [SMILES: CNc1cc2ncc(-c3cc(NC(=O)c4cncc(C(F)(F)F)c4)cnc3C)cc2cn1] |
| 22 | 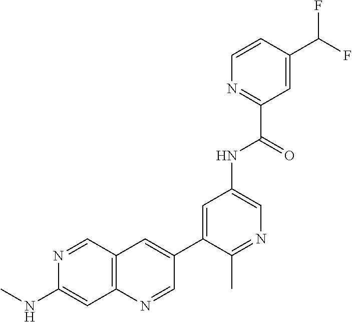 [SMILES: CNc1cc2ncc(-c3cc(NC(=O)c4cc(C(F)F)ccn4)cnc3C)cc2cn1] |
| 23 | 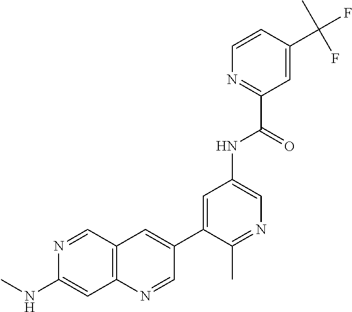 [SMILES: CNc1cc2ncc(-c3cc(NC(=O)c4cc(C(C)(F)F)ccn4)cnc3C)cc2cn1] |
| 24 | 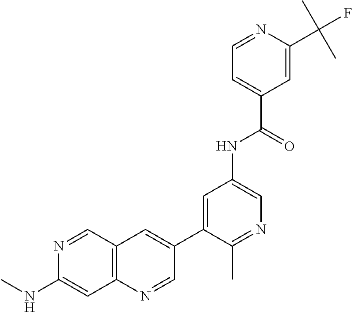 [SMILES: CNc1cc2ncc(-c3cc(NC(=O)c4ccnc(C(C)(C)F)c4)cnc3C)cc2cn1] |
| 25 | 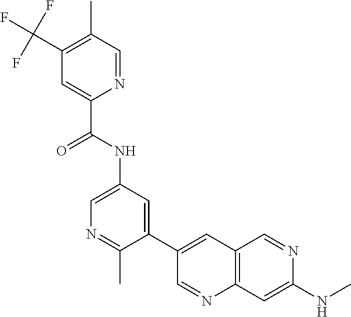 [SMILES: CNc1cc2ncc(-c3cc(NC(=O)c4cc(C(F)(F)F)c(C)cn4)cnc3C)cc2cn1] |
| 26 | 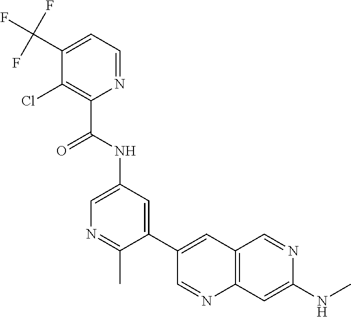 [SMILES: CNc1cc2ncc(-c3cc(NC(=O)c4nccc(C(F)(F)F)c4Cl)cnc3C)cc2cn1] |
| 27 | 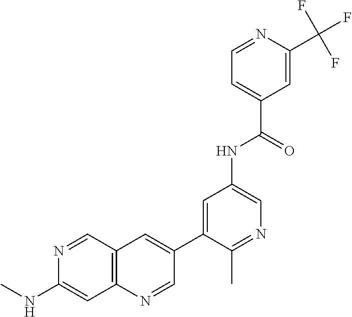 [SMILES: CNc1cc2ncc(-c3cc(NC(=O)c4ccnc(C(F)(F)F)c4)cnc3C)cc2cn1] |
| 28 | 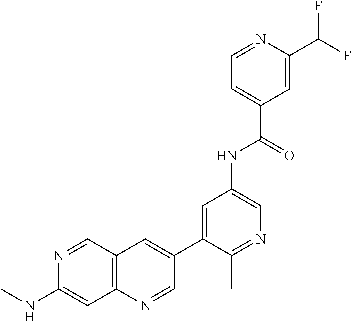 [SMILES: CNc1cc2ncc(-c3cc(NC(=O)c4ccnc(C(F)F)c4)cnc3C)cc2cn1] |
| 29 | 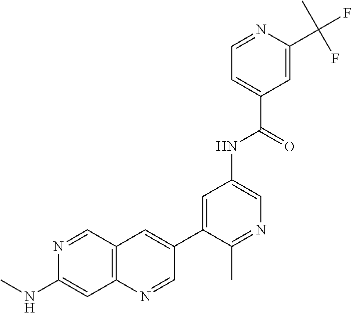 [SMILES: CNc1cc2ncc(-c3cc(NC(=O)c4ccnc(C(C)(F)F)c4)cnc3C)cc2cn1] |
| 30 | 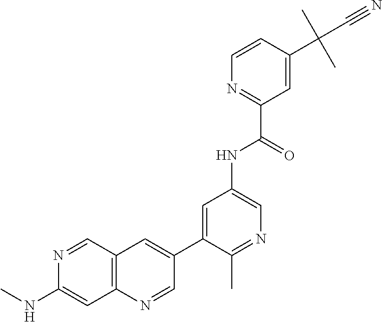 [SMILES: CNc1cc2ncc(-c3cc(NC(=O)c4cc(C(C)(C)C#N)ccn4)cnc3C)cc2cn1] |
| 31 | 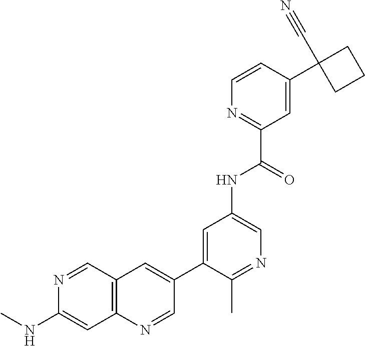 [SMILES: CNc1cc2ncc(-c3cc(NC(=O)c4cc(C5(C#N)CCC5)ccn4)cnc3C)cc2cn1] |
| 32 | 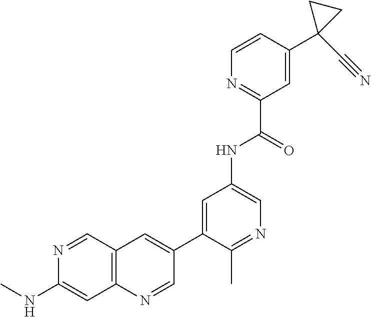 [SMILES: CNc1cc2ncc(-c3cc(NC(=O)c4cc(C5(C#N)CC5)ccn4)cnc3C)cc2cn1] |
| 33 | 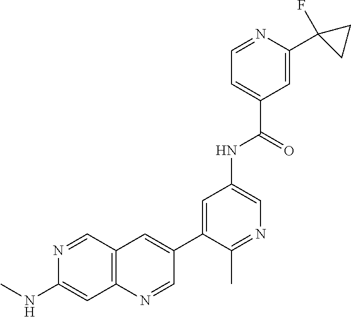 [SMILES: CNc1cc2ncc(-c3cc(NC(=O)c4ccnc(C5(F)CC5)c4)cnc3C)cc2cn1] |
| 34 | 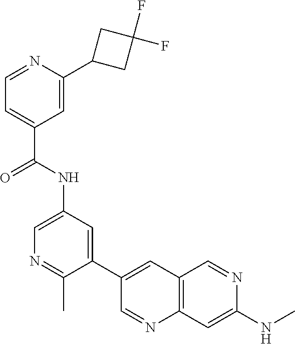 [SMILES: CNc1cc2ncc(-c3cc(NC(=O)c4ccnc(C5CC(F)(F)C5)c4)cnc3C)cc2cn1] |
| 35 | 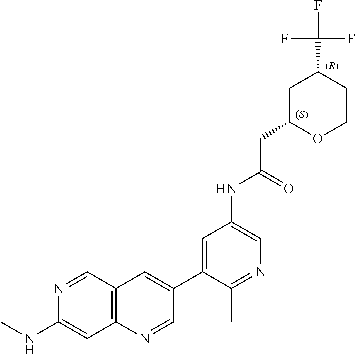 |
| 36 | 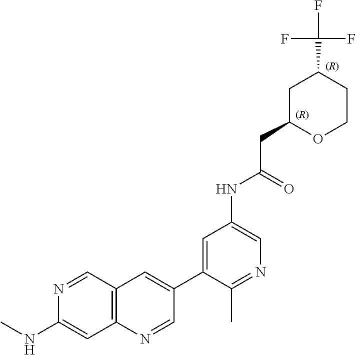 |
| 37 | 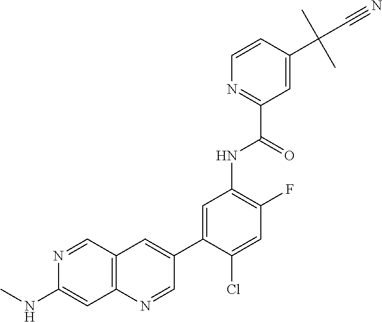 [SMILES: CNc1cc2ncc(-c3cc(NC(=O)c4cc(C(C)(C)C#N)ccn4)c(F)cc3Cl)cc2cn1] |
| 38 | 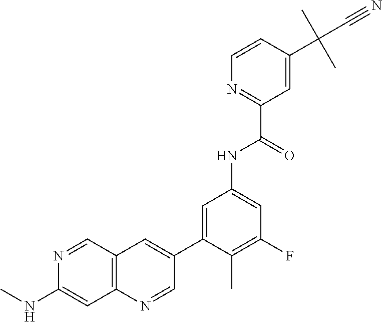 [SMILES: CNc1cc2ncc(-c3cc(NC(=O)c4cc(C(C)(C)C#N)ccn4)cc(F)c3C)cc2cn1] |
| 39 | 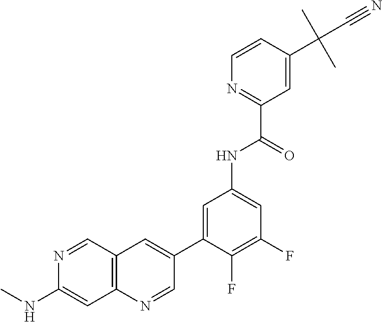 [SMILES: CNc1cc2ncc(-c3cc(NC(=O)c4cc(C(C)(C)C#N)ccn4)cc(F)c3F)cc2cn1] |
| 40 | 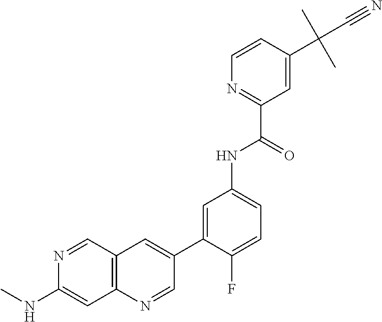 [SMILES: CNc1cc2ncc(-c3cc(NC(=O)c4cc(C(C)(C)C#N)ccn4)ccc3F)cc2cn1] |
| 41 | 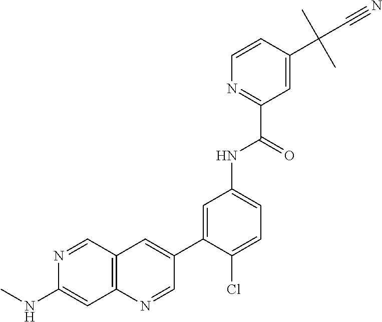 [SMILES: CNc1cc2ncc(-c3cc(NC(=O)c4cc(C(C)(C)C#N)ccn4)ccc3Cl)cc2cn1] |
| 42 | 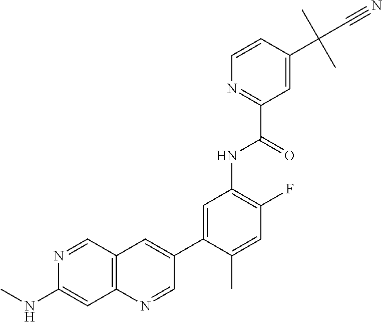 [SMILES: CNc1cc2ncc(-c3cc(NC(=O)c4cc(C(C)(C)C#N)ccn4)c(F)cc3C)cc2cn1] |
| 43 | 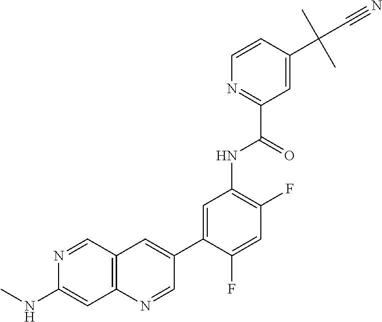 [SMILES: CNc1cc2ncc(-c3cc(NC(=O)c4cc(C(C)(C)C#N)ccn4)c(F)cc3F)cc2cn1] |
| 44 | 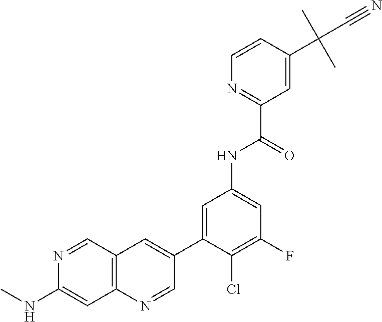 [SMILES: CNc1cc2ncc(-c3cc(NC(=O)c4cc(C(C)(C)C#N)ccn4)cc(F)c3Cl)cc2cn1] |
| 45 | 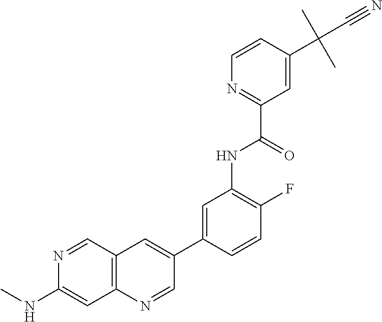 [SMILES: CNc1cc2ncc(-c3ccc(F)c(NC(=O)c4cc(C(C)(C)C#N)ccn4)c3)cc2cn1] |
| 46 | 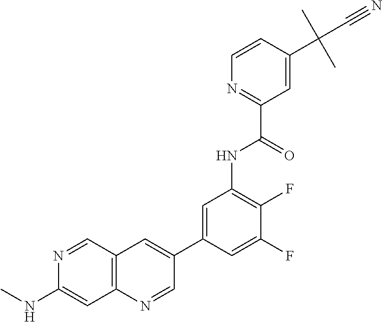 [SMILES: CNc1cc2ncc(-c3cc(F)c(F)c(NC(=O)c4cc(C(C)(C)C#N)ccn4)c3)cc2cn1] |
| 47 | 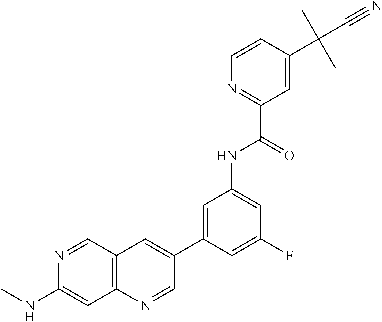 [SMILES: CNc1cc2ncc(-c3cc(F)cc(NC(=O)c4cc(C(C)(C)C#N)ccn4)c3)cc2cn1] |
| 48 | 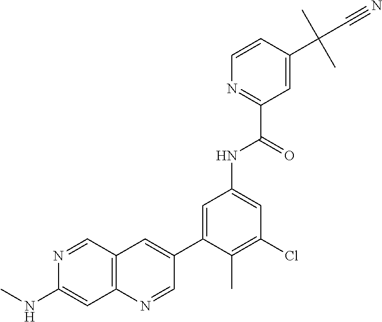 [SMILES: CNc1cc2ncc(-c3cc(NC(=O)c4cc(C(C)(C)C#N)ccn4)cc(Cl)c3C)cc2cn1] |
| 49 | 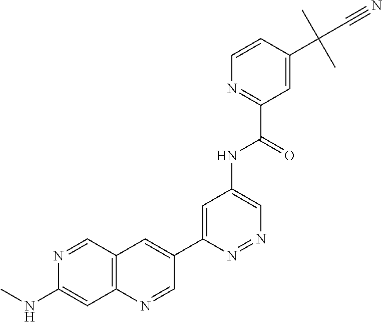 [SMILES: CNc1cc2ncc(-c3cc(NC(=O)c4cc(C(C)(C)C#N)ccn4)cnn3)cc2cn1] |
| 50 | 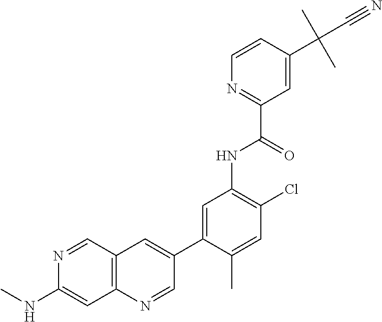 [SMILES: CNc1cc2ncc(-c3cc(NC(=O)c4cc(C(C)(C)C#N)ccn4)c(Cl)cc3C)cc2cn1] |
| 51 | 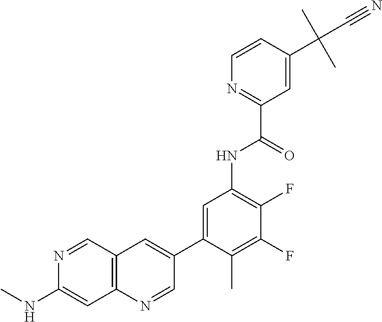 [SMILES: CNc1cc2ncc(-c3cc(NC(=O)c4cc(C(C)(C)C#N)ccn4)c(F)c(F)c3C)cc2cn1] |
| 52 | 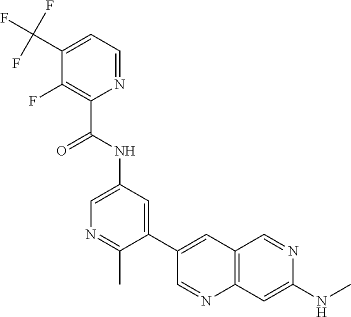 [SMILES: CNc1cc2ncc(-c3cc(NC(=O)c4nccc(C(F)(F)F)c4F)cnc3C)cc2cn1] |
| 53 | 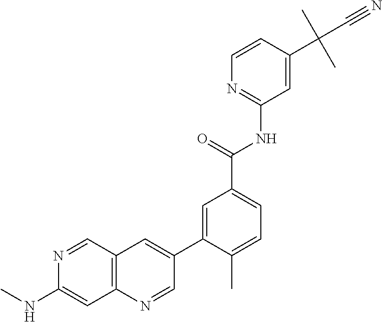 [SMILES: CNc1cc2ncc(-c3cc(C(=O)Nc4cc(C(C)(C)C#N)ccn4)ccc3C)cc2cn1] |
| 54 | 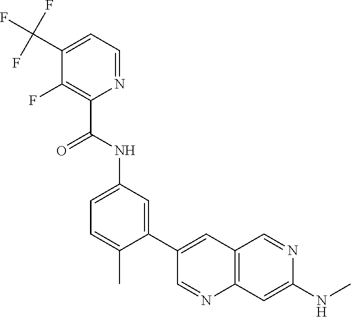 [SMILES: CNc1cc2ncc(-c3cc(NC(=O)c4nccc(C(F)(F)F)c4F)ccc3C)cc2cn1] |
| 55 | 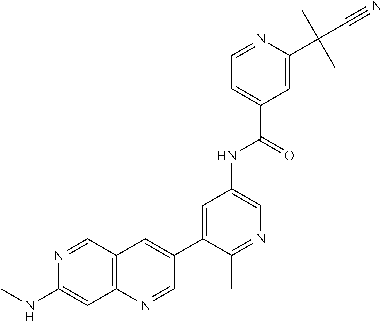 [SMILES: CNc1cc2ncc(-c3cc(NC(=O)c4ccnc(C(C)(C)C#N)c4)cnc3C)cc2cn1] |
| 56 | 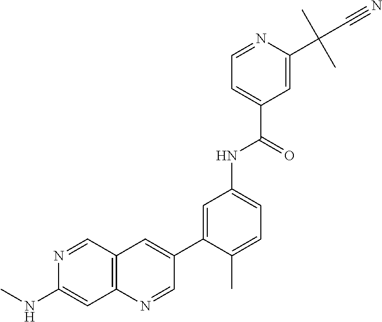 [SMILES: CNc1cc2ncc(-c3cc(NC(=O)c4ccnc(C(C)(C)C#N)c4)ccc3C)cc2cn1] |
| 57 | 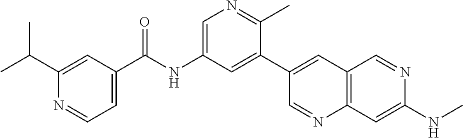 [SMILES: CNc1cc2ncc(-c3cc(NC(=O)c4ccnc(C(C)C)c4)cnc3C)cc2cn1] |
| 58 | 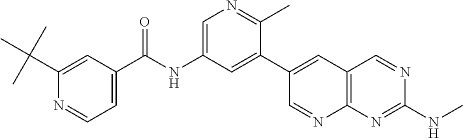 [SMILES: CNc1ncc2cc(-c3cc(NC(=O)c4ccnc(C(C)(C)C)c4)cnc3C)cnc2n1] |
| 59 | 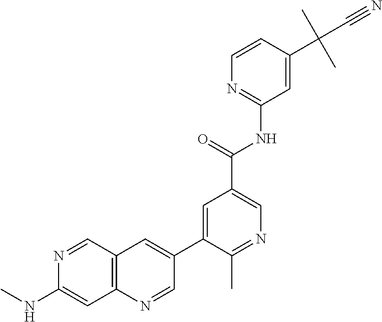 [SMILES: CNc1cc2ncc(-c3cc(C(=O)Nc4cc(C(C)(C)C#N)ccn4)cnc3C)cc2cn1] |
| 60 | 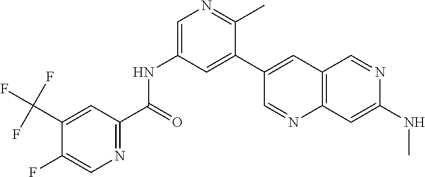 [SMILES: CNc1cc2ncc(-c3cc(NC(=O)c4cc(C(F)(F)F)c(F)cn4)cnc3C)cc2cn1] |
| 61 | 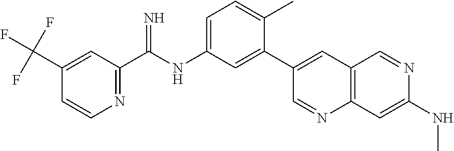 [SMILES: CNc1cc2ncc(-c3cc(NC(=N)c4cc(C(F)(F)F)ccn4)ccc3C)cc2cn1] |
| 62 | 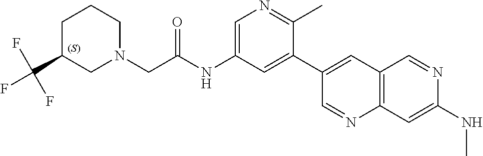 |
| 63 | 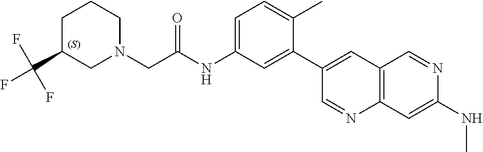 |
| 64 | 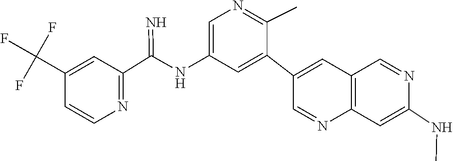 [SMILES: CNc1cc2ncc(-c3cc(NC(=N)c4cc(C(F)(F)F)ccn4)cnc3C)cc2cn1] |
| 65 | 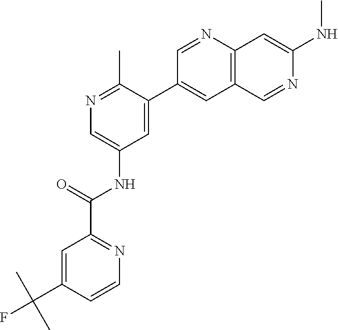 [SMILES: CNc1cc2ncc(-c3cc(NC(=O)c4cc(C(C)(C)F)ccn4)cnc3C)cc2cn1] |
| 66 | 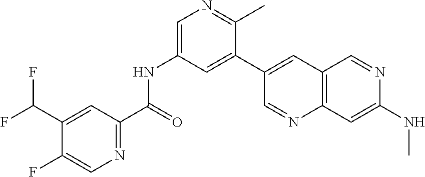 [SMILES: CNc1cc2ncc(-c3cc(NC(=O)c4cc(C(F)F)c(F)cn4)cnc3C)cc2cn1] |
| 67 | 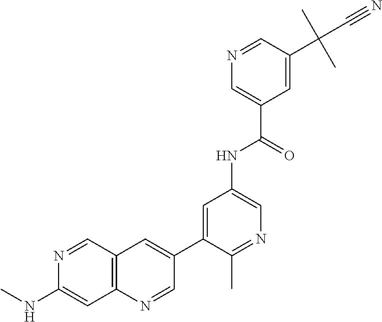 [SMILES: CNc1cc2ncc(-c3cc(NC(=O)c4cncc(C(C)(C)C#N)c4)cnc3C)cc2cn1] |
| 68 | 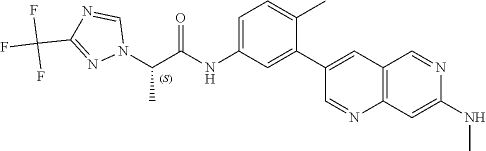 |
| 69 | 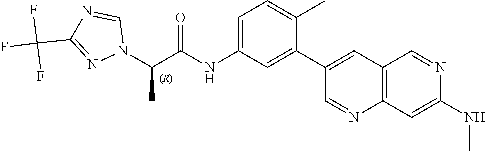 |
| 70 | 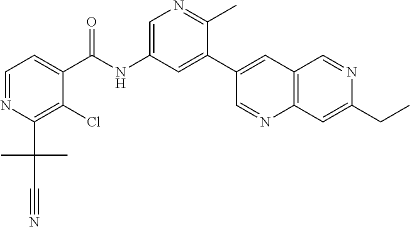 [SMILES: CCc1cc2ncc(-c3cc(NC(=O)c4ccnc(C(C)(C)C#N)c4Cl)cnc3C)cc2cn1] |
| 71 | 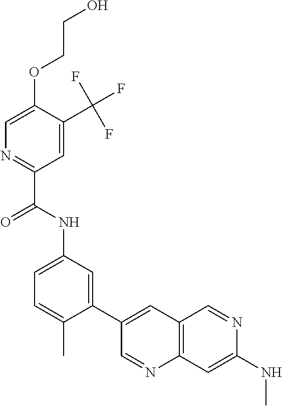 [SMILES: CNc1cc2ncc(-c3cc(NC(=O)c4cc(C(F)(F)F)c(OCCO)cn4)ccc3C)cc2cn1] |
| 72 | 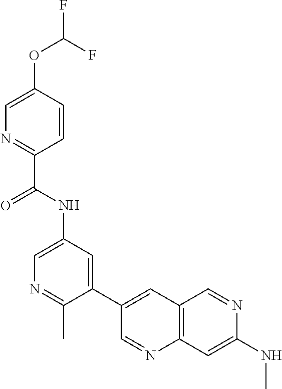 [SMILES: CNc1cc2ncc(-c3cc(NC(=O)c4ccc(OC(F)F)cn4)cnc3C)cc2cn1] |
| 73 | 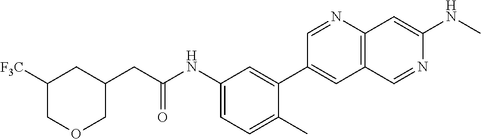 [SMILES: CNc1cc2ncc(-c3cc(NC(=O)CC4COCC(C(F)(F)F)C4)ccc3C)cc2cn1] |
| 74 | 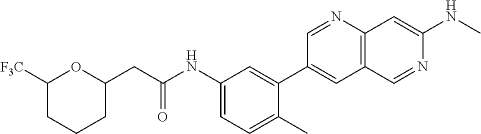 [SMILES: CNc1cc2ncc(-c3cc(NC(=O)CC4CCCC(C(F)(F)F)O4)ccc3C)cc2cn1] |
| 75 | 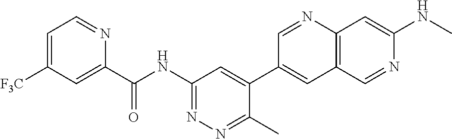 [SMILES: CNc1cc2ncc(-c3cc(NC(=O)c4cc(C(F)(F)F)ccn4)nnc3C)cc2cn1] |
| 76 | 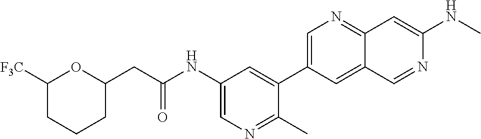 [SMILES: CNc1cc2ncc(-c3cc(NC(=O)CC4CCCC(C(F)(F)F)O4)cnc3C)cc2cn1] |
| 77 | 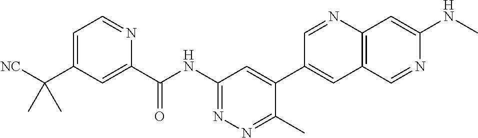 [SMILES: CNc1cc2ncc(-c3cc(NC(=O)c4cc(C(C)(C)C#N)ccn4)nnc3C)cc2cn1] |
| 78 | 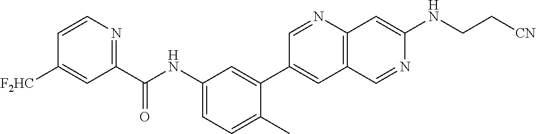 [SMILES: Cc1ccc(NC(=O)c2cc(C(F)F)ccn2)cc1-c1cnc2cc(NCCC#N)ncc2c1] |
| 79 | 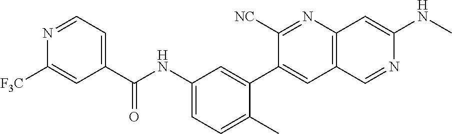 [SMILES: CNc1cc2nc(C#N)c(-c3cc(NC(=O)c4ccnc(C(F)(F)F)c4)ccc3C)cc2cn1] |
| 80 | 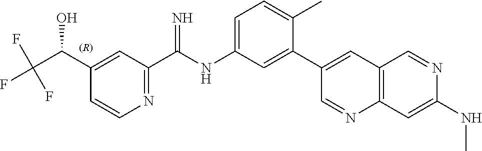 |
| 81 | 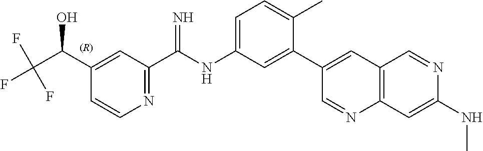 |
| 82 | 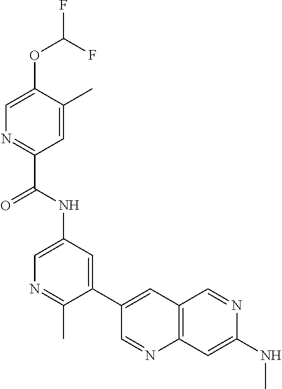 [SMILES: CNc1cc2ncc(-c3cc(NC(=O)c4cc(C)c(OC(F)F)cn4)cnc3C)cc2cn1] |
| 83 | 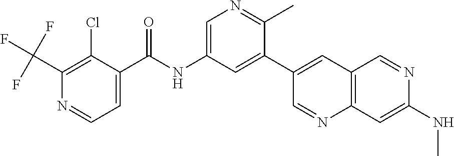 [SMILES: CNc1cc2ncc(-c3cc(NC(=O)c4ccnc(C(F)(F)F)c4Cl)cnc3C)cc2cn1] |
| 84 | 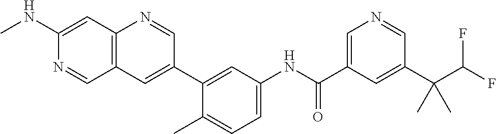 [SMILES: CNc1cc2ncc(-c3cc(NC(=O)c4cncc(C(C)(C)C(F)F)c4)ccc3C)cc2cn1] |
| 85 | 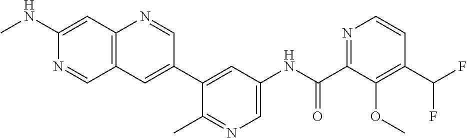 [SMILES: CNc1cc2ncc(-c3cc(NC(=O)c4nccc(C(F)F)c4OC)cnc3C)cc2cn1] |
| 86 | 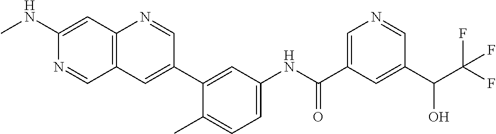 [SMILES: CNc1cc2ncc(-c3cc(NC(=O)c4cncc(C(O)C(F)(F)F)c4)ccc3C)cc2cn1] |
| 87 | 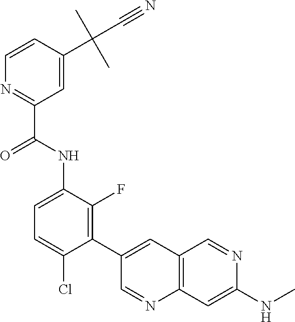 [SMILES: CNc1cc2ncc(-c3c(Cl)ccc(NC(=O)c4cc(C(C)(C)C#N)ccn4)c3F)cc2cn1] |
| 88 | 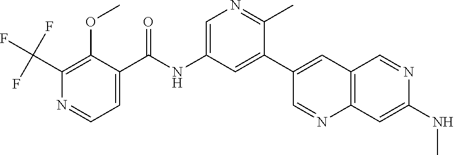 [SMILES: CNc1cc2ncc(-c3cc(NC(=O)c4ccnc(C(F)(F)F)c4OC)cnc3C)cc2cn1] |
| 89 | 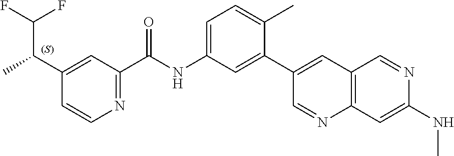 |
| 90 | 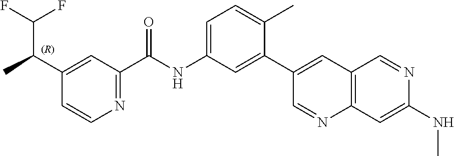 |
| 91 | 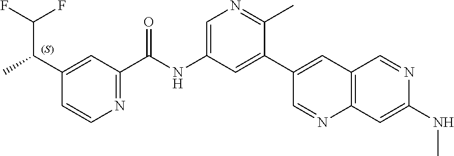 |
| 92 | 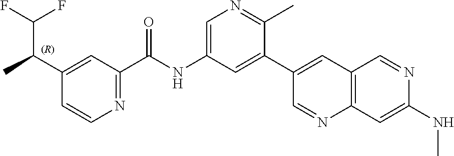 |
| 93 | 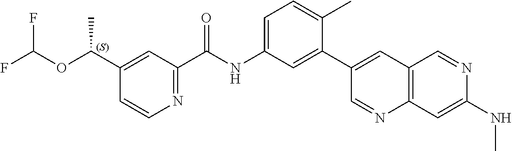 |
| 94 | 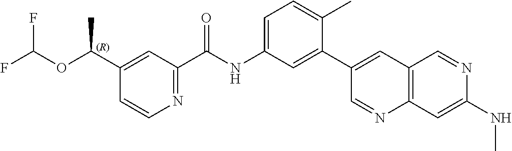 |
| 95 | 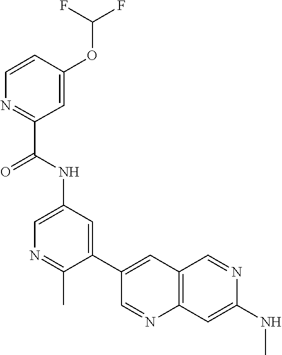 [SMILES: CNc1cc2ncc(-c3cc(NC(=O)c4cc(OC(F)F)ccn4)cnc3C)cc2cn1] |
| 96 | 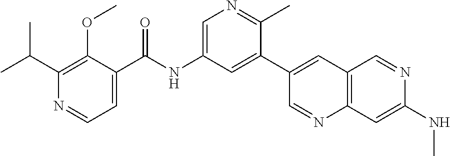 [SMILES: CNc1cc2ncc(-c3cc(NC(=O)c4ccnc(C(C)C)c4OC)cnc3C)cc2cn1] |
| 97 | 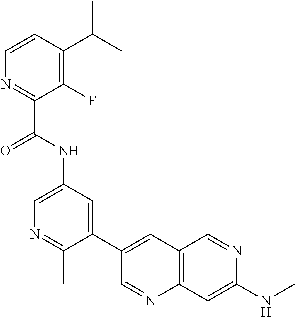 [SMILES: CNc1cc2ncc(-c3cc(NC(=O)c4nccc(C(C)C)c4F)cnc3C)cc2cn1] |
| 98 | 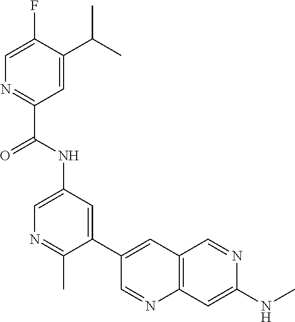 [SMILES: CNc1cc2ncc(-c3cc(NC(=O)c4cc(C(C)C)c(F)cn4)cnc3C)cc2cn1] |
| 99 | 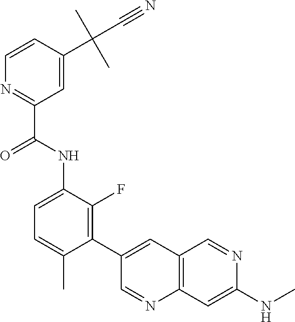 [SMILES: CNc1cc2ncc(-c3c(C)ccc(NC(=O)c4cc(C(C)(C)C#N)ccn4)c3F)cc2cn1] |
| 100 | 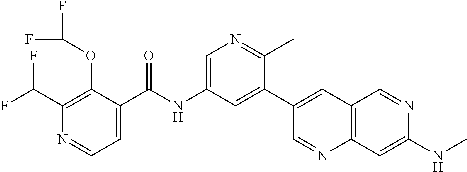 [SMILES: CNc1cc2ncc(-c3cc(NC(=O)c4ccnc(C(F)F)c4OC(F)F)cnc3C)cc2cn1] |
| 101 | 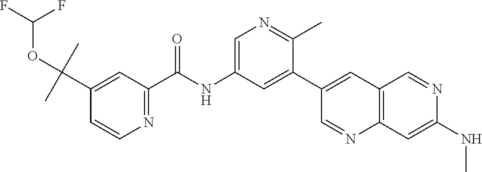 [SMILES: CNc1cc2ncc(-c3cc(NC(=O)c4cc(C(C)(C)OC(F)F)ccn4)cnc3C)cc2cn1] |
| 102 | 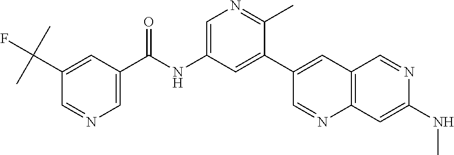 [SMILES: CNc1cc2ncc(-c3cc(NC(=O)c4cncc(C(C)(C)F)c4)cnc3C)cc2cn1] |
| 103 | 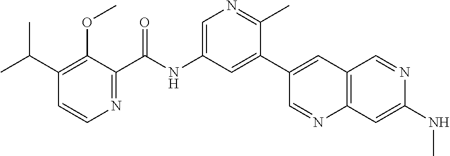 [SMILES: CNc1cc2ncc(-c3cc(NC(=O)c4nccc(C(C)C)c4OC)cnc3C)cc2cn1] |
| 104 | 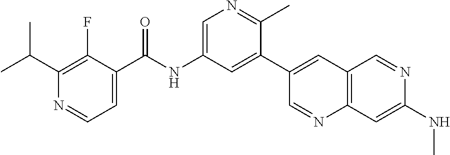 [SMILES: CNc1cc2ncc(-c3cc(NC(=O)c4ccnc(C(C)C)c4F)cnc3C)cc2cn1] |
| 105 | 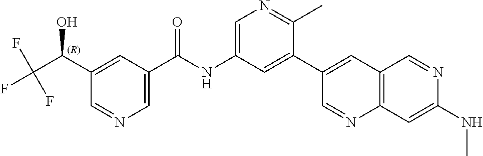 |
| 106 | 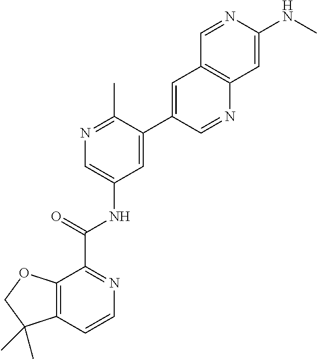 |
| 107 | 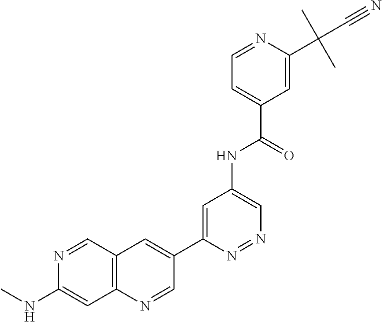 [SMILES: CNc1cc2ncc(-c3cc(NC(=O)c4ccnc(C(C)(C)C#N)c4)cnn3)cc2cn1] |
| 108 | 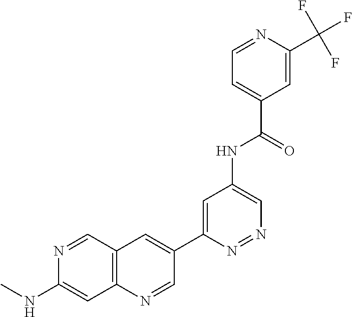 [SMILES: CNc1cc2ncc(-c3cc(NC(=O)c4ccnc(C(F)(F)F)c4)cnn3)cc2cn1] |
| 109 | 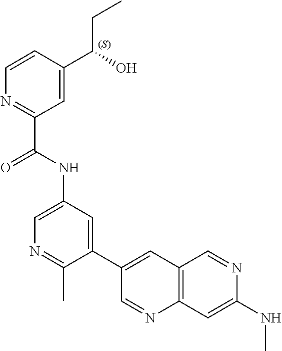 |
| 110 | 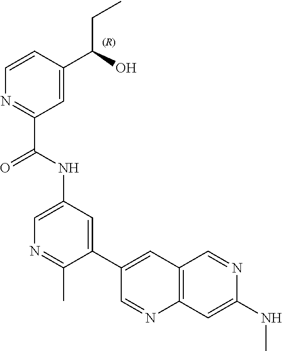 |
| 111 | 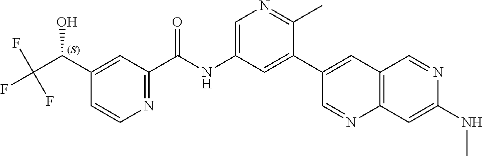 |
| 112 | 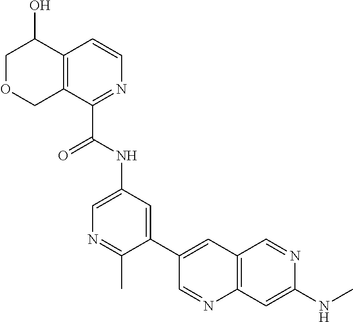 [SMILES: CNc1cc2ncc(-c3cc(NC(=O)c4nccc5c4COCC5O)cnc3C)cc2cn1] |
| 113 | 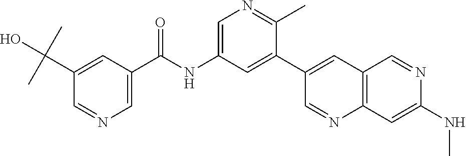 [SMILES: CNc1cc2ncc(-c3cc(NC(=O)c4cncc(C(C)(C)O)c4)cnc3C)cc2cn1] |
| 114 | 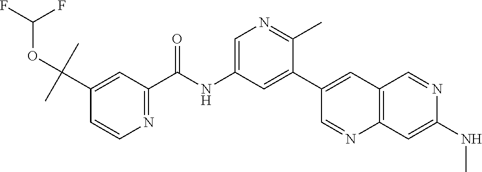 [SMILES: CNc1cc2ncc(-c3cc(NC(=O)c4cc(C(C)(C)OC(F)F)ccn4)cnc3C)cc2cn1] |
| 115 | 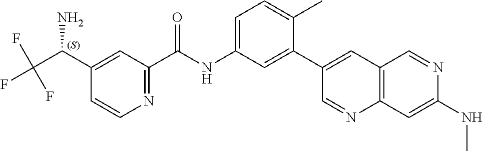 |
| 116 | 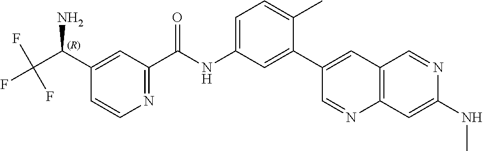 |
| 117 | 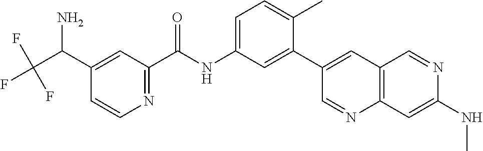 [SMILES: CNc1cc2ncc(-c3cc(NC(=O)c4cc(C(N)C(F)(F)F)ccn4)ccc3C)cc2cn1] |
| 118 | 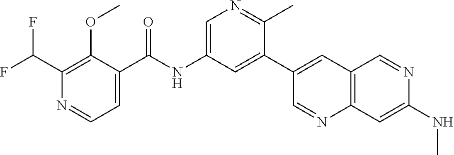 [SMILES: CNc1cc2ncc(-c3cc(NC(=O)c4ccnc(C(F)F)c4OC)cnc3C)cc2cn1] |
| 119 | 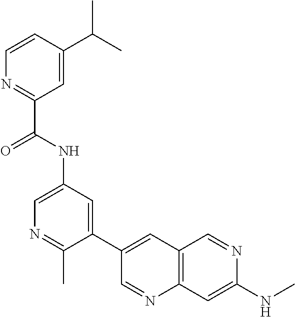 [SMILES: CNc1cc2ncc(-c3cc(NC(=O)c4cc(C(C)C)ccn4)cnc3C)cc2cn1] |
| 120 | 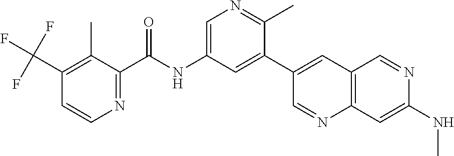 [SMILES: CNc1cc2ncc(-c3cc(NC(=O)c4nccc(C(F)(F)F)c4C)cnc3C)cc2cn1] |
| 121 | 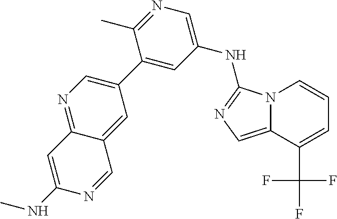 [SMILES: CNc1cc2ncc(-c3cc(Nc4ncc5c(C(F)(F)F)cccn45)cnc3C)cc2cn1] |
| 122 | 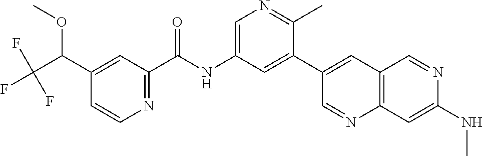 [SMILES: CNc1cc2ncc(-c3cc(NC(=O)c4cc(C(OC)C(F)(F)F)ccn4)cnc3C)cc2cn1] |
| 123 | 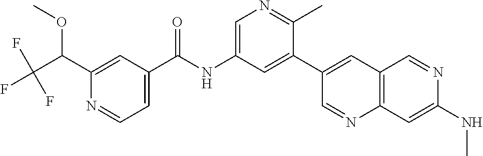 [SMILES: CNc1cc2ncc(-c3cc(NC(=O)c4ccnc(C(OC)C(F)(F)F)c4)cnc3C)cc2cn1] |
| 124 | 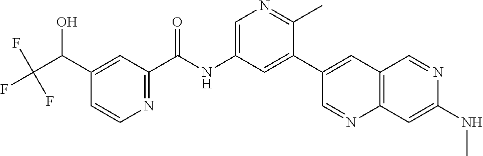 [SMILES: CNc1cc2ncc(-c3cc(NC(=O)c4cc(C(O)C(F)(F)F)ccn4)cnc3C)cc2cn1] |
| 125 | 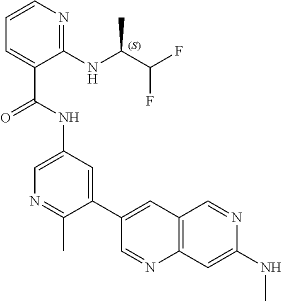 |
| 126 | 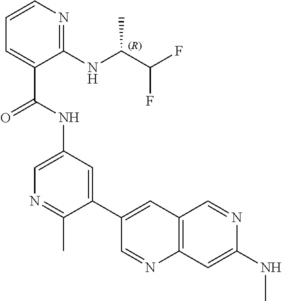 |
| 127 | 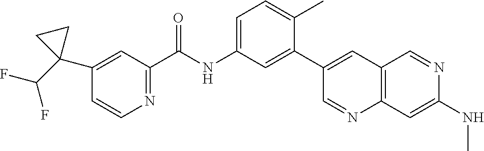 [SMILES: CNc1cc2ncc(-c3cc(NC(=O)c4cc(C5(C(F)F)CC5)ccn4)ccc3C)cc2cn1] |
| 128 | 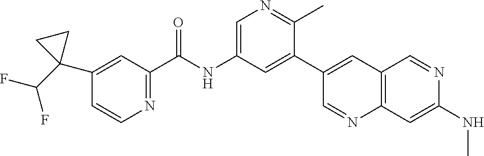 [SMILES: CNc1cc2ncc(-c3cc(NC(=O)c4cc(C5(C(F)F)CC5)ccn4)cnc3C)cc2cn1] |
| 129 | 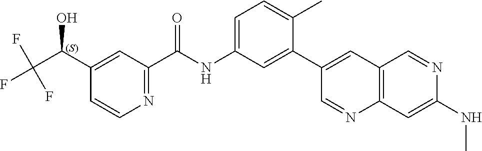 |
| 130 | 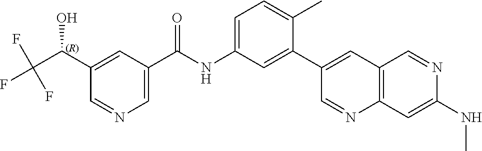 |
| 131 | 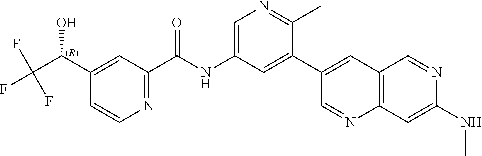 |
| 132 | 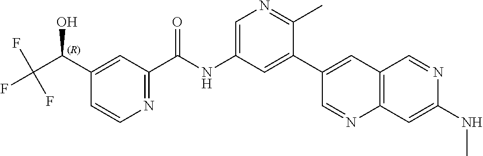 |
| 133 | 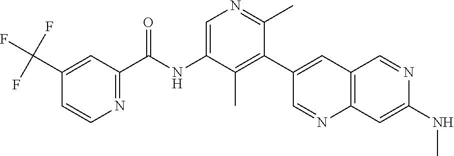 [SMILES: CNc1cc2ncc(-c3c(C)ncc(NC(=O)c4cc(C(F)(F)F)ccn4)c3C)cc2cn1] |
| 134 | 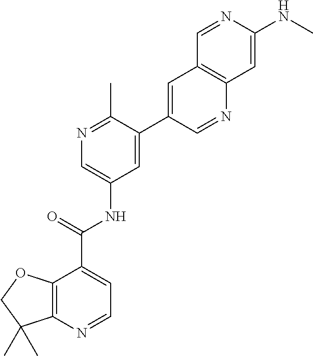 [SMILES: CNc1cc2ncc(-c3cc(NC(=O)c4ccnc5c4OCC5(C)C)cnc3C)cc2cn1] |
| 135 | 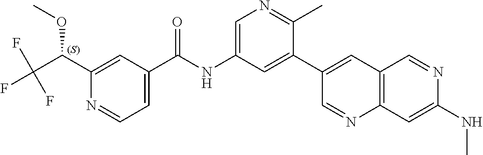 |
| 136 | 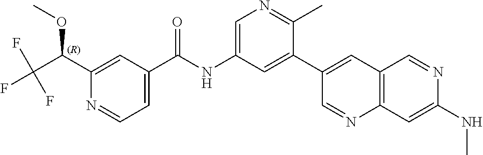 |
| 137 | 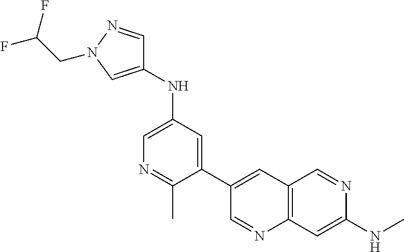 [SMILES: CNc1cc2ncc(-c3cc(Nc4cnn(CC(F)F)c4)cnc3C)cc2cn1] |
| 138 | 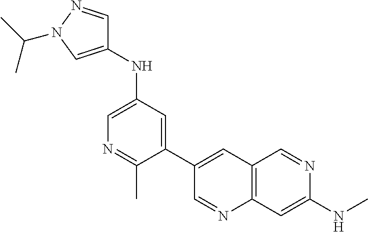 [SMILES: CNc1cc2ncc(-c3cc(Nc4cnn(C(C)C)c4)cnc3C)cc2cn1] |
| 139 | 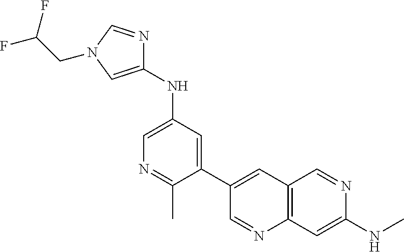 [SMILES: CNc1cc2ncc(-c3cc(Nc4cn(CC(F)F)cn4)cnc3C)cc2cn1] |
| 140 | 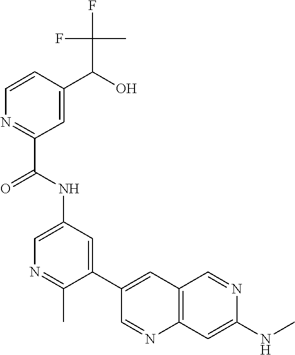 [SMILES: CNc1cc2ncc(-c3cc(NC(=O)c4cc(C(O)C(C)(F)F)ccn4)cnc3C)cc2cn1] |
| 141 | 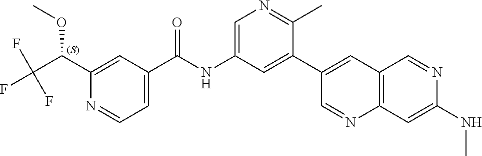 |
| 142 | 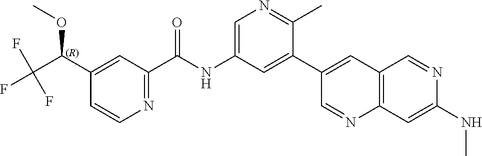 |
| 143 | 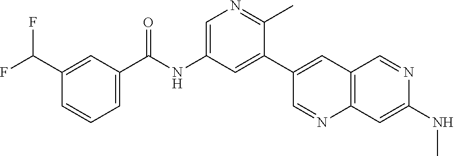 [SMILES: CNc1cc2ncc(-c3cc(NC(=O)c4cccc(C(F)F)c4)cnc3C)cc2cn1] |
| 144 | 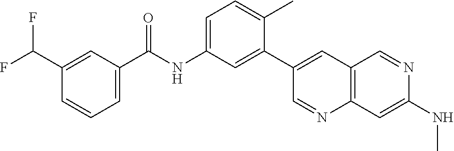 [SMILES: CNc1cc2ncc(-c3cc(NC(=O)c4cccc(C(F)F)c4)ccc3C)cc2cn1] |
| 145 | 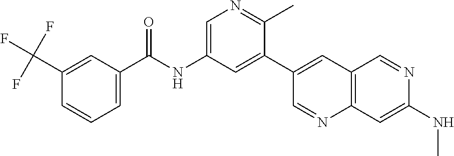 [SMILES: CNc1cc2ncc(-c3cc(NC(=O)c4cccc(C(F)(F)F)c4)cnc3C)cc2cn1] |
| 146 | 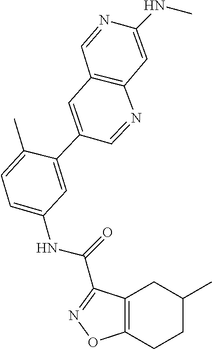 [SMILES: CNc1cc2ncc(-c3cc(NC(=O)c4noc5c4CC(C)CC5)ccc3C)cc2cn1] |
| 147 | 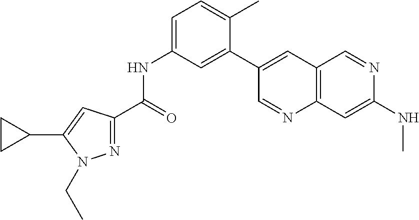 [SMILES: CCn1nc(C(=O)Nc2ccc(C)c(-c3cnc4cc(NC)ncc4c3)c2)cc1C1CC1] |
| 148 | 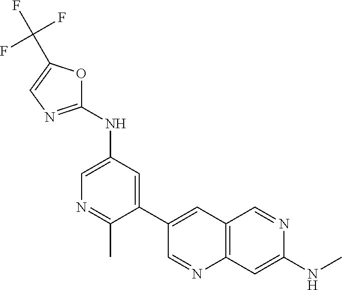 [SMILES: CNc1cc2ncc(-c3cc(Nc4ncc(C(F)(F)F)o4)cnc3C)cc2cn1] |
| 149 | 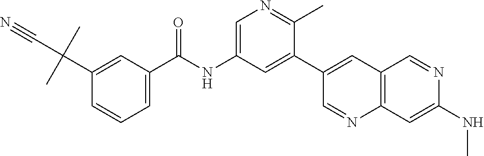 [SMILES: CNc1cc2ncc(-c3cc(NC(=O)c4cccc(C(C)(C)C#N)c4)cnc3C)cc2cn1] |
| 150 | 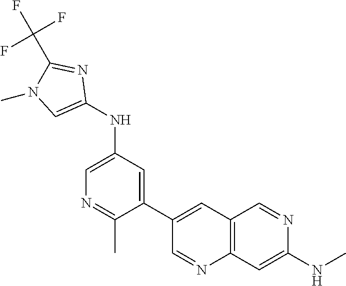 |
| 151 | 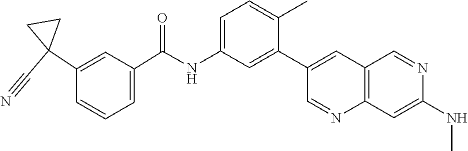 [SMILES: CNc1cc2ncc(-c3cc(NC(=O)c4cccc(C5(C#N)CC5)c4)ccc3C)cc2cn1] |
| 152 | 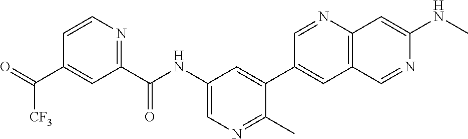 [SMILES: CNc1cc2ncc(-c3cc(NC(=O)c4cc(C(=O)C(F)(F)F)ccn4)cnc3C)cc2cn1] |
| 153 | 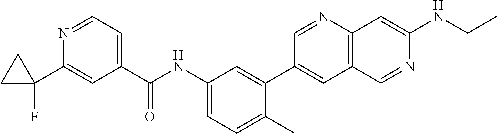 [SMILES: CCNc1cc2ncc(-c3cc(NC(=O)c4ccnc(C5(F)CC5)c4)ccc3C)cc2cn1] |
| 154 | 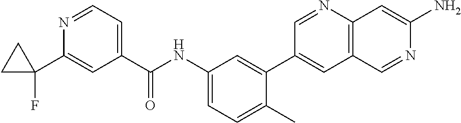 [SMILES: Cc1ccc(NC(=O)c2ccnc(C3(F)CC3)c2)cc1-c1cnc2cc(N)ncc2c1] |
| 155 | 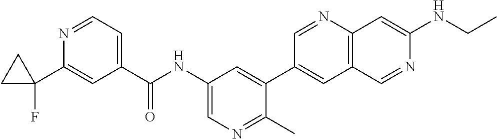 [SMILES: CCNc1cc2ncc(-c3cc(NC(=O)c4ccnc(C5(F)CC5)c4)cnc3C)cc2cn1] |
| 156 | 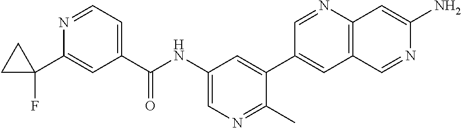 [SMILES: Cc1ncc(NC(=O)c2ccnc(C3(F)CC3)c2)cc1-c1cnc2cc(N)ncc2c1] |
| 157 | 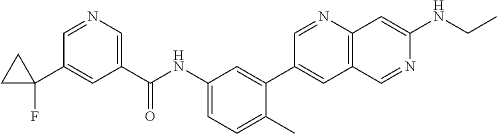 [SMILES: CCNc1cc2ncc(-c3cc(NC(=O)c4cncc(C5(F)CC5)c4)ccc3C)cc2cn1] |
| 158 | 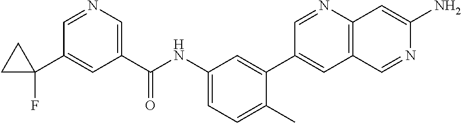 [SMILES: Cc1ccc(NC(=O)c2cncc(C3(F)CC3)c2)cc1-c1cnc2cc(N)ncc2c1] |
| 159 | 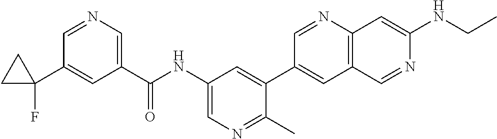 [SMILES: CCNc1cc2ncc(-c3cc(NC(=O)c4cncc(C5(F)CC5)c4)cnc3C)cc2cn1] |
| 160 | 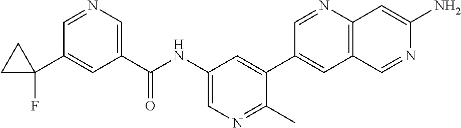 [SMILES: Cc1ncc(NC(=O)c2cncc(C3(F)CC3)c2)cc1-c1cnc2cc(N)ncc2c1] |
| 161 | 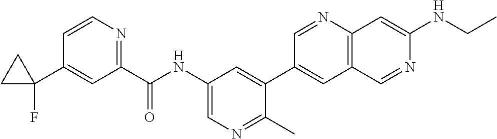 [SMILES: CCNc1cc2ncc(-c3cc(NC(=O)c4cc(C5(F)CC5)ccn4)cnc3C)cc2cn1] |
| 162 | 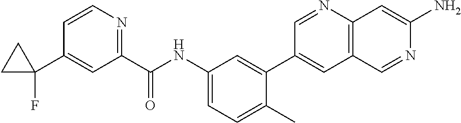 [SMILES: Cc1ccc(NC(=O)c2cc(C3(F)CC3)ccn2)cc1-c1cnc2cc(N)ncc2c1] |
| 163 | 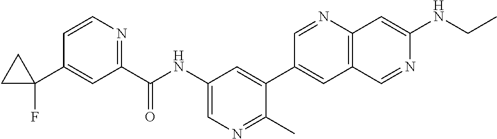 [SMILES: CCNc1cc2ncc(-c3cc(NC(=O)c4cc(C5(F)CC5)ccn4)cnc3C)cc2cn1] |
| 164 | 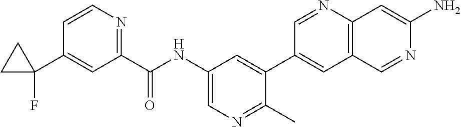 [SMILES: Cc1ncc(NC(=O)c2cc(C3(F)CC3)ccn2)cc1-c1cnc2cc(N)ncc2c1] |
| 165 | 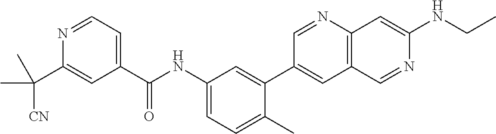 [SMILES: CCNc1cc2ncc(-c3cc(NC(=O)c4ccnc(C(C)(C)C#N)c4)ccc3C)cc2cn1] |
| 166 | 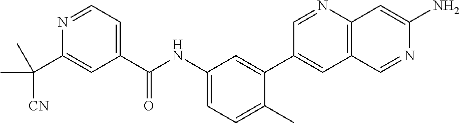 [SMILES: Cc1ccc(NC(=O)c2ccnc(C(C)(C)C#N)c2)cc1-c1cnc2cc(N)ncc2c1] |
| 167 | 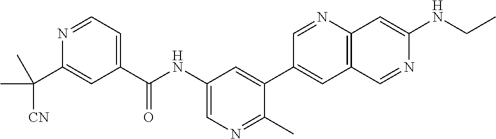 [SMILES: CCNc1cc2ncc(-c3cc(NC(=O)c4ccnc(C(C)(C)C#N)c4)cnc3C)cc2cn1] |
| 168 | 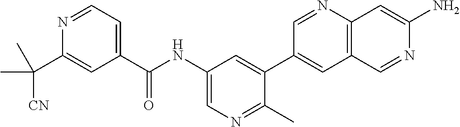 [SMILES: Cc1ncc(NC(=O)c2ccnc(C(C)(C)C#N)c2)cc1-c1cnc2cc(N)ncc2c1] |
| 169 | 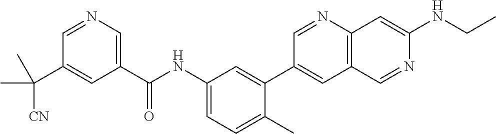 [SMILES: CCNc1cc2ncc(-c3cc(NC(=O)c4cncc(C(C)(C)C#N)c4)ccc3C)cc2cn1] |
| 170 | 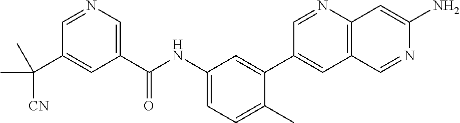 [SMILES: Cc1ccc(NC(=O)c2cncc(C(C)(C)C#N)c2)cc1-c1cnc2cc(N)ncc2c1] |
| 171 | 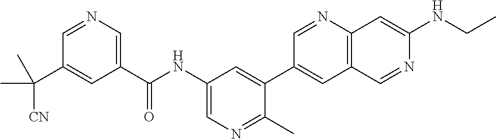 [SMILES: CCNc1cc2ncc(-c3cc(NC(=O)c4cncc(C(C)(C)C#N)c4)cnc3C)cc2cn1] |
| 172 | 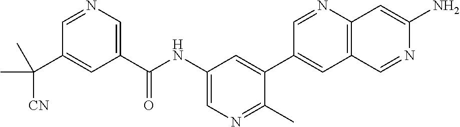 [SMILES: Cc1ncc(NC(=O)c2cncc(C(C)(C)C#N)c2)cc1-c1cnc2cc(N)ncc2c1] |
| 173 | 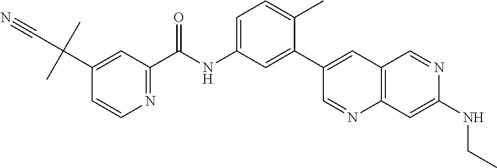 [SMILES: CCNc1cc2ncc(-c3cc(NC(=O)c4cc(C(C)(C)C#N)ccn4)ccc3C)cc2cn1] |
| 174 | 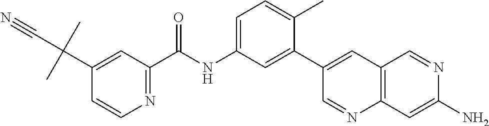 [SMILES: Cc1ccc(NC(=O)c2cc(C(C)(C)C#N)ccn2)cc1-c1cnc2cc(N)ncc2c1] |
| 175 | 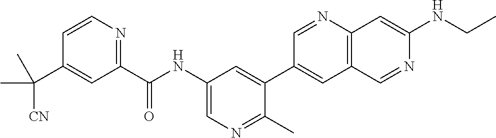 [SMILES: CCNc1cc2ncc(-c3cc(NC(=O)c4cc(C(C)(C)C#N)ccn4)cnc3C)cc2cn1] |
| 176 | 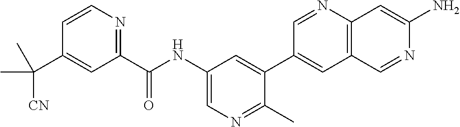 [SMILES: Cc1ncc(NC(=O)c2cc(C(C)(C)C#N)ccn2)cc1-c1cnc2cc(N)ncc2c1] |
| 177 | 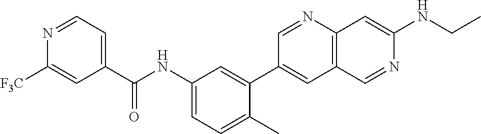 [SMILES: CCNc1cc2ncc(-c3cc(NC(=O)c4ccnc(C(F)(F)F)c4)ccc3C)cc2cn1] |
| 178 | 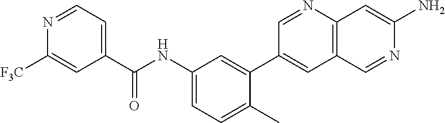 [SMILES: Cc1ccc(NC(=O)c2ccnc(C(F)(F)F)c2)cc1-c1cnc2cc(N)ncc2c1] |
| 179 | 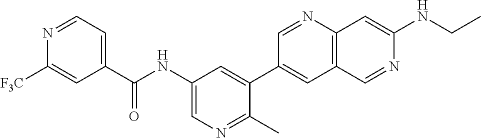 [SMILES: CCNc1cc2ncc(-c3cc(NC(=O)c4ccnc(C(F)(F)F)c4)cnc3C)cc2cn1] |
| 180 | 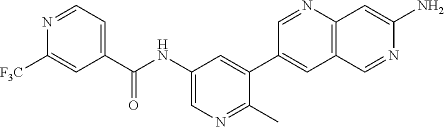 [SMILES: Cc1ncc(NC(=O)c2ccnc(C(F)(F)F)c2)cc1-c1cnc2cc(N)ncc2c1] |
| 181 | 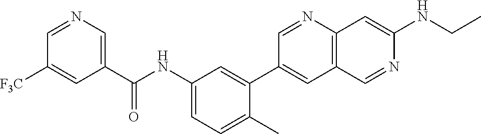 [SMILES: CCNc1cc2ncc(-c3cc(NC(=O)c4cncc(C(F)(F)F)c4)ccc3C)cc2cn1] |
| 182 | 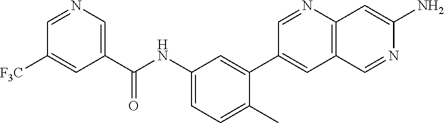 [SMILES: Cc1ccc(NC(=O)c2cncc(C(F)(F)F)c2)cc1-c1cnc2cc(N)ncc2c1] |
| 183 | 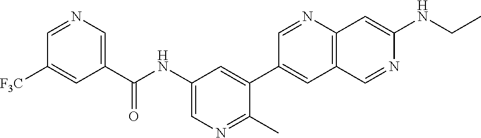 [SMILES: CCNc1cc2ncc(-c3cc(NC(=O)c4cncc(C(F)(F)F)c4)cnc3C)cc2cn1] |
| 184 | 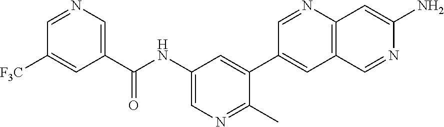 [SMILES: Cc1ncc(NC(=O)c2cncc(C(F)(F)F)c2)cc1-c1cnc2cc(N)ncc2c1] |
| 185 | 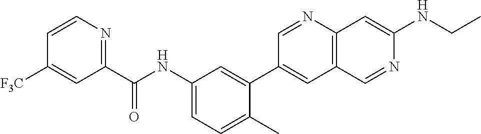 [SMILES: CCNc1cc2ncc(-c3cc(NC(=O)c4cc(C(F)(F)F)ccn4)ccc3C)cc2cn1] |
| 186 | 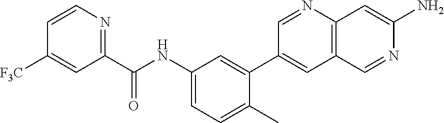 [SMILES: Cc1ccc(NC(=O)c2cc(C(F)(F)F)ccn2)cc1-c1cnc2cc(N)ncc2c1] |
| 187 | 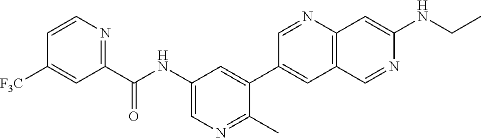 [SMILES: CCNc1cc2ncc(-c3cc(NC(=O)c4cc(C(F)(F)F)ccn4)cnc3C)cc2cn1] |
| 188 | 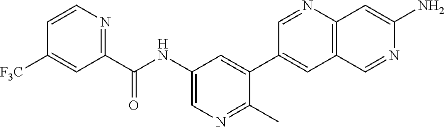 [SMILES: Cc1ncc(NC(=O)c2cc(C(F)(F)F)ccn2)cc1-c1cnc2cc(N)ncc2c1] |
| 189 | 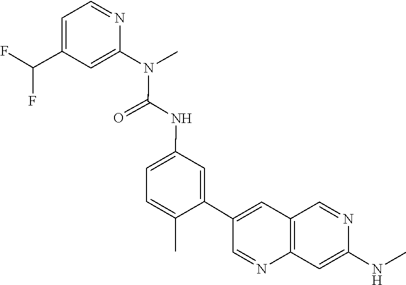 [SMILES: CNc1cc2ncc(-c3cc(NC(=O)N(C)c4cc(C(F)F)ccn4)ccc3C)cc2cn1] |
| 190 | 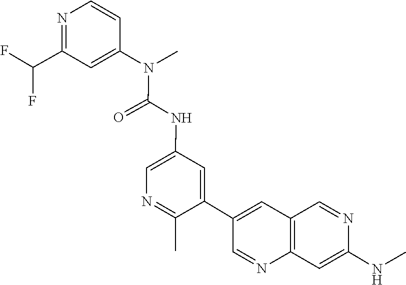 [SMILES: CNc1cc2ncc(-c3cc(NC(=O)N(C)c4ccnc(C(F)F)c4)cnc3C)cc2cn1] |
| 191 | 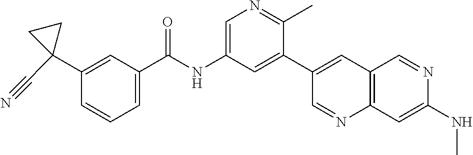 [SMILES: CNc1cc2ncc(-c3cc(NC(=O)c4cccc(C5(C#N)CC5)c4)cnc3C)cc2cn1] |
| 192 | 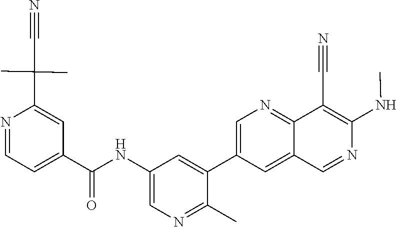 [SMILES: CNc1ncc2cc(-c3cc(NC(=O)c4ccnc(C(C)(C)C#N)c4)cnc3C)cnc2c1C#N] |
| 193 | 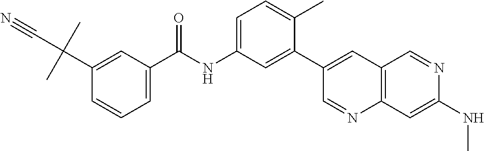 [SMILES: CNc1cc2ncc(-c3cc(NC(=O)c4cccc(C(C)(C)C#N)c4)ccc3C)cc2cn1] |
| 194 | 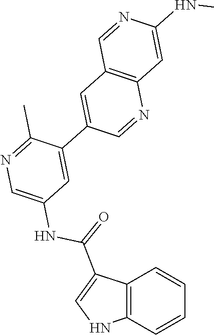 [SMILES: CNc1cc2ncc(-c3cc(NC(=O)c4c[nH]c5ccccc45)cnc3C)cc2cn1] |
| 195 | 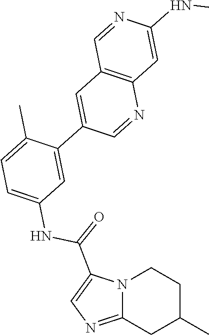 [SMILES: CNc1cc2ncc(-c3cc(NC(=O)c4cnc5n4CCC(C)C5)ccc3C)cc2cn1] |
| 196 | 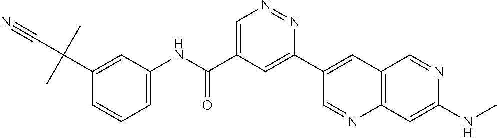 [SMILES: CNc1cc2ncc(-c3cc(C(=O)Nc4cccc(C(C)(C)C#N)c4)cnn3)cc2cn1] |
| 197 | 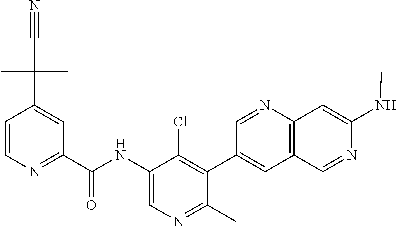 [SMILES: CNc1cc2ncc(-c3c(C)ncc(NC(=O)c4cc(C(C)(C)C#N)ccn4)c3Cl)cc2cn1] |
| 198 | 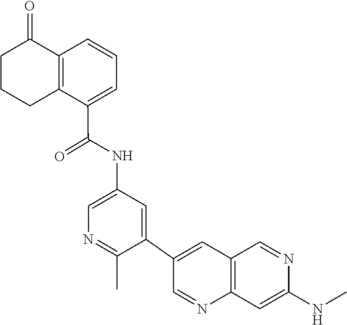 [SMILES: CNc1cc2ncc(-c3cc(NC(=O)c4cccc5c4CCCC5=O)cnc3C)cc2cn1] |
| 199 | 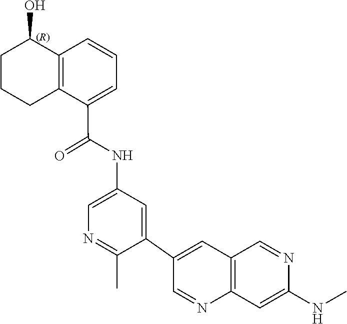 |
| 200 | 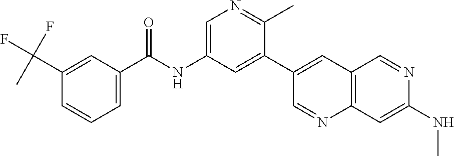 [SMILES: CNc1cc2ncc(-c3cc(NC(=O)c4cccc(C(C)(F)F)c4)cnc3C)cc2cn1] |
| 201 | 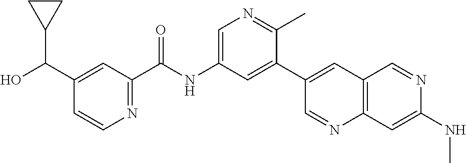 [SMILES: CNc1cc2ncc(-c3cc(NC(=O)c4cc(C(O)C5CC5)ccn4)cnc3C)cc2cn1] |
| 202 | 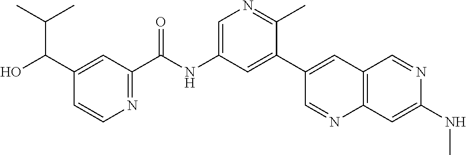 [SMILES: CNc1cc2ncc(-c3cc(NC(=O)c4cc(C(O)C(C)C)ccn4)cnc3C)cc2cn1] |
| 203 | 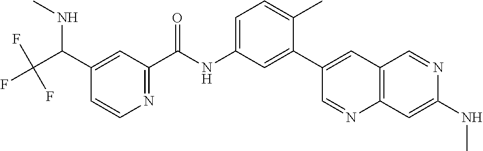 [SMILES: CNc1cc2ncc(-c3cc(NC(=O)c4cc(C(NC)C(F)(F)F)ccn4)ccc3C)cc2cn1] |
| 204 | 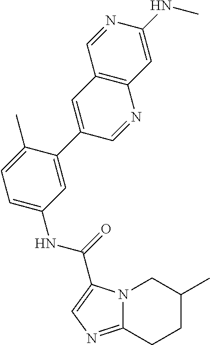 [SMILES: CNc1cc2ncc(-c3cc(NC(=O)c4cnc5n4CC(C)CC5)ccc3C)cc2cn1] |
| 205 | 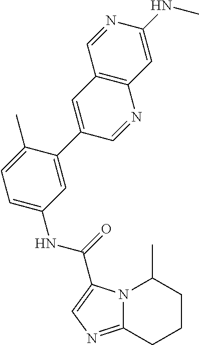 [SMILES: CNc1cc2ncc(-c3cc(NC(=O)c4cnc5n4C(C)CCC5)ccc3C)cc2cn1] |
| 206 | 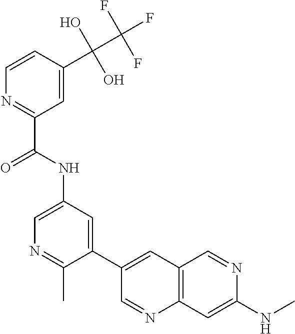 [SMILES: CNc1cc2ncc(-c3cc(NC(=O)c4cc(C(O)(O)C(F)(F)F)ccn4)cnc3C)cc2cn1] |
| 207 | 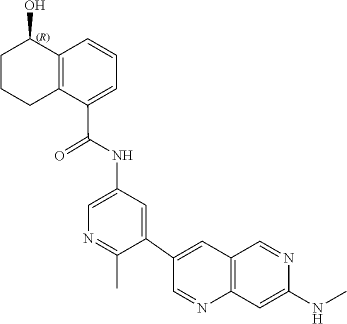 |
| 208 | 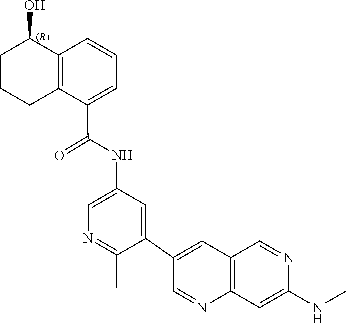 |
| 209 | 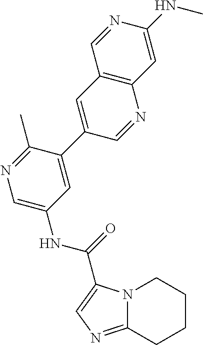 [SMILES: CNc1cc2ncc(-c3cc(NC(=O)c4cnc5n4CCCC5)cnc3C)cc2cn1] |
| 210 | 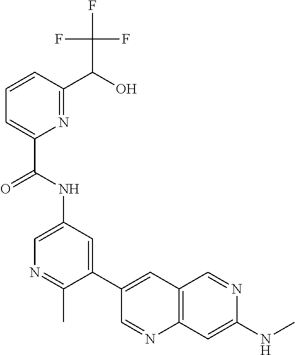 [SMILES: CNc1cc2ncc(-c3cc(NC(=O)c4cccc(C(O)C(F)(F)F)n4)cnc3C)cc2cn1] |
| 211 | 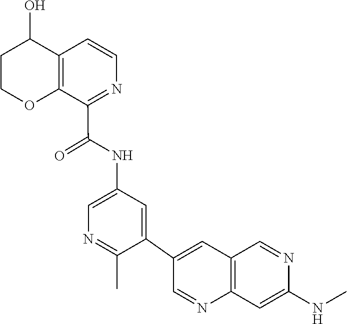 [SMILES: CNc1cc2ncc(-c3cc(NC(=O)c4nccc5c4OCCC5O)cnc3C)cc2cn1] |
| 212 | 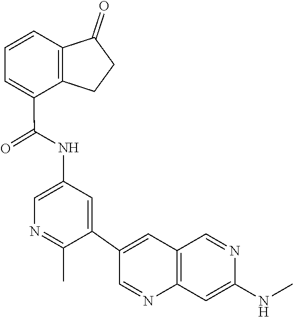 [SMILES: CNc1cc2ncc(-c3cc(NC(=O)c4cccc5c4CCC5=O)cnc3C)cc2cn1] |
| 213 | 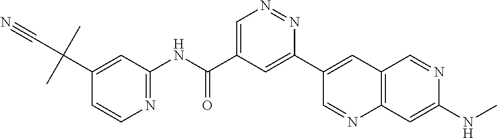 [SMILES: CNc1cc2ncc(-c3cc(C(=O)Nc4cc(C(C)(C)C#N)ccn4)cnn3)cc2cn1] |
| 214 | 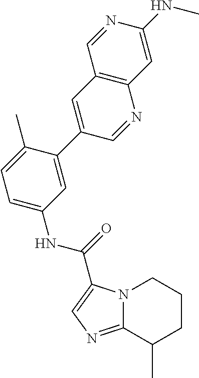 [SMILES: CNc1cc2ncc(-c3cc(NC(=O)c4cnc5n4CCCC5C)ccc3C)cc2cn1] |
| 215 | 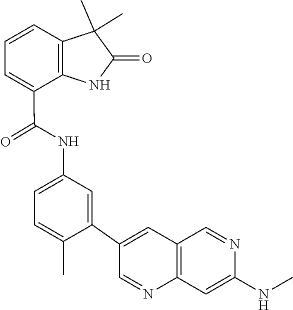 [SMILES: CNc1cc2ncc(-c3cc(NC(=O)c4cccc5c4NC(=O)C5(C)C)ccc3C)cc2cn1] |
| 216 | 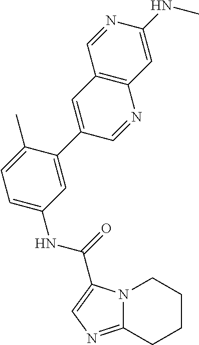 [SMILES: CNc1cc2ncc(-c3cc(NC(=O)c4cnc5n4CCCC5)ccc3C)cc2cn1] |
| 217 | 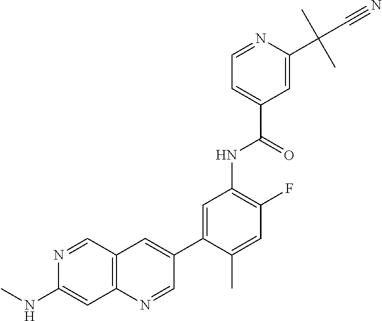 [SMILES: CNc1cc2ncc(-c3cc(NC(=O)c4ccnc(C(C)(C)C#N)c4)c(F)cc3C)cc2cn1] |
| 218 | 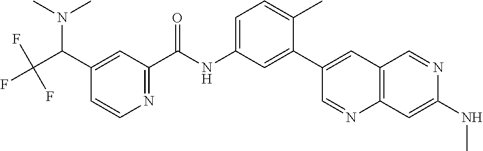 [SMILES: CNc1cc2ncc(-c3cc(NC(=O)c4cc(C(N(C)C)C(F)(F)F)ccn4)ccc3C)cc2cn1] |
| 219 | 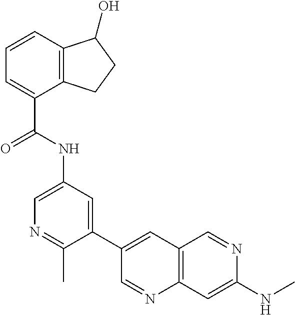 [SMILES: CNc1cc2ncc(-c3cc(NC(=O)c4cccc5c4CCC5O)cnc3C)cc2cn1] |
| 220 | 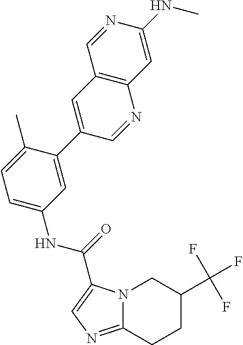 [SMILES: CNc1cc2ncc(-c3cc(NC(=O)c4cnc5n4CC(C(F)(F)F)CC5)ccc3C)cc2cn1] |
| 221 | 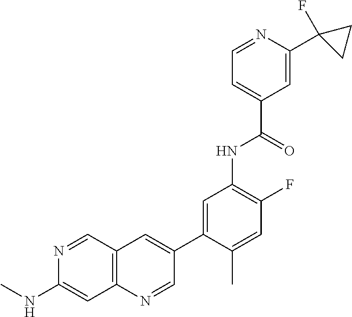 [SMILES: CNc1cc2ncc(-c3cc(NC(=O)c4ccnc(C5(F)CC5)c4)c(F)cc3C)cc2cn1] |
| 222 | 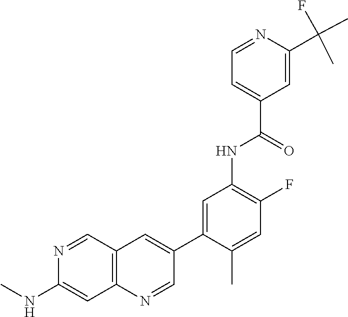 [SMILES: CNc1cc2ncc(-c3cc(NC(=O)c4ccnc(C(C)(C)F)c4)c(F)cc3C)cc2cn1] |
| 223 | 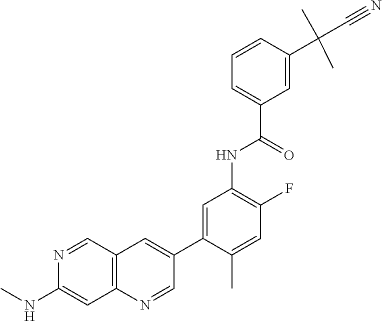 [SMILES: CNc1cc2ncc(-c3cc(NC(=O)c4cccc(C(C)(C)C#N)c4)c(F)cc3C)cc2cn1] |
| 224 | 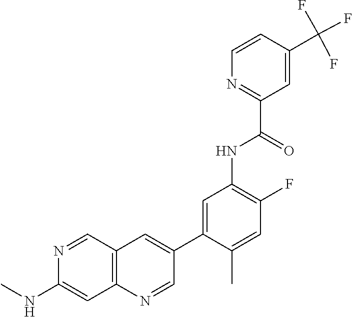 [SMILES: CNc1cc2ncc(-c3cc(NC(=O)c4cc(C(F)(F)F)ccn4)c(F)cc3C)cc2cn1] |
| 225 | 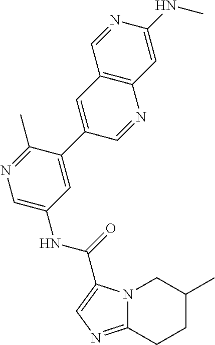 [SMILES: CNc1cc2ncc(-c3cc(NC(=O)c4cnc5n4CC(C)CC5)cnc3C)cc2cn1] |
| 226 | 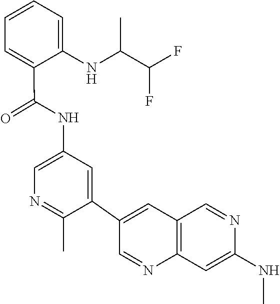 [SMILES: CNc1cc2ncc(-c3cc(NC(=O)c4ccccc4NC(C)C(F)F)cnc3C)cc2cn1] |
| 227 | 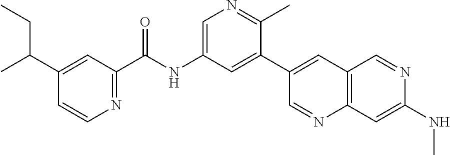 [SMILES: CCC(C)c1ccnc(C(=O)Nc2cnc(C)c(-c3cnc4cc(NC)ncc4c3)c2)c1] |
| 228 | 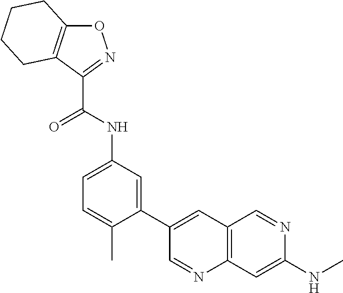 [SMILES: CNc1cc2ncc(-c3cc(NC(=O)c4noc5c4CCCC5)ccc3C)cc2cn1] |
| 229 | 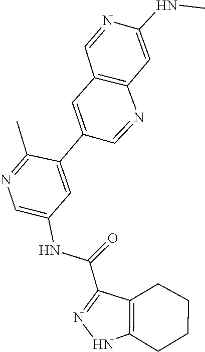 [SMILES: CNc1cc2ncc(-c3cc(NC(=O)c4n[nH]c5c4CCCC5)cnc3C)cc2cn1] |
| 230 | 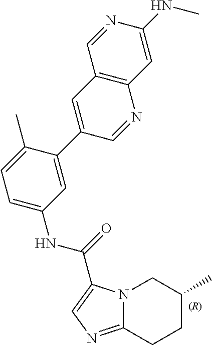 |
| 231 | 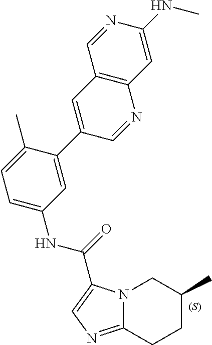 |
| 232 | 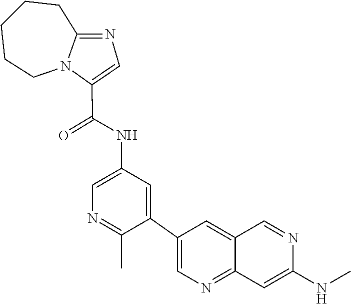 [SMILES: CNc1cc2ncc(-c3cc(NC(=O)c4cnc5n4CCCCC5)cnc3C)cc2cn1] |
| 233 | 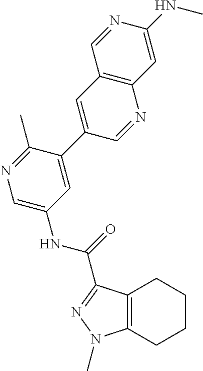 [SMILES: CNc1cc2ncc(-c3cc(NC(=O)c4nn(C)c5c4CCCC5)cnc3C)cc2cn1] |
| 234 | 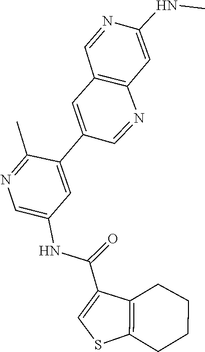 [SMILES: CNc1cc2ncc(-c3cc(NC(=O)c4csc5c4CCCC5)cnc3C)cc2cn1] |
| 235 | 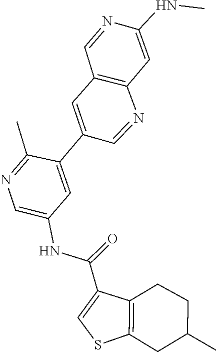 [SMILES: CNc1cc2ncc(-c3cc(NC(=O)c4csc5c4CCC(C)C5)cnc3C)cc2cn1] |
| 236 | 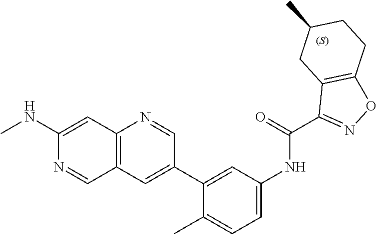 |
| 237 | 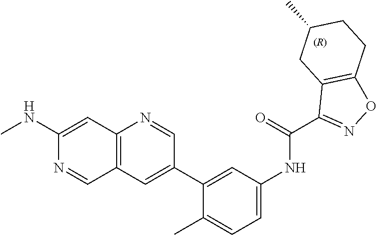 |
| 238 | 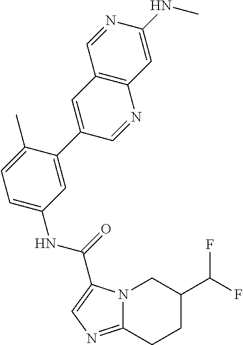 [SMILES: CNc1cc2ncc(-c3cc(NC(=O)c4cnc5n4CC(C(F)F)CC5)ccc3C)cc2cn1] |
| 239 | 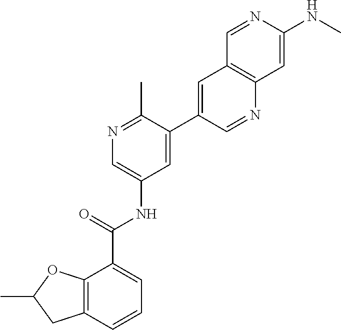 |
| 240 | 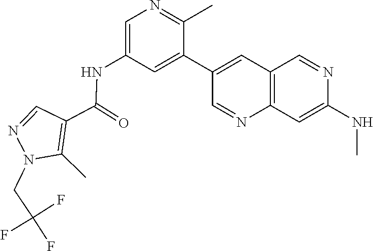 [SMILES: CNc1cc2ncc(-c3cc(NC(=O)c4cnn(CC(F)(F)F)c4C)cnc3C)cc2cn1] |
| 241 | 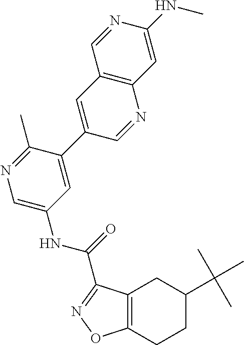 [SMILES: CNc1cc2ncc(-c3cc(NC(=O)c4noc5c4CC(C(C)(C)C)CC5)cnc3C)cc2cn1] |
| 242 | 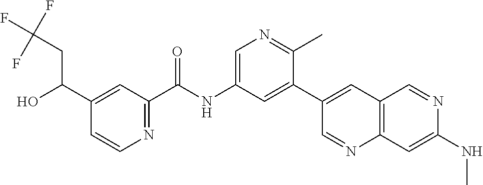 [SMILES: CNc1cc2ncc(-c3cc(NC(=O)c4cc(C(O)CC(F)(F)F)ccn4)cnc3C)cc2cn1] |
| 243 | 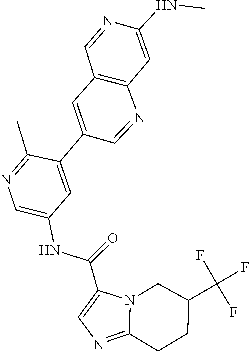 [SMILES: CNc1cc2ncc(-c3cc(NC(=O)c4cnc5n4CC(C(F)(F)F)CC5)cnc3C)cc2cn1] |
| 244 | 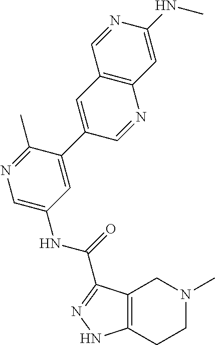 [SMILES: CNc1cc2ncc(-c3cc(NC(=O)c4n[nH]c5c4CN(C)CC5)cnc3C)cc2cn1] |
| 245 | 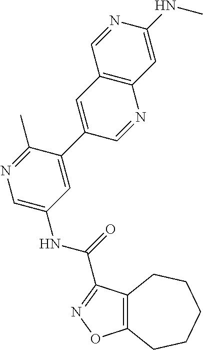 [SMILES: CNc1cc2ncc(-c3cc(NC(=O)c4noc5c4CCCCC5)cnc3C)cc2cn1] |
| 246 | 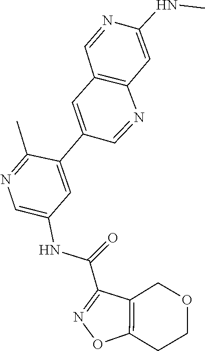 [SMILES: CNc1cc2ncc(-c3cc(NC(=O)c4noc5c4COCC5)cnc3C)cc2cn1] |
| 247 | 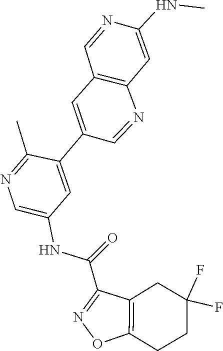 [SMILES: CNc1cc2ncc(-c3cc(NC(=O)c4noc5c4CC(F)(F)CC5)cnc3C)cc2cn1] |
| 248 | 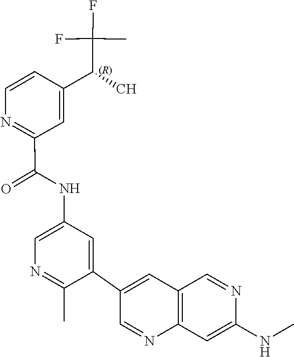 |
| 249 | 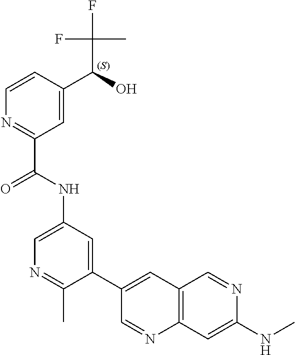 |
| 250 | 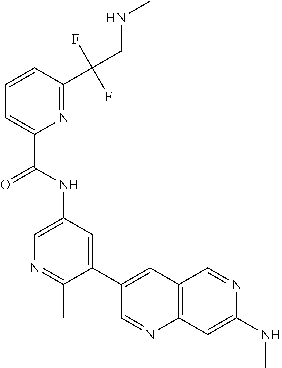 [SMILES: CNCC(F)(F)c1cccc(C(=O)Nc2cnc(C)c(-c3cnc4cc(NC)ncc4c3)c2)n1] |
| 251 | 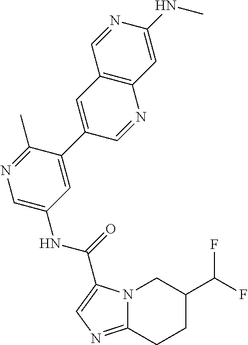 [SMILES: CNc1cc2ncc(-c3cc(NC(=O)c4cnc5n4CC(C(F)F)CC5)cnc3C)cc2cn1] |
| 252 | 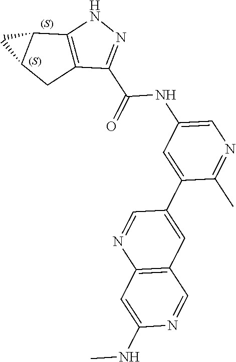 |
| 253 | 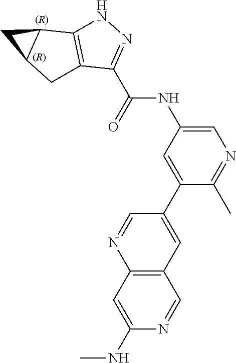 |
| 254 | 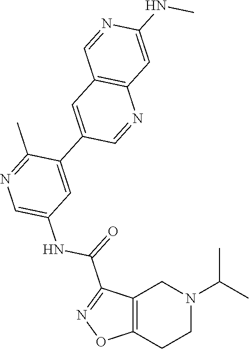 [SMILES: CNc1cc2ncc(-c3cc(NC(=O)c4noc5c4CN(C(C)C)CC5)cnc3C)cc2cn1] |
| 255 | 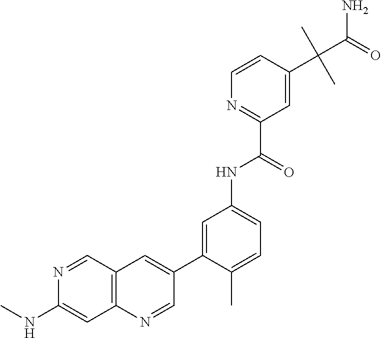 [SMILES: CNc1cc2ncc(-c3cc(NC(=O)c4cc(C(C)(C)C(N)=O)ccn4)ccc3C)cc2cn1] |
| 256 | 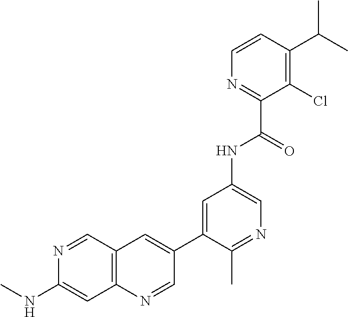 [SMILES: CNc1cc2ncc(-c3cc(NC(=O)c4nccc(C(C)C)c4Cl)cnc3C)cc2cn1] |
| 257 | 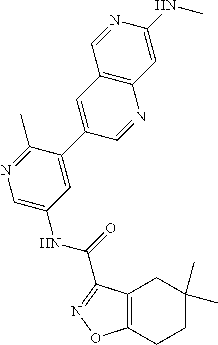 [SMILES: CNc1cc2ncc(-c3cc(NC(=O)c4noc5c4CC(C)(C)CC5)cnc3C)cc2cn1] |
| 258 | 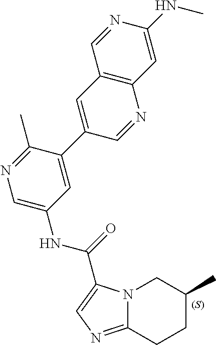 |
| 259 | 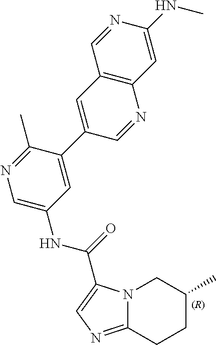 |
| 260 | 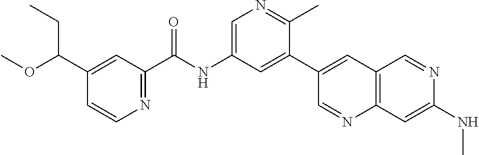 |
| 261 | 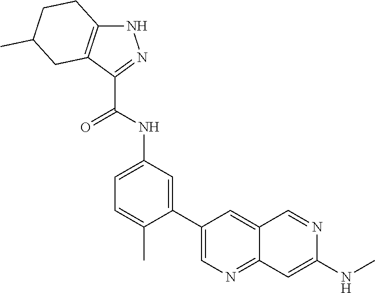 |
| 262 | 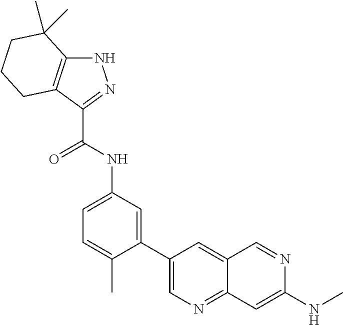 [SMILES: CNc1cc2ncc(-c3cc(NC(=O)c4n[nH]c5c4CCCC5(C)C)ccc3C)cc2cn1] |
| 263 | 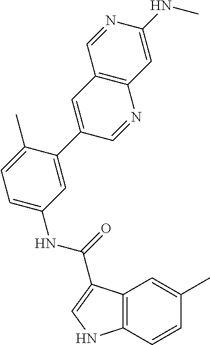 [SMILES: CNc1cc2ncc(-c3cc(NC(=O)c4c[nH]c5ccc(C)cc45)ccc3C)cc2cn1] |
| 264 | 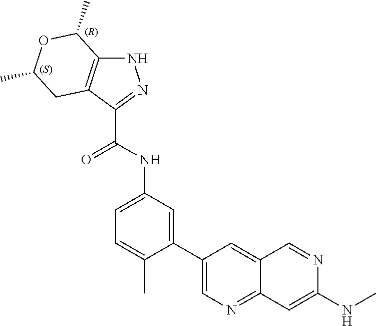 |
| 265 | 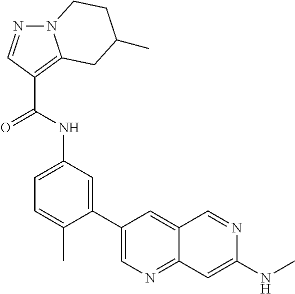 [SMILES: CNc1cc2ncc(-c3cc(NC(=O)c4cnn5c4CC(C)CC5)ccc3C)cc2cn1] |
| 266 | 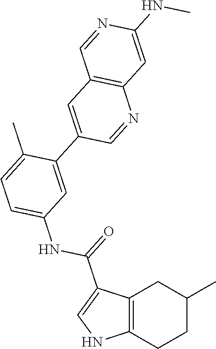 [SMILES: CNc1cc2ncc(-c3cc(NC(=O)c4c[nH]c5c4CC(C)CC5)ccc3C)cc2cn1] |
| 267 | 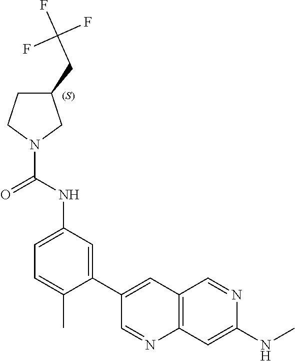 |
| 268 | 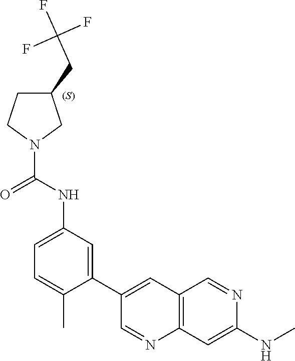 |
| 269 | 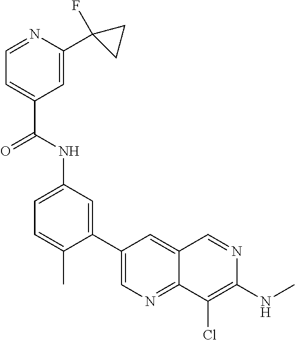 [SMILES: CNc1ncc2cc(-c3cc(NC(=O)c4ccnc(C5(F)CC5)c4)ccc3C)cnc2c1Cl] |
| 270 | 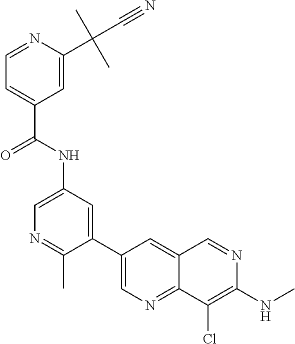 [SMILES: CNc1ncc2cc(-c3cc(NC(=O)c4ccnc(C(C)(C)C#N)c4)cnc3C)cnc2c1Cl] |
| 271 | 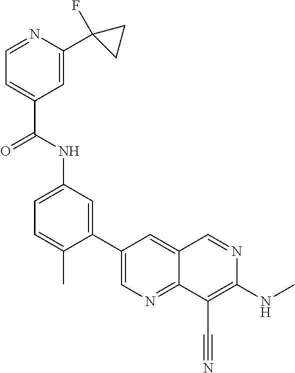 [SMILES: CNc1ncc2cc(-c3cc(NC(=O)c4ccnc(C5(F)CC5)c4)ccc3C)cnc2c1C#N] |
| 272 | 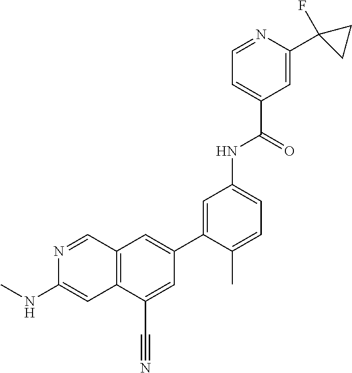 [SMILES: CNc1cc2c(C#N)cc(-c3cc(NC(=O)c4ccnc(C5(F)CC5)c4)ccc3C)cc2cn1] |
| 273 | 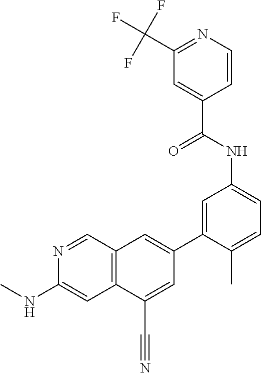 [SMILES: CNc1cc2c(C#N)cc(-c3cc(NC(=O)c4ccnc(C(F)(F)F)c4)ccc3C)cc2cn1] |
| 274 | 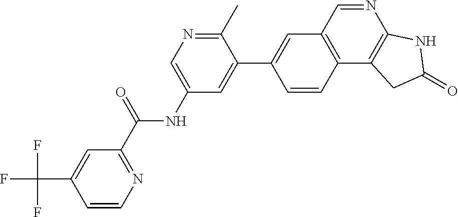 [SMILES: Cc1ncc(NC(=O)c2cc(C(F)(F)F)ccn2)cc1-c1ccc2c3c(ncc2c1)NC(=O)C3] |
| 275 | 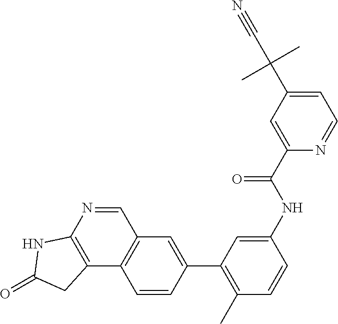 [SMILES: Cc1ccc(NC(=O)c2cc(C(C)(C)C#N)ccn2)cc1-c1ccc2c3c(ncc2c1)NC(=O)C3] |
| 276 | 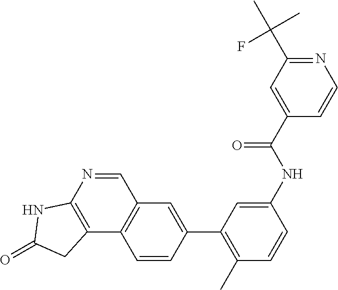 [SMILES: Cc1ccc(NC(=O)c2ccnc(C(C)(C)F)c2)cc1-c1ccc2c3c(ncc2c1)NC(=O)C3] |
| 277 | 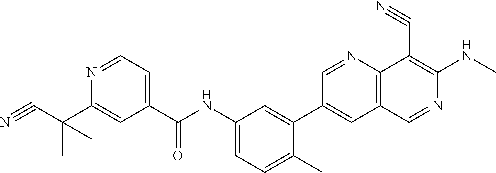 [SMILES: CNc1ncc2cc(-c3cc(NC(=O)c4ccnc(C(C)(C)C#N)c4)ccc3C)cnc2c1C#N] |
| 278 | 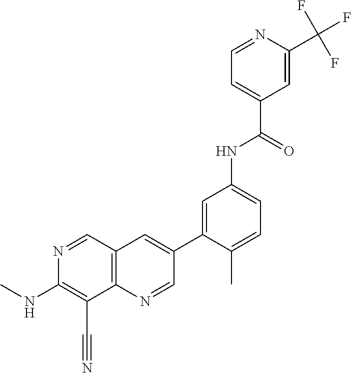 [SMILES: CNc1ncc2cc(-c3cc(NC(=O)c4ccnc(C(F)(F)F)c4)ccc3C)cnc2c1C#N] |
| 279 | 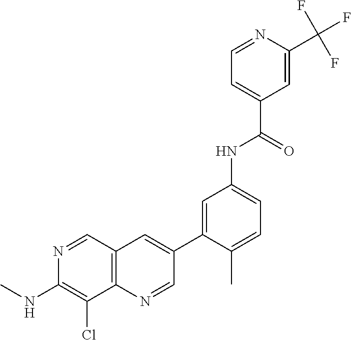 [SMILES: CNc1ncc2cc(-c3cc(NC(=O)c4ccnc(C(F)(F)F)c4)ccc3C)cnc2c1Cl] |
| 280 | 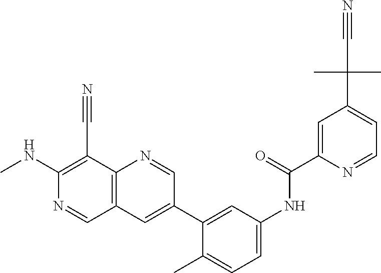 [SMILES: CNc1ncc2cc(-c3cc(NC(=O)c4cc(C(C)(C)C#N)ccn4)ccc3C)cnc2c1C#N] |
| 281 | 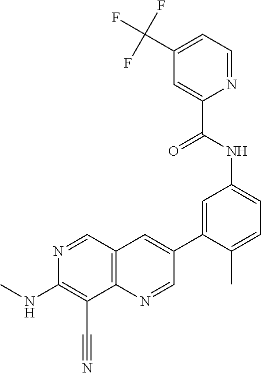 [SMILES: CNc1ncc2cc(-c3cc(NC(=O)c4cc(C(F)(F)F)ccn4)ccc3C)cnc2c1C#N] |
| 282 | 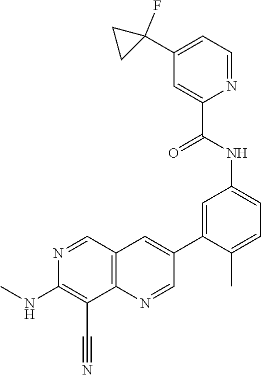 [SMILES: CNc1ncc2cc(-c3cc(NC(=O)c4cc(C5(F)CC5)ccn4)ccc3C)cnc2c1C#N] |
| 283 | 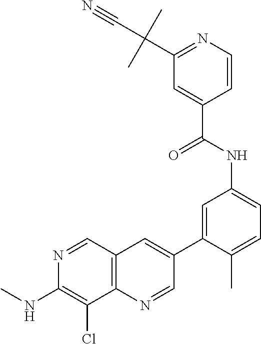 [SMILES: CNc1ncc2cc(-c3cc(NC(=O)c4ccnc(C(C)(C)C#N)c4)ccc3C)cnc2c1Cl] |
| 284 | 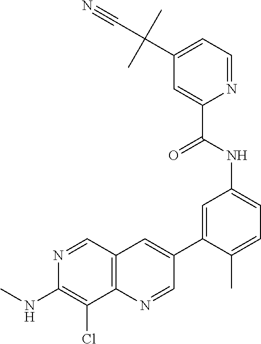 [SMILES: CNc1ncc2cc(-c3cc(NC(=O)c4cc(C(C)(C)C#N)ccn4)ccc3C)cnc2c1Cl] |
| 285 | 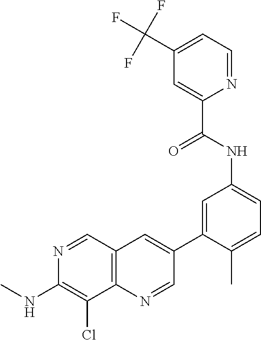 [SMILES: CNc1ncc2cc(-c3cc(NC(=O)c4cc(C(F)(F)F)ccn4)ccc3C)cnc2c1Cl] |
| 286 | 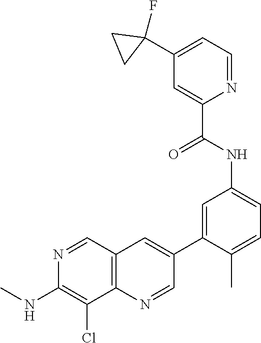 [SMILES: CNc1ncc2cc(-c3cc(NC(=O)c4cc(C5(F)CC5)ccn4)ccc3C)cnc2c1Cl] |
| 287 | 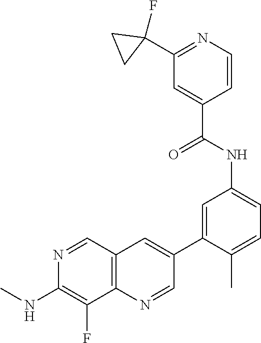 [SMILES: CNc1ncc2cc(-c3cc(NC(=O)c4ccnc(C5(F)CC5)c4)ccc3C)cnc2c1F] |
| 288 | 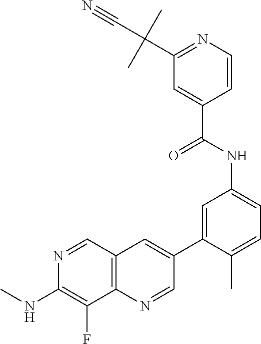 [SMILES: CNc1ncc2cc(-c3cc(NC(=O)c4ccnc(C(C)(C)C#N)c4)ccc3C)cnc2c1F] |
| 289 | 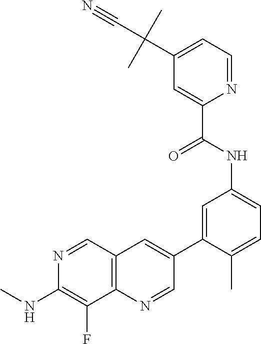 [SMILES: CNc1ncc2cc(-c3cc(NC(=O)c4cc(C(C)(C)C#N)ccn4)ccc3C)cnc2c1F] |
| 290 | 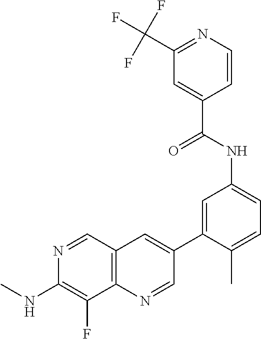 [SMILES: CNc1ncc2cc(-c3cc(NC(=O)c4ccnc(C(F)(F)F)c4)ccc3C)cnc2c1F] |
| 291 | 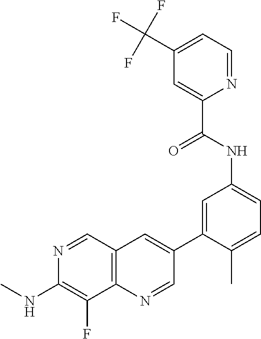 [SMILES: CNc1ncc2cc(-c3cc(NC(=O)c4cc(C(F)(F)F)ccn4)ccc3C)cnc2c1F] |
| 292 | 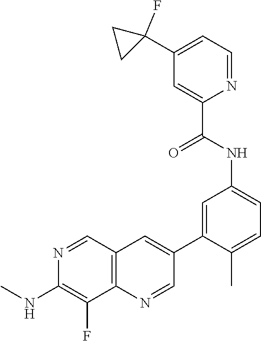 [SMILES: CNc1ncc2cc(-c3cc(NC(=O)c4cc(C5(F)CC5)ccn4)ccc3C)cnc2c1F] |
| 293 | 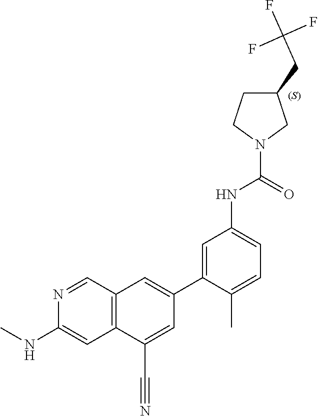 |
| 294 | 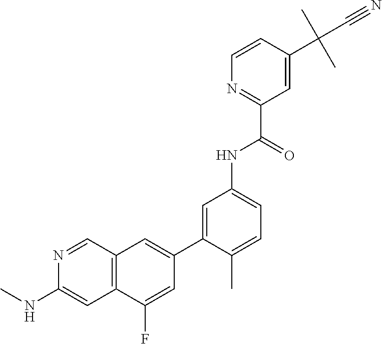 [SMILES: CNc1cc2c(F)cc(-c3cc(NC(=O)c4cc(C(C)(C)C#N)ccn4)ccc3C)cc2cn1] |
| 295 | 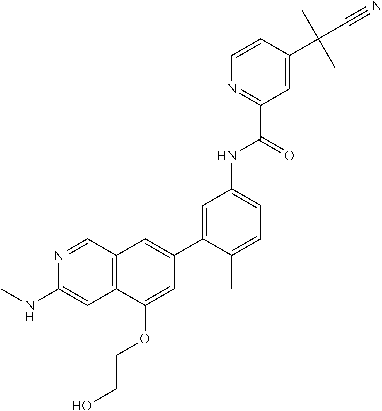 [SMILES: CNc1cc2c(OCCO)cc(-c3cc(NC(=O)c4cc(C(C)(C)C#N)ccn4)ccc3C)cc2cn1] |
| 296 | 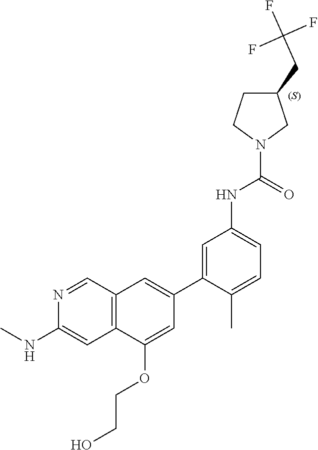 |
| 297 | 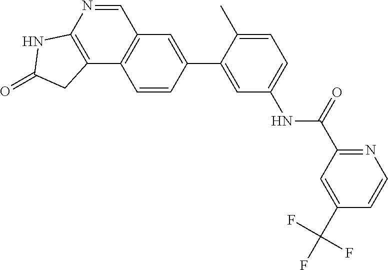 [SMILES: Cc1ccc(NC(=O)c2cc(C(F)(F)F)ccn2)cc1-c1ccc2c3c(ncc2c1)NC(=O)C3] |
| 298 | 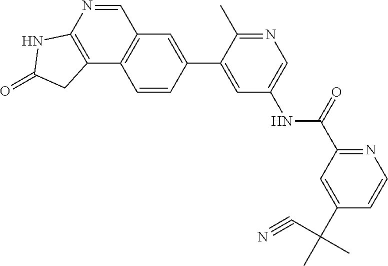 [SMILES: Cc1ncc(NC(=O)c2cc(C(C)(C)C#N)ccn2)cc1-c1ccc2c3c(ncc2c1)NC(=O)C3] |
| 299 | 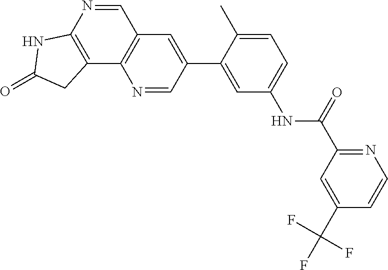 [SMILES: Cc1ccc(NC(=O)c2cc(C(F)(F)F)ccn2)cc1-c1cnc2c3c(ncc2c1)NC(=O)C3] |
| 300 | 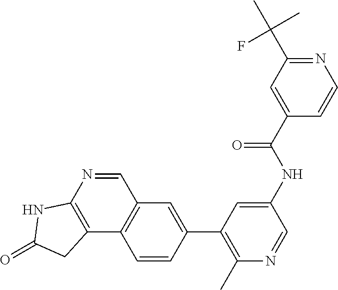 [SMILES: Cc1ncc(NC(=O)c2ccnc(C(C)(C)F)c2)cc1-c1ccc2c3c(ncc2c1)NC(=O)C3] |
| 301 | 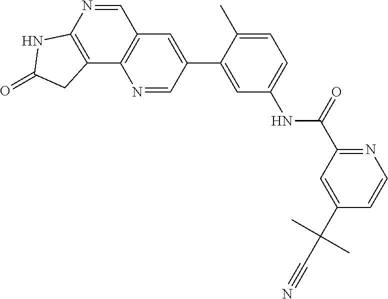 [SMILES: Cc1ccc(NC(=O)c2cc(C(C)(C)C#N)ccn2)cc1-c1cnc2c3c(ncc2c1)NC(=O)C3] |
| 302 | 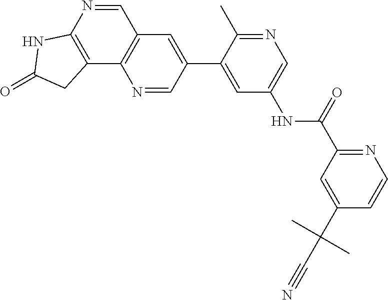 [SMILES: Cc1ncc(NC(=O)c2cc(C(C)(C)C#N)ccn2)cc1-c1cnc2c3c(ncc2c1)NC(=O)C3] |
| 303 | 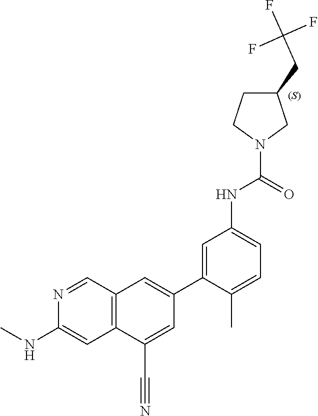 |
| 304 | 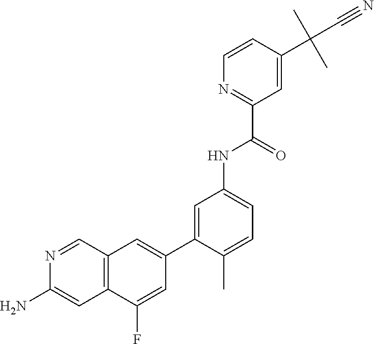 [SMILES: Cc1ccc(NC(=O)c2cc(C(C)(C)C#N)ccn2)cc1-c1cc(F)c2cc(N)ncc2c1] |
| 305 | 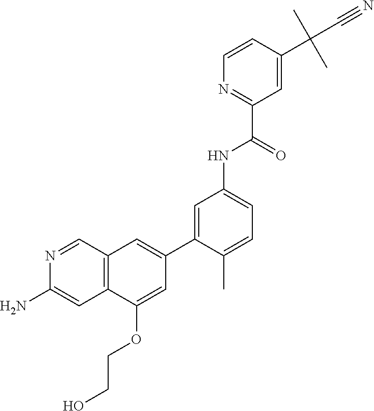 [SMILES: Cc1ccc(NC(=O)c2cc(C(C)(C)C#N)ccn2)cc1-c1cc(OCCO)c2cc(N)ncc2c1] |
| 306 | 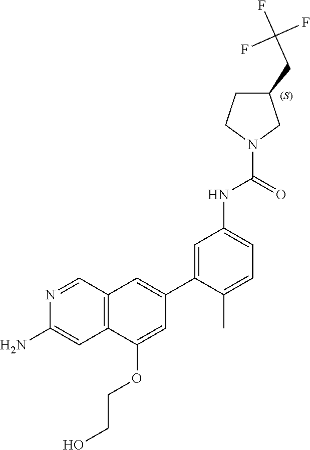 |
| 307 | 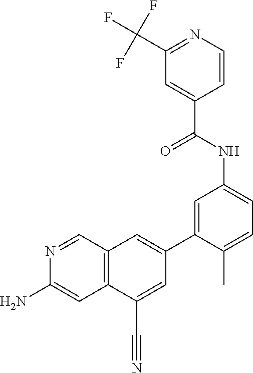 [SMILES: Cc1ccc(NC(=O)c2ccnc(C(F)(F)F)c2)cc1-c1cc(C#N)c2cc(N)ncc2c1] |
| 308 | 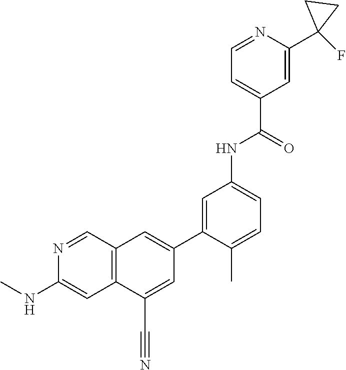 [SMILES: CNc1cc2c(C#N)cc(-c3cc(NC(=O)c4ccnc(C5(F)CC5)c4)ccc3C)cc2cn1] |
| 309 | 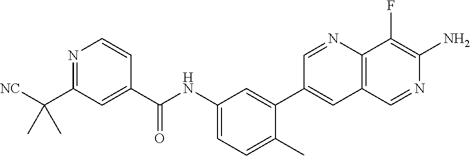 [SMILES: Cc1ccc(NC(=O)c2ccnc(C(C)(C)C#N)c2)cc1-c1cnc2c(F)c(N)ncc2c1] |
| 310 | 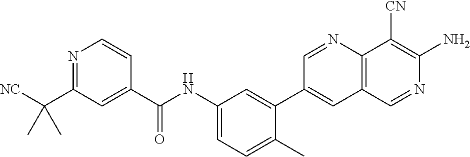 [SMILES: Cc1ccc(NC(=O)c2ccnc(C(C)(C)C#N)c2)cc1-c1cnc2c(C#N)c(N)ncc2c1] |
| 311 | 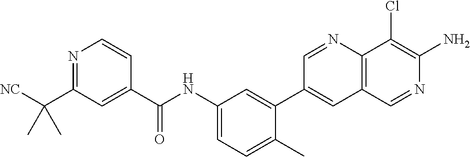 [SMILES: Cc1ccc(NC(=O)c2ccnc(C(C)(C)C#N)c2)cc1-c1cnc2c(Cl)c(N)ncc2c1] |
| 312 | 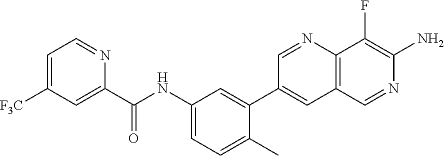 [SMILES: Cc1ccc(NC(=O)c2cc(C(F)(F)F)ccn2)cc1-c1cnc2c(F)c(N)ncc2c1] |
| 313 | 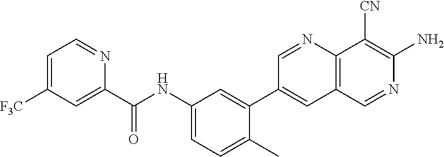 [SMILES: Cc1ccc(NC(=O)c2cc(C(F)(F)F)ccn2)cc1-c1cnc2c(C#N)c(N)ncc2c1] |
| 314 | 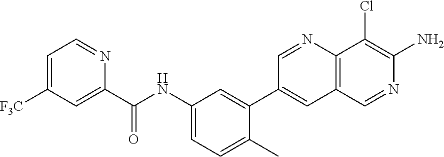 [SMILES: Cc1ccc(NC(=O)c2cc(C(F)(F)F)ccn2)cc1-c1cnc2c(Cl)c(N)ncc2c1] |
| 315 | 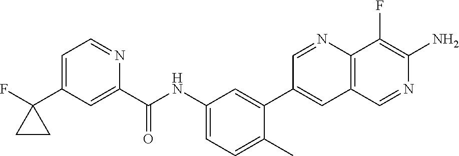 [SMILES: Cc1ccc(NC(=O)c2cc(C3(F)CC3)ccn2)cc1-c1cnc2c(F)c(N)ncc2c1] |
| 316 | 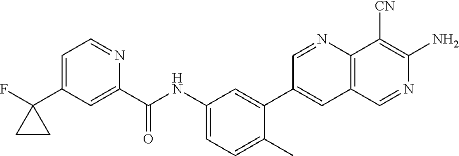 [SMILES: Cc1ccc(NC(=O)c2cc(C3(F)CC3)ccn2)cc1-c1cnc2c(C#N)c(N)ncc2c1] |
| 317 | 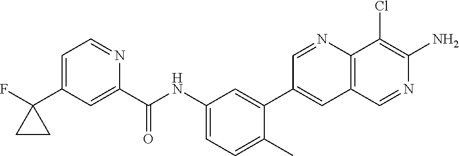 [SMILES: Cc1ccc(NC(=O)c2cc(C3(F)CC3)ccn2)cc1-c1cnc2c(Cl)c(N)ncc2c1] |
| 318 | 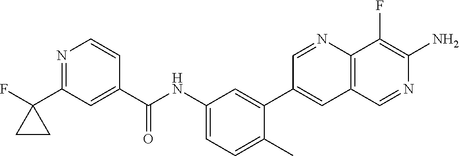 [SMILES: Cc1ccc(NC(=O)c2ccnc(C3(F)CC3)c2)cc1-c1cnc2c(F)c(N)ncc2c1] |
| 319 | 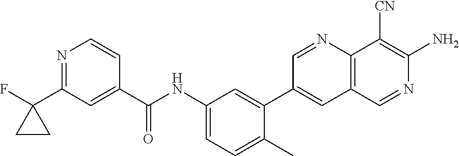 [SMILES: Cc1ccc(NC(=O)c2ccnc(C3(F)CC3)c2)cc1-c1cnc2c(C#N)c(N)ncc2c1] |
| 320 | 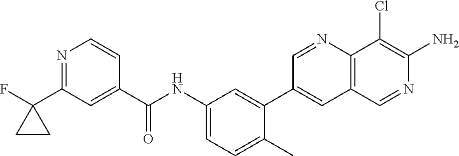 [SMILES: Cc1ccc(NC(=O)c2ccnc(C3(F)CC3)c2)cc1-c1cnc2c(Cl)c(N)ncc2c1] |
| 321 | 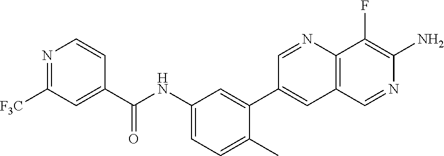 [SMILES: Cc1ccc(NC(=O)c2ccnc(C(F)(F)F)c2)cc1-c1cnc2c(F)c(N)ncc2c1] |
| 322 | 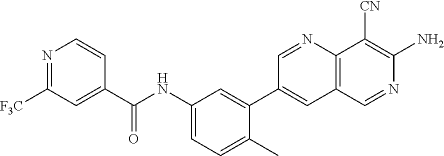 [SMILES: Cc1ccc(NC(=O)c2ccnc(C(F)(F)F)c2)cc1-c1cnc2c(C#N)c(N)ncc2c1] |
| 323 | 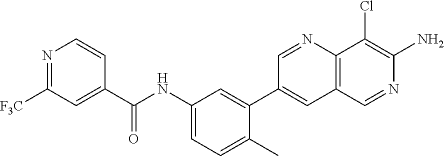 [SMILES: Cc1ccc(NC(=O)c2ccnc(C(F)(F)F)c2)cc1-c1cnc2c(Cl)c(N)ncc2c1] |
| 324 | 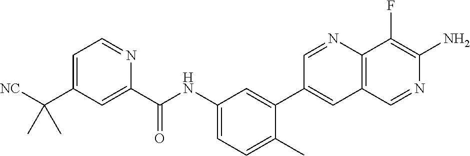 [SMILES: Cc1ccc(NC(=O)c2cc(C(C)(C)C#N)ccn2)cc1-c1cnc2c(F)c(N)ncc2c1] |
| 325 | 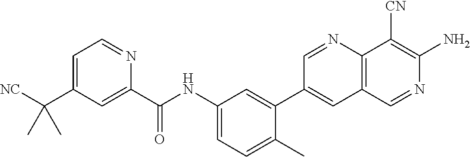 [SMILES: Cc1ccc(NC(=O)c2cc(C(C)(C)C#N)ccn2)cc1-c1cnc2c(C#N)c(N)ncc2c1] |
| 326 | 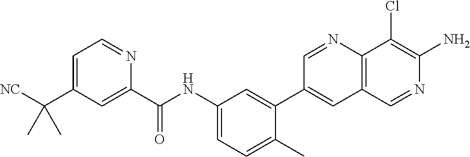 [SMILES: Cc1ccc(NC(=O)c2cc(C(C)(C)C#N)ccn2)cc1-c1cnc2c(Cl)c(N)ncc2c1] |

In some embodiments, the compound is selected from Compound Nos. 1, 2, 3, 4, 5, 6, 7, 8, 9, 10, 11, 12, 14, 15, 16, 17, 18, 19, 20, 21, 22, 23, 24, 25, 26, 27, 29, 30, 31, 32, 33, 34, 35, 37, 38, 39, 40, 41, 42, 43, 44, 49, 52, 53, 55, 56, 57, 58, 59, 60, 62, 63, 65, 66, 68, 71, 75, 77, 78, 79, 153, 154, 173, 174, 175, 176, 191, 193, 194, 200, 201, 202, 203, 204, 206, 214, 215, 217, 218, 220, 221, 222, 223, 225, 226, 227, 229, 230, 231, 233, 234, 235, 236, 237, 238, 242, 243, 245, 247, 248, 249, 252, 253, 255, 256, 257, 258, 259, 260, 261, 262, 263, 264, 265, 266, 267, 271, 272, 274, 275, and 276, and pharmaceutically acceptable salts and stereoisomers thereof.

In some embodiments, the compound is selected from Compound Nos. 1, 2, 3, 4, 5, 6, 7, 8, 9, 10, 11, 12, 14, 15, 16, 17, 18, 19, 20, 21, 22, 23, 24, 25, 26, 27, 29, 30, 31, 32, 33, 34, 35, 37, 38, 39, 40, 41, 42, 43, 44, 49, 52, 53, 55, 56, 57, 58, 59, 60, 62, 63, 65, 66, 68, 71, 75, 77, 78, 79, 153, 154, 173, 174, 175, 176, 191, 193, 194, 200, 201, 202, 203, 204, 206, 214, 215, 217, 218, 220, 221, 222, 223, 225, 226, 227, 229, 230, 231, 233, 234, 235, 236, 237, 238, 242, 243, 245, 247, 248, 249, 252, 253, 255, 256, 257, 258, 259, 260, 261, 262, 263, 264, 265, 266, 267, 271, 272, 274, 275, and 276, and pharmaceutically acceptable salts thereof.

In some embodiments, the compound is selected from Compound Nos. 1, 2, 3, 4, 5, 6, 7, 8, 9, 10, 11, 12, 14, 15, 16, 17, 18, 19, 20, 21, 22, 23, 24, 25, 26, 27, 29, 30, 31, 32, 33, 34, 35, 37, 38, 39, 40, 41, 42, 43, 44, 49, 52, 53, 55, 56, 57, 58, 59, 60, 62, 63, 65, 66, 68, 71, 75, 77, 78, 79, 153, 154, 173, 174, 175, 176, 191, 193, 194, 200, 201, 202, 203, 204, 206, 214, 215, 217, 218, 220, 221, 222, 223, 225, 226, 227, 229, 230, 231, 233, 234, 235, 236, 237, 238, 242, 243, 245, 247, 248, 249, 252, 253, 255, 256, 257, 258, 259, 260, 261, 262, 263, 264, 265, 266, 267, 271, 272, 274, 275, and 276.

In some embodiments, the compound is selected from Compound Nos. 1, 3, 4, 6, 7, 9, 10, 14, 16, 17, 18, 19, 20, 24, 27, 29, 32, 33, 49, 52, 55, 57, 60, 79, 154, 193, 200, 203, 204, 261, 263, 265, 267, 274, and 276, and pharmaceutically acceptable salts and stereoisomers thereof.

In some embodiments, the compound is selected from Compound Nos. 1, 3, 4, 6, 7, 9, 10, 14, 16, 17, 18, 19, 20, 24, 27, 29, 32, 33, 49, 52, 55, 57, 60, 79, 154, 193, 200, 203, 204, 261, 263, 265, 267, 274, and 276, and pharmaceutically acceptable salts thereof.

In some embodiments, the compound is selected from Compound Nos. 1, 3, 4, 6, 7, 9, 10, 14, 16, 17, 18, 19, 20, 24, 27, 29, 32, 33, 49, 52, 55, 57, 60, 79, 154, 193, 200, 203, 204, 261, 263, 265, 267, 274, and 276.

In some embodiments, the compound is Compound No. 1 or a pharmaceutically acceptable salt or stereoisomer thereof.

In some embodiments, the compound is Compound No. 1 or a pharmaceutically acceptable salt thereof.

In some embodiments, the compound is Compound No. 1.

In some embodiments, the compound is Compound No. 2 or a pharmaceutically acceptable salt or stereoisomer thereof.

In some embodiments, the compound is Compound No. 2 or a pharmaceutically acceptable salt thereof.

In some embodiments, the compound is Compound No. 2.

In some embodiments, the compound is Compound No. 3 or a pharmaceutically acceptable salt or stereoisomer thereof.

In some embodiments, the compound is Compound No. 3 or a pharmaceutically acceptable salt thereof.

In some embodiments, the compound is Compound No. 3.

In some embodiments, the compound is Compound No. 4 or a pharmaceutically acceptable salt or stereoisomer thereof.

In some embodiments, the compound is Compound No. 4 or a pharmaceutically acceptable salt thereof.

In some embodiments, the compound is Compound No. 4.

In some embodiments, the compound is Compound No. 5 or a pharmaceutically acceptable salt or stereoisomer thereof.

In some embodiments, the compound is Compound No. 5 or a pharmaceutically acceptable salt thereof.

In some embodiments, the compound is Compound No. 5.

In some embodiments, the compound is Compound No. 6 or a pharmaceutically acceptable salt or stereoisomer thereof.

In some embodiments, the compound is Compound No. 6 or a pharmaceutically acceptable salt thereof.

In some embodiments, the compound is Compound No. 6.

In some embodiments, the compound is Compound No. 7 or a pharmaceutically acceptable salt or stereoisomer thereof.

In some embodiments, the compound is Compound No. 7 or a pharmaceutically acceptable salt thereof.

In some embodiments, the compound is Compound No. 7.

In some embodiments, the compound is Compound No. 8 or a pharmaceutically acceptable salt or stereoisomer thereof.

In some embodiments, the compound is Compound No. 8 or a pharmaceutically acceptable salt thereof.

In some embodiments, the compound is Compound No. 8.

In some embodiments, the compound is Compound No. 9 or a pharmaceutically acceptable salt or stereoisomer thereof.

In some embodiments, the compound is Compound No. 9 or a pharmaceutically acceptable salt thereof.

In some embodiments, the compound is Compound No. 9.

In some embodiments, the compound is Compound No. 10 or a pharmaceutically acceptable salt or stereoisomer thereof.

In some embodiments, the compound is Compound No. 10 or a pharmaceutically acceptable salt thereof.

In some embodiments, the compound is Compound No. 10.

In some embodiments, the compound is Compound No. 11 or a pharmaceutically acceptable salt or stereoisomer thereof.

In some embodiments, the compound is Compound No. 11 or a pharmaceutically acceptable salt thereof.

In some embodiments, the compound is Compound No. 11.

In some embodiments, the compound is Compound No. 12 or a pharmaceutically acceptable salt or stereoisomer thereof.

In some embodiments, the compound is Compound No. 12 or a pharmaceutically acceptable salt thereof.

In some embodiments, the compound is Compound No. 12.

In some embodiments, the compound is Compound No. 14 or a pharmaceutically acceptable salt or stereoisomer thereof.

In some embodiments, the compound is Compound No. 14 or a pharmaceutically acceptable salt thereof.

In some embodiments, the compound is Compound No. 14.

In some embodiments, the compound is Compound No. 15 or a pharmaceutically acceptable salt or stereoisomer thereof.

In some embodiments, the compound is Compound No. 15 or a pharmaceutically acceptable salt thereof.

In some embodiments, the compound is Compound No. 15.

In some embodiments, the compound is Compound No. 16 or a pharmaceutically acceptable salt or stereoisomer thereof.

In some embodiments, the compound is Compound No. 16 or a pharmaceutically acceptable salt thereof.

In some embodiments, the compound is Compound No. 16.

In some embodiments, the compound is Compound No. 17 or a pharmaceutically acceptable salt or stereoisomer thereof.

In some embodiments, the compound is Compound No. 17 or a pharmaceutically acceptable salt thereof.

In some embodiments, the compound is Compound No. 17.

In some embodiments, the compound is Compound No. 18 or a pharmaceutically acceptable salt or stereoisomer thereof.

In some embodiments, the compound is Compound No. 18 or a pharmaceutically acceptable salt thereof.

In some embodiments, the compound is Compound No. 18.

In some embodiments, the compound is Compound No. 19 or a pharmaceutically acceptable salt or stereoisomer thereof.

In some embodiments, the compound is Compound No. 19 or a pharmaceutically acceptable salt thereof.

In some embodiments, the compound is Compound No. 19.

In some embodiments, the compound is Compound No. 20 or a pharmaceutically acceptable salt or stereoisomer thereof.

In some embodiments, the compound is Compound No. 20 or a pharmaceutically acceptable salt thereof.

In some embodiments, the compound is Compound No. 20.

In some embodiments, the compound is Compound No. 21 or a pharmaceutically acceptable salt or stereoisomer thereof.

In some embodiments, the compound is Compound No. 21 or a pharmaceutically acceptable salt thereof.

In some embodiments, the compound is Compound No. 21.

In some embodiments, the compound is Compound No. 22 or a pharmaceutically acceptable salt or stereoisomer thereof.

In some embodiments, the compound is Compound No. 22 or a pharmaceutically acceptable salt thereof.

In some embodiments, the compound is Compound No. 22.

In some embodiments, the compound is Compound No. 23 or a pharmaceutically acceptable salt or stereoisomer thereof.

In some embodiments, the compound is Compound No. 23 or a pharmaceutically acceptable salt thereof.

In some embodiments, the compound is Compound No. 23.

In some embodiments, the compound is Compound No. 24 or a pharmaceutically acceptable salt or stereoisomer thereof.

In some embodiments, the compound is Compound No. 24 or a pharmaceutically acceptable salt thereof.

In some embodiments, the compound is Compound No. 24.

In some embodiments, the compound is Compound No. 25 or a pharmaceutically acceptable salt or stereoisomer thereof.

In some embodiments, the compound is Compound No. 25 or a pharmaceutically acceptable salt thereof.

In some embodiments, the compound is Compound No. 25.

In some embodiments, the compound is Compound No. 26 or a pharmaceutically acceptable salt or stereoisomer thereof.

In some embodiments, the compound is Compound No. 26 or a pharmaceutically acceptable salt thereof.

In some embodiments, the compound is Compound No. 26.

In some embodiments, the compound is Compound No. 27 or a pharmaceutically acceptable salt or stereoisomer thereof.

In some embodiments, the compound is Compound No. 27 or a pharmaceutically acceptable salt thereof.

In some embodiments, the compound is Compound No. 27.

In some embodiments, the compound is Compound No. 29 or a pharmaceutically acceptable salt or stereoisomer thereof.

In some embodiments, the compound is Compound No. 29 or a pharmaceutically acceptable salt thereof.

In some embodiments, the compound is Compound No. 29.

In some embodiments, the compound is Compound No. 30 or a pharmaceutically acceptable salt or stereoisomer thereof.

In some embodiments, the compound is Compound No. 30 or a pharmaceutically acceptable salt thereof.

In some embodiments, the compound is Compound No. 30.

In some embodiments, the compound is Compound No. 31 or a pharmaceutically acceptable salt or stereoisomer thereof.

In some embodiments, the compound is Compound No. 31 or a pharmaceutically acceptable salt thereof.

In some embodiments, the compound is Compound No. 31.

In some embodiments, the compound is Compound No. 32 or a pharmaceutically acceptable salt or stereoisomer thereof.

In some embodiments, the compound is Compound No. 32 or a pharmaceutically acceptable salt thereof.

In some embodiments, the compound is Compound No. 32.

In some embodiments, the compound is Compound No. 33 or a pharmaceutically acceptable salt or stereoisomer thereof.

In some embodiments, the compound is Compound No. 33 or a pharmaceutically acceptable salt thereof.

In some embodiments, the compound is Compound No. 33.

In some embodiments, the compound is Compound No. 34 or a pharmaceutically acceptable salt or stereoisomer thereof.

In some embodiments, the compound is Compound No. 34 or a pharmaceutically acceptable salt thereof.

In some embodiments, the compound is Compound No. 34.

In some embodiments, the compound is Compound No. 35 or a pharmaceutically acceptable salt or stereoisomer thereof.

In some embodiments, the compound is Compound No. 35 or a pharmaceutically acceptable salt thereof.

In some embodiments, the compound is Compound No. 35.

In some embodiments, the compound is Compound No. 37 or a pharmaceutically acceptable salt or stereoisomer thereof.

In some embodiments, the compound is Compound No. 37 or a pharmaceutically acceptable salt thereof.

In some embodiments, the compound is Compound No. 37.

In some embodiments, the compound is Compound No. 38 or a pharmaceutically acceptable salt or stereoisomer thereof.

In some embodiments, the compound is Compound No. 38 or a pharmaceutically acceptable salt thereof.

In some embodiments, the compound is Compound No. 38.

In some embodiments, the compound is Compound No. 39 or a pharmaceutically acceptable salt or stereoisomer thereof.

In some embodiments, the compound is Compound No. 39 or a pharmaceutically acceptable salt thereof.

In some embodiments, the compound is Compound No. 39.

In some embodiments, the compound is Compound No. 40 or a pharmaceutically acceptable salt or stereoisomer thereof.

In some embodiments, the compound is Compound No. 40 or a pharmaceutically acceptable salt thereof.

In some embodiments, the compound is Compound No. 40.

In some embodiments, the compound is Compound No. 41 or a pharmaceutically acceptable salt or stereoisomer thereof.

In some embodiments, the compound is Compound No. 41 or a pharmaceutically acceptable salt thereof.

In some embodiments, the compound is Compound No. 41.

In some embodiments, the compound is Compound No. 42 or a pharmaceutically acceptable salt or stereoisomer thereof.

In some embodiments, the compound is Compound No. 42 or a pharmaceutically acceptable salt thereof.

In some embodiments, the compound is Compound No. 42.

In some embodiments, the compound is Compound No. 43 or a pharmaceutically acceptable salt or stereoisomer thereof.

In some embodiments, the compound is Compound No. 43 or a pharmaceutically acceptable salt thereof.

In some embodiments, the compound is Compound No. 43.

In some embodiments, the compound is Compound No. 44 or a pharmaceutically acceptable salt or stereoisomer thereof.

In some embodiments, the compound is Compound No. 44 or a pharmaceutically acceptable salt thereof.

In some embodiments, the compound is Compound No. 44.

In some embodiments, the compound is Compound No. 49 or a pharmaceutically acceptable salt or stereoisomer thereof.

In some embodiments, the compound is Compound No. 49 or a pharmaceutically acceptable salt thereof.

In some embodiments, the compound is Compound No. 49.

In some embodiments, the compound is Compound No. 52 or a pharmaceutically acceptable salt or stereoisomer thereof.

In some embodiments, the compound is Compound No. 52 or a pharmaceutically acceptable salt thereof.

In some embodiments, the compound is Compound No. 52.

In some embodiments, the compound is Compound No. 53 or a pharmaceutically acceptable salt or stereoisomer thereof.

In some embodiments, the compound is Compound No. 53 or a pharmaceutically acceptable salt thereof.

In some embodiments, the compound is Compound No. 53.

In some embodiments, the compound is Compound No. 55 or a pharmaceutically acceptable salt or stereoisomer thereof.

In some embodiments, the compound is Compound No. 55 or a pharmaceutically acceptable salt thereof.

In some embodiments, the compound is Compound No. 55.

In some embodiments, the compound is Compound No. 56 or a pharmaceutically acceptable salt or stereoisomer thereof.

In some embodiments, the compound is Compound No. 56 or a pharmaceutically acceptable salt thereof.

In some embodiments, the compound is Compound No. 56.

In some embodiments, the compound is Compound No. 57 or a pharmaceutically acceptable salt or stereoisomer thereof.

In some embodiments, the compound is Compound No. 57 or a pharmaceutically acceptable salt thereof.

In some embodiments, the compound is Compound No. 57.

In some embodiments, the compound is Compound No. 58 or a pharmaceutically acceptable salt or stereoisomer thereof.

In some embodiments, the compound is Compound No. 58 or a pharmaceutically acceptable salt thereof.

In some embodiments, the compound is Compound No. 58.

In some embodiments, the compound is Compound No. 59 or a pharmaceutically acceptable salt or stereoisomer thereof.

In some embodiments, the compound is Compound No. 59 or a pharmaceutically acceptable salt thereof.

In some embodiments, the compound is Compound No. 59.

In some embodiments, the compound is Compound No. 60 or a pharmaceutically acceptable salt or stereoisomer thereof.

In some embodiments, the compound is Compound No. 60 or a pharmaceutically acceptable salt thereof.

In some embodiments, the compound is Compound No. 60.

In some embodiments, the compound is Compound No. 62 or a pharmaceutically acceptable salt or stereoisomer thereof.

In some embodiments, the compound is Compound No. 62 or a pharmaceutically acceptable salt thereof.

In some embodiments, the compound is Compound No. 62.

In some embodiments, the compound is Compound No. 63 or a pharmaceutically acceptable salt or stereoisomer thereof.

In some embodiments, the compound is Compound No. 63 or a pharmaceutically acceptable salt thereof.

In some embodiments, the compound is Compound No. 63.

In some embodiments, the compound is Compound No. 65 or a pharmaceutically acceptable salt or stereoisomer thereof.

In some embodiments, the compound is Compound No. 65 or a pharmaceutically acceptable salt thereof.

In some embodiments, the compound is Compound No. 65.

In some embodiments, the compound is Compound No. 66 or a pharmaceutically acceptable salt or stereoisomer thereof.

In some embodiments, the compound is Compound No. 66 or a pharmaceutically acceptable salt thereof.

In some embodiments, the compound is Compound No. 66.

In some embodiments, the compound is Compound No. 68 or a pharmaceutically acceptable salt or stereoisomer thereof.

In some embodiments, the compound is Compound No. 68 or a pharmaceutically acceptable salt thereof.

In some embodiments, the compound is Compound No. 68.

In some embodiments, the compound is Compound No. 71 or a pharmaceutically acceptable salt or stereoisomer thereof.

In some embodiments, the compound is Compound No. 71 or a pharmaceutically acceptable salt thereof.

In some embodiments, the compound is Compound No. 71.

In some embodiments, the compound is Compound No. 75 or a pharmaceutically acceptable salt or stereoisomer thereof.

In some embodiments, the compound is Compound No. 75 or a pharmaceutically acceptable salt thereof.

In some embodiments, the compound is Compound No. 75.

In some embodiments, the compound is Compound No. 77 or a pharmaceutically acceptable salt or stereoisomer thereof.

In some embodiments, the compound is Compound No. 77 or a pharmaceutically acceptable salt thereof.

In some embodiments, the compound is Compound No. 77.

In some embodiments, the compound is Compound No. 78 or a pharmaceutically acceptable salt or stereoisomer thereof.

In some embodiments, the compound is Compound No. 78 or a pharmaceutically acceptable salt thereof.

In some embodiments, the compound is Compound No. 78.

In some embodiments, the compound is Compound No. 79 or a pharmaceutically acceptable salt or stereoisomer thereof.

In some embodiments, the compound is Compound No. 79 or a pharmaceutically acceptable salt thereof.

In some embodiments, the compound is Compound No. 79.

In some embodiments, the compound is Compound No. 153 or a pharmaceutically acceptable salt or stereoisomer thereof.

In some embodiments, the compound is Compound No. 153 or a pharmaceutically acceptable salt thereof.

In some embodiments, the compound is Compound No. 153.

In some embodiments, the compound is Compound No. 154 or a pharmaceutically acceptable salt or stereoisomer thereof.

In some embodiments, the compound is Compound No. 154 or a pharmaceutically acceptable salt thereof.

In some embodiments, the compound is Compound No. 154.

In some embodiments, the compound is Compound No. 173 or a pharmaceutically acceptable salt or stereoisomer thereof.

In some embodiments, the compound is Compound No. 173 or a pharmaceutically acceptable salt thereof.

In some embodiments, the compound is Compound No. 173.

In some embodiments, the compound is Compound No. 174 or a pharmaceutically acceptable salt or stereoisomer thereof.

In some embodiments, the compound is Compound No. 174 or a pharmaceutically acceptable salt thereof.

In some embodiments, the compound is Compound No. 174.

In some embodiments, the compound is Compound No. 175 or a pharmaceutically acceptable salt or stereoisomer thereof.

In some embodiments, the compound is Compound No. 175 or a pharmaceutically acceptable salt thereof.

In some embodiments, the compound is Compound No. 175.

In some embodiments, the compound is Compound No. 176 or a pharmaceutically acceptable salt or stereoisomer thereof.

In some embodiments, the compound is Compound No. 176 or a pharmaceutically acceptable salt thereof.

In some embodiments, the compound is Compound No. 176.

In some embodiments, the compound is Compound No. 191 or a pharmaceutically acceptable salt or stereoisomer thereof.

In some embodiments, the compound is Compound No. 191 or a pharmaceutically acceptable salt thereof.

In some embodiments, the compound is Compound No. 191.

In some embodiments, the compound is Compound No. 193 or a pharmaceutically acceptable salt or stereoisomer thereof.

In some embodiments, the compound is Compound No. 193 or a pharmaceutically acceptable salt thereof.

In some embodiments, the compound is Compound No. 193.

In some embodiments, the compound is Compound No. 194 or a pharmaceutically acceptable salt or stereoisomer thereof.

In some embodiments, the compound is Compound No. 194 or a pharmaceutically acceptable salt thereof.

In some embodiments, the compound is Compound No. 194.

In some embodiments, the compound is Compound No. 200 or a pharmaceutically acceptable salt or stereoisomer thereof.

In some embodiments, the compound is Compound No. 200 or a pharmaceutically acceptable salt thereof.

In some embodiments, the compound is Compound No. 200.

In some embodiments, the compound is Compound No. 201 or a pharmaceutically acceptable salt or stereoisomer thereof.

In some embodiments, the compound is Compound No. 201 or a pharmaceutically acceptable salt thereof.

In some embodiments, the compound is Compound No. 201.

In some embodiments, the compound is Compound No. 202 or a pharmaceutically acceptable salt or stereoisomer thereof.

In some embodiments, the compound is Compound No. 202 or a pharmaceutically acceptable salt thereof.

In some embodiments, the compound is Compound No. 202.

In some embodiments, the compound is Compound No. 203 or a pharmaceutically acceptable salt or stereoisomer thereof.

In some embodiments, the compound is Compound No. 203 or a pharmaceutically acceptable salt thereof.

In some embodiments, the compound is Compound No. 203.

In some embodiments, the compound is Compound No. 204 or a pharmaceutically acceptable salt or stereoisomer thereof.

In some embodiments, the compound is Compound No. 204 or a pharmaceutically acceptable salt thereof.

In some embodiments, the compound is Compound No. 204.

In some embodiments, the compound is Compound No. 206 or a pharmaceutically acceptable salt or stereoisomer thereof.

In some embodiments, the compound is Compound No. 206 or a pharmaceutically acceptable salt thereof.

In some embodiments, the compound is Compound No. 206.

In some embodiments, the compound is Compound No. 214 or a pharmaceutically acceptable salt or stereoisomer thereof.

In some embodiments, the compound is Compound No. 214 or a pharmaceutically acceptable salt thereof.

In some embodiments, the compound is Compound No. 214.

In some embodiments, the compound is Compound No. 215 or a pharmaceutically acceptable salt or stereoisomer thereof.

In some embodiments, the compound is Compound No. 215 or a pharmaceutically acceptable salt thereof.

In some embodiments, the compound is Compound No. 215.

In some embodiments, the compound is Compound No. 217 or a pharmaceutically acceptable salt or stereoisomer thereof.

In some embodiments, the compound is Compound No. 217 or a pharmaceutically acceptable salt thereof.

In some embodiments, the compound is Compound No. 217.

In some embodiments, the compound is Compound No. 218 or a pharmaceutically acceptable salt or stereoisomer thereof.

In some embodiments, the compound is Compound No. 218 or a pharmaceutically acceptable salt thereof.

In some embodiments, the compound is Compound No. 218.

In some embodiments, the compound is Compound No. 220 or a pharmaceutically acceptable salt or stereoisomer thereof.

In some embodiments, the compound is Compound No. 220 or a pharmaceutically acceptable salt thereof.

In some embodiments, the compound is Compound No. 220.

In some embodiments, the compound is Compound No. 221 or a pharmaceutically acceptable salt or stereoisomer thereof.

In some embodiments, the compound is Compound No. 221 or a pharmaceutically acceptable salt thereof.

In some embodiments, the compound is Compound No. 221.

In some embodiments, the compound is Compound No. 222 or a pharmaceutically acceptable salt or stereoisomer thereof.

In some embodiments, the compound is Compound No. 222 or a pharmaceutically acceptable salt thereof.

In some embodiments, the compound is Compound No. 222.

In some embodiments, the compound is Compound No. 223 or a pharmaceutically acceptable salt or stereoisomer thereof.

In some embodiments, the compound is Compound No. 223 or a pharmaceutically acceptable salt thereof.

In some embodiments, the compound is Compound No. 223.

In some embodiments, the compound is Compound No. 225 or a pharmaceutically acceptable salt or stereoisomer thereof.

In some embodiments, the compound is Compound No. 225 or a pharmaceutically acceptable salt thereof.

In some embodiments, the compound is Compound No. 225.

In some embodiments, the compound is Compound No. 226 or a pharmaceutically acceptable salt or stereoisomer thereof.

In some embodiments, the compound is Compound No. 226 or a pharmaceutically acceptable salt thereof.

In some embodiments, the compound is Compound No. 226.

In some embodiments, the compound is Compound No. 227 or a pharmaceutically acceptable salt or stereoisomer thereof.

In some embodiments, the compound is Compound No. 227 or a pharmaceutically acceptable salt thereof.

In some embodiments, the compound is Compound No. 227.

In some embodiments, the compound is Compound No. 229 or a pharmaceutically acceptable salt or stereoisomer thereof.

In some embodiments, the compound is Compound No. 229 or a pharmaceutically acceptable salt thereof.

In some embodiments, the compound is Compound No. 229.

In some embodiments, the compound is Compound No. 230 or a pharmaceutically acceptable salt or stereoisomer thereof.

In some embodiments, the compound is Compound No. 230 or a pharmaceutically acceptable salt thereof.

In some embodiments, the compound is Compound No. 230.

In some embodiments, the compound is Compound No. 231 or a pharmaceutically acceptable salt or stereoisomer thereof.

In some embodiments, the compound is Compound No. 231 or a pharmaceutically acceptable salt thereof.

In some embodiments, the compound is Compound No. 231.

In some embodiments, the compound is Compound No. 233 or a pharmaceutically acceptable salt or stereoisomer thereof.

In some embodiments, the compound is Compound No. 233 or a pharmaceutically acceptable salt thereof.

In some embodiments, the compound is Compound No. 233.

In some embodiments, the compound is Compound No. 234 or a pharmaceutically acceptable salt or stereoisomer thereof.

In some embodiments, the compound is Compound No. 234 or a pharmaceutically acceptable salt thereof.

In some embodiments, the compound is Compound No. 234.

In some embodiments, the compound is Compound No. 235 or a pharmaceutically acceptable salt or stereoisomer thereof.

In some embodiments, the compound is Compound No. 235 or a pharmaceutically acceptable salt thereof.

In some embodiments, the compound is Compound No. 235.

In some embodiments, the compound is Compound No. 236 or a pharmaceutically acceptable salt or stereoisomer thereof.

In some embodiments, the compound is Compound No. 236 or a pharmaceutically acceptable salt thereof.

In some embodiments, the compound is Compound No. 236.

In some embodiments, the compound is Compound No. 237 or a pharmaceutically acceptable salt or stereoisomer thereof.

In some embodiments, the compound is Compound No. 237 or a pharmaceutically acceptable salt thereof.

In some embodiments, the compound is Compound No. 237.

In some embodiments, the compound is Compound No. 238 or a pharmaceutically acceptable salt or stereoisomer thereof.

In some embodiments, the compound is Compound No. 238 or a pharmaceutically acceptable salt thereof.

In some embodiments, the compound is Compound No. 238.

In some embodiments, the compound is Compound No. 242 or a pharmaceutically acceptable salt or stereoisomer thereof.

In some embodiments, the compound is Compound No. 242 or a pharmaceutically acceptable salt thereof.

In some embodiments, the compound is Compound No. 242.

In some embodiments, the compound is Compound No. 243 or a pharmaceutically acceptable salt or stereoisomer thereof.

In some embodiments, the compound is Compound No. 243 or a pharmaceutically acceptable salt thereof.

In some embodiments, the compound is Compound No. 243.

In some embodiments, the compound is Compound No. 245 or a pharmaceutically acceptable salt or stereoisomer thereof.

In some embodiments, the compound is Compound No. 245 or a pharmaceutically acceptable salt thereof.

In some embodiments, the compound is Compound No. 245.

In some embodiments, the compound is Compound No. 247 or a pharmaceutically acceptable salt or stereoisomer thereof.

In some embodiments, the compound is Compound No. 247 or a pharmaceutically acceptable salt thereof.

In some embodiments, the compound is Compound No. 247.

In some embodiments, the compound is Compound No. 248 or a pharmaceutically acceptable salt or stereoisomer thereof.

In some embodiments, the compound is Compound No. 248 or a pharmaceutically acceptable salt thereof.

In some embodiments, the compound is Compound No. 248.

In some embodiments, the compound is Compound No. 249 or a pharmaceutically acceptable salt or stereoisomer thereof.

In some embodiments, the compound is Compound No. 249 or a pharmaceutically acceptable salt thereof.

In some embodiments, the compound is Compound No. 249.

In some embodiments, the compound is Compound No. 252 or a pharmaceutically acceptable salt or stereoisomer thereof.

In some embodiments, the compound is Compound No. 252 or a pharmaceutically acceptable salt thereof.

In some embodiments, the compound is Compound No. 252.

In some embodiments, the compound is Compound No. 253 or a pharmaceutically acceptable salt or stereoisomer thereof.

In some embodiments, the compound is Compound No. 253 or a pharmaceutically acceptable salt thereof.

In some embodiments, the compound is Compound No. 253.

In some embodiments, the compound is Compound No. 255 or a pharmaceutically acceptable salt or stereoisomer thereof.

In some embodiments, the compound is Compound No. 255 or a pharmaceutically acceptable salt thereof.

In some embodiments, the compound is Compound No. 255.

In some embodiments, the compound is Compound No. 256 or a pharmaceutically acceptable salt or stereoisomer thereof.

In some embodiments, the compound is Compound No. 256 or a pharmaceutically acceptable salt thereof.

In some embodiments, the compound is Compound No. 256.

In some embodiments, the compound is Compound No. 257 or a pharmaceutically acceptable salt or stereoisomer thereof.

In some embodiments, the compound is Compound No. 257 or a pharmaceutically acceptable salt thereof.

In some embodiments, the compound is Compound No. 257.

In some embodiments, the compound is Compound No. 258 or a pharmaceutically acceptable salt or stereoisomer thereof.

In some embodiments, the compound is Compound No. 258 or a pharmaceutically acceptable salt thereof.

In some embodiments, the compound is Compound No. 258.

In some embodiments, the compound is Compound No. 259 or a pharmaceutically acceptable salt or stereoisomer thereof.

In some embodiments, the compound is Compound No. 259 or a pharmaceutically acceptable salt thereof.

In some embodiments, the compound is Compound No. 259.

In some embodiments, the compound is Compound No. 260 or a pharmaceutically acceptable salt or stereoisomer thereof.

In some embodiments, the compound is Compound No. 260 or a pharmaceutically acceptable salt thereof.

In some embodiments, the compound is Compound No. 260.

In some embodiments, the compound is Compound No. 261 or a pharmaceutically acceptable salt or stereoisomer thereof.

In some embodiments, the compound is Compound No. 261 or a pharmaceutically acceptable salt thereof.

In some embodiments, the compound is Compound No. 261.

In some embodiments, the compound is Compound No. 262 or a pharmaceutically acceptable salt or stereoisomer thereof.

In some embodiments, the compound is Compound No. 262 or a pharmaceutically acceptable salt thereof.

In some embodiments, the compound is Compound No. 262.

In some embodiments, the compound is Compound No. 263 or a pharmaceutically acceptable salt or stereoisomer thereof.

In some embodiments, the compound is Compound No. 263 or a pharmaceutically acceptable salt thereof.

In some embodiments, the compound is Compound No. 263.

In some embodiments, the compound is Compound No. 264 or a pharmaceutically acceptable salt or stereoisomer thereof.

In some embodiments, the compound is Compound No. 264 or a pharmaceutically acceptable salt thereof.

In some embodiments, the compound is Compound No. 264.

In some embodiments, the compound is Compound No. 265 or a pharmaceutically acceptable salt or stereoisomer thereof.

In some embodiments, the compound is Compound No. 265 or a pharmaceutically acceptable salt thereof.

In some embodiments, the compound is Compound No. 265.

In some embodiments, the compound is Compound No. 266 or a pharmaceutically acceptable salt or stereoisomer thereof.

In some embodiments, the compound is Compound No. 266 or a pharmaceutically acceptable salt thereof.

In some embodiments, the compound is Compound No. 266.

In some embodiments, the compound is Compound No. 267 or a pharmaceutically acceptable salt or stereoisomer thereof.

In some embodiments, the compound is Compound No. 267 or a pharmaceutically acceptable salt thereof.

In some embodiments, the compound is Compound No. 267.

In some embodiments, the compound is Compound No. 271 or a pharmaceutically acceptable salt or stereoisomer thereof.

In some embodiments, the compound is Compound No. 271 or a pharmaceutically acceptable salt thereof.

In some embodiments, the compound is Compound No. 271.

In some embodiments, the compound is Compound No. 272 or a pharmaceutically acceptable salt or stereoisomer thereof.

In some embodiments, the compound is Compound No. 272 or a pharmaceutically acceptable salt thereof.

In some embodiments, the compound is Compound No. 272.

In some embodiments, the compound is Compound No. 274 or a pharmaceutically acceptable salt or stereoisomer thereof.

In some embodiments, the compound is Compound No. 274 or a pharmaceutically acceptable salt thereof.

In some embodiments, the compound is Compound No. 274.

In some embodiments, the compound is Compound No. 275 or a pharmaceutically acceptable salt or stereoisomer thereof.

In some embodiments, the compound is Compound No. 275 or a pharmaceutically acceptable salt thereof.

In some embodiments, the compound is Compound No. 275.

In some embodiments, the compound is Compound No. 276 or a pharmaceutically acceptable salt or stereoisomer thereof.

In some embodiments, the compound is Compound No. 276 or a pharmaceutically acceptable salt thereof.

In some embodiments, the compound is Compound No. 276.

A compound of the present disclosure or a pharmaceutically acceptable salt thereof that contains the aforementioned deuterium atom(s) is within the scope of the disclosure. Further, substitution with deuterium (i.e., ²H) may afford certain therapeutic advantages resulting from greater metabolic stability, e.g., increased in vivo half-life or reduced dosage requirements.

For the avoidance of doubt it is to be understood that, where in this specification a group is qualified by “described herein”, the said group encompasses the first occurring and broadest definition as well as each and all of the particular definitions for that group.

A suitable pharmaceutically acceptable salt of a compound of the disclosure is, for example, an acid-addition salt of a compound of the disclosure which is sufficiently basic, for example, an acid-addition salt with, for example, an inorganic or organic acid, for example hydrochloric, hydrobromic, sulfuric, phosphoric, trifluoroacetic, formic, citric methane sulfonate or maleic acid. In addition, a suitable pharmaceutically acceptable salt of a compound of the disclosure which is sufficiently acidic is an alkali metal salt, for example a sodium or potassium salt, an alkaline earth metal salt, for example a calcium or magnesium salt, an ammonium salt or a salt with an organic base which affords a pharmaceutically acceptable cation, for example a salt with methylamine, dimethylamine, diethylamine, trimethylamine, piperidine, morpholine or tris-(2-hydroxyethyl)amine.

It will be understood that the compounds of the present disclosure and any pharmaceutically acceptable salts thereof, comprise stereoisomers, mixtures of stereoisomers, polymorphs of all isomeric forms of said compounds.

As used herein, the term “isomerism” means compounds that have identical molecular formulae but differ in the sequence of bonding of their atoms or in the arrangement of their atoms in space. Isomers that differ in the arrangement of their atoms in space are termed “stereoisomers.” Stereoisomers that are not mirror images of one another are termed “diastereoisomers,” and stereoisomers that are non-superimposable mirror images of each other are termed “enantiomers” or sometimes optical isomers. A mixture containing equal amounts of individual enantiomeric forms of opposite chirality is termed a “racemic mixture.”

As used herein, the term “chiral center” refers to a carbon atom bonded to four nonidentical substituents.

As used herein, the term “chiral isomer” means a compound with at least one chiral center. Compounds with more than one chiral center may exist either as an individual diastereomer or as a mixture of diastereomers, termed “diastereomeric mixture.” When one chiral center is present, a stereoisomer may be characterized by the absolute configuration (R or S) of that chiral center. Absolute configuration refers to the arrangement in space of the substituents attached to the chiral center. The substituents attached to the chiral center under consideration are ranked in accordance with the Sequence Rule of Cahn, Ingold and Prelog. (Cahn et al., Angew. Chem. Inter. Edit. 1966, 5, 385; errata 511; Cahn et al., Angew. Chem. 1966, 78, 413; Cahn and Ingold, J. Chem. Soc. 1951 (London), 612; Cahn et al., Experientia 1956, 12, 81; Cahn, J. Chem. Educ. 1964, 41, 116).

As used herein, the term “geometric isomer” means the diastereomers that owe their existence to hindered rotation about double bonds or a cycloalkyl linker (e.g., 1,3-cyclobutyl). These configurations are differentiated in their names by the prefixes cis and trans, or Z and E, which indicate that the groups are on the same or opposite side of the double bond in the molecule according to the Cahn-Ingold-Prelog rules.

It is to be understood that the compounds of the present disclosure may be depicted as different chiral isomers or geometric isomers. It is also to be understood that when compounds have chiral isomeric or geometric isomeric forms, all isomeric forms are intended to be included in the scope of the present disclosure, and the naming of the compounds does not exclude any isomeric forms, it being understood that not all isomers may have the same level of activity.

It is to be understood that the structures and other compounds discussed in this disclosure include all atropic isomers thereof. It is also to be understood that not all atropic isomers may have the same level of activity.

As used herein, the term “atropic isomers” are a type of stereoisomer in which the atoms of two isomers are arranged differently in space. Atropic isomers owe their existence to a restricted rotation caused by hindrance of rotation of large groups about a central bond. Such atropic isomers typically exist as a mixture, however as a result of recent advances in chromatography techniques, it has been possible to separate mixtures of two atropic isomers in select cases.

As used herein, the term “tautomer” is one of two or more structural isomers that exist in equilibrium and is readily converted from one isomeric form to another. This conversion results in the formal migration of a hydrogen atom accompanied by a switch of adjacent conjugated double bonds. Tautomers exist as a mixture of a tautomeric set in solution. In solutions where tautomerisation is possible, a chemical equilibrium of the tautomers will be reached. The exact ratio of the tautomers depends on several factors, including temperature, solvent and pH. The concept of tautomers that are interconvertible by tautomerisations is called tautomerism. Of the various types of tautomerism that are possible, two are commonly observed. In keto-enol tautomerism a simultaneous shift of electrons and a hydrogen atom occurs. Ring-chain tautomerism arises as a result of the aldehyde group (—CHO) in a sugar chain molecule reacting with one of the hydroxy groups (—OH) in the same molecule to give it a cyclic (ring-shaped) form as exhibited by glucose.

It is to be understood that the compounds of the present disclosure may be depicted as different tautomers. It should also be understood that when compounds have tautomeric forms, all tautomeric forms are intended to be included in the scope of the present disclosure, and the naming of the compounds does not exclude any tautomer form. It will be understood that certain tautomers may have a higher level of activity than others.

Compounds that have the same molecular formula but differ in the nature or sequence of bonding of their atoms or the arrangement of their atoms in space are termed “isomers”. Isomers that differ in the arrangement of their atoms in space are termed “stereoisomers”. Stereoisomers that are not mirror images of one another are termed “diastereomers” and those that are non-superimposable mirror images of each other are termed “enantiomers”. When a compound has an asymmetric center, for example, it is bonded to four different groups, a pair of enantiomers is possible. An enantiomer can be characterized by the absolute configuration of its asymmetric center and is described by the R- and S-sequencing rules of Cahn and Prelog, or by the manner in which the molecule rotates the plane of polarised light and designated as dextrorotatory or levorotatory (i.e., as (+) or (−)-isomers respectively). A chiral compound can exist as either individual enantiomer or as a mixture thereof. A mixture containing equal proportions of the enantiomers is called a “racemic mixture”.

The compounds of this disclosure may possess one or more asymmetric centers; such compounds can therefore be produced as individual (R)- or (S)-stereoisomers or as mixtures thereof. Unless indicated otherwise, the description or naming of a particular compound in the specification and claims is intended to include both individual enantiomers and mixtures, racemic or otherwise, thereof. The methods for the determination of stereochemistry and the separation of stereoisomers are well-known in the art (see discussion in Chapter 4 of “Advanced Organic Chemistry”, 4th edition J. March, John Wiley and Sons, New York, 2001), for example by synthesis from optically active starting materials or by resolution of a racemic form. Some of the compounds of the disclosure may have geometric isomeric centers (E- and Z-isomers). It is to be understood that the present disclosure encompasses all optical, diastereoisomers and geometric isomers and mixtures thereof that possess anti-tumor activity.

The present disclosure also encompasses compounds of the disclosure as defined herein which comprise one or more isotopic substitutions.

It is to be understood that the compounds of any Formula described herein include the compounds themselves, as well as their salts, and their solvates, if applicable. A salt, for example, can be formed between an anion and a positively charged group (e.g., amino) on a substituted compound disclosed herein. Suitable anions include chloride, bromide, iodide, sulfate, bisulfate, sulfamate, nitrate, phosphate, citrate, methanesulfonate, trifluoroacetate, glutamate, glucuronate, glutarate, malate, maleate, succinate, fumarate, tartrate, tosylate, salicylate, lactate, naphthalenesulfonate, and acetate (e.g., trifluoroacetate).

As used herein, the term “pharmaceutically acceptable anion” refers to an anion suitable for forming a pharmaceutically acceptable salt. Likewise, a salt can also be formed between a cation and a negatively charged group (e.g., carboxylate) on a substituted compound disclosed herein. Suitable cations include sodium ion, potassium ion, magnesium ion, calcium ion, and an ammonium cation such as tetramethylammonium ion or diethylamine ion. The substituted compounds disclosed herein also include those salts containing quaternary nitrogen atoms.

It is to be understood that the compounds of the present disclosure, for example, the salts of the compounds, can exist in either hydrated or unhydrated (the anhydrous) form or as solvates with other solvent molecules. Nonlimiting examples of hydrates include monohydrates, dihydrates, etc. Nonlimiting examples of solvates include ethanol solvates, acetone solvates, etc.

As used herein, the term “solvate” means solvent addition forms that contain either stoichiometric or non-stoichiometric amounts of solvent. Some compounds have a tendency to trap a fixed molar ratio of solvent molecules in the crystalline solid state, thus forming a solvate. If the solvent is water the solvate formed is a hydrate; and if the solvent is alcohol, the solvate formed is an alcoholate. Hydrates are formed by the combination of one or more molecules of water with one molecule of the substance in which the water retains its molecular state as H₂O.

As used herein, the term “analog” refers to a chemical compound that is structurally similar to another but differs slightly in composition (as in the replacement of one atom by an atom of a different element or in the presence of a particular functional group, or the replacement of one functional group by another functional group). Thus, an analog is a compound that is similar or comparable in function and appearance, but not in structure or origin to the reference compound.

As used herein, the term “derivative” refers to compounds that have a common core structure and are substituted with various groups as described herein.

As used herein, the term “bioisostere” refers to a compound resulting from the exchange of an atom or of a group of atoms with another, broadly similar, atom or group of atoms. The objective of a bioisosteric replacement is to create a new compound with similar biological properties to the parent compound. The bioisosteric replacement may be physicochemically or topologically based. Examples of carboxylic acid bioisosteres include, but are not limited to, acyl sulfonamides, tetrazoles, sulfonates and phosphonates. See, e.g., Patani and LaVoie, Chem. Rev. 96, 3147-3176, 1996.

It is also to be understood that certain compounds of the present disclosure may exist in solvated as well as unsolvated forms such as, for example, hydrated forms. A suitable pharmaceutically acceptable solvate is, for example, a hydrate such as hemi-hydrate, a mono-hydrate, a di-hydrate or a tri-hydrate. It is to be understood that the disclosure encompasses all such solvated forms that possess anti-tumor activity.

It is also to be understood that certain compounds of the present disclosure may exhibit polymorphism, and that the disclosure encompasses all such forms, or mixtures thereof, which possess anti-tumor activity. It is generally known that crystalline materials may be analysed using conventional techniques such as X-Ray Powder Diffraction analysis, Differential Scanning Calorimetry, Thermal Gravimetric Analysis, Diffuse Reflectance Infrared Fourier Transform (DRIFT) spectroscopy, Near Infrared (NTR) spectroscopy, solution and/or solid state nuclear magnetic resonance spectroscopy. The water content of such crystalline materials may be determined by Karl Fischer analysis.

Compounds of the present disclosure may exist in a number of different tautomeric forms and references to compounds of the present disclosure include all such forms. For the avoidance of doubt, where a compound can exist in one of several tautomeric forms, and only one is specifically described or shown, all others are nevertheless embraced by the Formulae disclosed. Examples of tautomeric forms include keto-, enol-, and enolate-forms, as in, for example, the following tautomeric pairs: keto/enol (illustrated below), imine/enamine, amide/imino alcohol, amidine/amidine, nitroso/oxime, thioketone/enethiol, and nitro/aci-nitro.

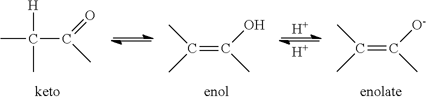

Compounds of the present disclosure containing an amine function may also form N-oxides. A reference herein to a compound disclosed herein that contains an amine function also includes the N-oxide. Where a compound contains several amine functions, one or more than one nitrogen atom may be oxidised to form an N-oxide. Particular examples of N-oxides are the N-oxides of a tertiary amine or a nitrogen atom of a nitrogen-containing heterocycle. N-oxides can be formed by treatment of the corresponding amine with an oxidising agent such as hydrogen peroxide or a peracid (e.g. a peroxycarboxylic acid), see for example Advanced Organic Chemistry, by Jerry March, 4th Edition, Wiley Interscience, pages. More particularly, N-oxides can be made by the procedure of L. W. Deady (Syn. Comm. 1977, 7, 509-514) in which the amine compound is reacted with meta-chloroperoxybenzoic acid (mCPBA), for example, in an inert solvent such as dichloromethane.

The compounds of the present disclosure may be administered in the form of a prodrug which is broken down in the human or animal body to release a compound of the disclosure. A prodrug may be used to alter the physical properties and/or the pharmacokinetic properties of a compound of the disclosure. A prodrug can be formed when the compound of the disclosure contains a suitable group or substituent to which a property-modifying group can be attached. Examples of prodrugs include derivatives containing in vivo cleavable alkyl or acyl substituents at the sulfonylurea group in a compound of the any one of the Formulae disclosed herein.

Accordingly, the present disclosure includes those compounds of the present disclosure as defined hereinbefore when made available by organic synthesis and when made available within the human or animal body by way of cleavage of a prodrug thereof. Accordingly, the present disclosure includes those compounds of the present disclosure that are produced by organic synthetic means and also such compounds that are produced in the human or animal body by way of metabolism of a precursor compound, that is a compound of the present disclosure may be a synthetically-produced compound or a metabolically-produced compound.

A suitable pharmaceutically acceptable prodrug of a compound of the present disclosure is one that is based on reasonable medical judgment as being suitable for administration to the human or animal body without undesirable pharmacological activities and without undue toxicity. Various forms of prodrug have been described, for example in the following documents: a) Methods in Enzymology, Vol. 42, p. 309-396, edited by K. Widder, et al. (Academic Press, 1985); b) Design of Pro-drugs, edited by H. Bundgaard, (Elsevier, 1985); c) A Textbook of Drug Design and Development, edited by Krogsgaard-Larsen and H. Bundgaard, Chapter 5 “Design and Application of Pro-drugs”, by H. Bundgaard p. 113-191 (1991); d) H. Bundgaard, Advanced Drug Delivery Reviews, 8, 1-38 (1992); e) H. Bundgaard, et al., Journal of Pharmaceutical Sciences, 77, 285 (1988); f) N. Kakeya, et al., Chem. Pharm. Bull., 32, 692 (1984); g) T. Higuchi and V. Stella, “Pro-Drugs as Novel Delivery Systems”, A.C.S. Symposium Series, Volume 14; and h) E. Roche (editor), “Bioreversible Carriers in Drug Design”, Pergamon Press, 1987.

A suitable pharmaceutically acceptable prodrug of a compound of the present disclosure that possesses a hydroxy group is, for example, an in vivo cleavable ester or ether thereof. An in vivo cleavable ester or ether of a compound of the present disclosure containing a hydroxy group is, for example, a pharmaceutically acceptable ester or ether which is cleaved in the human or animal body to produce the parent hydroxy compound. Suitable pharmaceutically acceptable ester forming groups for a hydroxy group include inorganic esters such as phosphate esters (including phosphoramidic cyclic esters). Further suitable pharmaceutically acceptable ester forming groups for a hydroxy group include C₁-C₁₀ alkanoyl groups such as acetyl, benzoyl, phenylacetyl and substituted benzoyl and phenylacetyl groups, C₁-C₁₀ alkoxycarbonyl groups such as ethoxycarbonyl, N,N—(C₁-C₆ alkyl)₂carbamoyl, 2-dialkylaminoacetyl and 2-carboxyacetyl groups. Examples of ring substituents on the phenylacetyl and benzoyl groups include aminomethyl, N-alkylaminomethyl, N,N-dialkylaminomethyl, morpholinomethyl, piperazin-1-ylmethyl and 4-(C₁-C₄ alkyl)piperazin-1-ylmethyl. Suitable pharmaceutically acceptable ether forming groups for a hydroxy group include α-acyloxyalkyl groups such as acetoxymethyl and pivaloyloxymethyl groups.

A suitable pharmaceutically acceptable prodrug of a compound of the present disclosure that possesses a carboxy group is, for example, an in vivo cleavable amide thereof, for example an amide formed with an amine such as ammonia, a C₁₋₄alkylamine such as methylamine, a (C₁-C₄ alkyl)₂amine such as dimethylamine, N-ethyl-N-methylamine or diethylamine, a C₁-C₄ alkoxy-C₂-C₄ alkylamine such as 2-methoxyethylamine, a phenyl-C₁-C₄ alkylamine such as benzylamine and amino acids such as glycine or an ester thereof.

A suitable pharmaceutically acceptable prodrug of a compound of the present disclosure that possesses an amino group is, for example, an in vivo cleavable amide derivative thereof. Suitable pharmaceutically acceptable amides from an amino group include, for example an amide formed with C₁-C₁₀ alkanoyl groups such as an acetyl, benzoyl, phenylacetyl and substituted benzoyl and phenylacetyl groups. Examples of ring substituents on the phenylacetyl and benzoyl groups include aminomethyl, N-alkylaminomethyl, N,N-dialkylaminomethyl, morpholinomethyl, piperazin-1-ylmethyl, and 4-(C₁-C₄ alkyl)piperazin-1-ylmethyl.

The in vivo effects of a compound of the present disclosure may be exerted in part by one or more metabolites that are formed within the human or animal body after administration of a compound of the present disclosure. As stated hereinbefore, the in vivo effects of a compound of the present disclosure may also be exerted by way of metabolism of a precursor compound (a prodrug).

### Methods of Synthesis

In some aspects, the present disclosure provides a method of preparing a compound disclosed herein.

In some aspects, the present disclosure provides a method of preparing a compound, comprising one or more steps as described herein.

In some aspects, the present disclosure provides a compound obtainable by, or obtained by, or directly obtained by a method for preparing a compound described herein.

In some aspects, the present disclosure provides an intermediate being suitable for use in a method for preparing a compound described herein.

The compounds of the present disclosure can be prepared by any suitable technique known in the art. Particular processes for the preparation of these compounds are described further in the accompanying examples.

In the description of the synthetic methods described herein and in any referenced synthetic methods that are used to prepare the starting materials, it is to be understood that all proposed reaction conditions, including choice of solvent, reaction atmosphere, reaction temperature, duration of the experiment and workup procedures, can be selected by a person skilled in the art.

It is understood by one skilled in the art of organic synthesis that the functionality present on various portions of the molecule must be compatible with the reagents and reaction conditions utilised.

It will be appreciated that during the synthesis of the compounds of the disclosure in the processes defined herein, or during the synthesis of certain starting materials, it may be desirable to protect certain substituent groups to prevent their undesired reaction. The skilled chemist will appreciate when such protection is required, and how such protecting groups may be put in place, and later removed. For examples of protecting groups see one of the many general texts on the subject, for example, ‘Protective Groups in Organic Synthesis’ by Theodora Green (publisher: John Wiley & Sons). Protecting groups may be removed by any convenient method described in the literature or known to the skilled chemist as appropriate for the removal of the protecting group in question, such methods being chosen so as to effect removal of the protecting group with the minimum disturbance of groups elsewhere in the molecule. Thus, if reactants include, for example, groups such as amino, carboxy or hydroxy it may be desirable to protect the group in some of the reactions mentioned herein.

By way of example, a suitable protecting group for an amino or alkylamino group is, for example, an acyl group, for example an alkanoyl group such as acetyl, an alkoxycarbonyl group, for example a methoxycarbonyl, ethoxycarbonyl, or t-butoxycarbonyl group, an arylmethoxycarbonyl group, for example benzyloxycarbonyl, or an aroyl group, for example benzoyl. The deprotection conditions for the above protecting groups necessarily vary with the choice of protecting group. Thus, for example, an acyl group such as an alkanoyl or alkoxycarbonyl group or an aroyl group may be removed by, for example, hydrolysis with a suitable base such as an alkali metal hydroxide, for example lithium or sodium hydroxide. Alternatively an acyl group such as a tert-butoxycarbonyl group may be removed, for example, by treatment with a suitable acid as hydrochloric, sulfuric or phosphoric acid or trifluoroacetic acid and an arylmethoxycarbonyl group such as a benzyloxycarbonyl group may be removed, for example, by hydrogenation over a catalyst such as palladium on carbon, or by treatment with a Lewis acid for example boron tris(trifluoroacetate). A suitable alternative protecting group for a primary amino group is, for example, a phthaloyl group which may be removed by treatment with an alkylamine, for example dimethylaminopropylamine, or with hydrazine.

A suitable protecting group for a hydroxy group is, for example, an acyl group, for example an alkanoyl group such as acetyl, an aroyl group, for example benzoyl, or an arylmethyl group, for example benzyl. The deprotection conditions for the above protecting groups will necessarily vary with the choice of protecting group. Thus, for example, an acyl group such as an alkanoyl or an aroyl group may be removed, for example, by hydrolysis with a suitable base such as an alkali metal hydroxide, for example lithium, sodium hydroxide or ammonia. Alternatively an arylmethyl group such as a benzyl group may be removed, for example, by hydrogenation over a catalyst such as palladium on carbon.

A suitable protecting group for a carboxy group is, for example, an esterifying group, for example a methyl or an ethyl group which may be removed, for example, by hydrolysis with a base such as sodium hydroxide, or for example a tert-butyl group which may be removed, for example, by treatment with an acid, for example an organic acid such as trifluoroacetic acid, or for example a benzyl group which may be removed, for example, by hydrogenation over a catalyst such as palladium on carbon.

Once a compound of the present disclosure has been synthesized by any one of the processes defined herein, the processes may then further comprise the additional steps of: (i) removing any protecting groups present; (ii) converting the compound of the present disclosure into another compound of the present disclosure; (iii) forming a pharmaceutically acceptable salt, hydrate or solvate thereof; and/or (iv) forming a prodrug thereof.

The resultant compounds of the present disclosure can be isolated and purified using techniques well known in the art.

Conveniently, the reaction of the compounds is carried out in the presence of a suitable solvent, which is preferably inert under the respective reaction conditions. Examples of suitable solvents comprise but are not limited to hydrocarbons, such as hexane, petroleum ether, benzene, toluene or xylene; chlorinated hydrocarbons, such as trichlorethylene, 1,2-dichloroethane, tetrachloromethane, chloroform or dichloromethane; alcohols, such as methanol, ethanol, isopropanol, n-propanol, n-butanol or tert-butanol; ethers, such as diethyl ether, diisopropyl ether, tetrahydrofuran (THF), 2-methyltetrahydrofuran, cyclopentylmethyl ether (CPME), methyl tert-butyl ether (MTBE) or dioxane; glycol ethers, such as ethylene glycol monomethyl or monoethyl ether or ethylene glycol dimethyl ether (diglyme); ketones, such as acetone, methylisobutylketone (MIBK) or butanone; amides, such as acetamide, dimethylacetamide, dimethylformamide (DMF) or N-methylpyrrolidinone (NMP); nitriles, such as acetonitrile; sulfoxides, such as dimethyl sulfoxide (DMSO); nitro compounds, such as nitromethane or nitrobenzene; esters, such as ethyl acetate or methyl acetate, or mixtures of the said solvents or mixtures with water.

The reaction temperature is suitably between about −100 °C and 300 °C, depending on the reaction step and the conditions used.

Reaction times are generally in the range between a fraction of a minute and several days, depending on the reactivity of the respective compounds and the respective reaction conditions. Suitable reaction times are readily determinable by methods known in the art, for example reaction monitoring. Based on the reaction temperatures given above, suitable reaction times generally lie in the range between 10 minutes and 48 hours.

Moreover, by utilizing the procedures described herein, in conjunction with ordinary skills in the art, additional compounds of the present disclosure can be readily prepared. Those skilled in the art will readily understand that known variations of the conditions and processes of the following preparative procedures can be used to prepare these compounds.

As will be understood by the person skilled in the art of organic synthesis, compounds of the present disclosure are readily accessible by various synthetic routes, some of which are exemplified in the accompanying examples. The skilled person will easily recognize which kind of reagents and reactions conditions are to be used and how they are to be applied and adapted in any particular instance—wherever necessary or useful—in order to obtain the compounds of the present disclosure. Furthermore, some of the compounds of the present disclosure can readily be synthesized by reacting other compounds of the present disclosure under suitable conditions, for instance, by converting one particular functional group being present in a compound of the present disclosure, or a suitable precursor molecule thereof, into another one by applying standard synthetic methods, like reduction, oxidation, addition or substitution reactions; those methods are well known to the skilled person. Likewise, the skilled person will apply—whenever necessary or useful—synthetic protecting (or protective) groups; suitable protecting groups as well as methods for introducing and removing them are well-known to the person skilled in the art of chemical synthesis and are described, in more detail, in, e.g., P. G. M. Wuts, T. W. Greene, “Greene's Protective Groups in Organic Synthesis”, 4th edition (2006) (John Wiley & Sons).

General routes for the preparation of a compound of the application are described in Schemes I-X.

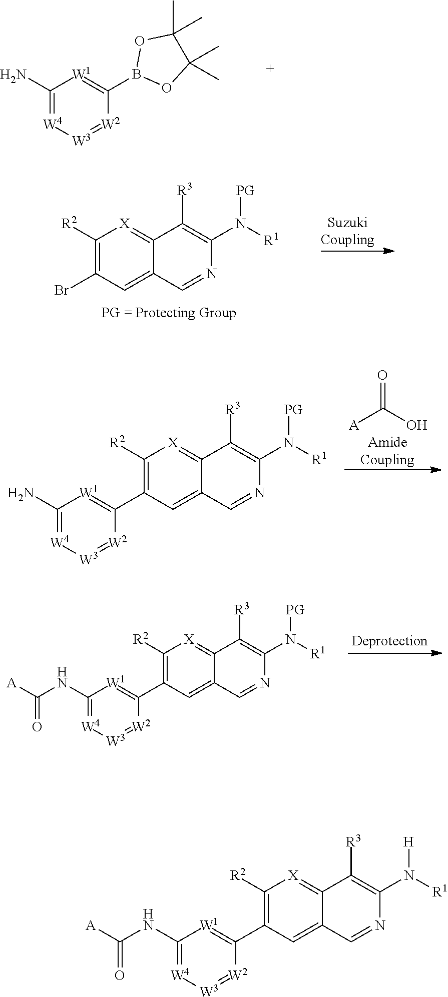

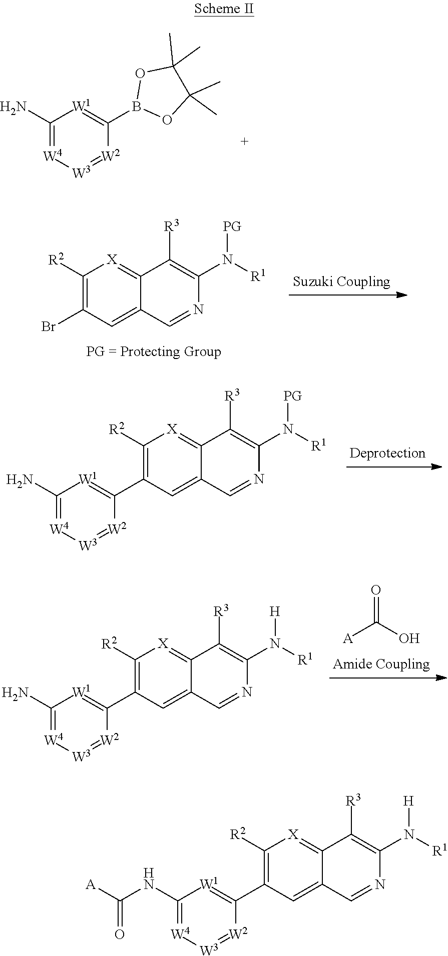

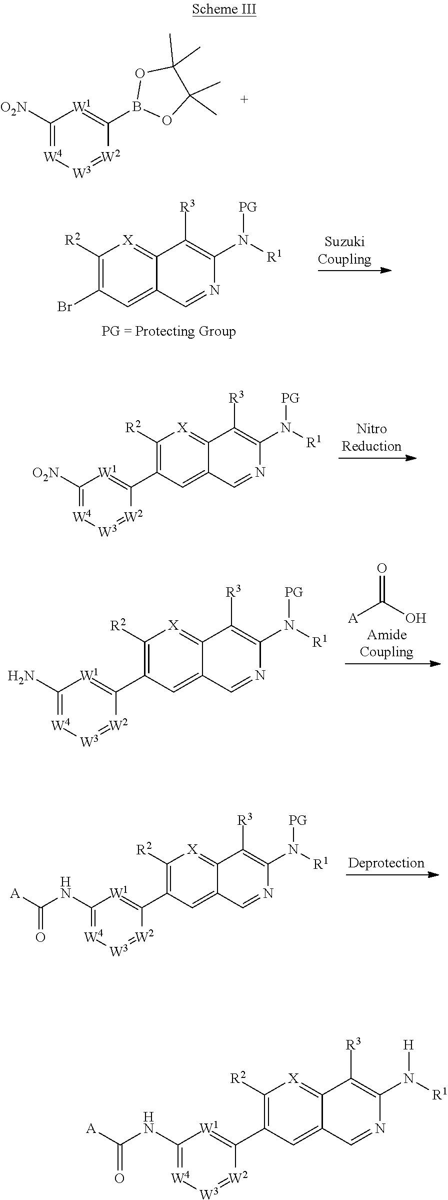

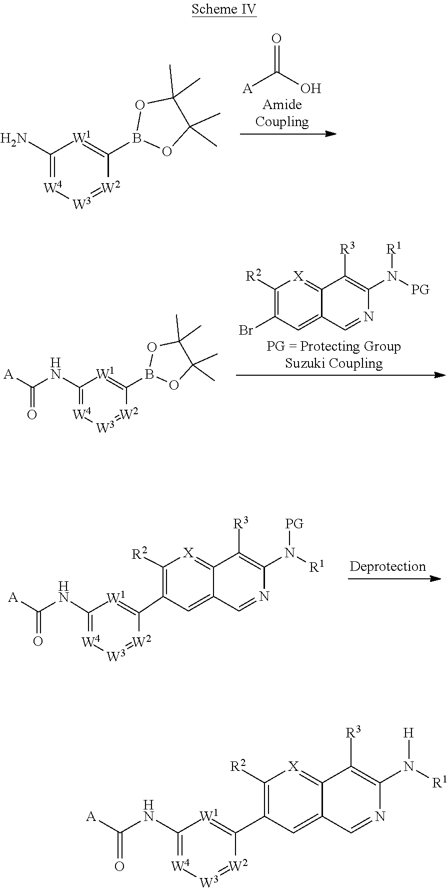

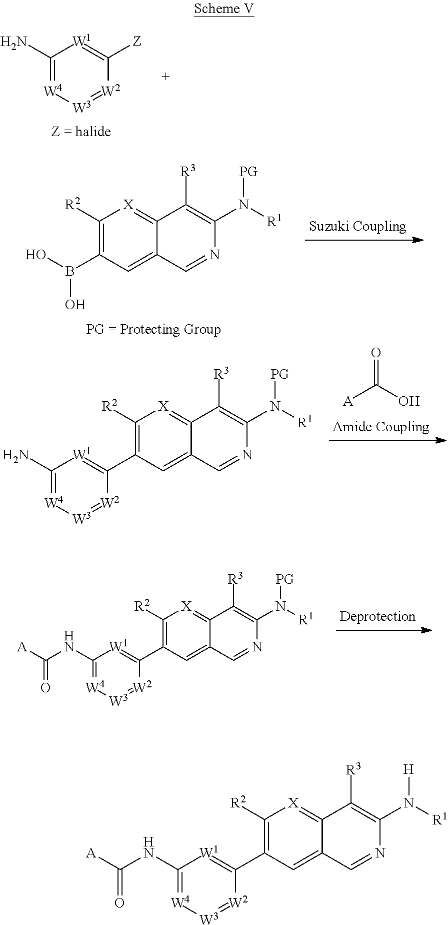

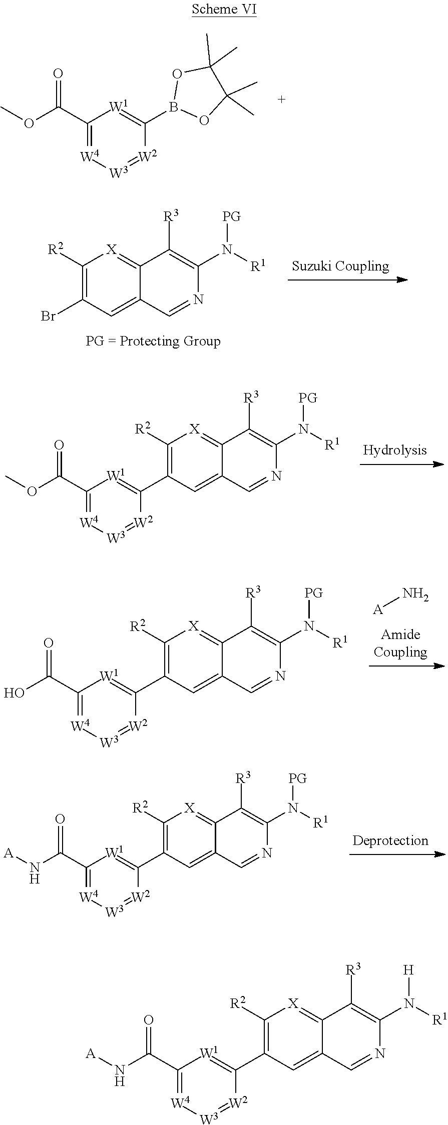

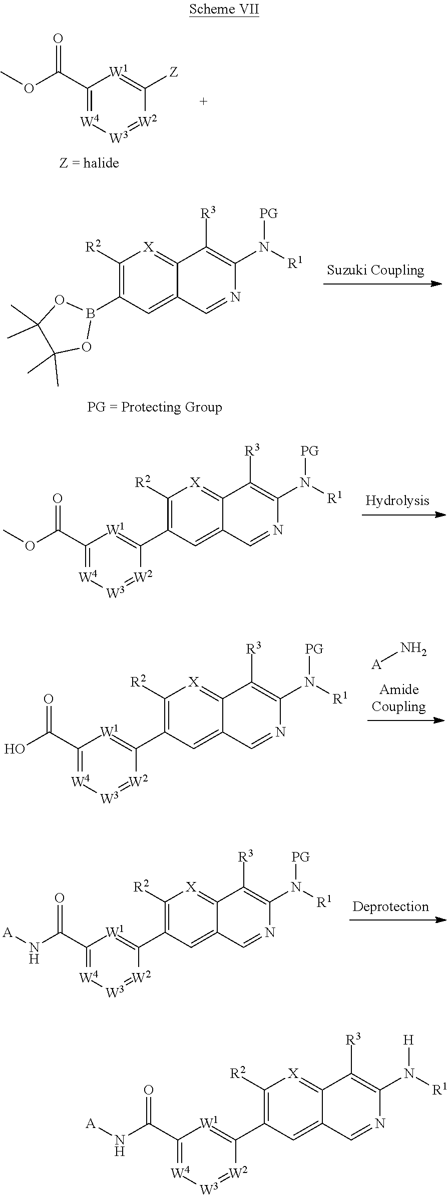

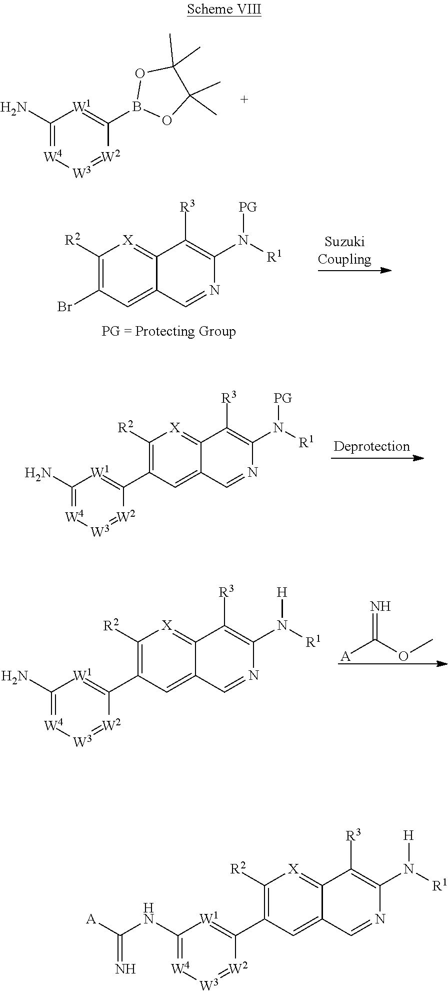

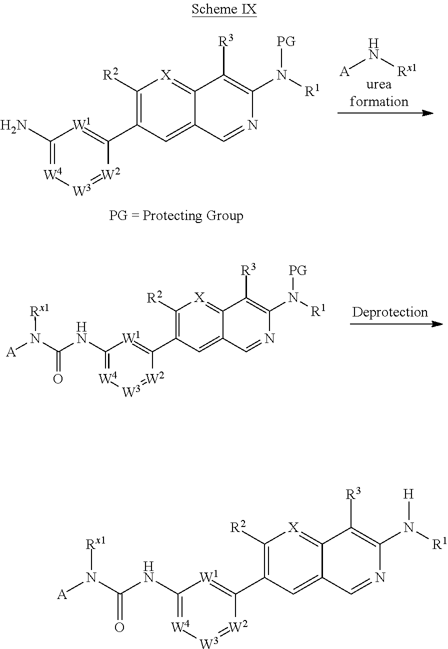

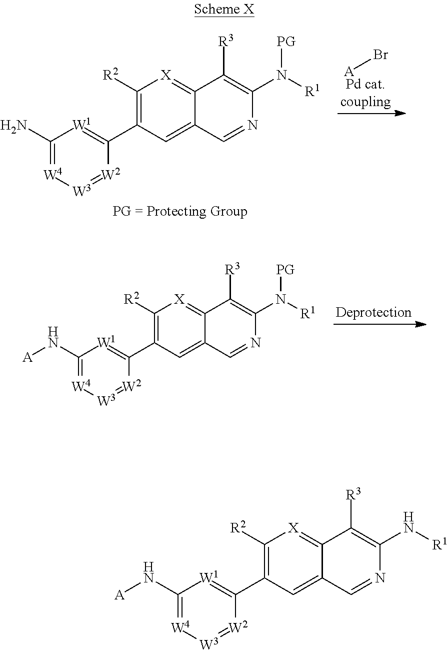

### Biological Assays

Compounds designed, selected and/or optimized by methods described above, once produced, can be characterized using a variety of assays known to those skilled in the art to determine whether the compounds have biological activity. For example, the molecules can be characterized by conventional assays, including but not limited to those assays described below, to determine whether they have a predicted activity, binding activity and/or binding specificity.

Furthermore, high-throughput screening can be used to speed up analysis using such assays. As a result, it can be possible to rapidly screen the molecules described herein for activity, using techniques known in the art. General methodologies for performing high-throughput screening are described, for example, in Devlin (1998) High Throughput Screening, Marcel Dekker; and U.S. Pat. No. 5,763,263. High-throughput assays can use one or more different assay techniques including, but not limited to, those described below.

Various in vitro or in vivo biological assays may be suitable for detecting the effect of the compounds of the present disclosure. These in vitro or in vivo biological assays can include, but are not limited to, enzymatic activity assays, electrophoretic mobility shift assays, reporter gene assays, in vitro cell viability assays, and the assays described herein.

In some embodiments the biological assay may involve retroviral production.

In some embodiments, a fusion mutant (e.g., BRAF-KIAA1549) may be subcloned into a retroviral expression vector (e.g., pMXs-IRES-Blasticidin), wherein the retrovirus may be produced by transfection of cells (e.g., HEK 293T) with retroviral plasmids (e.g., retroviral BRAF mutant expression vector).

In some embodiments, the cells (e.g., BEK 293T) may be plated and incubated. In some embodiments, the retroviral plasmids (e.g., BRAF-KIAA fusion mutant) may added to a transfection reagent and then added to cells (e.g., HEK 293T), wherein the cells may be harvested.

In some embodiments, the biological assay may involve the generation of a fusion stable cell line (e.g., a BRAF-KIAA1549 fusion stable cell line).

In some embodiments, cells (e.g., BaF3) may be transduced with a viral supernatant (e.g., BRAF-KIAA1549 fusion viral supernatant) and the cells may be sampled for viability (e.g., by Luminescent Cell Viability Assay such as CellTiterGlo). In some embodiments, the fusion stable cell line may undergo cell banking and sequence confirmation (e.g., sanger sequencing).

In some embodiments, the biological assay is for cell proliferation.

In some embodiments, cells (e.g., BaF3 BRAF-KIAA1549 fusion cells) are suspended and dispensed in plates. In some embodiments, to determine the effect of compounds of the present disclosure on cell proliferation, the cells (e.g., BaF3 BRAF-KIAA1549 fusion cells) may be incubated in the presence of vehicle control (e.g., DMSO) or a compound of the present disclosure at varying concentrations and the inhibition of cell growth may be determined by luminescent quantification (e.g., of intracellular ATP content using CellTiterGlo), according to the manufacturers protocol. In some embodiments, to determine the IC₅₀ values, the vehicle-treated cells were normalized as viable cells and analyzed using a software (e.g., the CDD Vault (Collaborative Drug Discovery, Burlingame, CA) using an algorithm (e.g., the Levenberg-Marquardt algorithm; Levenberg, K, 1994; Marquardt, D., 1963).

### Pharmaceutical Compositions

In some aspects, the present disclosure provides a pharmaceutical composition comprising a compound of the present disclosure as an active ingredient.

In some embodiments, the present disclosure provides a pharmaceutical composition comprising a compound described herein and one or more pharmaceutically acceptable carriers or excipients. In some embodiments, the present disclosure provides a pharmaceutical composition comprising at least one compound selected from Table I and Table II. In some embodiments, the present disclosure provides a pharmaceutical composition comprising at least one compound selected from Table I. In some embodiments, the present disclosure provides a pharmaceutical composition comprising at least one compound selected from Table II.

As used herein, the term “composition” is intended to encompass a product comprising the specified ingredients in the specified amounts, as well as any product which results, directly or indirectly, from combination of the specified ingredients in the specified amounts.

The compounds of present disclosure can be formulated for oral administration in forms such as tablets, capsules (each of which includes sustained release or timed release formulations), pills, powders, granules, elixirs, tinctures, suspensions, syrups and emulsions. The compounds of present disclosure on can also be formulated for intravenous (bolus or in-fusion), intraperitoneal, topical, subcutaneous, intramuscular or transdermal (e.g., patch) administration, all using forms well known to those of ordinary skill in the pharmaceutical arts.

The formulation of the present disclosure may be in the form of an aqueous solution comprising an aqueous vehicle. The aqueous vehicle component may comprise water and at least one pharmaceutically acceptable excipient. Suitable acceptable excipients include those selected from the group consisting of a solubility enhancing agent, chelating agent, preservative, tonicity agent, viscosity/suspending agent, buffer, and pH modifying agent, and a mixture thereof.

Any suitable solubility enhancing agent can be used. Examples of a solubility enhancing agent include cyclodextrin, such as those selected from the group consisting of hydroxypropyl-β-cyclodextrin, methyl-β-cyclodextrin, randomly methylated-β-cyclodextrin, ethylated-β-cyclodextrin, triacetyl-β-cyclodextrin, peracetylated-β-cyclodextrin, carboxymethyl-β-cyclodextrin, hydroxyethyl-β-cyclodextrin, 2-hydroxy-3-(trimethylammonio)propyl-β-cyclodextrin, glucosyl-β-cyclodextrin, sulfated β-cyclodextrin (S-β-CD), maltosyl-β-cyclodextrin, β-cyclodextrin sulfobutyl ether, branched-β-cyclodextrin, hydroxypropyl-γ-cyclodextrin, randomly methylated-γ-cyclodextrin, and trimethyl-γ-cyclodextrin, and mixtures thereof.

Any suitable chelating agent can be used. Examples of a suitable chelating agent include those selected from the group consisting of ethylenediaminetetraacetic acid and metal salts thereof, disodium edetate, trisodium edetate, and tetrasodium edetate, and mixtures thereof.

Any suitable preservative can be used. Examples of a preservative include those selected from the group consisting of quaternary ammonium salts such as benzalkonium halides (preferably benzalkonium chloride), chlorhexidine gluconate, benzethonium chloride, cetyl pyridinium chloride, benzyl bromide, phenylmercury nitrate, phenylmercury acetate, phenylmercury neodecanoate, merthiolate, methylparaben, propylparaben, sorbic acid, potassium sorbate, sodium benzoate, sodium propionate, ethyl p-hydroxybenzoate, propylaminopropyl biguanide, and butyl-p-hydroxybenzoate, and sorbic acid, and mixtures thereof.

The aqueous vehicle may also include a tonicity agent to adjust the tonicity (osmotic pressure). The tonicity agent can be selected from the group consisting of a glycol (such as propylene glycol, diethylene glycol, triethylene glycol), glycerol, dextrose, glycerin, mannitol, potassium chloride, and sodium chloride, and a mixture thereof.

The aqueous vehicle may also contain a viscosity/suspending agent. Suitable viscosity/suspending agents include those selected from the group consisting of cellulose derivatives, such as methyl cellulose, ethyl cellulose, hydroxyethylcellulose, polyethylene glycols (such as polyethylene glycol 300, polyethylene glycol 400), carboxymethyl cellulose, hydroxypropylmethyl cellulose, and cross-linked acrylic acid polymers (carbomers), such as polymers of acrylic acid cross-linked with polyalkenyl ethers or divinyl glycol (Carbopols—such as Carbopol 934, Carbopol 934P, Carbopol 971, Carbopol 974 and Carbopol 974P), and a mixture thereof.

In order to adjust the formulation to an acceptable pH (typically a pH range of about 5.0 to about 9.0, more preferably about 5.5 to about 8.5, particularly about 6.0 to about 8.5, about 7.0 to about 8.5, about 7.2 to about 7.7, about 7.1 to about 7.9, or about 7.5 to about 8.0), the formulation may contain a pH modifying agent. The pH modifying agent is typically a mineral acid or metal hydroxide base, selected from the group of potassium hydroxide, sodium hydroxide, and hydrochloric acid, and mixtures thereof, and preferably sodium hydroxide and/or hydrochloric acid. These acidic and/or basic pH modifying agents are added to adjust the formulation to the target acceptable pH range. Hence it may not be necessary to use both acid and base—depending on the formulation, the addition of one of the acid or base may be sufficient to bring the mixture to the desired pH range.

The aqueous vehicle may also contain a buffering agent to stabilize the pH. When used, the buffer is selected from the group consisting of a phosphate buffer (such as sodium dihydrogen phosphate and disodium hydrogen phosphate), a borate buffer (such as boric acid, or salts thereof including disodium tetraborate), a citrate buffer (such as citric acid, or salts thereof including sodium citrate), and ε-aminocaproic acid, and mixtures thereof.

The formulation may further comprise a wetting agent. Suitable classes of wetting agents include those selected from the group consisting of polyoxypropylene-polyoxyethylene block copolymers (poloxamers), polyethoxylated ethers of castor oils, polyoxyethylenated sorbitan esters (polysorbates), polymers of oxyethylated octyl phenol (Tyloxapol), polyoxyl 40 stearate, fatty acid glycol esters, fatty acid glyceryl esters, sucrose fatty esters, and polyoxyethylene fatty esters, and mixtures thereof.

Oral compositions generally include an inert diluent or an edible pharmaceutically acceptable carrier. They can be enclosed in gelatin capsules or compressed into tablets. For the purpose of oral therapeutic administration, the active compound can be incorporated with excipients and used in the form of tablets, troches, or capsules. Oral compositions can also be prepared using a fluid carrier for use as a mouthwash, wherein the compound in the fluid carrier is applied orally and swished and expectorated or swallowed. Pharmaceutically compatible binding agents, and/or adjuvant materials can be included as part of the composition. The tablets, pills, capsules, troches and the like can contain any of the following ingredients, or compounds of a similar nature: a binder such as microcrystalline cellulose, gum tragacanth or gelatin; an excipient such as starch or lactose, a disintegrating agent such as alginic acid, Primogel, or corn starch; a lubricant such as magnesium stearate or Sterotes; a glidant such as colloidal silicon dioxide; a sweetening agent such as sucrose or saccharin; or a flavoring agent such as peppermint, methyl salicylate, or orange flavoring.

According to a further aspect of the disclosure there is provided a pharmaceutical composition which comprises a compound of the present disclosure, or a pharmaceutically acceptable salt thereof, in association with a pharmaceutically acceptable diluent or carrier.

The compositions of the disclosure may be in a form suitable for oral use (for example as tablets, lozenges, hard or soft capsules, aqueous or oily suspensions, emulsions, dispersible powders or granules, syrups or elixirs), for topical use (for example as creams, ointments, gels, or aqueous or oily solutions or suspensions), for administration by inhalation (for example as a finely divided powder or a liquid aerosol), for administration by insufflation (for example as a finely divided powder) or for parenteral administration (for example as a sterile aqueous or oily solution for intravenous, subcutaneous, intramuscular, intraperitoneal or intramuscular dosing or as a suppository for rectal dosing).

The compositions of the disclosure may be obtained by conventional procedures using conventional pharmaceutical excipients, well known in the art. Thus, compositions intended for oral use may contain, for example, one or more coloring, sweetening, flavoring and/or preservative agents.

The size of the dose for therapeutic or prophylactic purposes of a compound of the present disclosure will naturally vary according to the nature and severity of the conditions, the age and sex of the animal, subject, or patient and the route of administration, according to well-known principles of medicine.

### EXEMPLARY EMBODIMENTS

Exemplary Embodiment No. 1. A method of treating or preventing cancer in a subject, the method comprising administering to the subject a compound of Formula (0):

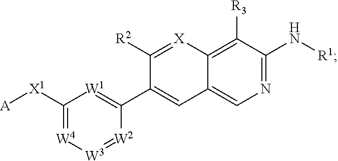

an isomer thereof, or a pharmaceutically acceptable salt thereof, wherein:
X is CR^{X} or N;
R^{X} is H, halogen, cyano, oxo, OH, C₁-C₆ alkyl, C₂-C₆ alkenyl, C₂-C₆ alkynyl, or C₁-C₆ alkoxy, wherein the C₁-C₆ alkyl, C₂-C₆ alkenyl, C₂-C₆ alkynyl, or C₁-C₆ alkoxy is optionally substituted with one or more halogen, cyano, oxo, or OH;
W¹ is N or CR^{W1};
R^{W1} is H, halogen, C₁-C₆ alkyl, C₂-C₆ alkenyl, or C₂-C₆ alkynyl;
W² is N or CR^{W2};
R^{W2} is H, halogen, C₁-C₆ alkyl, C₂-C₆ alkenyl, or C₂-C₆ alkynyl, wherein the C₁-C₆ alkyl, C₂-C₆ alkenyl, or C₂-C₆ alkynyl is optionally substituted with one or more halogen;
W³ is N or CR^{W3};
R^{W3} is H, halogen, C₁-C₆ alkyl, C₂-C₆ alkenyl, or C₂-C₆ alkynyl;
W⁴ is N or CR^{W4};
R^{W4} is H, halogen, C₁-C₆ alkyl, C₂-C₆ alkenyl, C₂-C₆ alkynyl, or S(C₁-C₆ alkyl);
R¹ is H, C₁-C₆ alkyl, C₂-C₆ alkenyl, C₂-C₆ alkynyl, C₁-C₆ alkoxy, C₃-C₁₂ cycloalkyl, 3- to 12-membered heterocycloalkyl, C₆-C₁₀ aryl, or 5- to 10-membered heteroaryl, wherein the C₁-C₆ alkyl, C₂-C₆ alkenyl, C₂-C₆ alkynyl, C₁-C₆ alkoxy, C₃-C₁₂ cycloalkyl, 3- to 12-membered heterocycloalkyl, C₆-C₁₀ aryl, or 5- to 10-membered heteroaryl is optionally substituted with one or more R^{1a};
each R^{1a} independently is halogen, cyano, oxo, OH, NH₂, NHC(═O)O(C₁-C₆ alkyl), N(C₁-C₆ alkyl)₂, C₁-C₆ alkyl, C₂-C₆ alkenyl, C₂-C₆ alkynyl, C₁-C₆ alkoxy, C₃-C₁₂ cycloalkyl, 3- to 12-membered heterocycloalkyl, C₆-C₁₀ aryl, or 5- to 10-membered heteroaryl;
R² is H, halogen, cyano, oxo, OH, C₁-C₆ alkyl, C₂-C₆ alkenyl, C₂-C₆ alkynyl, or C₁-C₆ alkoxy;
R³ is H, halogen, cyano, oxo, OH, C₁-C₆ alkyl, C₂-C₆ alkenyl, C₂-C₆ alkynyl, or C₁-C₆ alkoxy; or
R¹ and R³, together with the intervening atoms, form a 4- to 12-membered heterocycloalkyl optionally substituted with one or more oxo;
X¹ is —NR^{X1}—*, —C(═O)NR^{X1}—*, —NR^{X1}C(═O)—*, —NR^{X1}C(═O)O—*, —NR^{X1}N═C—*, —NR^{X1}C(═NR^{X1})—*, —N C(═NH)NR^{X1}—*, —NR^{X1}C(═O)NR^{X1}—*, —S(═O)₂NR^{X1}—*, or —NR^{X1}S(═O)₂—*, wherein * denotes attachment to A;
R^{X1} independently is H, S(═O)₂R^{X1a}, C₁-C₆ alkyl, C₂-C₆ alkenyl, C₂-C₆ alkynyl, C₁-C₆ alkoxy, C₃-C₁₂ cycloalkyl, 3- to 12-membered heterocycloalkyl, C₆-C₁₀ aryl, or 5- to 10-membered heteroaryl, wherein the C₁-C₆ alkyl, C₂-C₆ alkenyl, C₂-C₆ alkynyl, C₁-C₆ alkoxy, C₃-C₁₂ cycloalkyl, 3- to 12-membered heterocycloalkyl, C₆-C₁₀ aryl, or 5- to 10-membered heteroaryl is optionally substituted with one or more R^{X1a};
each R^{X1a} independently is halogen, C₁-C₆ alkyl, or 3- to 12-membered heterocycloalkyl, wherein the C₁-C₆ alkyl, or 3- to 12-membered heterocycloalkyl is optionally substituted with one or more halogen;
A is C₁-C₆ alkyl, C₃-C₁₂ cycloalkyl, 3- to 12-membered heterocycloalkyl, C₆-C₁₀ aryl, 5- to 10-membered heteroaryl, —(C₁-C₆ alkyl)-(C₃-C₁₂ cycloalkyl), —(C₁-C₆ alkyl)-(3- to 12-membered heterocycloalkyl), —(C₁-C₆ alkyl)-(C₆-C₁₀ aryl), or —(C₁-C₆ alkyl)-(5- to 10-membered heteroaryl), wherein the C₁-C₆ alkyl, C₃-C₁₂ cycloalkyl, 3- to 12-membered heterocycloalkyl, C₆-C₁₀ aryl, 5- to 10-membered heteroaryl, —(C₁-C₆ alkyl)-(C₃-C₁₂ cycloalkyl), —(C₁-C₆ alkyl)-(3- to 12-membered heterocycloalkyl), —(C₁-C₆ alkyl)-(C₆-C₁₀ aryl), or —(C₁-C₆ alkyl)-(5- to 10-membered heteroaryl) is optionally substituted with one or more R^{A};
each R^{A} independently is halogen, cyano, oxo, OH, OR^{A1}, NH₂, NHR^{A1}, N(R^{A1})₂, (═N)R^{A1}, C₁-C₆ alkyl, C₂-C₆ alkenyl, C₂-C₆ alkynyl, C₁-C₆ alkoxy, C₃-C₁₂ cycloalkyl, 3- to 12-membered heterocycloalkyl, C₆-C₁₀ aryl, or 5- to 10-membered heteroaryl, wherein the C₁-C₆ alkyl, C₂-C₆ alkenyl, C₂-C₆ alkynyl, C₁-C₆ alkoxy, C₃-C₁₂ cycloalkyl, 3- to 12-membered heterocycloalkyl, C₆-C₁₀ aryl, or 5- to 10-membered heteroaryl is optionally substituted with one or more R^{A1};
each R^{A1} independently is halogen, cyano, oxo, OH, OR^{A2}, NH₂, NHR^{A2}, N(R^{A2})₂, C(═O)R^{A2}, C₁-C₆ alkyl, C₂-C₆ alkenyl, C₂-C₆ alkynyl, C₁-C₆ alkoxy, C₃-C₁₂ cycloalkyl, 3- to 12-membered heterocycloalkyl, C₆-C₁₀ aryl, or 5- to 10-membered heteroaryl, wherein the C₁-C₆ alkyl, C₂-C₆ alkenyl, C₂-C₆ alkynyl, C₁-C₆ alkoxy, C₃-C₁₂ cycloalkyl, 3- to 12-membered heterocycloalkyl, C₆-C₁₀ aryl, or 5- to 10-membered heteroaryl is optionally substituted with one or more R^{A2}; and
each R^{A2} independently is halogen, cyano, OH, NH₂, N(R^{A3})₂, C(═O)R^{A3}, C₁-C₆ alkyl, C₂-C₆ alkenyl, or C₂-C₆ alkynyl,
wherein R^{A3} is C₁-C₆ alkyl, C₂-C₆ alkenyl, or C₂-C₆ alkynyl.

Exemplary Embodiment No. 2. A compound of Formula (0), an isomer thereof, or a pharmaceutically acceptable salt thereof for treating or preventing cancer in a subject.

Exemplary Embodiment No. 3. Use of a compound of Formula (0), an isomer thereof, or a pharmaceutically acceptable salt thereof in the manufacture of a medicament for treating or preventing cancer in a subject.

Exemplary Embodiment No. 4. A combination comprising: (i) a compound of Formula (0), an isomer thereof, or a pharmaceutically acceptable salt thereof, and (ii) one or more inhibitors of the MAPK pathways.

Exemplary Embodiment No. 5. A method of treating or preventing cancer in a subject, the method comprising administering to the subject a combination comprising: (i) a compound of Formula (0), an isomer thereof, or a pharmaceutically acceptable salt thereof, and (ii) one or more inhibitors of the MAPK pathways.

Exemplary Embodiment No. 6. A combination comprising: (i) a compound of Formula (0), an isomer thereof, or a pharmaceutically acceptable salt thereof, and (ii) one or more inhibitors of the MAPK pathways, for treating or preventing cancer in a subject.

Exemplary Embodiment No. 7. Use of a combination comprising: (i) a compound of Formula (0), an isomer thereof, or a pharmaceutically acceptable salt thereof, and (ii) one or more inhibitors of the MAPK pathways, in the manufacture of a medicament for treating or preventing cancer in a subject.

Exemplary Embodiment No. 8. The method, compound, combination, or use of any one of the preceding Exemplary Embodiments, wherein the subject is a human.

Exemplary Embodiment No. 9. The method, compound, combination, or use of any one of the preceding Exemplary Embodiments, wherein the cancer is characterized by at least one oncogenic mutation in the BRAF gene.

Exemplary Embodiment No. 10. The method, compound, combination, or use of any one of the preceding Exemplary Embodiments, wherein the cancer is characterized by at least one oncogenic variant of B-Raf.

Exemplary Embodiment No. 11. The method, compound, combination, or use of any one of the preceding Exemplary Embodiments, wherein the subject has at least one oncogenic mutation in the BRAF gene.

Exemplary Embodiment No. 12. The method, compound, combination, or use of any one of the preceding Exemplary Embodiments, wherein the subject has at least one tumor and/or cancerous cell that expresses an oncogenic variant of B-Raf.

Exemplary Embodiment No. 13. The method, compound, combination, or use of any one of the preceding Exemplary Embodiments, wherein the oncogenic mutation of B-Raf is any of the B-Raf mutations put forth in Table 1a.

Exemplary Embodiment No. 14. The method, compound, combination, or use of any one of the preceding Exemplary Embodiments, wherein the oncogenic variant of B-Raf can be any of the B-Raf variants put forth in Table 1b.

Exemplary Embodiment No. 15. The method, compound, combination, or use of any one of the preceding Exemplary Embodiments, wherein the cancer is characterized by at least one oncogenic mutation in the KRAS gene.

Exemplary Embodiment No. 16. The method, compound, combination, or use of any one of the preceding Exemplary Embodiments, wherein the cancer is characterized by at least one oncogenic variant of K-Ras.

Exemplary Embodiment No. 17. The method, compound, combination, or use of any one of the preceding Exemplary Embodiments, wherein the subject has at least one oncogenic mutation in the KRAS gene.

Exemplary Embodiment No. 18. The method, compound, combination, or use of any one of the preceding Exemplary Embodiments, wherein the subject has at least one tumor and/or cancerous cell that expresses an oncogenic variant of K-Ras.

Exemplary Embodiment No. 19. The method, compound, combination, or use of any one of the preceding Exemplary Embodiments, wherein the cancer is characterized by at least one oncogenic mutation in the NRAS gene.

Exemplary Embodiment No. 20. The method, compound, combination, or use of any one of the preceding Exemplary Embodiments, wherein the cancer is characterized by at least one oncogenic variant of N-Ras.

Exemplary Embodiment No. 21. The method, compound, combination, or use of any one of the preceding Exemplary Embodiments, wherein the subject has at least one oncogenic mutation in the NRAS gene.

Exemplary Embodiment No. 22. The method, compound, combination, or use of any one of the preceding Exemplary Embodiments, wherein the subject has at least one tumor and/or cancerous cell that expresses an oncogenic variant of N-Ras.

Exemplary Embodiment No. 23. The method, compound, combination, or use of any one of the preceding Exemplary Embodiments, wherein the cancer is characterized by at least one oncogenic mutation in the NF1 gene.

Exemplary Embodiment No. 24. The method, compound, combination, or use of any one of the preceding Exemplary Embodiments, wherein the cancer is characterized by at least one oncogenic variant of NF1.

Exemplary Embodiment No. 25. The method, compound, combination, or use of any one of the preceding Exemplary Embodiments, wherein the subject has at least one oncogenic mutation in the NF1 gene.

Exemplary Embodiment No. 26. The method, compound, combination, or use of any one of the preceding Exemplary Embodiments, wherein the subject has at least one tumor and/or cancerous cell that expresses an oncogenic variant of NF1.

Exemplary Embodiment No. 27. The method, compound, combination, or use of any one of the preceding Exemplary Embodiments, wherein the cancer is a carcinoma, a lymphoma, a blastoma, a sarcoma, a leukemia, a brain cancer, a breast cancer, a blood cancer, a bone cancer, a lung cancer, a skin cancer, a liver cancer, an ovarian cancer, a bladder cancer, a renal cancer, a kidney cancer, a gastric cancer, a thyroid cancer, a pancreatic cancer, an esophageal cancer, a prostate cancer, a cervical cancer, a uterine cancer, a stomach cancer, a soft tissue cancer, a laryngeal cancer, a small intestine cancer, a testicular cancer, an anal cancer, a vulvar cancer, a joint cancer, an oral cancer, a pharynx cancer or a colorectal cancer.

Exemplary Embodiment No. 28. The method, compound, combination, or use of any one of the preceding Exemplary Embodiments, wherein the cancer is adrenocortical carcinoma, bladder urothelial carcinoma, breast invasive carcinoma, cervical squamous cell carcinoma, endocervical adenocarcinoma, cholangiocarcinoma, colon adenocarcinoma, lymphoid neoplasm diffuse large B-cell lymphoma, esophageal carcinoma, glioblastoma multiforme, head and neck squamous cell carcinoma, kidney chromophobe, kidney renal clear cell carcinoma, kidney renal papillary cell carcinoma, acute myeloid leukemia, brain lower grade glioma, liver hepatocellular carcinoma, lung adenocarcinoma, lung squamous cell carcinoma, mesothelioma, ovarian serous cystadenocarcinoma, pancreatic adenocarcinoma, pheochromocytoma, paraganglioma, prostate adenocarcinoma, rectum adenocarcinoma, sarcoma, skin cutaneous melanoma, stomach adenocarcinoma, testicular germ cell tumors, thyroid carcinoma, thymoma, uterine carcinosarcoma, uveal melanoma. Other examples include breast cancer, lung cancer, lymphoma, melanoma, liver cancer, colorectal cancer, ovarian cancer, bladder cancer, renal cancer or gastric cancer. Further examples of cancer include neuroendocrine cancer, non-small cell lung cancer (NSCLC), small cell lung cancer, thyroid cancer, endometrial cancer, biliary cancer, esophageal cancer, anal cancer, salivary, cancer, vulvar cancer, cervical cancer, Acute lymphoblastic leukemia (ALL), Acute myeloid leukemia (AML), Adrenal gland tumors, Anal cancer, Bile duct cancer, Bladder cancer, Bone cancer, Bowel cancer, Brain tumors, Breast cancer, Cancer of unknown primary (CUP), Cancer spread to bone, Cancer spread to brain, Cancer spread to liver, Cancer spread to lung, Carcinoid, Cervical cancer, Children's cancers, Chronic lymphocytic leukemia (CLL), Chrome myeloid leukemia (CML), Colorectal cancer, Ear cancer, Endometrial cancer, Eye cancer, Follicular dendritic cell sarcoma, Gallbladder cancer, Gastric cancer, Gastro esophageal junction cancers, Germ cell tumors, Gestational trophoblastic disease (GIT)), Hairy cell leukemia, Head and neck cancer, Hodgkin lymphoma, Kaposi's sarcoma, Kidney cancer, Laryngeal cancer, Leukemia, Gastric linitis plastica, Liver cancer, Lung cancer, Lymphoma, Malignant schwannoma, Mediastinal germ cell tumors, Melanoma skin cancer, Men's cancer, Merkel cell skin cancer, Mesothelioma, Molar pregnancy, Mouth and oropharyngeal cancer, Myeloma, Nasal and paranasal sinus cancer, Nasopharyngeal cancer, Neuroblastoma, Neuroendocrine tumors, Non-Hodgkin lymphoma (NHL), Esophageal cancer, Ovarian cancer, Pancreatic cancer, Penile cancer, Persistent trophoblastic disease and choriocarcinoma, Pheochromocytoma, Prostate cancer, Pseudomyxoma peritonei, Rectal cancer. Retinoblastoma, Salivary gland cancer, Secondary' cancer, Signet cell cancer, Skin cancer, Small bowel cancer, Soft tissue sarcoma, Stomach cancer, T cell childhood non Hodgkin lymphoma (NHL), Testicular cancer, Thymus gland cancer, Thyroid cancer, Tongue cancer, Tonsil cancer, Tumors of the adrenal gland, Uterine cancer. Vaginal cancer, Vulval cancer, Wilms' tumor, Womb cancer and Gynaecological cancer. Examples of cancer also include, but are not limited to, Hematologic malignancies, Lymphoma, Cutaneous T-cell lymphoma, Peripheral T-cell lymphoma, Hodgkin's lymphoma, Non-Hodgkin's lymphoma, Multiple myeloma, Chrome lymphocytic leukemia, chronic myeloid leukemia, acute myeloid leukemia, Myelodysplastic syndromes, Myelofibrosis, Biliary tract cancer, Hepatocellular cancer, Colorectal cancer, Breast cancer, Lung cancer, Non-small cell lung cancer, Ovarian cancer, Thyroid Carcinoma, Renal Cell Carcinoma, Pancreatic cancer, Bladder cancer, skin cancer, malignant melanoma, merkel cell carcinoma, Uveal Melanoma or Glioblastoma multiforme.

Exemplary Embodiment No. 29. The method, compound, combination, or use of any one of the preceding Exemplary Embodiments, wherein the cancer is a hematological cancer.

Exemplary Embodiment No. 30. The method, compound, combination, or use of any one of the preceding Exemplary Embodiments, wherein the cancer is a solid cancer.

Exemplary Embodiment No. 31. The method, compound, combination, or use of any one of the preceding Exemplary Embodiments, wherein the cancer is melanoma, breast cancer, head and neck cancer, esophagogastric cancer, stomach and small intestine cancer, lung cancer, mesothelioma, hepatobiliary cancer, pancreatic cancer, kidney cancer, colorectal cancer, endometrial cancer, cervical cancer, ovarian cancer, bladder cancer, prostate cancer, soft tissue sarcoma, CNS and brain cancer, or thyroid cancer.

Exemplary Embodiment No. 32. The method, compound, combination, or use of any one of the preceding Exemplary Embodiments, wherein the cancer is non-small cell lung cancer (NSCLC), colorectal cancer, melanoma, thyroid cancer, histiocytosis, small bowel cancer, gastrointestinal neuroendocrine cancer, carcinoma of unknown primary, non-melanoma skin cancer, prostate cancer, gastric cancer, non-Hodgkin's lymphoma, papillary thyroid carcinoma or glioblastoma.

Exemplary Embodiment No. 33. The method, compound, combination, or use of any one of the preceding Exemplary Embodiments, wherein the cancer is glioma.

Exemplary Embodiment No. 34. The method, compound, combination, or use of any one of the preceding Exemplary Embodiments, wherein the cancer is low-grade glioma.

Exemplary Embodiment No. 35. The method, compound, combination, or use of any one of the preceding Exemplary Embodiments, wherein the cancer is glioblastoma.

Exemplary Embodiment No. 36. The method, compound, combination, or use of any one of the preceding Exemplary Embodiments, wherein the cancer is insensitive or resistant to treatment with one or more inhibitors of the MAPK pathways.

Exemplary Embodiment No. 37. The method, compound, combination, or use of any one of the preceding Exemplary Embodiments, wherein the cancer is insensitive or resistant to treatment with a BRAF inhibitor, a MEK inhibitor, or a combination thereof.

Exemplary Embodiment No. 38. The method, compound, combination, or use of any one of the preceding Exemplary Embodiments, wherein the subject has an adverse reaction to treatment with one or more inhibitors of the MAPK pathways.

Exemplary Embodiment No. 39. The method, compound, combination, or use of any one of the preceding Exemplary Embodiments, wherein the subject has an adverse reaction to treatment with a BRAF inhibitor, a MEK inhibitor, or a combination thereof.

Exemplary Embodiment No. 40. The method, compound, combination, or use of any one of the preceding Exemplary Embodiments, wherein the subject has been previously administered one or more inhibitors of the MAPK pathways, and the subject has experienced disease progression despite the previous administration.

Exemplary Embodiment No. 41. The method, compound, combination, or use of any one of the preceding Exemplary Embodiments, wherein the subject has been previously administered a BRAF inhibitor, a MEK inhibitor, or a combination thereof, and the subject has experienced disease progression despite the previous administration.

Exemplary Embodiment No. 42. The method, compound, combination, or use of any one of the preceding Exemplary Embodiments, wherein the BRAF inhibitor is vemurafenib, dabrafenib, or encorafenib.

Exemplary Embodiment No. 43. The method, compound, combination, or use of any one of the preceding Exemplary Embodiments, wherein the MEK inhibitor is trametinib, cobimetinib, or binimetinib.

Exemplary Embodiment No. 44. The method, compound, combination, or use of any one of the preceding Exemplary Embodiments, wherein the compound is administered to the subject by oral administration or parenteral administration.

Exemplary Embodiment No. 45. The method, compound, combination, or use of any one of the preceding Exemplary Embodiments, wherein a pharmaceutical composition comprising the compound is administered to the subject.

Exemplary Embodiment No. 46. The method, compound, combination, or use of any one of the preceding Exemplary Embodiments, wherein the compound is administered at a dosage (e.g., a daily dosage) of:
about 6±3 mg, about 6±2 mg, about 6±1 mg, about 6±0.9 mg, about 6±0.8 mg, about 6±0.7 mg, about 6±0.6 mg, about 6±0.5 mg, about 6±0.4 mg, about 6±0.3 mg, about 6±0.2 mg, or about 6±0.1 mg (e.g., about 6 mg);
about 12±6 mg, about 12±5 mg, about 12±4 mg, about 12±3 mg, about 12±2 mg, about 12±1 mg, about 12±0.9 mg, about 12±0.8 mg, about 12±0.7 mg, about 12±0.6 mg, about 12±0.5 mg, about 12±0.4 mg, about 12±0.3 mg, about 12±0.2 mg, or about 12±0.1 mg (e.g., about 12 mg);
about 25±10 mg, about 25±9 mg, about 25±8 mg, about 25±7 mg, about 25±6 mg, about 25±5 mg, about 25±4 mg, about 25±3 mg, about 25±2 mg, or about 25±1 mg (e.g., about 25 mg);
about 50±20 mg, about 50±10 mg, about 50±9 mg, about 50±8 mg, about 50±7 mg, about 50±6 mg, about 50±5 mg, about 50±4 mg, about 50±3 mg, about 50±2 mg, or about 50±1 mg (e.g., about 50 mg);
about 100±50 mg, about 100±40 mg, about 100±30 mg, about 100±20 mg, about 100±10 mg, about 100±9 mg, about 100±8 mg, about 100±7 mg, about 100±6 mg, about 100±5 mg, about 100±4 mg, about 100±3 mg, about 100±2 mg, or about 100±1 mg (e.g., about 100 mg);
about 200±100 mg, about 200±90 mg, about 200±80 mg, about 200±70 mg, about 200±60 mg, about 200±50 mg, about 200±40 mg, about 200±30 mg, about 200±20 mg, or about 200±10 mg (e.g., about 200 mg);
about 400±200 mg, about 400±100 mg, about 400±90 mg, about 400±80 mg, about 400±70 mg, about 400±60 mg, about 400±50 mg, about 400±40 mg, about 400±30 mg, about 400±20 mg, or about 400±10 mg (e.g., about 400 mg);
about 600±300 mg, about 600±200 mg, about 600±100 mg, about 600±90 mg, about 600±80 mg, about 600±70 mg, about 600±60 mg, about 600±50 mg, about 600±40 mg, about 600±30 mg, about 600±20 mg, or about 600±10 mg (e.g., about 600 mg);
about 800±400 mg, about 800±300 mg, about 800±200 mg, about 800±100 mg, about 800±90 mg, about 800±80 mg, about 800±70 mg, about 800±60 mg, about 800±50 mg, about 800±40 mg, about 800±30 mg, about 800±20 mg, or about 800±10 mg (e.g., about 800 mg);
about 1000±500 mg, about 1000±400 mg, about 1000±300 mg, about 1000±200 mg, about 1000±100 mg, about 1000±90 mg, about 1000±80 mg, about 1000±70 mg, about 1000±60 mg, about 1000±50 mg, about 1000±40 mg, about 1000±30 mg, about 1000±20 mg, or about 1000±10 mg (e.g., about 1000 mg); or
about 1200±600 mg, about 1200±500 mg, about 1200±400 mg, about 1200±300 mg, about 1200±200 mg, about 1200±100 mg, about 1200±90 mg, about 1200±80 mg, about 1200±70 mg, about 1200±60 mg, about 1200±50 mg, about 1200±40 mg, about 1200±30 mg, about 1200±20 mg, or about 1200±10 mg (e.g., about 1200 mg).

Exemplary Embodiment No. 47. The method, compound, or use of any one of the preceding Exemplary Embodiments, wherein one or more inhibitors of the MAPK pathways are further administered to the subject.

Exemplary Embodiment No. 48. The method, compound, combination, or use of any one of the preceding Exemplary Embodiments, wherein the compound and the one or more inhibitors of the MAPK pathways are administered simultaneously.

Exemplary Embodiment No. 49. The method, compound, combination, or use of any one of the preceding Exemplary Embodiments, wherein the compound and the one or more inhibitors of the MAPK pathways are administered sequentially.

Exemplary Embodiment No. 50. The method, compound, combination, or use of any one of the preceding Exemplary Embodiments, wherein the compound and the one or more inhibitors of the MAPK pathways are administered in temporal proximity.

Exemplary Embodiment No. 51. The method, compound, combination, or use of any one of the preceding Exemplary Embodiments, wherein the compound and the one or more inhibitors of the MAPK pathways are administered in alternation.

Exemplary Embodiment No. 52. The method, compound, combination, or use of any one of the preceding Exemplary Embodiments, wherein the compound and the one or more inhibitors of the MAPK pathways are administered by a same route of administration.

Exemplary Embodiment No. 53. The method, compound, combination, or use of any one of the preceding Exemplary Embodiments, wherein at least two inhibitors of the MAPK pathways are administered.

Exemplary Embodiment No. 54. The method, compound, combination, or use of any one of the preceding Exemplary Embodiments, wherein the MEK inhibitor binimetinib.

Exemplary Embodiment No. 55. The method, compound, combination, or use of any one of the preceding Exemplary Embodiments, wherein the compound is administered at a dosage (e.g., a daily dosage) of about 6±3 mg, about 6±2 mg, about 6±1 mg, about 6±0.9 mg, about 6±0.8 mg, about 6±0.7 mg, about 6±0.6 mg, about 6±0.5 mg, about 6±0.4 mg, about 6±0.3 mg, about 6±0.2 mg, or about 6±0.1 mg (e.g., about 6 mg); about 12±6 mg, about 12±5 mg, about 12±4 mg, about 12±3 mg, about 12±2 mg, about 12±1 mg, about 12±0.9 mg, about 12±0.8 mg, about 12±0.7 mg, about 12±0.6 mg, about 12±0.5 mg, about 12±0.4 mg, about 12±0.3 mg, about 12±0.2 mg, or about 12±0.1 mg (e.g., about 12 mg); about 25±10 mg, about 25±9 mg, about 25±8 mg, about 25±7 mg, about 25±6 mg, about 25±5 mg, about 25±4 mg, about 25±3 mg, about 25±2 mg, or about 25±1 mg (e.g., about 25 mg); about 50±20 mg, about 50±10 mg, about 50±9 mg, about 50±8 mg, about 50±7 mg, about 50±6 mg, about 50±5 mg, about 50±4 mg, about 50±3 mg, about 50±2 mg, or about 50±1 mg (e.g., about 50 mg); about 100±50 mg, about 100±40 mg, about 100±30 mg, about 100±20 mg, about 100±10 mg, about 100±9 mg, about 100±8 mg, about 100±7 mg, about 100±6 mg, about 100±5 mg, about 100±4 mg, about 100±3 mg, about 100±2 mg, or about 100±1 mg (e.g., about 100 mg); about 150±70 mg, about 150±60 mg, about 150±50 mg, about 150±40 mg, about 150±30 mg, about 150±20 mg, about 150±10 mg, about 150±5 mg, about 150±4 mg, or about 150±3 mg (e.g., about 150 mg); about 200±100 mg, about 200±90 mg, about 200±80 mg, about 200±70 mg, about 200±60 mg, about 200±50 mg, about 200±40 mg, about 200±30 mg, about 200±20 mg, or about 200±10 mg (e.g., about 200 mg); about 300±150 mg, about 300±120 mg, about 300±100 mg, about 300±80 mg, about 300±60 mg, about 300±50 mg, about 300±40 mg, about 300±30 mg, about 300±20 mg, or about 300±10 mg (e.g., about 300 mg); about 400±200 mg, about 400±100 mg, about 400±90 mg, about 400±80 mg, about 400±70 mg, about 400±60 mg, about 400±50 mg, about 400±40 mg, about 400±30 mg, about 400±20 mg, or about 400±10 mg (e.g., about 400 mg); about 600±300 mg, about 600±200 mg, about 600±100 mg, about 600±90 mg, about 600±80 mg, about 600±70 mg, about 600±60 mg, about 600±50 mg, about 600±40 mg, about 600±30 mg, about 600±20 mg, or about 600±10 mg (e.g., about 600 mg); about 800±400 mg, about 800±300 mg, about 800±200 mg, about 800±100 mg, about 800±90 mg, about 800±80 mg, about 800±70 mg, about 800±60 mg, about 800±50 mg, about 800±40 mg, about 800±30 mg, about 800±20 mg, or about 800±10 mg (e.g., about 800 mg); about 1000±500 mg, about 1000±400 mg, about 1000±300 mg, about 1000±200 mg, about 1000±100 mg, about 1000±90 mg, about 1000±80 mg, about 1000±70 mg, about 1000±60 mg, about 1000±50 mg, about 1000±40 mg, about 1000±30 mg, about 1000±20 mg, or about 1000±10 mg (e.g., about 1000 mg); or about 1200±600 mg, about 1200±500 mg, about 1200±400 mg, about 1200±300 mg, about 1200±200 mg, about 1200±100 mg, about 1200±90 mg, about 1200±80 mg, about 1200±70 mg, about 1200±60 mg, about 1200±50 mg, about 1200±40 mg, about 1200±30 mg, about 1200±20 mg, or about 1200±10 mg (e.g., about 1200 mg); and binimetinib is administered at a dosage (e.g., a daily dosage) of about 15±6 mg, about 15±5 mg, about 15±4 mg, about 15±3 mg, about 15±2 mg, about 15±1 mg, about 15±0.9 mg, about 15±0.8 mg, about 15±0.7 mg, about 15±0.6 mg, about 15±0.5 mg, about 15±0.4 mg, about 15±0.3 mg, about 15±0.2 mg, or about 15±0.1 mg (e.g., about 15 mg); about 30±10 mg, about 30±9 mg, about 30±8 mg, about 30±7 mg, about 30±6 mg, about 30±5 mg, about 30±4 mg, about 30±3 mg, about 30±2 mg, or about 30±1 mg (e.g., about 30 mg); about 45±20 mg, about 45±10 mg, about 45±9 mg, about 45±8 mg, about 45±7 mg, about 45±6 mg, about 45±5 mg, about 45±4 mg, about 45±3 mg, about 45±2 mg, or about 45±1 mg (e.g., about 45 mg); about 60±30 mg, about 60±20 mg, about 60±10 mg, about 60±9 mg, about 60±8 mg, about 60±7 mg, about 60±6 mg, about 60±5 mg, about 60±4 mg, about 60±3 mg, about 60±2 mg, or about 60±1 mg (e.g., about 60 mg); about 75±40 mg, about 75±30 mg, about 75±20 mg, about 75±10 mg, about 75±9 mg, about 75±8 mg, about 75±7 mg, about 75±6 mg, about 75±5 mg, about 75±4 mg, about 75±3 mg, about 75±2 mg, or about 75±1 mg (e.g., about 75 mg); or about 90±50 mg, about 90±40 mg, about 90±30 mg, about 90±20 mg, about 90±10 mg, about 90±9 mg, about 90±8 mg, about 90±7 mg, about 90±6 mg, about 90±5 mg, about 90±4 mg, about 90±3 mg, about 90±2 mg, or about 90±1 mg (e.g., about 90 mg).

### DEFINITIONS

Unless otherwise stated, the following terms used in the specification and claims have the following meanings set out below.

As would be appreciated by the skilled artisan, the BRAF gene is commonly referred to as one of BRAF, B-RAF1, BRAF1, NS7, RAFB1, B-Raf proto-oncogene, proto-oncogene B-raf, v-Raf murine sarcoma viral oncogene homolog B, and v-Raf murine sarcoma viral oncogene homolog B1. Thus, these terms are used herein interchangeably to refer to the BRAF gene.

As would be appreciated by the skilled artisan, the B-Raf protein, encoded by the BRAF gene, is commonly referred to as one of BRAF, B-Raf, serine/threonine-protein kinase B-Raf, proto-oncogene B-Raf, p94 and v-Raf murine sarcoma viral oncogene homolog B1. Thus, these terms are used herein interchangeably to refer to the B-Raf gene.

Without wishing to be limited by this statement, it is understood that, while various options for variables are described herein, the disclosure intends to encompass operable embodiments having combinations of the options. The disclosure may be interpreted as excluding the non-operable embodiments caused by certain combinations of the options.

It is to be understood that a compound of the present disclosure may be depicted in a neutral form, a cationic form (e.g., carrying one or more positive charges), or an anionic form (e.g., carrying one or more negative charges), all of which are intended to be included in the scope of the present disclosure. For example, when a compound of the present disclosure is depicted in an anionic form, it should be understood that such depiction also refers to the various neutral forms, cationic forms, and anionic forms of the compound. For another example, when a compound the present disclosure is depicted in an anionic form, it should be understood that such depiction also refers to various salts (e.g., sodium salt) of the anionic form of the compound.

A “therapeutically effective amount” means the amount of a compound that, when administered to a mammal for treating a disease, is sufficient to effect such treatment for the disease. The “therapeutically effective amount” will vary depending on the compound, the disease and its severity and the age, weight, etc., of the mammal to be treated.

As used herein, “alkyl”, “C₁, C₂, C₃, C₄, C₅ or C₆ alkyl” or “C₁-C₆ alkyl” is intended to include C₁, C₂, C₃, C₄, C₅ or C₆ straight chain (linear) saturated aliphatic hydrocarbon groups and C₃, C₄, C₅ or C₆ branched saturated aliphatic hydrocarbon groups. For example, C₁-C₆ alkyl is intends to include C₁, C₂, C₃, C₄, C₅ and C₆ alkyl groups. Examples of alkyl include, moieties having from one to six carbon atoms, such as, but not limited to, methyl, ethyl, n-propyl, i-propyl, n-butyl, s-butyl, t-butyl, n-pentyl, i-pentyl or n-hexyl. In some embodiments, a straight chain or branched alkyl has six or fewer carbon atoms (e.g., C₁-C₆ for straight chain, C₃-C₆ for branched chain), and in another embodiment, a straight chain or branched alkyl has four or fewer carbon atoms.

As used herein, the term “optionally substituted alkyl” refers to unsubstituted alkyl or alkyl having designated substituents replacing one or more hydrogen atoms on one or more carbons of the hydrocarbon backbone. Such substituents can include, for example, alkyl, alkenyl, alkynyl, halogen, hydroxyl, alkylcarbonyloxy, arylcarbonyloxy, alkoxycarbonyloxy, aryloxycarbonyloxy, carboxylate, alkylcarbonyl, arylcarbonyl, alkoxycarbonyl, aminocarbonyl, alkylaminocarbonyl, dialkylaminocarbonyl, alkylthiocarbonyl, alkoxyl, phosphate, phosphonato, phosphinato, amino (including alkylamino, dialkylamino, arylamino, diarylamino and alkylarylamino), acylamino (including alkylcarbonylamino, arylcarbonylamino, carbamoyl and ureido), amidino, imino, sulfhydryl, alkylthio, arylthio, thiocarboxylate, sulfates, alkylsulfinyl, sulfonato, sulfamoyl, sulfonamido, nitro, trifluoromethyl, cyano, azido, heterocyclyl, alkylaryl, or an aromatic or heteroaromatic moiety.

As used herein, the term “alkenyl” includes unsaturated aliphatic groups analogous in length and possible substitution to the alkyls described above, but that contain at least one double bond. For example, the term “alkenyl” includes straight chain alkenyl groups (e.g., ethenyl, propenyl, butenyl, pentenyl, hexenyl, heptenyl, octenyl, nonenyl, decenyl), and branched alkenyl groups. In certain embodiments, a straight chain or branched alkenyl group has six or fewer carbon atoms in its backbone (e.g., C₂-C₆ for straight chain, C₃-C₆ for branched chain). The term “C₂-C₆” includes alkenyl groups containing two to six carbon atoms. The term “C₃-C₆” includes alkenyl groups containing three to six carbon atoms.

As used herein, the term “optionally substituted alkenyl” refers to unsubstituted alkenyl or alkenyl having designated substituents replacing one or more hydrogen atoms on one or more hydrocarbon backbone carbon atoms. Such substituents can include, for example, alkyl, alkenyl, alkynyl, halogen, hydroxyl, alkylcarbonyloxy, arylcarbonyloxy, alkoxycarbonyloxy, aryloxycarbonyloxy, carboxylate, alkylcarbonyl, arylcarbonyl, alkoxycarbonyl, aminocarbonyl, alkylaminocarbonyl, dialkylaminocarbonyl, alkylthiocarbonyl, alkoxyl, phosphate, phosphonato, phosphinato, amino (including alkylamino, dialkylamino, arylamino, diarylamino and alkylarylamino), acylamino (including alkylcarbonylamino, arylcarbonylamino, carbamoyl and ureido), amidino, imino, sulfhydryl, alkylthio, arylthio, thiocarboxylate, sulfates, alkylsulfinyl, sulfonato, sulfamoyl, sulfonamido, nitro, trifluoromethyl, cyano, heterocyclyl, alkylaryl, or an aromatic or heteroaromatic moiety.

As used herein, the term “alkynyl” includes unsaturated aliphatic groups analogous in length and possible substitution to the alkyls described above, but which contain at least one triple bond. For example, “alkynyl” includes straight chain alkynyl groups (e.g., ethynyl, propynyl, butynyl, pentynyl, hexynyl, heptynyl, octynyl, nonynyl, decynyl), and branched alkynyl groups. In certain embodiments, a straight chain or branched alkynyl group has six or fewer carbon atoms in its backbone (e.g., C₂-C₆ for straight chain, C₃-C₆ for branched chain). The term “C₂-C₆” includes alkynyl groups containing two to six carbon atoms. The term “C₃-C₆” includes alkynyl groups containing three to six carbon atoms. As used herein, “C₂-C₆ alkenylene linker” or “C₂-C₆ alkynylene linker” is intended to include C₂, C₃, C₄, C₅ or C₆ chain (linear or branched) divalent unsaturated aliphatic hydrocarbon groups. For example, C₂-C₆ alkenylene linker is intended to include C₂, C₃, C₄, C₅ and C₆ alkenylene linker groups.

As used herein, the term “optionally substituted alkynyl” refers to unsubstituted alkynyl or alkynyl having designated substituents replacing one or more hydrogen atoms on one or more hydrocarbon backbone carbon atoms. Such substituents can include, for example, alkyl, alkenyl, alkynyl, halogen, hydroxyl, alkylcarbonyloxy, arylcarbonyloxy, alkoxycarbonyloxy, aryloxycarbonyloxy, carboxylate, alkylcarbonyl, arylcarbonyl, alkoxycarbonyl, aminocarbonyl, alkylaminocarbonyl, dialkylaminocarbonyl, alkylthiocarbonyl, alkoxyl, phosphate, phosphonato, phosphinato, amino (including alkylamino, dialkylamino, arylamino, diarylamino and alkylarylamino), acylamino (including alkylcarbonylamino, arylcarbonylamino, carbamoyl and ureido), amidino, imino, sulfhydryl, alkylthio, arylthio, thiocarboxylate, alkylsulfinyl, sulfonato, sulfamoyl, sulfonamido, nitro, trifluoromethyl, cyano, azido, heterocyclyl, alkylaryl, or an aromatic or heteroaromatic moiety.

Other optionally substituted moieties (such as optionally substituted cycloalkyl, heterocycloalkyl, aryl, or heteroaryl) include both the unsubstituted moieties and the moieties having one or more of the designated substituents. For example, substituted heterocycloalkyl includes those substituted with one or more alkyl groups, such as 2,2,6,6-tetramethyl-piperidinyl and 2,2,6,6-tetramethyl-1,2,3,6-tetrahydropyridinyl.

As used herein, the term “cycloalkyl” refers to a saturated or partially unsaturated hydrocarbon monocyclic or polycyclic (e.g., fused, bridged, or spiro rings) system having 3 to 30 carbon atoms (e.g., C₃-C₁₂, C₃-C₁₀, or C₃-C₈). Examples of cycloalkyl include, but are not limited to, cyclopropyl, cyclobutyl, cyclopentyl, cyclohexyl, cycloheptyl, cyclooctyl, cyclopentenyl, cyclohexenyl, cycloheptenyl, 1,2,3,4-tetrahydronaphthalenyl, and adamantyl. In the case of polycyclic cycloalkyl, only one of the rings in the cycloalkyl needs to be non-aromatic.

As used herein, the term “heterocycloalkyl” refers to a saturated or partially unsaturated 3-8 membered monocyclic, 7-12 membered bicyclic (fused, bridged, or spiro rings), or 11-14 membered tricyclic ring system (fused, bridged, or spiro rings) having one or more heteroatoms (such as O, N, S, P, or Se), e.g., 1 or 1-2 or 1-3 or 1-4 or 1-5 or 1-6 heteroatoms, or e.g., 1, 2, 3, 4, 5, or 6 heteroatoms, independently selected from the group consisting of nitrogen, oxygen and sulfur, unless specified otherwise. Examples of heterocycloalkyl groups include, but are not limited to, piperidinyl, piperazinyl, pyrrolidinyl, dioxanyl, tetrahydrofuranyl, isoindolinyl, indolinyl, imidazolidinyl, pyrazolidinyl, oxazolidinyl, isoxazolidinyl, triazolidinyl, oxiranyl, azetidinyl, oxetanyl, thietanyl, 1,2,3,6-tetrahydropyridinyl, tetrahydropyranyl, dihydropyranyl, pyranyl, morpholinyl, tetrahydrothiopyranyl, 1,4-diazepanyl, 1,4-oxazepanyl, 2-oxa-5-azabicyclo[2.2.1]heptanyl, 2,5-diazabicyclo[2.2.1]heptanyl, 2-oxa-6-azaspiro[3.3]heptanyl, 2,6-diazaspiro[3.3]heptanyl, 1,4-dioxa-8-azaspiro[4.5]decanyl, 1,4-dioxaspiro[4.5]decanyl, 1-oxaspiro[4.5]decanyl, 1-azaspiro[4.5]decanyl, 3′H-spiro[cyclohexane-1,1′-isobenzofuran]-yl, 7′H-spiro[cyclohexane-1,5′-furo[3,4-b]pyridin]-yl, 3′H-spiro[cyclohexane-1,1′-furo[3,4-c]pyridin]-yl, 3-azabicyclo[3.1.0]hexanyl, 3-azabicyclo[3.1.0]hexan-3-yl, 1,4,5,6-tetrahydropyrrolo[3,4-c]pyrazolyl, 3,4,5,6,7,8-hexahydropyrido[4,3-d]pyrimidinyl, 4,5,6,7-tetrahydro-1H-pyrazolo[3,4-c]pyridinyl, 5,6,7,8-tetrahydroimidazo[1,2-a]pyridinyl, 6,7,8,9-tetrahydro-5H-imidazo[1,2-a]azepinyl, 5,6,7,8-tetrahydropyrido[4,3-d]pyrimidinyl, 2-azaspiro[3.3]heptanyl, 2-methyl-2-azaspiro[3.3]heptanyl, 2-azaspiro[3.5]nonanyl, 2-methyl-2-azaspiro[3.5]nonanyl, 2-azaspiro[4.5]decanyl, 2-methyl-2-azaspiro[4.5]decanyl, 2-oxa-azaspiro[3.4]octanyl, 2-oxa-azaspiro[3.4]octan-6-yl, and the like. In the case of multicyclic heterocycloalkyl, only one of the rings in the heterocycloalkyl needs to be non-aromatic (e.g., 4,5,6,7-tetrahydrobenzo[c]isoxazolyl).

As used herein, the term “aryl” includes groups with aromaticity, including “conjugated,” or multicyclic systems with one or more aromatic rings and do not contain any heteroatom in the ring structure. The term aryl includes both monovalent species and divalent species. Examples of aryl groups include, but are not limited to, phenyl, biphenyl, naphthyl and the like. Conveniently, an aryl is phenyl.

As used herein, the term “heteroaryl” is intended to include a stable 5-, 6-, or 7-membered monocyclic or 7-, 8-, 9-, 10-, 11- or 12-membered bicyclic aromatic heterocyclic ring which consists of carbon atoms and one or more heteroatoms, e.g., 1 or 1-2 or 1-3 or 1-4 or 1-5 or 1-6 heteroatoms, or e.g., 1, 2, 3, 4, 5, or 6 heteroatoms, independently selected from the group consisting of nitrogen, oxygen and sulfur. The nitrogen atom may be substituted or unsubstituted (i.e., N or NR wherein R is H or other substituents, as defined). The nitrogen and sulfur heteroatoms may optionally be oxidised (i.e., N→O and S(O)ₚ, where p=1 or 2). It is to be noted that total number of S and O atoms in the aromatic heterocycle is not more than 1. Examples of heteroaryl groups include pyrrole, furan, thiophene, thiazole, isothiazole, imidazole, triazole, tetrazole, pyrazole, oxazole, isoxazole, pyridine, pyrazine, pyridazine, pyrimidine, and the like. Heteroaryl groups can also be fused or bridged with alicyclic or heterocyclic rings, which are not aromatic so as to form a multicyclic system (e.g., 4,5,6,7-tetrahydrobenzo[c]isoxazolyl).

Furthermore, the terms “aryl” and “heteroaryl” include multicyclic aryl and heteroaryl groups, e.g., tricyclic, bicyclic, e.g., naphthalene, benzoxazole, benzodioxazole, benzothiazole, benzoimidazole, benzothiophene, quinoline, isoquinoline, naphthyridine, indole, benzofuran, purine, deazapurine, indolizine.

The cycloalkyl, heterocycloalkyl, aryl, or heteroaryl ring can be substituted at one or more ring positions (e.g., the ring-forming carbon or heteroatom such as N) with such substituents as described above, for example, alkyl, alkenyl, alkynyl, halogen, hydroxyl, alkoxy, alkylcarbonyloxy, arylcarbonyloxy, alkoxycarbonyloxy, aryloxycarbonyloxy, carboxylate, alkylcarbonyl, alkylaminocarbonyl, aralkylaminocarbonyl, alkenylaminocarbonyl, alkylcarbonyl, arylcarbonyl, aralkylcarbonyl, alkenylcarbonyl, alkoxycarbonyl, aminocarbonyl, alkylthiocarbonyl, phosphate, phosphonato, phosphinato, amino (including alkylamino, dialkylamino, arylamino, diarylamino and alkylarylamino), acylamino (including alkylcarbonylamino, arylcarbonylamino, carbamoyl and ureido), amidino, imino, sulfhydryl, alkylthio, arylthio, thiocarboxylate, sulfates, alkylsulfinyl, sulfonato, sulfamoyl, sulfonamido, nitro, trifluoromethyl, cyano, azido, heterocyclyl, alkylaryl, or an aromatic or heteroaromatic moiety. Aryl and heteroaryl groups can also be fused or bridged with alicyclic or heterocyclic rings, which are not aromatic so as to form a multicyclic system (e.g., tetralin, methylenedioxyphenyl such as benzo[d][1,3]dioxole-5-yl).

As used herein, the term “substituted,” means that any one or more hydrogen atoms on the designated atom is replaced with a selection from the indicated groups, provided that the designated atom's normal valency is not exceeded, and that the substitution results in a stable compound. When a substituent is oxo or keto (i.e., ═O), then 2 hydrogen atoms on the atom are replaced. Keto substituents are not present on aromatic moieties. Ring double bonds, as used herein, are double bonds that are formed between two adjacent ring atoms (e.g., C═C, C═N or N═N). “Stable compound” and “stable structure” are meant to indicate a compound that is sufficiently robust to survive isolation to a useful degree of purity from a reaction mixture, and formulation into an efficacious therapeutic agent.

When a bond to a substituent is shown to cross a bond connecting two atoms in a ring, then such substituent may be bonded to any atom in the ring. When a substituent is listed without indicating the atom via which such substituent is bonded to the rest of the compound of a given formula, then such substituent may be bonded via any atom in such formula. Combinations of substituents and/or variables are permissible, but only if such combinations result in stable compounds.

When any variable (e.g., R) occurs more than one time in any constituent or formula for a compound, its definition at each occurrence is independent of its definition at every other occurrence. Thus, for example, if a group is shown to be substituted with 0-2 R moieties, then the group may optionally be substituted with up to two R moieties and R at each occurrence is selected independently from the definition of R. Also, combinations of substituents and/or variables are permissible, but only if such combinations result in stable compounds.

As used herein, the term “hydroxy” or “hydroxyl” includes groups with an —OH or —O⁻.

As used herein, the term “halo” or “halogen” refers to fluoro, chloro, bromo and iodo.

The term “haloalkyl” or “haloalkoxyl” refers to an alkyl or alkoxyl substituted with one or more halogen atoms.

As used herein, the term “optionally substituted haloalkyl” refers to unsubstituted haloalkyl having designated substituents replacing one or more hydrogen atoms on one or more hydrocarbon backbone carbon atoms. Such substituents can include, for example, alkyl, alkenyl, alkynyl, halogen, hydroxyl, alkylcarbonyloxy, arylcarbonyloxy, alkoxycarbonyloxy, aryloxycarbonyloxy, carboxylate, alkylcarbonyl, arylcarbonyl, alkoxycarbonyl, aminocarbonyl, alkylaminocarbonyl, dialkylaminocarbonyl, alkylthiocarbonyl, alkoxyl, phosphate, phosphonato, phosphinato, amino (including alkylamino, dialkylamino, arylamino, diarylamino and alkylarylamino), acylamino (including alkylcarbonylamino, arylcarbonylamino, carbamoyl and ureido), amidino, imino, sulfhydryl, alkylthio, arylthio, thiocarboxylate, sulfates, alkylsulfinyl, sulfonato, sulfamoyl, sulfonamido, nitro, trifluoromethyl, cyano, azido, heterocyclyl, alkylaryl, or an aromatic or heteroaromatic moiety.

As used herein, the term “alkoxy” or “alkoxyl” includes substituted and unsubstituted alkyl, alkenyl and alkynyl groups covalently linked to an oxygen atom. Examples of alkoxy groups or alkoxyl radicals include, but are not limited to, methoxy, ethoxy, isopropyloxy, propoxy, butoxy and pentoxy groups. Examples of substituted alkoxy groups include halogenated alkoxy groups. The alkoxy groups can be substituted with groups such as alkenyl, alkynyl, halogen, hydroxyl, alkylcarbonyloxy, arylcarbonyloxy, alkoxycarbonyloxy, aryloxycarbonyloxy, carboxylate, alkylcarbonyl, arylcarbonyl, alkoxycarbonyl, aminocarbonyl, alkylaminocarbonyl, dialkylaminocarbonyl, alkylthiocarbonyl, alkoxyl, phosphate, phosphonato, phosphinato, amino (including alkylamino, dialkylamino, arylamino, diarylamino, and alkylarylamino), acylamino (including alkylcarbonylamino, arylcarbonylamino, carbamoyl and ureido), amidino, imino, sulfhydryl, alkylthio, arylthio, thiocarboxylate, sulfates, alkylsulfinyl, sulfonato, sulfamoyl, sulfonamido, nitro, trifluoromethyl, cyano, azido, heterocyclyl, alkylaryl, or an aromatic or heteroaromatic moieties. Examples of halogen substituted alkoxy groups include, but are not limited to, fluoromethoxy, difluoromethoxy, trifluoromethoxy, chloromethoxy, dichloromethoxy and trichloromethoxy.

As used herein, the expressions “one or more of A, B, or C,” “one or more A, B, or C,” “one or more of A, B, and C,” “one or more A, B, and C,” “selected from the group consisting of A, B, and C”, “selected from A, B, and C”, and the like are used interchangeably and all refer to a selection from a group consisting of A, B, and/or C, i.e., one or more As, one or more Bs, one or more Cs, or any combination thereof, unless indicated otherwise.

It is to be understood that the present disclosure provides methods for the synthesis of the compounds of any of the Formulae described herein. The present disclosure also provides detailed methods for the synthesis of various disclosed compounds of the present disclosure according to the following schemes as well as those shown in the Examples.

It is to be understood that, throughout the description, where compositions are described as having, including, or comprising specific components, it is contemplated that compositions also consist essentially of, or consist of, the recited components. Similarly, where methods or processes are described as having, including, or comprising specific process steps, the processes also consist essentially of, or consist of, the recited processing steps. Further, it should be understood that the order of steps or order for performing certain actions is immaterial so long as the invention remains operable. Moreover, two or more steps or actions can be conducted simultaneously.

It is to be understood that the synthetic processes of the disclosure can tolerate a wide variety of functional groups, therefore various substituted starting materials can be used. The processes generally provide the desired final compound at or near the end of the overall process, although it may be desirable in certain instances to further convert the compound to a pharmaceutically acceptable salt thereof.

It is to be understood that compounds of the present disclosure can be prepared in a variety of ways using commercially available starting materials, compounds known in the literature, or from readily prepared intermediates, by employing standard synthetic methods and procedures either known to those skilled in the art, or which will be apparent to the skilled artisan in light of the teachings herein. Standard synthetic methods and procedures for the preparation of organic molecules and functional group transformations and manipulations can be obtained from the relevant scientific literature or from standard textbooks in the field. Although not limited to any one or several sources, classic texts such as Smith, M. B., March, J., March's Advanced Organic Chemistry: Reactions, Mechanisms, and Structure, 5^{th} edition, John Wiley & Sons: New York, 2001; Greene, T. W., Wuts, P. G. M., Protective Groups in Organic Synthesis, 3^{rd} edition, John Wiley & Sons: New York, 1999; R. Larock, Comprehensive Organic Transformations, VCH Publishers (1989); L. Fieser and M. Fieser, Fieser and Fieser's Reagents for Organic Synthesis, John Wiley and Sons (1994); and L. Paquette, ed., Encyclopedia of Reagents for Organic Synthesis, John Wiley and Sons (1995), incorporated by reference herein, are useful and recognized reference textbooks of organic synthesis known to those in the art

One of ordinary skill in the art will note that, during the reaction sequences and synthetic schemes described herein, the order of certain steps may be changed, such as the introduction and removal of protecting groups. One of ordinary skill in the art will recognize that certain groups may require protection from the reaction conditions via the use of protecting groups. Protecting groups may also be used to differentiate similar functional groups in molecules. A list of protecting groups and how to introduce and remove these groups can be found in Greene, T. W., Wuts, P. G. M., Protective Groups in Organic Synthesis, 3^{rd} edition, John Wiley & Sons: New York, 1999.

It is to be understood that, unless otherwise stated, any description of a method of treatment includes use of the compounds to provide such treatment or prophylaxis as is described herein, as well as use of the compounds to prepare a medicament to treat or prevent such condition. The treatment includes treatment of human or non-human animals including rodents and other disease models.

As used herein, the term “subject” includes human and non-human animals, as well as cell lines, cell cultures, tissues, and organs. In some embodiments, the subject is a mammal. The mammal can be e.g., a human or appropriate non-human mammal, such as primate, mouse, rat, dog, cat, cow, horse, goat, camel, sheep or a pig. The subject can also be a bird or fowl. In some embodiments, the subject is a human.

As used herein, the term “subject in need thereof” refers to a subject having a disease or having an increased risk of developing the disease. A subject in need thereof can be one who has been previously diagnosed or identified as having a disease or disorder disclosed herein. A subject in need thereof can also be one who is suffering from a disease or disorder disclosed herein. Alternatively, a subject in need thereof can be one who has an increased risk of developing such disease or disorder relative to the population at large (i.e., a subject who is predisposed to developing such disorder relative to the population at large). A subject in need thereof can have a refractory or resistant a disease or disorder disclosed herein (i.e., a disease or disorder disclosed herein that does not respond or has not yet responded to treatment). The subject may be resistant at start of treatment or may become resistant during treatment. In some embodiments, the subject in need thereof received and failed all known effective therapies for a disease or disorder disclosed herein. In some embodiments, the subject in need thereof received at least one prior therapy.

As used herein, the term “treating” or “treat” describes the management and care of a patient for the purpose of combating a disease, condition, or disorder and includes the administration of a compound of the present disclosure, or a pharmaceutically acceptable salt thereof, to alleviate the symptoms or complications of a disease, condition or disorder, or to eliminate the disease, condition or disorder. The term “treat” can also include treatment of a cell in vitro or an animal model. It is to be appreciated that references to “treating” or “treatment” include the alleviation of established symptoms of a condition. “Treating” or “treatment” of a state, disorder or condition therefore includes: (1) preventing or delaying the appearance of clinical symptoms of the state, disorder or condition developing in a human that may be afflicted with or predisposed to the state, disorder or condition but does not yet experience or display clinical or subclinical symptoms of the state, disorder or condition, (2) inhibiting the state, disorder or condition, i.e., arresting, reducing or delaying the development of the disease or a relapse thereof (in case of maintenance treatment) or at least one clinical or subclinical symptom thereof, or (3) relieving or attenuating the disease, i.e., causing regression of the state, disorder or condition or at least one of its clinical or subclinical symptoms.

As used herein, the term “preventing,” “prevent,” or “protecting against” describes reducing or eliminating the onset of the symptoms or complications of such disease, condition or disorder.

As used herein, the term “temporal proximity” refers to that administration of one therapeutic agent (the compound of the present disclosure) occurs within a time period before or after the administration of another therapeutic agent (one or more inhibitors of the MAPK pathways), such that the therapeutic effect of the one therapeutic agent overlaps with the therapeutic effect of the other therapeutic agent. In some embodiments, the therapeutic effect of the one therapeutic agent completely overlaps with the therapeutic effect of the other therapeutic agent. In some embodiments, “temporal proximity” means that administration of one therapeutic agent occurs within a time period before or after the administration of another therapeutic agent, such that there is a synergistic effect between the one therapeutic agent and the other therapeutic agent. “Temporal proximity” may vary according to various factors, including but not limited to, the age, gender, weight, genetic background, medical condition, disease history, and treatment history of the subject to which the therapeutic agents are to be administered; the disease or condition to be treated or ameliorated; the therapeutic outcome to be achieved; the dosage, dosing frequency, and dosing duration of the therapeutic agents; the pharmacokinetics and pharmacodynamics of the therapeutic agents; and the route(s) through which the therapeutic agents are administered. In some embodiments, “temporal proximity” means within 15 minutes, within 30 minutes, within an hour, within two hours, within four hours, within six hours, within eight hours, within 12 hours, within 18 hours, within 24 hours, within 36 hours, within 2 days, within 3 days, within 4 days, within 5 days, within 6 days, within a week, within 2 weeks, within 3 weeks, within 4 weeks, with 6 weeks, or within 8 weeks. In some embodiments, multiple administration of one therapeutic agent can occur in temporal proximity to a single administration of another therapeutic agent. In some embodiments, temporal proximity may change during a treatment cycle or within a dosing regimen.

It is to be understood that a compound of the present disclosure, or a pharmaceutically acceptable salt thereof, can or may also be used to prevent a relevant disease, condition, or disorder, or used to identify suitable candidates for such purposes.

All percentages and ratios used herein, unless otherwise indicated, are by weight. Other features and advantages of the present disclosure are apparent from the different examples. The provided examples illustrate different components and methodology useful in practicing the present disclosure. The examples do not limit the claimed disclosure. Based on the present disclosure the skilled artisan can identify and employ other components and methodology useful for practicing the present disclosure.

In the synthetic schemes described herein, compounds may be drawn with one particular configuration for simplicity. Such particular configurations are not to be construed as limiting the disclosure to one or another isomer, tautomer, regioisomer or stereoisomer, nor does it exclude mixtures of isomers, tautomers, regioisomers or stereoisomers; however, it will be understood that a given isomer, tautomer, regioisomer or stereoisomer may have a higher level of activity than another isomer, tautomer, regioisomer or stereoisomer.

It is to be understood that one skilled in the art may refer to general reference texts for detailed descriptions of known techniques discussed herein or equivalent techniques. These texts include Ausubel et al., Current Protocols in Molecular Biology, John Wiley and Sons, Inc. (2005); Sambrook et al., Molecular Cloning, A Laboratory Manual (3^{rd} edition), Cold Spring Harbor Press, Cold Spring Harbor, New York (2000); Coligan et al., Current Protocols in Immunology, John Wiley & Sons, N.Y.; Enna et al., Current Protocols in Pharmacology, John Wiley & Sons, N.Y.; Fingl et al., The Pharmacological Basis of Therapeutics (1975), Remington's Pharmaceutical Sciences, Mack Publishing Co., Easton, PA, 18^{th} edition (1990). These texts can, of course, also be referred to in making or using an aspect of the disclosure.

It is to be understood that the present disclosure also provides pharmaceutical compositions comprising any compound described herein in combination with at least one pharmaceutically acceptable excipient or carrier.

As used herein, the term “pharmaceutical composition” is a formulation containing the compounds of the present disclosure in a form suitable for administration to a subject. In one embodiment, the pharmaceutical composition is in bulk or in unit dosage form. The unit dosage form is any of a variety of forms, including, for example, a capsule, an IV bag, a tablet, a single pump on an aerosol inhaler or a vial. The quantity of active ingredient (e.g., a formulation of the disclosed compound or salt thereof) in a unit dose of composition is an effective amount and is varied according to the particular treatment involved. One skilled in the art will appreciate that it is sometimes necessary to make routine variations to the dosage depending on the age and condition of the patient. The dosage will also depend on the route of administration. A variety of routes are contemplated, including oral, pulmonary, rectal, parenteral, transdermal, subcutaneous, intravenous, intramuscular, intraperitoneal, inhalational, buccal, sublingual, intrapleural, intrathecal, intranasal, and the like. Dosage forms for the topical or transdermal administration of a compound of this disclosure include powders, sprays, ointments, pastes, creams, lotions, gels, solutions, patches and inhalants. In one embodiment, the active compound is mixed under sterile conditions with a pharmaceutically acceptable carrier, and with any preservatives, buffers, or propellants that are required.

As used herein, the term “pharmaceutically acceptable” refers to those compounds, anions, cations, materials, compositions, carriers, and/or dosage forms which are, within the scope of sound medical judgment, suitable for use in contact with the tissues of human beings and animals without excessive toxicity, irritation, allergic response, or other problem or complication, commensurate with a reasonable benefit/risk ratio.

As used herein, the term “pharmaceutically acceptable excipient” means an excipient that is useful in preparing a pharmaceutical composition that is generally safe, non-toxic and neither biologically nor otherwise undesirable, and includes excipient that is acceptable for veterinary use as well as human pharmaceutical use. A “pharmaceutically acceptable excipient” as used in the specification and claims includes both one and more than one such excipient.

It is to be understood that a pharmaceutical composition of the disclosure is formulated to be compatible with its intended route of administration. Examples of routes of administration include parenteral, e.g., intravenous, intradermal, subcutaneous, oral (e.g., ingestion), inhalation, transdermal (topical), and transmucosal administration. Solutions or suspensions used for parenteral, intradermal, or subcutaneous application can include the following components: a sterile diluent such as water for injection, saline solution, fixed oils, polyethylene glycols, glycerine, propylene glycol or other synthetic solvents; antibacterial agents such as benzyl alcohol or methyl parabens; antioxidants such as ascorbic acid or sodium bisulfite; chelating agents such as ethylenediaminetetraacetic acid; buffers such as acetates, citrates or phosphates, and agents for the adjustment of tonicity such as sodium chloride or dextrose. The pH can be adjusted with acids or bases, such as hydrochloric acid or sodium hydroxide. The parenteral preparation can be enclosed in ampoules, disposable syringes or multiple dose vials made of glass or plastic.

It is to be understood that a compound or pharmaceutical composition of the disclosure can be administered to a subject in many of the well-known methods currently used for chemotherapeutic treatment. For example, a compound of the disclosure may be injected into the blood stream or body cavities or taken orally or applied through the skin with patches. The dose chosen should be sufficient to constitute effective treatment but not so high as to cause unacceptable side effects. The state of the disease condition (e.g., a disease or disorder disclosed herein) and the health of the patient should preferably be closely monitored during and for a reasonable period after treatment.

As used herein, the term “therapeutically effective amount”, refers to an amount of a pharmaceutical agent to treat, ameliorate, or prevent an identified disease or condition, or to exhibit a detectable therapeutic or inhibitory effect. The effect can be detected by any assay method known in the art. The precise effective amount for a subject will depend upon the subject's body weight, size, and health; the nature and extent of the condition; and the therapeutic or combination of therapeutics selected for administration. Therapeutically effective amounts for a given situation can be determined by routine experimentation that is within the skill and judgment of the clinician.

It is to be understood that, for any compound, the therapeutically effective amount can be estimated initially either in cell culture assays, e.g., of neoplastic cells, or in animal models, usually rats, mice, rabbits, dogs, or pigs. The animal model may also be used to determine the appropriate concentration range and route of administration. Such information can then be used to determine useful doses and routes for administration in humans. Therapeutic/prophylactic efficacy and toxicity may be determined by standard pharmaceutical procedures in cell cultures or experimental animals, e.g., ED₅₀ (the dose therapeutically effective in 50% of the population) and LD₅₀ (the dose lethal to 50% of the population). The dose ratio between toxic and therapeutic effects is the therapeutic index, and it can be expressed as the ratio, LD₅₀/ED₅₀. Pharmaceutical compositions that exhibit large therapeutic indices are preferred. The dosage may vary within this range depending upon the dosage form employed, sensitivity of the patient, and the route of administration.

Dosage and administration are adjusted to provide sufficient levels of the active agent(s) or to maintain the desired effect. Factors which may be taken into account include the severity of the disease state, general health of the subject, age, weight, and gender of the subject, diet, time and frequency of administration, drug combination(s), reaction sensitivities, and tolerance/response to therapy. Long-acting pharmaceutical compositions may be administered every 3 to 4 days, every week, or once every two weeks depending on half-life and clearance rate of the particular formulation.

The pharmaceutical compositions containing active compounds of the present disclosure may be manufactured in a manner that is generally known, e.g., by means of conventional mixing, dissolving, granulating, dragee-making, levigating, emulsifying, encapsulating, entrapping, or lyophilising processes. Pharmaceutical compositions may be formulated in a conventional manner using one or more pharmaceutically acceptable carriers comprising excipients and/or auxiliaries that facilitate processing of the active compounds into preparations that can be used pharmaceutically. Of course, the appropriate formulation is dependent upon the route of administration chosen.

Pharmaceutical compositions suitable for injectable use include sterile aqueous solutions (where water soluble) or dispersions and sterile powders for the extemporaneous preparation of sterile injectable solutions or dispersion. For intravenous administration, suitable carriers include physiological saline, bacteriostatic water, Cremophor EL™ (BASF, Parsippany, N.J.) or phosphate buffered saline (PBS). In all cases, the composition must be sterile and should be fluid to the extent that easy syringeability exists. It must be stable under the conditions of manufacture and storage and must be preserved against the contaminating action of microorganisms such as bacteria and fungi. The carrier can be a solvent or dispersion medium containing, for example, water, ethanol, polyol (for example, glycerol, propylene glycol, and liquid polyethylene glycol, and the like), and suitable mixtures thereof. The proper fluidity can be maintained, for example, by the use of a coating such as lecithin, by the maintenance of the required particle size in the case of dispersion and by the use of surfactants. Prevention of the action of microorganisms can be achieved by various antibacterial and antifungal agents, for example, parabens, chlorobutanol, phenol, ascorbic acid, thimerosal, and the like. In many cases, it will be preferable to include isotonic agents, for example, sugars, polyalcohols such as mannitol and sorbitol, and sodium chloride in the composition. Prolonged absorption of the injectable compositions can be brought about by including in the composition an agent which delays absorption, for example, aluminum monostearate and gelatin.

Sterile injectable solutions can be prepared by incorporating the active compound in the required amount in an appropriate solvent with one or a combination of ingredients enumerated above, as required, followed by filtered sterilisation. Generally, dispersions are prepared by incorporating the active compound into a sterile vehicle that contains a basic dispersion medium and the required other ingredients from those enumerated above. In the case of sterile powders for the preparation of sterile injectable solutions, methods of preparation are vacuum drying and freeze-drying that yields a powder of the active ingredient plus any additional desired ingredient from a previously sterile-filtered solution thereof.

Oral compositions generally include an inert diluent or an edible pharmaceutically acceptable carrier. They can be enclosed in gelatin capsules or compressed into tablets. For the purpose of oral therapeutic administration, the active compound can be incorporated with excipients and used in the form of tablets, troches, or capsules. Oral compositions can also be prepared using a fluid carrier for use as a mouthwash, wherein the compound in the fluid carrier is applied orally and swished and expectorated or swallowed. Pharmaceutically compatible binding agents, and/or adjuvant materials can be included as part of the composition. The tablets, pills, capsules, troches and the like can contain any of the following ingredients, or compounds of a similar nature: a binder such as microcrystalline cellulose, gum tragacanth or gelatin; an excipient such as starch or lactose, a disintegrating agent such as alginic acid, Primogel, or corn starch; a lubricant such as magnesium stearate or Sterotes; a glidant such as colloidal silicon dioxide; a sweetening agent such as sucrose or saccharin; or a flavoring agent such as peppermint, methyl salicylate, or orange flavoring.

For administration by inhalation, the compounds are delivered in the form of an aerosol spray from pressured container or dispenser, which contains a suitable propellant, e.g., a gas such as carbon dioxide, or a nebuliser.

Systemic administration can also be by transmucosal or transdermal means. For transmucosal or transdermal administration, penetrants appropriate to the barrier to be permeated are used in the formulation. Such penetrants are generally known in the art, and include, for example, for transmucosal administration, detergents, bile salts, and fusidic acid derivatives. Transmucosal administration can be accomplished through the use of nasal sprays or suppositories. For transdermal administration, the active compounds are formulated into ointments, salves, gels, or creams as generally known in the art.

The active compounds can be prepared with pharmaceutically acceptable carriers that will protect the compound against rapid elimination from the body, such as a controlled release formulation, including implants and microencapsulated delivery systems. Biodegradable, biocompatible polymers can be used, such as ethylene vinyl acetate, polyanhydrides, polyglycolic acid, collagen, polyorthoesters, and polylactic acid. Methods for preparation of such formulations will be apparent to those skilled in the art. The materials can also be obtained commercially from Alza Corporation and Nova Pharmaceuticals, Inc. Liposomal suspensions (including liposomes targeted to infected cells with monoclonal antibodies to viral antigens) can also be used as pharmaceutically acceptable carriers. These can be prepared according to methods known to those skilled in the art, for example, as described in U.S. Pat. No. 4,522,811.

It is especially advantageous to formulate oral or parenteral compositions in dosage unit form for ease of administration and uniformity of dosage. Dosage unit form as used herein refers to physically discrete units suited as unitary dosages for the subject to be treated; each unit containing a predetermined quantity of active compound calculated to produce the desired therapeutic effect in association with the required pharmaceutical carrier. The specification for the dosage unit forms of the disclosure are dictated by and directly dependent on the unique characteristics of the active compound and the particular therapeutic effect to be achieved.

In therapeutic applications, the dosages of the pharmaceutical compositions used in accordance with the disclosure vary depending on the agent, the age, weight, and clinical condition of the recipient patient, and the experience and judgment of the clinician or practitioner administering the therapy, among other factors affecting the selected dosage. Generally, the dose should be sufficient to result in slowing, and preferably regressing, the symptoms of the disease or disorder disclosed herein and also preferably causing complete regression of the disease or disorder. Dosages can range from about 0.01 mg/kg per day to about 5000 mg/kg per day. In preferred aspects, dosages can range from about 1 mg/kg per day to about 1000 mg/kg per day. In an aspect, the dose will be in the range of about 0.1 mg/day to about 50 g/day; about 0.1 mg/day to about 25 g/day; about 0.1 mg/day to about 10 g/day; about 0.1 mg to about 3 g/day; or about 0.1 mg to about 1 g/day, in single, divided, or continuous doses (which dose may be adjusted for the patient's weight in kg, body surface area in m², and age in years). An effective amount of a pharmaceutical agent is that which provides an objectively identifiable improvement as noted by the clinician or other qualified observer. Improvement in survival and growth indicates regression. As used herein, the term “dosage effective manner” refers to amount of an active compound to produce the desired biological effect in a subject or cell.

As used herein, the term “daily dosage” refers to the total amount of dosage of an agent being administered in a day. Such total amount can be administered in a single dose during the day (e.g., once daily), or can be administered across two or more distributive doses throughout the day (e.g., twice daily).

It is to be understood that the pharmaceutical compositions can be included in a container, pack, or dispenser together with instructions for administration.

It is to be understood that, for the compounds of the present disclosure being capable of further forming salts, all of these forms are also contemplated within the scope of the claimed disclosure.

As used herein, the term “pharmaceutically acceptable salts” refer to derivatives of the compounds of the present disclosure wherein the parent compound is modified by making acid or base salts thereof. Examples of pharmaceutically acceptable salts include, but are not limited to, mineral or organic acid salts of basic residues such as amines, alkali or organic salts of acidic residues such as carboxylic acids, and the like. The pharmaceutically acceptable salts include the conventional non-toxic salts or the quaternary ammonium salts of the parent compound formed, for example, from non-toxic inorganic or organic acids. For example, such conventional non-toxic salts include, but are not limited to, those derived from inorganic and organic acids selected from 2-acetoxybenzoic, 2-hydroxyethane sulfonic, acetic, ascorbic, benzene sulfonic, benzoic, bicarbonic, carbonic, citric, edetic, ethane disulfonic, 1,2-ethane sulfonic, fumaric, glucoheptonic, gluconic, glutamic, glycolic, glycollyarsanilic, hexylresorcinic, hydrabamic, hydrobromic, hydrochloric, hydroiodic, hydroxymaleic, hydroxynaphthoic, isethionic, lactic, lactobionic, lauryl sulfonic, maleic, malic, mandelic, methane sulfonic, napsylic, nitric, oxalic, pamoic, pantothenic, phenylacetic, phosphoric, polygalacturonic, propionic, salicylic, stearic, subacetic, succinic, sulfamic, sulfanilic, sulfuric, tannic, tartaric, toluene sulfonic, and the commonly occurring amine acids, e.g., glycine, alanine, phenylalanine, arginine, etc.

In some embodiments, the pharmaceutically acceptable salt is a sodium salt, a potassium salt, a calcium salt, a magnesium salt, a diethylamine salt, a choline salt, a meglumine salt, a benzathine salt, a tromethamine salt, an ammonia salt, an arginine salt, or a lysine salt.

Other examples of pharmaceutically acceptable salts include hexanoic acid, cyclopentane propionic acid, pyruvic acid, malonic acid, 3-(4-hydroxybenzoyl)benzoic acid, cinnamic acid, 4-chlorobenzenesulfonic acid, 2-naphthalenesulfonic acid, 4-toluenesulfonic acid, camphorsulfonic acid, 4-methylbicyclo-[2.2.2]-oct-2-ene-1-carboxylic acid, 3-phenylpropionic acid, trimethylacetic acid, tertiary butylacetic acid, muconic acid, and the like. The present disclosure also encompasses salts formed when an acidic proton present in the parent compound either is replaced by a metal ion, e.g., an alkali metal ion, an alkaline earth ion, or an aluminum ion; or coordinates with an organic base such as ethanolamine, diethanolamine, triethanolamine, tromethamine, N-methylglucamine, and the like. In the salt form, it is understood that the ratio of the compound to the cation or anion of the salt can be 1:1, or any ratio other than 1:1, e.g., 3:1, 2:1, 1:2, or 1:3.

It is to be understood that all references to pharmaceutically acceptable salts include solvent addition forms (solvates) or crystal forms (polymorphs) as defined herein, of the same salt.

The compounds, or pharmaceutically acceptable salts thereof, are administered orally, nasally, transdermally, pulmonary, inhalationally, buccally, sublingually, intraperitoneally, subcutaneously, intramuscularly, intravenously, rectally, intrapleurally, intrathecally and parenterally. In one embodiment, the compound is administered orally. One skilled in the art will recognize the advantages of certain routes of administration.

The dosage regimen utilising the compounds is selected in accordance with a variety of factors including type, species, age, weight, sex and medical condition of the patient; the severity of the condition to be treated; the route of administration; the renal and hepatic function of the patient; and the particular compound or salt thereof employed. An ordinarily skilled physician or veterinarian can readily determine and prescribe the effective amount of the drug required to prevent, counter, or arrest the progress of the condition.

Techniques for formulation and administration of the disclosed compounds of the disclosure can be found in Remington: the Science and Practice of Pharmacy, 19^{th} edition, Mack Publishing Co., Easton, PA (1995). In some embodiments, the compounds described herein, and the pharmaceutically acceptable salts thereof, are used in pharmaceutical preparations in combination with a pharmaceutically acceptable carrier or diluent. Suitable pharmaceutically acceptable carriers include inert solid fillers or diluents and sterile aqueous or organic solutions. The compounds will be present in such pharmaceutical compositions in amounts sufficient to provide the desired dosage amount in the range described herein.

All percentages and ratios used herein, unless otherwise indicated, are by weight. Other features and advantages of the present disclosure are apparent from the different examples. The provided examples illustrate different components and methodology useful in practicing the present disclosure. The examples do not limit the claimed disclosure. Based on the present disclosure the skilled artisan can identify and employ other components and methodology useful for practicing the present disclosure.

In the synthetic schemes described herein, compounds may be drawn with one particular configuration for simplicity. Such particular configurations are not to be construed as limiting the disclosure to one or another isomer, tautomer, regioisomer or stereoisomer, nor does it exclude mixtures of isomers, tautomers, regioisomers or stereoisomers; however, it will be understood that a given isomer, tautomer, regioisomer or stereoisomer may have a higher level of activity than another isomer, tautomer, regioisomer or stereoisomer.

All publications and patent documents cited herein are incorporated herein by reference as if each such publication or document was specifically and individually indicated to be incorporated herein by reference. Citation of publications and patent documents is not intended as an admission that any is pertinent prior art, nor does it constitute any admission as to the contents or date of the same. The invention having now been described by way of written description, those of skill in the art will recognize that the invention can be practiced in a variety of embodiments and that the foregoing description and examples below are for purposes of illustration and not limitation of the claims that follow.

### EXAMPLES

### Example 1. Biological Activity of Exemplary Compounds

Retroviral Production: The BRAF-KIAA1549 fusion mutant was subcloned into pMXs-IRES-Blasticidin (RTV-016, Cell Biolabs, San Diego, CA). Retrovirus was produced by transfection of HEK 293T cells with the retroviral BRAF mutant expression vector pMXs-IRES-Blasticidin (RTV-016, Cell Biolabs), pCMV-Gag-Pol vector and pCMV-VSV-G-Envelope vector. Briefly, HEK 293T cells were plated in Poly-D-lysine coated 6-well plate (2.5×10⁵ cells per well) and incubated overnight. The next day, retroviral plasmids (1 μg of BRAF-KIAA fusion mutant, 0.32 μg of pCMV-Gag-Pol and 0.165 μg pCMV-VSV-G) were mixed in 300 μL of Optimem (31985, Life Technologies). The mixture was incubated at room temperature for 5 minutes and then added to Optimem containing transfection reagent Lipofectamine (11668, Invitrogen) and incubated for 20 minutes. The mixture was then added dropwise to HEK 293T cells. The next day the medium was replaced with fresh culture medium and retrovirus was harvested at 24 hours.

Generation of BRAF-KIAA1549 fusion stable cell line: BaF3 cells were plated in V-bottom 96 well plate (COSTAR #3894) in 80 μL of RPMI/10% FBS and 0.01 ng/mL mouse IL-3, supplemented with 8 μg/mL polybrene (4×10⁴ cells/well). Cells were transduced with 20 μL of BRAF-KIAA1549 fusion viral supernatant by centrifuging for 30 minutes at 1000 rpm. Cells were placed in a 37 °C incubator overnight. Next day 100 μL of RPMI/10% FBS and 0.01 ng/mL mouse IL-3 was added. After 24 hours, 100 μL of cells were transferred to a 24 well plate containing 900 μL of RPMI/10% FBS and 15 μg/mL Blasticidin. Contents of the 24 well plate were sampled for cell viability via CellTiterGlo (Promega) over a 2-week period. Cells that were observed to have greater than 1-fold viability over day of plating were expanded into T25 flasks for cell banking and Sanger sequence confirmation.

Assay for cell proliferation: BaF3 BRAF-KIAA1549 fusion cells were resuspended at 1.5×10⁵ c/mL in RPMI containing 10% Heat Inactivated FBS, 1% L-glutamine and dispensed in duplicate (7×10⁴ c/well) into 384 well plates. To determine the effect of compound on cell proliferation, BaF3 BRAF-KIAA1549 fusion cells were incubated for 96 hours in the presence of vehicle control (DMSO) or compound at varying concentrations. Inhibition of cell growth was determined by luminescent quantification (Envision by Perkin Elmer) of intracellular ATP content using CellTiterGlo (Promega), according to the protocol provided by the manufacturer. To determine the IC₅₀ values, the vehicle-treated cells were normalized as viable cells and analyzed using the CDD Vault, Collaborative Drug Discovery, Burlingame, CA (the Levenberg-Marquardt algorithm; Levenberg, K, 1994; Marquardt, D., 1963).

Table A assigns each compound a code for potency in the BaF3 BRAF-KIAA1549 fusion cell proliferation assay: A, B, C, or D. According to the code, A represents an IC₅₀ value<20 nM; B represents an IC₅₀ value≥20 nM and <100 nM; C represents an IC₅₀ value≥100 nM and <500 nM; and D represents an IC₅₀ value≥500 nM.

TABLE A

| Compound No. | BaF3-BRAF:KIAA1549 Fusion |
|---|---|
| 1 | A |
| 2 | B |
| 3 | A |
| 4 | A |
| 5 | B |
| 6 | A |
| 7 | A |
| 8 | B |
| 9 | A |
| 10 | A |
| 11 | B |
| 12 | B |
| 13 | C |
| 14 | A |
| 15 | B |
| 16 | A |
| 17 | A |
| 18 | A |
| 19 | A |
| 20 | A |
| 21 | B |
| 22 | B |
| 23 | B |
| 24 | A |
| 25 | B |
| 26 | B |
| 27 | A |
| 28 | C |
| 29 | A |
| 30 | B |
| 31 | B |
| 32 | A |
| 33 | A |
| 34 | B |
| 35 | B |
| 36 | C |
| 37 | B |
| 38 | B |
| 39 | B |
| 40 | B |
| 41 | B |
| 42 | B |
| 43 | B |
| 44 | B |
| 45 | C |
| 46 | C |
| 47 | C |
| 48 | D |
| 49 | A |
| 50 | C |
| 51 | D |
| 52 | A |
| 53 | B |
| 55 | A |
| 56 | B |
| 57 | A |
| 58 | B |
| 59 | B |
| 60 | A |
| 61 | C |
| 62 | B |
| 63 | B |
| 64 | D |
| 65 | B |
| 66 | B |
| 68 | B |
| 69 | C |
| 71 | B |
| 72 | D |
| 75 | B |
| 77 | B |
| 78 | B |
| 79 | A |
| 153 | B |
| 154 | A |
| 173 | B |
| 174 | B |
| 175 | B |
| 176 | B |
| 189 | C |
| 190 | C |
| 191 | B |
| 192 | C |
| 193 | A |
| 194 | B |
| 195 | D |
| 196 | D |
| 197 | D |
| 198 | D |
| 199 | D |
| 200 | A |
| 201 | B |
| 202 | B |
| 203 | A |
| 204 | A |
| 205 | C |
| 206 | B |
| 207 | D |
| 208 | D |
| 209 | D |
| 210 | D |
| 211 | D |
| 212 | D |
| 213 | D |
| 214 | B |
| 215 | B |
| 216 | C |
| 217 | B |
| 218 | B |
| 219 | D |
| 220 | B |
| 221 | B |
| 222 | B |
| 223 | B |
| 224 | C |
| 225 | B |
| 226 | B |
| 227 | B |
| 228 | C |
| 229 | B |
| 230 | B |
| 231 | B |
| 232 | C |
| 233 | B |
| 234 | B |
| 235 | B |
| 236 | B |
| 237 | B |
| 238 | B |
| 239 | D |
| 240 | D |
| 241 | C |
| 242 | B |
| 243 | B |
| 244 | D |
| 245 | B |
| 246 | D |
| 247 | B |
| 248 | B |
| 249 | B |
| 250 | D |
| 251 | C |
| 252 | B |
| 253 | B |
| 254 | C |
| 255 | B |
| 256 | B |
| 257 | B |
| 258 | B |
| 259 | B |
| 260 | B |
| 261 | A |
| 262 | B |
| 263 | A |
| 264 | B |
| 265 | A |
| 266 | B |
| 267 | A |
| 269 | C |
| 270 | D |
| 271 | B |
| 272 | B |
| 273 | C |
| 274 | A |
| 275 | B |
| 276 | A |

### Example 2. Nonclinical Studies of a Compound of the Current Disclosure

Pharmacologic activity of a compound of the current disclosure was studied in both in vitro and in a variety of in vivo animal models. The results of in vitro cell-based assays showed that this compound potently inhibited cell proliferation of a wide spectrum of oncogenic BRAF or NRAS expressing cell lines while sparing wild-type BRAF (>18-fold selectivity) and NRAS (8- to 43-fold selectivity) and avoiding paradoxical activation. The compound inhibited the cell proliferation of BRAF V600E (Class I) cell lines resistant to combined BRAF and MEK inhibitors (dabrafenib and trametinib, and encorafenib and binimetinib). The exposure in plasma, tumor, and brain increased with increasing dose. Increased tumor exposure to the compound correlated with increased inhibition of cellular ERK phosphorylation, which suggests on-target activity.

Anti-tumor activity of a compound of the present disclosure was investigated in mice with tumor models driven by BRAF Class I, II, and III mutations, and KRAS G12D and G12V mutations (Table B). Anti-tumor activity was demonstrated across subcutaneous tumor models representing all three classes of BRAF mutations as well as KRAS mutations.

Cancer cell line that endogenously harbor BRAF mutation or patient-derived tumor with KRAS mutation was implanted subcutaneously on the flank of nude mouse. When the tumor volume reached approximately 200 mm³ (170-270 mm³ depending on the model), animals were randomized into vehicle/control or test article groups. Mice were then administered with vehicle/control or test article once or two times daily for 14, 21, or 28 days. Tumor volume was measured 2-3 times a week during study period.

TABLE B

| | | Tumor Growth Inhibition (%) |
|---|---|---|
| | Dose | at the end of study |
| Mutation Model | (mg/kg) | (14, 21, or 28 days) |
| BRAF V600E | 10 QD | 108 |
| Class 1 | | |
| BRAF ΔL485-P490 + Y/F | 5 QD | 117 |
| Class 2 | | |
| BRAF G466V | 5 QD | 108 |
| Class 3 | | |
| KRAS G12V | 10 QD | 107 |
| KRAS G12D | 10 QD | 139 |
| | 5 BID | 141 |

Tumor Growth Inhibition (TGI) value is calculated using the formula:

TGI (%)=[1−(Dₜ−D₀)/(Cₜ−C₀)]×100

Dₜ=medium tumor volume of compound treated group at time t. D₀=medium tumor volume of compound treated group at time 0. Cₜ=medium tumor volume of control (vehicle) group at time t. C₀=medium tumor volume of compound treated group at time 0.

It is understood that the values presented in Table B are approximate, and are subject to experimental and instrumental variations.

### Example 3. Clinical Study of a Compound of the Current Disclosure

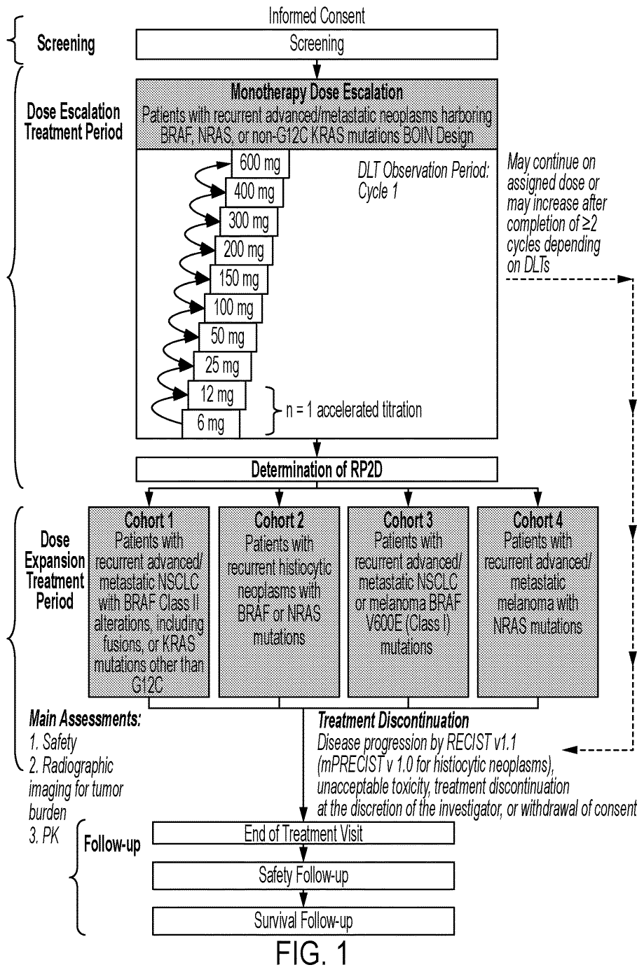

This is a first in human, Phase 1, multicenter, open-label, dose escalation and expansion study designed to evaluate the safety and tolerability, determine the RP2D, and evaluate the preliminary antitumor activity in patients with advanced/metastatic NSCLC harboring BRAF Class I and II alterations or KRAS non-G12C mutations, advanced/metastatic melanoma harboring BRAF Class I mutations or NRAS mutations, histiocytic neoplasms harboring BRAF or NRAS mutations, and other select tumors with Class I, II, and III mutations (FIG. 1). The study is conducted in 2 parts.

Part 1: The Dose Escalation part of the study employs Bayesian optimal interval design (BOIN) dose escalation including 2 single patient accelerated titration dose levels. Patients with advanced/metastatic tumors or neoplasms harboring BRAF, KRAS non-G12, or NRAS mutations that have progression, resistance, or intolerance of prior standard of care systemic therapy are eligible.

Part 2: The Dose Expansion part of the study includes 4 disease-specific cohorts:
Cohort 1: Patients with recurrent advanced/metastatic NSCLC with BRAF Class II alterations, including fusions, or KRAS non-G12C mutations.
Cohort 2: Patients with recurrent and intolerant histiocytic neoplasms with BRAF or NRAS mutations.
Cohort 3: Patients with recurrent advanced/metastatic NSCLC or melanoma with BRAF V600E (Class I) mutations.
Cohort 4: Patients with recurrent advanced/metastatic melanoma with NRAS mutations.

### Monotherapy Dose Escalation

The dose escalation part of the study includes an accelerated single patient dose escalation. The dose-limiting toxicity (DLT) Observation Period is defined from first dose on day 1 of cycle 1 (C1D1) through to the end of the first cycle (28-day cycle period).

Dose escalation cohorts:
Melanoma with BRAF (Class I, II, or III) or NRAS mutations
NSCLC with BRAF (Class I, II or III) or KRAS non-G12C mutations
Histiocytic neoplasms with BRAF (Class I, II, or III) or NRAS mutations
Thyroid carcinoma with BRAF (Class I, II, or III) mutations
Colorectal carcinoma with BRAF (Class II or III) mutations
Other solid tumors with BRAF Class I mutations after prior treatment with a BRAF/MEK inhibitor

The selection of the RP2D occurs after a sufficient number of patients have enrolled and completed PK and safety assessments during dose escalation, and the data have been reviewed for safety. Upon determination of RP2D, the dose expansion part of the study is initiated in additional patients with select neoplasms with or without brain metastases based on known BRAF, KRAS non-G12C, or NRAS alterations to further evaluate the safety, tolerability, and preliminary evidence of antitumor activity. If no RP2D is established at the end of dose escalation, expansion cohorts may be opened to obtain additional PK, safety, and tolerability data to support further dose optimization, including investigation of several dose levels to support exposure response analysis and to define RP2D.

### Dose Expansion

Patients are enrolled in each disease-specific expansion cohort to receive treatment (Cohorts 1, 2, 3 and 4). Enrollment may be extended for each dose expansion cohort if determined that additional safety, PK, and early efficacy data (if available) are required to support further dose optimization. Additional cohorts may be added based on emerging data from the dose escalation part of the study. Safety data is reviewed across all expansion cohorts on an ongoing basis. Significant safety findings may lead to early stopping for any expansion cohort and will be considered when deciding to initiate a new disease-specific expansion cohort.

### Dose Expansion Cohorts:

Cohort 1: Recurrent NSCLC with BRAF Class II alterations or KRAS non-G12C mutations without small cell lung cancer transformation with progressive disease confirmed by radiographic assessment
Cohort 2: Recurrent and intolerant histiocytic neoplasms with BRAF or NRAS alterations with progressive disease confirmed by radiographic assessment.
Cohort 3: Recurrent NSCLC (without small cell lung cancer transformation) or melanoma with BRAF Class I mutations with progressive disease confirmed by radiographic assessment.
Cohort 4: Recurrent melanoma with NRAS mutations with progressive disease confirmed by radiographic assessment.

Combination therapies with a MEK inhibitor are explored. A pilot food effect sub-study is conducted to evaluate the effect of food on the PK.

### Example 4. Study of a Compound of the Current Disclosure in Combination with Binimetinib

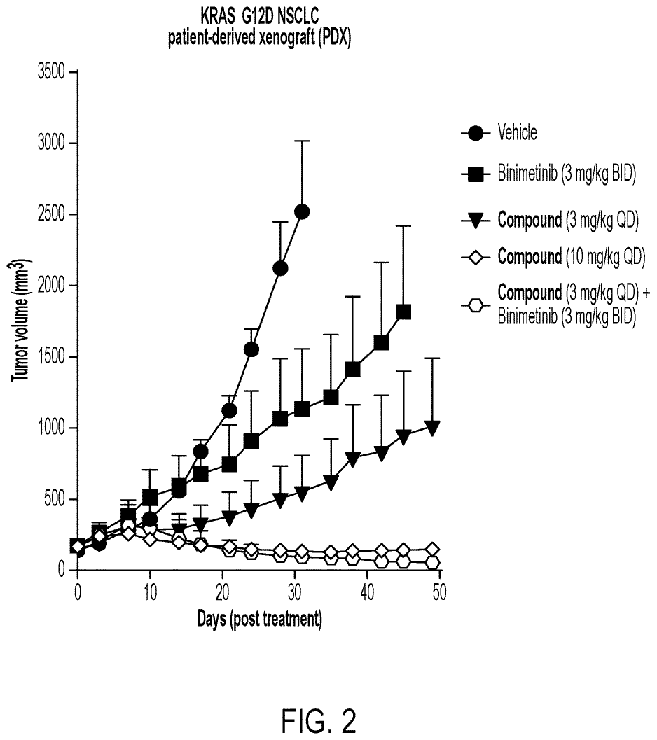

Anti-tumor activity of a compound of the present disclosure in combination with binimetinib was investigated in mice with KRAS G12D NSCLC patient-derived xenograft (PDX) model. Nu/Nu mice implanted with KRAS G12D patient-derived NSCLC tumor were randomized to control or treatment group when the overall average tumor mean volume was 172 mm³. Mice were administered twice daily approximately 12 hours apart with vehicle, twice daily approximately 12 hours apart with binimetinib, and once daily with the compound of the present disclosure, or the combination for 50 days by oral gavage. No significant body weight loss was observed for all groups. For representative results of the study, see FIG. 2.

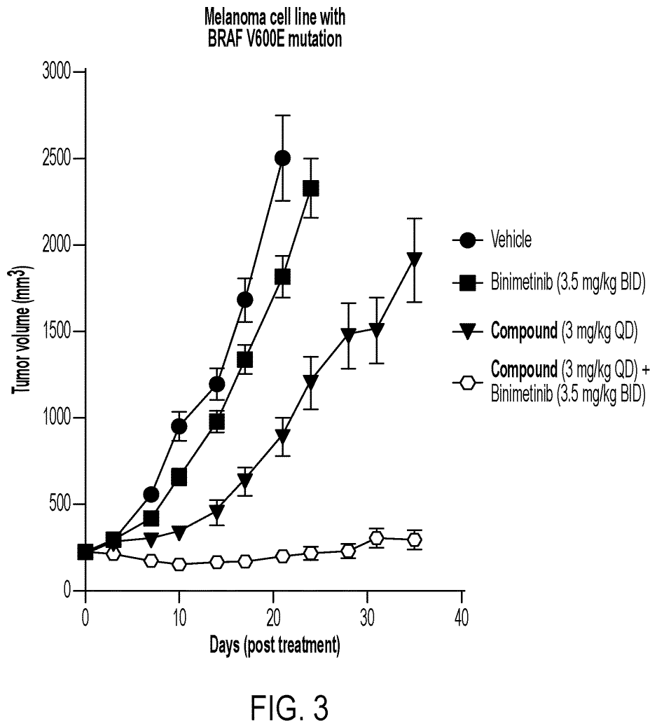

Anti-tumor activity of a compound of the present disclosure in combination with binimetinib was also investigated in mice with melanoma cell line with BRAF V600E mutation. BALB/c nude mice implanted with A375-Luc melanoma cells were randomized to control or treatment group when the overall average tumor mean volume was 224 mm³. Mice were administered twice daily approximately 12 hours apart with vehicle, twice daily approximately 12 hours apart with binimetinib, and once daily with the compound of the present disclosure, or the combination for 35 days by oral gavage. No significant body weight loss was observed for all groups. For representative results of the study, see FIG. 3.

### EQUIVALENTS

The details of one or more embodiments of the disclosure are set forth in the accompanying description above. Although any methods and materials similar or equivalent to those described herein can be used in the practice or testing of the present disclosure, the preferred methods and materials are now described. Other features, objects, and advantages of the disclosure will be apparent from the description and from the claims. In the specification and the appended claims, the singular forms include plural referents unless the context clearly dictates otherwise. Unless defined otherwise, all technical and scientific terms used herein have the same meaning as commonly understood by one of ordinary skill in the art to which this disclosure belongs. All patents and publications cited in this specification are incorporated by reference.

The foregoing description has been presented only for the purposes of illustration and is not intended to limit the disclosure to the precise form disclosed, but by the claims appended hereto.

1. A method of treating or preventing cancer in a subject, the method comprising administering to the subject a compound of Formula (0): 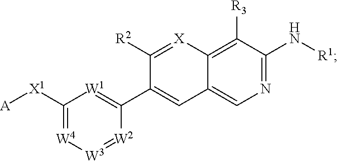
an isomer thereof, or a pharmaceutically acceptable salt thereof, wherein:
X is CR^{X} or N;
R^{X} is H, halogen, cyano, oxo, OH, C₁-C₆ alkyl, C₂-C₆ alkenyl, C₂-C₆ alkynyl, or C₁-C₆ alkoxy, wherein the C₁-C₆ alkyl, C₂-C₆ alkenyl, C₂-C₆ alkynyl, or C₁-C₆ alkoxy is optionally substituted with one or more halogen, cyano, oxo, or OH;
W¹ is N or CR^{W1};
R^{W1} is H, halogen, C₁-C₆ alkyl, C₂-C₆ alkenyl, or C₂-C₆ alkynyl;
W² is N or CR^{W2};
R^{W2} is H, halogen, C₁-C₆ alkyl, C₂-C₆ alkenyl, or C₂-C₆ alkynyl, wherein the C₁-C₆ alkyl, C₂-C₆ alkenyl, or C₂-C₆ alkynyl is optionally substituted with one or more halogen;
W³ is N or CR^{W3};
R^{W3} is H, halogen, C₁-C₆ alkyl, C₂-C₆ alkenyl, or C₂-C₆ alkynyl;
W⁴ is N or CR^{W4};
R^{W4} is H, halogen, C₁-C₆ alkyl, C₂-C₆ alkenyl, C₂-C₆ alkynyl, or S(C₁-C₆ alkyl);
R¹ is H, C₁-C₆ alkyl, C₂-C₆ alkenyl, C₂-C₆ alkynyl, C₁-C₆ alkoxy, C₃-C₁₂ cycloalkyl, 3- to 12-membered heterocycloalkyl, C₆-C₁₀ aryl, or 5- to 10-membered heteroaryl, wherein the C₁-C₆ alkyl, C₂-C₆ alkenyl, C₂-C₆ alkynyl, C₁-C₆ alkoxy, C₃-C₁₂ cycloalkyl, 3- to 12-membered heterocycloalkyl, C₆-C₁₀ aryl, or 5- to 10-membered heteroaryl is optionally substituted with one or more R^{1a};
each R^{1a} independently is halogen, cyano, oxo, OH, NH₂, NHC(═O)O(C₁-C₆ alkyl), N(C₁-C₆ alkyl)₂, C₁-C₆ alkyl, C₂-C₆ alkenyl, C₂-C₆ alkynyl, C₁-C₆ alkoxy, C₃-C₁₂ cycloalkyl, 3- to 12-membered heterocycloalkyl, C₆-C₁₀ aryl, or 5- to 10-membered heteroaryl;
R² is H, halogen, cyano, oxo, OH, C₁-C₆ alkyl, C₂-C₆ alkenyl, C₂-C₆ alkynyl, or C₁-C₆ alkoxy;
R³ is H, halogen, cyano, oxo, OH, C₁-C₆ alkyl, C₂-C₆ alkenyl, C₂-C₆ alkynyl, or C₁-C₆ alkoxy; or
R¹ and R³, together with the intervening atoms, form a 4- to 12-membered heterocycloalkyl optionally substituted with one or more oxo;
X¹ is —NR^{X1}—*, —C(═O)NR^{X1}—*, —NR^{X1}C(═O)—*, —NR^{X1}C(═O)O—*, —NR^{X1}N═C—*, —NR^{X1}C(═NR^{X1})—*, —N C(═NH)NR^{X1}—*, —NR^{X1}C(═O)NR^{X1}—*, —S(═O)₂NR^{X1}—*, or —NR^{X1}S(═O)₂—*, wherein * denotes attachment to A;
R^{X1} independently is H, S(═O)₂R^{X1a}, C₁-C₆ alkyl, C₂-C₆ alkenyl, C₂-C₆ alkynyl, C₁-C₆ alkoxy, C₃-C₁₂ cycloalkyl, 3- to 12-membered heterocycloalkyl, C₆-C₁₀ aryl, or 5- to 10-membered heteroaryl, wherein the C₁-C₆ alkyl, C₂-C₆ alkenyl, C₂-C₆ alkynyl, C₁-C₆ alkoxy, C₃-C₁₂ cycloalkyl, 3- to 12-membered heterocycloalkyl, C₆-C₁₀ aryl, or 5- to 10-membered heteroaryl is optionally substituted with one or more R^{X1a};
each R^{X1a} independently is halogen, C₁-C₆ alkyl, or 3- to 12-membered heterocycloalkyl, wherein the C₁-C₆ alkyl, or 3- to 12-membered heterocycloalkyl is optionally substituted with one or more halogen;
A is C₁-C₆ alkyl, C₃-C₁₂ cycloalkyl, 3- to 12-membered heterocycloalkyl, C₆-C₁₀ aryl, 5- to 10-membered heteroaryl, —(C₁-C₆ alkyl)-(C₃-C₁₂ cycloalkyl), —(C₁-C₆ alkyl)-(3- to 12-membered heterocycloalkyl), —(C₁-C₆ alkyl)-(C₆-C₁₀ aryl), or —(C₁-C₆ alkyl)-(5- to 10-membered heteroaryl), wherein the C₁-C₆ alkyl, C₃-C₁₂ cycloalkyl, 3- to 12-membered heterocycloalkyl, C₆-C₁₀ aryl, 5- to 10-membered heteroaryl, —(C₁-C₆ alkyl)-(C₃-C₁₂ cycloalkyl), —(C₁-C₆ alkyl)-(3- to 12-membered heterocycloalkyl), —(C₁-C₆ alkyl)-(C₆-C₁₀ aryl), or —(C₁-C₆ alkyl)-(5- to 10-membered heteroaryl) is optionally substituted with one or more R^{A};
each R^{A} independently is halogen, cyano, oxo, OH, OR^{A1}, NH₂, NHR^{A1}, N(R^{A1})₂, (═N)R^{A1}, C₁-C₆ alkyl, C₂-C₆ alkenyl, C₂-C₆ alkynyl, C₁-C₆ alkoxy, C₃-C₁₂ cycloalkyl, 3- to 12-membered heterocycloalkyl, C₆-C₁₀ aryl, or 5- to 10-membered heteroaryl, wherein the C₁-C₆ alkyl, C₂-C₆ alkenyl, C₂-C₆ alkynyl, C₁-C₆ alkoxy, C₃-C₁₂ cycloalkyl, 3- to 12-membered heterocycloalkyl, C₆-C₁₀ aryl, or 5- to 10-membered heteroaryl is optionally substituted with one or more R^{A1};
each R^{A1} independently is halogen, cyano, oxo, OH, OR^{A2}, NH₂, NHR^{A2}, N(R^{A2})₂, C(═O)R^{A2}, C₁-C₆ alkyl, C₂-C₆ alkenyl, C₂-C₆ alkynyl, C₁-C₆ alkoxy, C₃-C₁₂ cycloalkyl, 3- to 12-membered heterocycloalkyl, C₆-C₁₀ aryl, or 5- to 10-membered heteroaryl, wherein the C₁-C₆ alkyl, C₂-C₆ alkenyl, C₂-C₆ alkynyl, C₁-C₆ alkoxy, C₃-C₁₂ cycloalkyl, 3- to 12-membered heterocycloalkyl, C₆-C₁₀ aryl, or 5- to 10-membered heteroaryl is optionally substituted with one or more R^{A2}; and
each R^{A2} independently is halogen, cyano, OH, NH₂, N(R^{A3})₂, C(═O)R^{A3}, C₁-C₆ alkyl, C₂-C₆ alkenyl, or C₂-C₆ alkynyl,
wherein R^{A3} is C₁-C₆ alkyl, C₂-C₆ alkenyl, or C₂-C₆ alkynyl.

2. A compound of Formula (0), an isomer thereof, or a pharmaceutically acceptable salt thereof for treating or preventing cancer in a subject.

3. Use of a compound of Formula (0), an isomer thereof, or a pharmaceutically acceptable salt thereof in the manufacture of a medicament for treating or preventing cancer in a subject.

4. A combination comprising: (i) a compound of Formula (0), an isomer thereof, or a pharmaceutically acceptable salt thereof, and (ii) one or more additional therapeutic agents.

5. A method of treating or preventing cancer in a subject, the method comprising administering to the subject a combination comprising: (i) a compound of Formula (0), an isomer thereof, or a pharmaceutically acceptable salt thereof, and (ii) one or more additional therapeutic agents.

6. A combination comprising: (i) a compound of Formula (0), an isomer thereof, or a pharmaceutically acceptable salt thereof, and (ii) one or more additional therapeutic agents, for treating or preventing cancer in a subject.

7. Use of a combination comprising: (i) a compound of Formula (0), an isomer thereof, or a pharmaceutically acceptable salt thereof, and (ii) one or more additional therapeutic agents, in the manufacture of a medicament for treating or preventing cancer in a subject.

8. The method, compound, combination, or use of any one of the preceding claims, wherein the subject is a human.

9. The method, compound, combination, or use of any one of the preceding claims, wherein the cancer is a carcinoma, a lymphoma, a blastoma, a sarcoma, a leukemia, a brain cancer, a breast cancer, a blood cancer, a bone cancer, a lung cancer, a skin cancer, a liver cancer, an ovarian cancer, a bladder cancer, a renal cancer, a kidney cancer, a gastric cancer, a thyroid cancer, a pancreatic cancer, an esophageal cancer, a prostate cancer, a cervical cancer, a uterine cancer, a stomach cancer, a soft tissue cancer, a laryngeal cancer, a small intestine cancer, a testicular cancer, an anal cancer, a vulvar cancer, a joint cancer, an oral cancer, a pharynx cancer or a colorectal cancer.

10. The method, compound, combination, or use of any one of the preceding claims, wherein the cancer is adrenocortical carcinoma, bladder urothelial carcinoma, breast invasive carcinoma, cervical squamous cell carcinoma, endocervical adenocarcinoma, cholangiocarcinoma, colon adenocarcinoma, lymphoid neoplasm diffuse large B-cell lymphoma, esophageal carcinoma, glioblastoma multiforme, head and neck squamous cell carcinoma, kidney chromophobe, kidney renal clear cell carcinoma, kidney renal papillary cell carcinoma, acute myeloid leukemia, brain lower grade glioma, liver hepatocellular carcinoma, lung adenocarcinoma, lung squamous cell carcinoma, mesothelioma, ovarian serous cystadenocarcinoma, pancreatic adenocarcinoma, pheochromocytoma, paraganglioma, prostate adenocarcinoma, rectum adenocarcinoma, sarcoma, skin cutaneous melanoma, stomach adenocarcinoma, testicular germ cell tumors, thyroid carcinoma, thymoma, uterine carcinosarcoma, uveal melanoma. Other examples include breast cancer, lung cancer, lymphoma, melanoma, liver cancer, colorectal cancer, ovarian cancer, bladder cancer, renal cancer or gastric cancer. Further examples of cancer include neuroendocrine cancer, non-small cell lung cancer (NSCLC), small cell lung cancer, thyroid cancer, endometrial cancer, biliary cancer, esophageal cancer, anal cancer, salivary, cancer, vulvar cancer, cervical cancer, Acute lymphoblastic leukemia (ALL), Acute myeloid leukemia (AML), Adrenal gland tumors, Anal cancer, Bile duct cancer, Bladder cancer, Bone cancer, Bowel cancer, Brain tumors, Breast cancer, Cancer of unknown primary (CUP), Cancer spread to bone, Cancer spread to brain, Cancer spread to liver, Cancer spread to lung, Carcinoid, Cervical cancer, Children's cancers, Chronic lymphocytic leukemia (CLL), Chrome myeloid leukemia (CML), Colorectal cancer, Ear cancer, Endometrial cancer, Eye cancer, Follicular dendritic cell sarcoma, Gallbladder cancer, Gastric cancer, Gastro esophageal junction cancers, Germ cell tumors, Gestational trophoblastic disease (GIT)), Hairy cell leukemia, Head and neck cancer, Hodgkin lymphoma, Kaposi's sarcoma, Kidney cancer, Laryngeal cancer, Leukemia, Gastric linitis plastica, Liver cancer, Lung cancer, Lymphoma, Malignant schwannoma, Mediastinal germ cell tumors, Melanoma skin cancer, Men's cancer, Merkel cell skin cancer, Mesothelioma, Molar pregnancy, Mouth and oropharyngeal cancer, Myeloma, Nasal and paranasal sinus cancer, Nasopharyngeal cancer, Neuroblastoma, Neuroendocrine tumors, Non-Hodgkin lymphoma (NHL), Esophageal cancer, Ovarian cancer, Pancreatic cancer, Penile cancer, Persistent trophoblastic disease and choriocarcinoma, Pheochromocytoma, Prostate cancer, Pseudomyxoma peritonei, Rectal cancer. Retinoblastoma, Salivary gland cancer, Secondary' cancer, Signet cell cancer, Skin cancer, Small bowel cancer, Soft tissue sarcoma, Stomach cancer, T cell childhood non Hodgkin lymphoma (NHL), Testicular cancer, Thymus gland cancer, Thyroid cancer, Tongue cancer, Tonsil cancer, Tumors of the adrenal gland, Uterine cancer. Vaginal cancer, Vulval cancer, Wilms' tumor, Womb cancer and Gynaecological cancer. Examples of cancer also include, but are not limited to, Hematologic malignancies, Lymphoma, Cutaneous T-cell lymphoma, Peripheral T-cell lymphoma, Hodgkin's lymphoma, Non-Hodgkin's lymphoma, Multiple myeloma, Chrome lymphocytic leukemia, chronic myeloid leukemia, acute myeloid leukemia, Myelodysplastic syndromes, Myelofibrosis, Biliary tract cancer, Hepatocellular cancer, Colorectal cancer, Breast cancer, Lung cancer, Non-small cell lung cancer, Ovarian cancer, Thyroid Carcinoma, Renal Cell Carcinoma, Pancreatic cancer, Bladder cancer, skin cancer, malignant melanoma, merkel cell carcinoma, Uveal Melanoma or Glioblastoma multiforme.

11. The method, compound, combination, or use of any one of the preceding claims, wherein the cancer is a hematological cancer.

12. The method, compound, combination, or use of any one of the preceding claims, wherein the cancer is a solid cancer.

13. The method, compound, combination, or use of any one of the preceding claims, wherein the cancer is melanoma, breast cancer, head and neck cancer, esophagogastric cancer, stomach and small intestine cancer, lung cancer, mesothelioma, hepatobiliary cancer, pancreatic cancer, kidney cancer, colorectal cancer, endometrial cancer, cervical cancer, ovarian cancer, bladder cancer, prostate cancer, soft tissue sarcoma, CNS and brain cancer, or thyroid cancer.

14. The method, compound, combination, or use of any one of the preceding claims, wherein the cancer is non-small cell lung cancer (NSCLC), colorectal cancer, melanoma, thyroid cancer, histiocytosis, small bowel cancer, gastrointestinal neuroendocrine cancer, carcinoma of unknown primary, non-melanoma skin cancer, prostate cancer, gastric cancer, non-Hodgkin's lymphoma, papillary thyroid carcinoma or glioblastoma.

15. The method, compound, combination, or use of any one of the preceding claims, wherein the cancer is glioma.

16. The method, compound, combination, or use of any one of the preceding claims, wherein the cancer is glioblastoma.

17. The method, compound, combination, or use of any one of the preceding claims, wherein the cancer is lung cancer.

18. The method, compound, combination, or use of any one of the preceding claims, wherein the cancer is non-small cell lung cancer (NSCLC).

19. The method, compound, combination, or use of any one of the preceding claims, wherein the compound is administered to the subject by oral administration or parenteral administration.

20. The method, compound, combination, or use of any one of the preceding claims, wherein a pharmaceutical composition comprising the compound is administered to the subject.

21. The method, compound, combination, or use of any one of the preceding claims, wherein the compound is administered at a dosage (e.g., a daily dosage) of:
about 6±3 mg, about 6±2 mg, about 6±1 mg, about 6±0.9 mg, about 6±0.8 mg, about 6±0.7 mg, about 6±0.6 mg, about 6±0.5 mg, about 6±0.4 mg, about 6±0.3 mg, about 6±0.2 mg, or about 6±0.1 mg (e.g., about 6 mg);
about 12±6 mg, about 12±5 mg, about 12±4 mg, about 12±3 mg, about 12±2 mg, about 12±1 mg, about 12±0.9 mg, about 12±0.8 mg, about 12±0.7 mg, about 12±0.6 mg, about 12±0.5 mg, about 12±0.4 mg, about 12±0.3 mg, about 12±0.2 mg, or about 12±0.1 mg (e.g., about 12 mg);
about 25±10 mg, about 25±9 mg, about 25±8 mg, about 25±7 mg, about 25±6 mg, about 25±5 mg, about 25±4 mg, about 25±3 mg, about 25±2 mg, or about 25±1 mg (e.g., about 25 mg);
about 50±20 mg, about 50±10 mg, about 50±9 mg, about 50±8 mg, about 50±7 mg, about 50±6 mg, about 50±5 mg, about 50±4 mg, about 50±3 mg, about 50±2 mg, or about 50±1 mg (e.g., about 50 mg);
about 100±50 mg, about 100±40 mg, about 100±30 mg, about 100±20 mg, about 100±10 mg, about 100±9 mg, about 100±8 mg, about 100±7 mg, about 100±6 mg, about 100±5 mg, about 100±4 mg, about 100±3 mg, about 100±2 mg, or about 100±1 mg (e.g., about 100 mg);
about 200±100 mg, about 200±90 mg, about 200±80 mg, about 200±70 mg, about 200±60 mg, about 200±50 mg, about 200±40 mg, about 200±30 mg, about 200±20 mg, or about 200±10 mg (e.g., about 200 mg);
about 400±200 mg, about 400±100 mg, about 400±90 mg, about 400±80 mg, about 400±70 mg, about 400±60 mg, about 400±50 mg, about 400±40 mg, about 400±30 mg, about 400±20 mg, or about 400±10 mg (e.g., about 400 mg);
about 600±300 mg, about 600±200 mg, about 600±100 mg, about 600±90 mg, about 600±80 mg, about 600±70 mg, about 600±60 mg, about 600±50 mg, about 600±40 mg, about 600±30 mg, about 600±20 mg, or about 600±10 mg (e.g., about 600 mg);
about 800±400 mg, about 800±300 mg, about 800±200 mg, about 800±100 mg, about 800±90 mg, about 800±80 mg, about 800±70 mg, about 800±60 mg, about 800±50 mg, about 800±40 mg, about 800±30 mg, about 800±20 mg, or about 800±10 mg (e.g., about 800 mg);
about 1000±500 mg, about 1000±400 mg, about 1000±300 mg, about 1000±200 mg, about 1000±100 mg, about 1000±90 mg, about 1000±80 mg, about 1000±70 mg, about 1000±60 mg, about 1000±50 mg, about 1000±40 mg, about 1000±30 mg, about 1000±20 mg, or about 1000±10 mg (e.g., about 1000 mg); or
about 1200±600 mg, about 1200±500 mg, about 1200±400 mg, about 1200±300 mg, about 1200±200 mg, about 1200±100 mg, about 1200±90 mg, about 1200±80 mg, about 1200±70 mg, about 1200±60 mg, about 1200±50 mg, about 1200±40 mg, about 1200±30 mg, about 1200±20 mg, or about 1200±10 mg (e.g., about 1200 mg).

22. The method, compound, combination, or use of any one of the preceding claims, wherein the one or more additional therapeutic agents comprise one or more SHP2 (src homology-2 domain-containing protein tyrosine phosphatase-2) inhibitors, one or more SOS1 inhibitors, one or more KRAS (kirsten rat sarcoma virus) inhibitors, one or more ERK (extracellular signal-regulated kinase) inhibitors, one or more immune checkpoint inhibitors, one or more chemotherapies, one or more EGFR (epidermal growth factor receptor) inhibitors, one or more MET (mesenchymal-epithelial transition) inhibitors, one or more TEAD (transcriptional enhanced associate domain) inhibitors, one or more YAP (yes-associated protein) inhibitors, one or more PI3K (phosphoinositide 3-kinase) inhibitors, one or more mTOR (mammalian target of rapamycin) inhibitors, one or more metabolic inhibitors, or one or more MEK (mitogen-activated protein kinase kinase) inhibitors.

23. The method, compound, combination, or use of any one of the preceding claims, wherein the one or more additional therapeutic agents comprise one or more SHP2 inhibitors;
optionally, the one or more SHP2 inhibitors comprises JAB-3068, JAB-3312, TNO-155, RLY-1971, or RMC-4630.

24. The method, compound, combination, or use of any one of the preceding claims, wherein the one or more additional therapeutic agents comprise one or more SOS1 inhibitors;
optionally, the one or more SOS1 inhibitors comprises BI-1701963, BI-1703880, or MRTX0902.

25. The method, compound, combination, or use of any one of the preceding claims, wherein the one or more additional therapeutic agents comprise one or more KRAS inhibitors;
optionally, the one or more KRAS inhibitors comprises sotorasib, adagrasib, LY3537982, divarasib, JDQ443, BI-1823911, MRTX1133, RMC-9805, or RMC-6236.

26. The method, compound, combination, or use of any one of the preceding claims, wherein the one or more additional therapeutic agents comprise one or more ERK inhibitors;
optionally, the one or more ERK inhibitors comprises ulixertinib, MK-8353, LY3214996, ASTX029, ASN007, LTT462, or KO-947.

27. The method, compound, combination, or use of any one of the preceding claims, wherein
the one or more additional therapeutic agents comprise one or more immune checkpoint inhibitors;
optionally, the one or more immune checkpoint inhibitors comprises pembrolizumab, ipilimumab, nivolumab, or atezolizumab.

28. The method, compound, combination, or use of any one of the preceding claims, wherein
the one or more additional therapeutic agents comprise one or more chemotherapies;
optionally, the one or more chemotherapies comprises oxaliplatin or irinotecan.

29. The method, compound, combination, or use of any one of the preceding claims, wherein
the one or more additional therapeutic agents comprise one or more EGFR inhibitors;
optionally, the one or more EGFR inhibitors comprises erlotinib, osimertinib, neratinib, gefitinib, cetuximab, panitumumab, dacomitinib, lapatinib, necitumumab, mobocertinib, or vandetanib.

30. The method, compound, combination, or use of any one of the preceding claims, wherein the one or more additional therapeutic agents comprise one or more MET inhibitors.
optionally, the one or more MET inhibitors comprises crizotinib, capmatinib, tepotinib, savolitinib. Cabozantinib, glesatinib, foretinib, merestinib, tivantinib, SAR125844, onartuzumab, telisotuzumab, or JNJ-61186372.

31. The method, compound, combination, or use of any one of the preceding claims, wherein the one or more additional therapeutic agents comprise one or more TEAD inhibitors;
optionally, the one or more TEAD inhibitors comprises VT3989, IK-930, or IAG933.

32. The method, compound, combination, or use of any one of the preceding claims, wherein
the one or more additional therapeutic agents comprise one or more YAP inhibitors;
optionally, the one or more YAP inhibitors comprises verteporfin.

33. The method, compound, combination, or use of any one of the preceding claims, wherein
the one or more additional therapeutic agents comprise one or more PI3K inhibitors;
optionally, the one or more PI3K inhibitors comprises idelalisib, alpelisib, alpelisib, leniolisib, duvelisib, or copanlisib.

34. The method, compound, combination, or use of any one of the preceding claims, wherein
the one or more additional therapeutic agents comprise one or more mTOR inhibitors;
optionally, the one or more mTOR inhibitors comprises everolimus, sirolimus, temsirolimus, everolimus, sirolimus, sirolimus protein-bound, or everolimus.

35. The method, compound, combination, or use of any one of the preceding claims, wherein the one or more additional therapeutic agents comprise one or more metabolic inhibitors;
optionally, the one or more metabolic inhibitors comprises trifluridine, gemcitabine, fluorouracil, pentostatin, clofarabine, azacitidine, cytarabine, mercaptopurine, fludarabine, or capecitabine.

36. The method, compound, combination, or use of any one of the preceding claims, wherein
the one or more metabolic inhibitors comprises one or more MEK inhibitors.
optionally, the one or more MEK inhibitors comprises trametinib, cobimetinib, or binimetinib.
